SEQUENCE LISTING (94 sequences)

SEQ ID NO: 1 - RNA, 20 nt, synthetic construct
  source 1..20: mol_type = other RNA
ctttcttggaaagtatattc

SEQ ID NO: 2 - RNA, 20 nt, synthetic construct
  source 1..20: mol_type = other RNA
gctcagagctacagcaagtc

SEQ ID NO: 3 - RNA, 20 nt, synthetic construct
  source 1..20: mol_type = other RNA
gctgcagggaaagagttctt

SEQ ID NO: 4 - RNA, 20 nt, synthetic construct
  source 1..20: mol_type = other RNA
gatggagacagtgtgtatct

SEQ ID NO: 5 - RNA, 20 nt, synthetic construct
  source 1..20: mol_type = other RNA
gcagatggggcccaaatcct

SEQ ID NO: 6 - RNA, 20 nt, synthetic construct
  source 1..20: mol_type = other RNA
gccacagtgacagcaaattc

SEQ ID NO: 7 - RNA, 20 nt, synthetic construct
  source 1..20: mol_type = other RNA
gccacagactcgagtatcag

SEQ ID NO: 8 - RNA, 20 nt, synthetic construct
  source 1..20: mol_type = other RNA
ccaaacgtgacttggctcct

SEQ ID NO: 9 - RNA, 20 nt, synthetic construct
  source 1..20: mol_type = other RNA
tatggacacacatgggtgta

SEQ ID NO: 10 - RNA, 20 nt, synthetic construct
  source 1..20: mol_type = other RNA
actaaagacgtgtcttttgg

SEQ ID NO: 11 - RNA, 20 nt, synthetic construct
  source 1..20: mol_type = other RNA
gtcatctctaggtgacttcc

SEQ ID NO: 12 - DNA, 20 bp, synthetic construct
  source 1..20: mol_type = other DNA
  modified_base 1: mod_base = OTHER; note = 2'-O-methoxyethyl 5-methylcytidine with a 3'-phosphorothioate linkage
  modified_base 2: mod_base = OTHER; note = 2'-O-methoxyethyl 5-methyluridine
  modified_base 3: mod_base = OTHER; note = 2'-O-methoxyethyl 5-methyluridine
  modified_base 4: mod_base = OTHER; note = 2'-O-methoxyethyl 5-methyluridine
  modified_base 5: mod_base = OTHER; note = 2'-O-methoxyethyl 5-methylcytidine with a 3'-phosphorothioate linkage
  modified_base 6: mod_base = OTHER; note = thymidine with a 3'-phosphorothioate linkage
  modified_base 7: mod_base = OTHER; note = thymidine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = thymidine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethyl 5-methyluridine
  modified_base 17: mod_base = OTHER; note = 2'-O-methoxyethyladenosine
  modified_base 18: mod_base = OTHER; note = 2'-O-methoxyethyl 5-methyluridine with a 3'-phosphorothioate linkage
  modified_base 19: mod_base = OTHER; note = 2'-O-methoxyethyl 5-methyluridine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
ctttcttggaaagtatattc

SEQ ID NO: 13 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyl 5-methyluridine
  modified_base 4: mod_base = OTHER; note = 2'-O-methoxyethyl 5-methylcytidine
  modified_base 5: mod_base = OTHER; note = 2'-O-methoxyethyladenosine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = thymidine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxy 5-methylcytidine with a 3'-phosphorothioate linkage
  modified_base 13: mod_base = OTHER; note = 2'-deoxyadenosine with a 3'-phosphorothioate linkage
  modified_base 14: mod_base = OTHER; note = 2'-deoxyguanosine with a 3'-phosphorothioate linkage
  modified_base 15: mod_base = OTHER; note = 2'-deoxy 5-methylcytidine with a 3'-phosphorothioate linkage
  modified_base 16: mod_base = OTHER; note = 2'-O-methoxyethyladenosine
  modified_base 17: mod_base = OTHER; note = 2'-O-methoxyethyladenosine
  modified_base 18: mod_base = OTHER; note = 2'-O-methoxyethylguanosine with a 3'-phosphorothioate linkage
  modified_base 19: mod_base = OTHER; note = 2'-O-methoxyethyl 5-methyluridine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
gctcagagctacagcaagtc

SEQ ID NO: 14 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyl 5-methyluridine
  modified_base 4: mod_base = OTHER; note = 2'-O-methoxyethylguanosine
  modified_base 5: mod_base = OTHER; note = 2'-O-methoxyethyl 5-methylcytidine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxyadenosine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethyl 5-methyluridine
  modified_base 17: mod_base = OTHER; note = 2'-O-methoxyethyl 5-methyluridine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethyl 5-methyluridine with a 3'-phosphorothioate linkage
  modified_base 20: mod_base = OTHER; note = 2'-O-methoxyethyl 5-methyluridine
  misc_feature 1..5: note = RNA
  misc_feature 6..15: note = DNA
  misc_feature 16..20: note = RNA
gctgcagggaaagagttctt

SEQ ID NO: 15 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyladenosine
  modified_base 3: mod_base = OTHER; note = 2'-O-methoxyethyl 5-methyluridine
  modified_base 4: mod_base = OTHER; note = 2'-O-methoxyethylguanosine
  modified_base 5: mod_base = OTHER; note = 2'-O-methoxyethylguanosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyguanosine with a 3'-phosphorothioate linkage
  modified_base 12: mod_base = OTHER; note = thymine with a 3'-phosphorothioate linkage
  modified_base 13: mod_base = OTHER; note = 2'-deoxyguanosine with a 3'-phosphorothioate linkage
  modified_base 14: mod_base = OTHER; note = thymidine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethyl 5-methyluridine
  modified_base 17: mod_base = OTHER; note = 2'-O-methoxyethyladenosine
  modified_base 18: mod_base = OTHER; note = 2'-O-methoxyethyl 5-methyluridine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethyl 5-methyluridine
  misc_feature 1..5: note = RNA
  misc_feature 6..15: note = DNA
  misc_feature 16..20: note = RNA
gatggagacagtgtgtatct

SEQ ID NO: 16 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyladenosine
  modified_base 4: mod_base = OTHER; note = 2'-O-methoxyethylguanosine
  modified_base 5: mod_base = OTHER; note = 2'-O-methoxyethyladenosine with a 3'-phosphorothioate linkage
  modified_base 6: mod_base = OTHER; note = thymidine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxy 5-methylcytidine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = 2'-deoxyadenosine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethyladenosine
  modified_base 17: mod_base = OTHER; note = 2'-O-methoxyethyl 5-methyluridine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethyl 5-methyluridine
  misc_feature 1..5: note = RNA
  misc_feature 6..15: note = DNA
  misc_feature 16..20: note = RNA
gcagatggggcccaaatcct

SEQ ID NO: 17 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyl 5-methylcytidine
  modified_base 4: mod_base = OTHER; note = 2'-O-methoxyethyladenosine
  modified_base 5: mod_base = OTHER; note = 2'-O-methoxyethyl 5-methylcytidine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = thymidine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxy 5-methylcytidine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethyladenosine
  modified_base 17: mod_base = OTHER; note = 2'-O-methoxyethyladenosine
  modified_base 18: mod_base = OTHER; note = 2'-O-methoxyethyl 5-methyluridine with a 3'-phosphorothioate linkage
  modified_base 19: mod_base = OTHER; note = 2'-O-methoxyethyl 5-methyluridine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
gccacagtgacagcaaattc

SEQ ID NO: 18 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyl 5-methylcytidine
  modified_base 4: mod_base = OTHER; note = 2'-O-methoxyethyladenosine
  modified_base 5: mod_base = OTHER; note = 2'-O-methoxyethyl 5-methylcytidine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = thymidine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxyadenosine with a 3'-phosphorothioate linkage
  modified_base 14: mod_base = OTHER; note = 2'-deoxyguanosine with a 3'-phosphorothioate linkage
  modified_base 15: mod_base = OTHER; note = thymidine with a 3'-phosphorothioate linkage
  modified_base 16: mod_base = OTHER; note = 2'-O-methoxyethyladenosine
  modified_base 17: mod_base = OTHER; note = 2'-O-methoxyethyl 5-methyluridine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethyladenosine with a 3'-phosphorothioate linkage
  modified_base 20: mod_base = OTHER; note = 2'-O-methoxyethylguanosine
  misc_feature 1..5: note = RNA
  misc_feature 6..15: note = DNA
  misc_feature 16..20: note = RNA
gccacagactcgagtatcag

SEQ ID NO: 19 - DNA, 20 bp, synthetic construct
  source 1..20: mol_type = other DNA
  modified_base 1: mod_base = OTHER; note = 2'-O-methoxyethyl 5-methylcytidine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyladenosine
  modified_base 4: mod_base = OTHER; note = 2'-O-methoxyethyladenosine
  modified_base 5: mod_base = OTHER; note = 2'-O-methoxyethyladenosine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = 2'-deoxyguanosine with a 3'-phosphorothioate linkage
  modified_base 8: mod_base = OTHER; note = thymidine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = thymidine with a 3'-phosphorothioate linkage
  modified_base 13: mod_base = OTHER; note = thymidine with a 3'-phosphorothioate linkage
  modified_base 14: mod_base = OTHER; note = 2'-deoxyguanosine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethyl 5-methylcytidine
  modified_base 17: mod_base = OTHER; note = 2'-O-methoxyethyl 5-methyluridine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethyl 5-methyluridine
  misc_feature 1..5: note = RNA
  misc_feature 6..15: note = DNA
  misc_feature 16..20: note = RNA
ccaaacgtgacttggctcct

SEQ ID NO: 20 - DNA, 20 bp, synthetic construct
  source 1..20: mol_type = other DNA
  modified_base 1: mod_base = OTHER; note = 2'-O-methoxyethyl 5-methyluridine with a 3'-phosphorothioate linkage
  modified_base 2: mod_base = OTHER; note = 2'-O-methoxyethyladenosine
  modified_base 3: mod_base = OTHER; note = 2'-O-methoxyethyl 5-methyluridine
  modified_base 4: mod_base = OTHER; note = 2'-O-methoxyethylguanosine
  modified_base 5: mod_base = OTHER; note = 2'-O-methoxyethylguanosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxy 5-methylcytidine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxyadenosine with a 3'-phosphorothioate linkage
  modified_base 13: mod_base = OTHER; note = thymidine with a 3'-phosphorothioate linkage
  modified_base 14: mod_base = OTHER; note = 2'-deoxyguanosine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethylguanosine
  modified_base 17: mod_base = OTHER; note = 2'-O-methoxyethyl 5-methyluridine
  modified_base 18: mod_base = OTHER; note = 2'-O-methoxyethylguanosine with a 3'-phosphorothioate linkage
  modified_base 19: mod_base = OTHER; note = 2'-O-methoxyethyl 5-methyluridine with a 3'-phosphorothioate linkage
  modified_base 20: mod_base = OTHER; note = 2'-O-methoxyethyladenosine
  misc_feature 1..5: note = RNA
  misc_feature 6..15: note = DNA
  misc_feature 16..20: note = RNA
tatggacacacatgggtgta

SEQ ID NO: 21 - DNA, 20 bp, synthetic construct
  source 1..20: mol_type = other DNA
  modified_base 1: mod_base = OTHER; note = 2'-O-methoxyethyladenosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyl 5-methyluridine
  modified_base 4: mod_base = OTHER; note = 2'-O-methoxyethyladenosine
  modified_base 5: mod_base = OTHER; note = 2'-O-methoxyethyladenosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxyguanosine with a 3'-phosphorothioate linkage
  modified_base 11: mod_base = OTHER; note = thymidine with a 3'-phosphorothioate linkage
  modified_base 12: mod_base = OTHER; note = 2'-deoxyguanosine with a 3'-phosphorothioate linkage
  modified_base 13: mod_base = OTHER; note = thymidine with a 3'-phosphorothioate linkage
  modified_base 14: mod_base = OTHER; note = 2'-deoxy 5-methylcytidine with a 3'-phosphorothioate linkage
  modified_base 15: mod_base = OTHER; note = thymidine with a 3'-phosphorothioate linkage
  modified_base 16: mod_base = OTHER; note = 2'-O-methoxyethyl 5-methyluridine
  modified_base 17: mod_base = OTHER; note = 2'-O-methoxyethyl 5-methyluridine
  modified_base 18: mod_base = OTHER; note = 2'-O-methoxyethyl 5-methyluridine with a 3'-phosphorothioate linkage
  modified_base 19: mod_base = OTHER; note = 2'-O-methoxyethylguanosine with a 3'-phosphorothioate linkage
  modified_base 20: mod_base = OTHER; note = 2'-O-methoxyethylguanosine
  misc_feature 1..5: note = RNA
  misc_feature 6..15: note = DNA
  misc_feature 16..20: note = RNA
actaaagacgtgtcttttgg

SEQ ID NO: 22 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methyluridine
  modified_base 3: mod_base = OTHER; note = 2'-O-methoxyethyl 5-methylcytidine
  modified_base 4: mod_base = OTHER; note = 2'-O-methoxyethyladenosine
  modified_base 5: mod_base = OTHER; note = 2'-O-methoxyethyl 5-methyluridine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = thymidine with a 3'-phosphorothioate linkage
  modified_base 8: mod_base = OTHER; note = 2'-deoxy 5-methylcytidine with a 3'-phosphorothioate linkage
  modified_base 9: mod_base = OTHER; note = thymidine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxyguanosine with a 3'-phosphorothioate linkage
  modified_base 13: mod_base = OTHER; note = thymidine with a 3'-phosphorothioate linkage
  modified_base 14: mod_base = OTHER; note = 2'-deoxyguanosine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethyl 5-methylcytidine
  modified_base 17: mod_base = OTHER; note = 2'-O-methoxyethyl 5-methyluridine
  modified_base 18: mod_base = OTHER; note = 2'-O-methoxyethyl 5-methyluridine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
gtcatctctaggtgacttcc

SEQ ID NO: 23 - RNA, 133781 nt, Homo sapiens
  source 1..133781: mol_type = genomic RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatattttttttatatatataatatatattaaaatataatatatatatttatataatataatatataaatatattatatattatatattttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatattatatatatttttatatatataatatgtataatatataatatatataaaaacatatataatatatattatatattatatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaaattaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagaattttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgattttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccccccttccctccttccctctttccctccttccccccttccacccttccccccttccccccttccctccttccttccttccttcctgcctgccttccttccttccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtgcaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactgcttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaatatatatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttctgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctgcaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattctgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgggcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatatatatatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacgtgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatcccctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggcgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagtgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcctggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatgccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaaggatcatatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcatcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagagagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcctggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcattgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaagaaaggaaaaaaaaaactcatgcctgtaatcccagcactttggggaccggggtgggcagatcacgaggtcaggagatcaagactatcctggccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctacccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcagaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatcttcgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgctgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccgagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagaggccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtggtatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcagtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcgtttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagatgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggggtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgtggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaattggactggctaacatggtgaaaccccgtctctactaaaaatacaaaaattagctgggcatggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaataaaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaataaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcaacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagctgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccaccaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcctggctaattttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagctgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 24 - RNA, 6644 nt, Homo sapiens
  source 1..6644: mol_type = genomic RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 25 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 26 - RNA, 5552 nt, Homo sapiens
  source 1..5552: mol_type = genomic RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 27 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 28 - RNA, 5372 nt, Homo sapiens
  source 1..5372: mol_type = genomic RNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 29 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 30 - RNA, 20 nt, synthetic construct
  source 1..20: mol_type = other RNA
gcctttgcgagcgtgcacag

SEQ ID NO: 31 - RNA, 20 nt, synthetic construct
  source 1..20: mol_type = other RNA
tttcttgttgggcaactggg

SEQ ID NO: 32 - RNA, 20 nt, synthetic construct
  source 1..20: mol_type = other RNA
ctggtgtgggtgttattcct

SEQ ID NO: 33 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyl 5-methylcytidine
  modified_base 4: mod_base = OTHER; note = 2'-O-methoxyethyl 5-methyluridine
  modified_base 5: mod_base = OTHER; note = 2'-O-methoxyethyl 5-methyluridine with a 3'-phosphorothioate linkage
  modified_base 6: mod_base = OTHER; note = thymidine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxy 5-methylcytidine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxy 5-methylcytidine with a 3'-phosphorothioate linkage
  modified_base 13: mod_base = OTHER; note = 2'-deoxyguanosine with a 3'-phosphorothioate linkage
  modified_base 14: mod_base = OTHER; note = thymidine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethyl 5-methylcytidine
  modified_base 17: mod_base = OTHER; note = 2'-O-methoxyethyladenosine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethyladenosine with a 3'-phosphorothioate linkage
  modified_base 20: mod_base = OTHER; note = 2'-O-methoxyethylguanosine
  misc_feature 1..5: note = RNA
  misc_feature 6..15: note = DNA
  misc_feature 16..20: note = RNA
gcctttgcgagcgtgcacag

SEQ ID NO: 34 - DNA, 20 bp, synthetic construct
  source 1..20: mol_type = other DNA
  modified_base 1: mod_base = OTHER; note = 2'-O-methoxyethyl 5-methyluridine with a 3'-phosphorothioate linkage
  modified_base 2: mod_base = OTHER; note = 2'-O-methoxyethyl 5-methyluridine
  modified_base 3: mod_base = OTHER; note = 2'-O-methoxyethyl 5-methyluridine
  modified_base 4: mod_base = OTHER; note = 2'-O-methoxyethyl 5-methylcytidine
  modified_base 5: mod_base = OTHER; note = 2'-O-methoxyethyl 5-methyluridine with a 3'-phosphorothioate linkage
  modified_base 6: mod_base = OTHER; note = thymidine with a 3'-phosphorothioate linkage
  modified_base 7: mod_base = OTHER; note = 2'-deoxyguanosine with a 3'-phosphorothioate linkage
  modified_base 8: mod_base = OTHER; note = thymidine with a 3'-phosphorothioate linkage
  modified_base 9: mod_base = OTHER; note = thymidine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = 2'-deoxyadenosine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethyl 5-methylcytidine
  modified_base 17: mod_base = OTHER; note = 2'-O-methoxyethyl 5-methyluridine
  modified_base 18: mod_base = OTHER; note = 2'-O-methoxyethylguanosine with a 3'-phosphorothioate linkage
  modified_base 19: mod_base = OTHER; note = 2'-O-methoxyethylguanosine with a 3'-phosphorothioate linkage
  modified_base 20: mod_base = OTHER; note = 2'-O-methoxyethylguanosine
  misc_feature 1..5: note = RNA
  misc_feature 6..15: note = DNA
  misc_feature 16..20: note = RNA
tttcttgttgggcaactggg

SEQ ID NO: 35 - DNA, 20 bp, synthetic construct
  source 1..20: mol_type = other DNA
  modified_base 1: mod_base = OTHER; note = 2'-O-methoxyethyl 5-methylcytidine with a 3'-phosphorothioate linkage
  modified_base 2: mod_base = OTHER; note = 2'-O-methoxyethyl 5-methyluridine
  modified_base 3: mod_base = OTHER; note = 2'-O-methoxyethylguanosine
  modified_base 4: mod_base = OTHER; note = 2'-O-methoxyethylguanosine
  modified_base 5: mod_base = OTHER; note = 2'-O-methoxyethyl 5-methyluridine with a 3'-phosphorothioate linkage
  modified_base 6: mod_base = OTHER; note = 2'-deoxyguanosine with a 3'-phosphorothioate linkage
  modified_base 7: mod_base = OTHER; note = thymidine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyguanosine with a 3'-phosphorothioate linkage
  modified_base 11: mod_base = OTHER; note = thymidine with a 3'-phosphorothioate linkage
  modified_base 12: mod_base = OTHER; note = 2'-deoxyguanosine with a 3'-phosphorothioate linkage
  modified_base 13: mod_base = OTHER; note = thymidine with a 3'-phosphorothioate linkage
  modified_base 14: mod_base = OTHER; note = thymidine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethyl 5-methyluridine
  modified_base 17: mod_base = OTHER; note = 2'-O-methoxyethyl 5-methyluridine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethyl 5-methyluridine
  misc_feature 1..5: note = RNA
  misc_feature 6..15: note = DNA
  misc_feature 16..20: note = RNA
ctggtgtgggtgttattcct

SEQ ID NO: 36 - DNA, 20 bp, synthetic construct
  source 1..20: mol_type = other DNA
gtggatctagttctcatcca

SEQ ID NO: 37 - DNA, 20 bp, synthetic construct
  source 1..20: mol_type = other DNA
ggatagaatccagttaatta

SEQ ID NO: 38 - DNA, 20 bp, synthetic construct
  source 1..20: mol_type = other DNA
tgtcacatctagaaaccacc

SEQ ID NO: 39 - DNA, 20 bp, synthetic construct
  source 1..20: mol_type = other DNA
gcctggacagagggatgagc

SEQ ID NO: 40 - DNA, 20 bp, synthetic construct
  source 1..20: mol_type = other DNA
aagctccaggtgattgagac

SEQ ID NO: 41 - DNA, 20 bp, synthetic construct
  source 1..20: mol_type = other DNA
ttctgatacattgtcaaagt

SEQ ID NO: 42 - DNA, 20 bp, synthetic construct
  source 1..20: mol_type = other DNA
agcagcttgaaggagccggc

SEQ ID NO: 43 - DNA, 20 bp, synthetic construct
  source 1..20: mol_type = other DNA
tggctgatgataccatttgg

SEQ ID NO: 44 - DNA, 16 bp, synthetic construct
  source 1..16: mol_type = other DNA
ctttcttggaaagtat

SEQ ID NO: 45 - DNA, 16 bp, synthetic construct
  source 1..16: mol_type = other DNA
tttcttggaaagtata

SEQ ID NO: 46 - DNA, 16 bp, synthetic construct
  source 1..16: mol_type = other DNA
ttcttggaaagtatat

SEQ ID NO: 47 - DNA, 16 bp, synthetic construct
  source 1..16: mol_type = other DNA
tcttggaaagtatatt

SEQ ID NO: 48 - DNA, 16 bp, synthetic construct
  source 1..16: mol_type = other DNA
cttggaaagtatattc

SEQ ID NO: 49 - DNA, 16 bp, synthetic construct
  source 1..16: mol_type = other DNA
gtggatctagttctca

SEQ ID NO: 50 - DNA, 16 bp, synthetic construct
  source 1..16: mol_type = other DNA
tggatctagttctcat

SEQ ID NO: 51 - DNA, 16 bp, synthetic construct
  source 1..16: mol_type = other DNA
ggatctagttctcatc

SEQ ID NO: 52 - DNA, 16 bp, synthetic construct
  source 1..16: mol_type = other DNA
gatctagttctcatcc

SEQ ID NO: 53 - DNA, 16 bp, synthetic construct
  source 1..16: mol_type = other DNA
atctagttctcatcca

SEQ ID NO: 54 - DNA, 16 bp, synthetic construct
  source 1..16: mol_type = other DNA
ttctgatacattgtca

SEQ ID NO: 55 - DNA, 16 bp, synthetic construct
  source 1..16: mol_type = other DNA
tctgatacattgtcaa

SEQ ID NO: 56 - DNA, 16 bp, synthetic construct
  source 1..16: mol_type = other DNA
ctgatacattgtcaaa

SEQ ID NO: 57 - DNA, 16 bp, synthetic construct
  source 1..16: mol_type = other DNA
tgatacattgtcaaag

SEQ ID NO: 58 - DNA, 16 bp, synthetic construct
  source 1..16: mol_type = other DNA
gatacattgtcaaagt

SEQ ID NO: 59 - DNA, 16 bp, synthetic construct
  source 1..16: mol_type = other DNA
agcagcttgaaggagc

SEQ ID NO: 60 - DNA, 16 bp, synthetic construct
  source 1..16: mol_type = other DNA
gcagcttgaaggagcc

SEQ ID NO: 61 - DNA, 16 bp, synthetic construct
  source 1..16: mol_type = other DNA
cagcttgaaggagccg

SEQ ID NO: 62 - DNA, 16 bp, synthetic construct
  source 1..16: mol_type = other DNA
agcttgaaggagccgg

SEQ ID NO: 63 - DNA, 16 bp, synthetic construct
  source 1..16: mol_type = other DNA
gcttgaaggagccggc

SEQ ID NO: 64 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethylthymine
  modified_base 3: mod_base = OTHER; note = 2'-O-methoxyethylguanosine with a 3'-phosphorothioate linkage
  modified_base 4: mod_base = OTHER; note = 2'-O-methoxyethylguanosine with a 3'-phosphorothioate linkage
  modified_base 5: mod_base = OTHER; note = 2'-O-methoxyethyladenosine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxy 5-methylcytidine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = 2'-deoxythymine with a 3'-phosphorothioate linkage
  modified_base 15: mod_base = OTHER; note = 2'-deoxy 5-methylcytidine with a 3'-phosphorothioate linkage
  modified_base 16: mod_base = OTHER; note = 2'-O-methoxyethyladenosine with a 3'-phosphorothioate linkage
  modified_base 17: mod_base = OTHER; note = 2'-O-methoxyethylthymine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethyladenosine
  misc_feature 1..5: note = RNA
  misc_feature 6..15: note = DNA
  misc_feature 16..20: note = RNA
gtggatctagttctcatcca

SEQ ID NO: 65 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethylguanosine
  modified_base 3: mod_base = OTHER; note = 2'-O-methoxyethyladenosine with a 3'-phosphorothioate linkage
  modified_base 4: mod_base = OTHER; note = 2'-O-methoxyethylthymine with a 3'-phosphorothioate linkage
  modified_base 5: mod_base = OTHER; note = 2'-O-methoxyethyladenosine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxythymine with a 3'-phosphorothioate linkage
  modified_base 10: mod_base = OTHER; note = 2'-deoxy 5-methylcytidine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxythymine with a 3'-phosphorothioate linkage
  modified_base 15: mod_base = OTHER; note = 2'-deoxythymine with a 3'-phosphorothioate linkage
  modified_base 16: mod_base = OTHER; note = 2'-O-methoxyethyladenosine with a 3'-phosphorothioate linkage
  modified_base 17: mod_base = OTHER; note = 2'-O-methoxyethyladenosine
  modified_base 18: mod_base = OTHER; note = 2'-O-methoxyethylthymine with a 3'-phosphorothioate linkage
  modified_base 19: mod_base = OTHER; note = 2'-O-methoxyethylthymine with a 3'-phosphorothioate linkage
  modified_base 20: mod_base = OTHER; note = 2'-O-methoxyethyladenosine
  misc_feature 1..5: note = RNA
  misc_feature 6..15: note = DNA
  misc_feature 16..20: note = RNA
ggatagaatccagttaatta

SEQ ID NO: 66 - DNA, 20 bp, synthetic construct
  source 1..20: mol_type = other DNA
  modified_base 1: mod_base = OTHER; note = 2'-O-methoxyethylthymine with a 3'-phosphorothioate linkage
  modified_base 2: mod_base = OTHER; note = 2'-O-methoxyethylguanosine
  modified_base 3: mod_base = OTHER; note = 2'-O-methoxyethylthymine with a 3'-phosphorothioate linkage
  modified_base 4: mod_base = OTHER; note = 2'-O-methoxyethyl 5-methylcytidine with a 3'-phosphorothioate linkage
  modified_base 5: mod_base = OTHER; note = 2'-O-methoxyethyladenosine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxyadenosine with a 3'-phosphorothioate linkage
  modified_base 14: mod_base = OTHER; note = 2'-deoxyadenosine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethyl 5-methylcytidine with a 3'-phosphorothioate linkage
  modified_base 17: mod_base = OTHER; note = 2'-O-methoxyethyl 5-methylcytidine
  modified_base 18: mod_base = OTHER; note = 2'-O-methoxyethyladenosine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
tgtcacatctagaaaccacc

SEQ ID NO: 67 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethyl 5-methylcytidine with a 3'-phosphorothioate linkage
  modified_base 4: mod_base = OTHER; note = 2'-O-methoxyethylthymine with a 3'-phosphorothioate linkage
  modified_base 5: mod_base = OTHER; note = 2'-O-methoxyethylguanosine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxy 5-methylcytidine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxyguanosine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethylthymine with a 3'-phosphorothioate linkage
  modified_base 17: mod_base = OTHER; note = 2'-O-methoxyethylguanosine
  modified_base 18: mod_base = OTHER; note = 2'-O-methoxyethyladenosine with a 3'-phosphorothioate linkage
  modified_base 19: mod_base = OTHER; note = 2'-O-methoxyethylguanosine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
gcctggacagagggatgagc

SEQ ID NO: 68 - DNA, 20 bp, synthetic construct
  source 1..20: mol_type = other DNA
  modified_base 1: mod_base = OTHER; note = 2'-O-methoxyethyladenosine with a 3'-phosphorothioate linkage
  modified_base 2: mod_base = OTHER; note = 2'-O-methoxyethyladenosine
  modified_base 3: mod_base = OTHER; note = 2'-O-methoxyethylguanosine with a 3'-phosphorothioate linkage
  modified_base 4: mod_base = OTHER; note = 2'-O-methoxyethyl 5-methylcytidine with a 3'-phosphorothioate linkage
  modified_base 5: mod_base = OTHER; note = 2'-O-methoxyethylthymine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = 2'-deoxy 5-methylcytidine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxyadenosine with a 3'-phosphorothioate linkage
  modified_base 14: mod_base = OTHER; note = 2'-deoxythymine with a 3'-phosphorothioate linkage
  modified_base 15: mod_base = OTHER; note = 2'-deoxythymine with a 3'-phosphorothioate linkage
  modified_base 16: mod_base = OTHER; note = 2'-O-methoxyethylguanosine with a 3'-phosphorothioate linkage
  modified_base 17: mod_base = OTHER; note = 2'-O-methoxyethyladenosine
  modified_base 18: mod_base = OTHER; note = 2'-O-methoxyethylguanosine with a 3'-phosphorothioate linkage
  modified_base 19: mod_base = OTHER; note = 2'-O-methoxyethyladenosine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
aagctccaggtgattgagac

SEQ ID NO: 69 - DNA, 20 bp, synthetic construct
  source 1..20: mol_type = other DNA
  modified_base 1: mod_base = OTHER; note = 2'-O-methoxyethylthymine with a 3'-phosphorothioate linkage
  modified_base 2: mod_base = OTHER; note = 2'-O-methoxyethylthymine
  modified_base 3: mod_base = OTHER; note = 2'-O-methoxyethyl 5-methylcytidine with a 3'-phosphorothioate linkage
  modified_base 4: mod_base = OTHER; note = 2'-O-methoxyethylthymine with a 3'-phosphorothioate linkage
  modified_base 5: mod_base = OTHER; note = 2'-O-methoxyethylguanosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxythymine with a 3'-phosphorothioate linkage
  modified_base 15: mod_base = OTHER; note = 2'-deoxy 5-methylcytidine with a 3'-phosphorothioate linkage
  modified_base 16: mod_base = OTHER; note = 2'-O-methoxyethyladenosine with a 3'-phosphorothioate linkage
  modified_base 17: mod_base = OTHER; note = 2'-O-methoxyethyladenosine
  modified_base 18: mod_base = OTHER; note = 2'-O-methoxyethyladenosine with a 3'-phosphorothioate linkage
  modified_base 19: mod_base = OTHER; note = 2'-O-methoxyethylguanosine with a 3'-phosphorothioate linkage
  modified_base 20: mod_base = OTHER; note = 2'-O-methoxyethylthymine
  misc_feature 1..5: note = RNA
  misc_feature 6..15: note = DNA
  misc_feature 16..20: note = RNA
ttctgatacattgtcaaagt

SEQ ID NO: 70 - DNA, 20 bp, synthetic construct
  source 1..20: mol_type = other DNA
  modified_base 1: mod_base = OTHER; note = 2'-O-methoxyethyladenosine with a 3'-phosphorothioate linkage
  modified_base 2: mod_base = OTHER; note = 2'-O-methoxyethylguanosine
  modified_base 3: mod_base = OTHER; note = 2'-O-methoxyethyl 5-methylcytidine with a 3'-phosphorothioate linkage
  modified_base 4: mod_base = OTHER; note = 2'-O-methoxyethyladenosine with a 3'-phosphorothioate linkage
  modified_base 5: mod_base = OTHER; note = 2'-O-methoxyethylguanosine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxyadenosine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethyl 5-methylcytidine with a 3'-phosphorothioate linkage
  modified_base 17: mod_base = OTHER; note = 2'-O-methoxyethyl 5-methylcytidine
  modified_base 18: mod_base = OTHER; note = 2'-O-methoxyethylguanosine with a 3'-phosphorothioate linkage
  modified_base 19: mod_base = OTHER; note = 2'-O-methoxyethylguanosine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
agcagcttgaaggagccggc

SEQ ID NO: 71 - DNA, 20 bp, synthetic construct
  source 1..20: mol_type = other DNA
  modified_base 1: mod_base = OTHER; note = 2'-O-methoxyethylthymine with a 3'-phosphorothioate linkage
  modified_base 2: mod_base = OTHER; note = 2'-O-methoxyethylguanosine
  modified_base 3: mod_base = OTHER; note = 2'-O-methoxyethylguanosine with a 3'-phosphorothioate linkage
  modified_base 4: mod_base = OTHER; note = 2'-O-methoxyethyl 5-methylcytidine with a 3'-phosphorothioate linkage
  modified_base 5: mod_base = OTHER; note = 2'-O-methoxyethylthymine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = 2'-deoxy 5-methylcytidine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethylthymine with a 3'-phosphorothioate linkage
  modified_base 17: mod_base = OTHER; note = 2'-O-methoxyethylthymine
  modified_base 18: mod_base = OTHER; note = 2'-O-methoxyethylthymine with a 3'-phosphorothioate linkage
  modified_base 19: mod_base = OTHER; note = 2'-O-methoxyethylguanosine with a 3'-phosphorothioate linkage
  modified_base 20: mod_base = OTHER; note = 2'-O-methoxyethylguanosine
  misc_feature 1..5: note = RNA
  misc_feature 6..15: note = DNA
  misc_feature 16..20: note = RNA
tggctgatgataccatttgg

SEQ ID NO: 72 - DNA, 16 bp, synthetic construct
  source 1..16: mol_type = other DNA
  modified_base 1: mod_base = OTHER; note = Locked 5-methylcytidine with a 3'-phosphorothioate linkage
  modified_base 2: mod_base = OTHER; note = Locked thymine with a 3'-phosphorothioate linkage
  modified_base 3: mod_base = OTHER; note = Locked thymine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxy 5-methylcytidine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = Locked thymine with a 3'-phosphorothioate linkage
  modified_base 15: mod_base = OTHER; note = Locked adenosine with a 3'-phosphorothioate linkage
  modified_base 16: mod_base = OTHER; note = Locked thymine
ctttcttggaaagtat

SEQ ID NO: 73 - DNA, 16 bp, synthetic construct
  source 1..16: mol_type = other DNA
  modified_base 1: mod_base = OTHER; note = Locked thymine with a 3'-phosphorothioate linkage
  modified_base 2: mod_base = OTHER; note = Locked thymine with a 3'-phosphorothioate linkage
  modified_base 3: mod_base = OTHER; note = Locked thymine with a 3'-phosphorothioate linkage
  modified_base 4: mod_base = OTHER; note = 2'-deoxy 5-methylcytidine with a 3'-phosphorothioate linkage
  modified_base 5: mod_base = OTHER; note = 2'-deoxythymine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxythymine with a 3'-phosphorothioate linkage
  modified_base 14: mod_base = OTHER; note = Locked adenosine with a 3'-phosphorothioate linkage
  modified_base 15: mod_base = OTHER; note = Locked thymine with a 3'-phosphorothioate linkage
  modified_base 16: mod_base = OTHER; note = Locked adenosine
tttcttggaaagtata

SEQ ID NO: 74 - DNA, 16 bp, synthetic construct
  source 1..16: mol_type = other DNA
  modified_base 1: mod_base = OTHER; note = Locked thymine with a 3'-phosphorothioate linkage
  modified_base 2: mod_base = OTHER; note = Locked thymine with a 3'-phosphorothioate linkage
  modified_base 3: mod_base = OTHER; note = Locked 5-methylcytidine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxythymine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxyadenosine with a 3'-phosphorothioate linkage
  modified_base 14: mod_base = OTHER; note = Locked thymine with a 3'-phosphorothioate linkage
  modified_base 15: mod_base = OTHER; note = Locked adenosine with a 3'-phosphorothioate linkage
  modified_base 16: mod_base = OTHER; note = Locked thymine
ttcttggaaagtatat

SEQ ID NO: 75 - DNA, 16 bp, synthetic construct
  source 1..16: mol_type = other DNA
  modified_base 1: mod_base = OTHER; note = Locked thymine with a 3'-phosphorothioate linkage
  modified_base 2: mod_base = OTHER; note = Locked 5-methylcytidine with a 3'-phosphorothioate linkage
  modified_base 3: mod_base = OTHER; note = Locked thymine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxyguanosine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxythymine with a 3'-phosphorothioate linkage
  modified_base 14: mod_base = OTHER; note = Locked adenosine with a 3'-phosphorothioate linkage
  modified_base 15: mod_base = OTHER; note = Locked thymine with a 3'-phosphorothioate linkage
  modified_base 16: mod_base = OTHER; note = Locked thymine
tcttggaaagtatatt

SEQ ID NO: 76 - DNA, 16 bp, synthetic construct
  source 1..16: mol_type = other DNA
  modified_base 1: mod_base = OTHER; note = Locked 5-methylcytidine with a 3'-phosphorothioate linkage
  modified_base 2: mod_base = OTHER; note = Locked thymine with a 3'-phosphorothioate linkage
  modified_base 3: mod_base = OTHER; note = Locked thymine with a 3'-phosphorothioate linkage
  modified_base 4: mod_base = OTHER; note = 2'-deoxyguanosine with a 3'-phosphorothioate linkage
  modified_base 5: mod_base = OTHER; note = 2'-deoxyguanosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxyadenosine with a 3'-phosphorothioate linkage
  modified_base 14: mod_base = OTHER; note = Locked thymine with a 3'-phosphorothioate linkage
  modified_base 15: mod_base = OTHER; note = Locked thymine with a 3'-phosphorothioate linkage
  modified_base 16: mod_base = OTHER; note = Locked 5-methylcytidine
cttggaaagtatattc

SEQ ID NO: 77 - DNA, 16 bp, synthetic construct
  source 1..16: mol_type = other DNA
  modified_base 1: mod_base = OTHER; note = Locked guanosine with a 3'-phosphorothioate linkage
  modified_base 2: mod_base = OTHER; note = Locked thymine with a 3'-phosphorothioate linkage
  modified_base 3: mod_base = OTHER; note = Locked guanosine with a 3'-phosphorothioate linkage
  modified_base 4: mod_base = OTHER; note = 2'-deoxyguanosine with a 3'-phosphorothioate linkage
  modified_base 5: mod_base = OTHER; note = 2'-deoxyadenosine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxy 5-methylcytidine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = Locked thymine with a 3'-phosphorothioate linkage
  modified_base 15: mod_base = OTHER; note = Locked 5-methylcytidine with a 3'-phosphorothioate linkage
  modified_base 16: mod_base = OTHER; note = Locked adenosine
gtggatctagttctca

SEQ ID NO: 78 - DNA, 16 bp, synthetic construct
  source 1..16: mol_type = other DNA
  modified_base 1: mod_base = OTHER; note = Locked thymine with a 3'-phosphorothioate linkage
  modified_base 2: mod_base = OTHER; note = Locked guanosine with a 3'-phosphorothioate linkage
  modified_base 3: mod_base = OTHER; note = Locked guanosine with a 3'-phosphorothioate linkage
  modified_base 4: mod_base = OTHER; note = 2'-deoxyadenosine with a 3'-phosphorothioate linkage
  modified_base 5: mod_base = OTHER; note = 2'-deoxythymine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxy 5-methylcytidine with a 3'-phosphorothioate linkage
  modified_base 13: mod_base = OTHER; note = 2'-deoxythymine with a 3'-phosphorothioate linkage
  modified_base 14: mod_base = OTHER; note = Locked 5-methylcytidine with a 3'-phosphorothioate linkage
  modified_base 15: mod_base = OTHER; note = Locked adenosine with a 3'-phosphorothioate linkage
  modified_base 16: mod_base = OTHER; note = Locked thymine
tggatctagttctcat

SEQ ID NO: 79 - DNA, 16 bp, synthetic construct
  source 1..16: mol_type = other DNA
  modified_base 1: mod_base = OTHER; note = Locked guanosine with a 3'-phosphorothioate linkage
  modified_base 2: mod_base = OTHER; note = Locked guanosine with a 3'-phosphorothioate linkage
  modified_base 3: mod_base = OTHER; note = Locked adenosine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxy 5-methylcytidine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxythymine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = Locked adenosine with a 3'-phosphorothioate linkage
  modified_base 15: mod_base = OTHER; note = Locked thymine with a 3'-phosphorothioate linkage
  modified_base 16: mod_base = OTHER; note = Locked 5-methylcytidine
ggatctagttctcatc

SEQ ID NO: 80 - DNA, 16 bp, synthetic construct
  source 1..16: mol_type = other DNA
  modified_base 1: mod_base = OTHER; note = Locked guanosine with a 3'-phosphorothioate linkage
  modified_base 2: mod_base = OTHER; note = Locked adenosine with a 3'-phosphorothioate linkage
  modified_base 3: mod_base = OTHER; note = Locked thymine with a 3'-phosphorothioate linkage
  modified_base 4: mod_base = OTHER; note = 2'-deoxy 5-methylcytidine with a 3'-phosphorothioate linkage
  modified_base 5: mod_base = OTHER; note = 2'-deoxythymine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxythymine with a 3'-phosphorothioate linkage
  modified_base 10: mod_base = OTHER; note = 2'-deoxy 5-methylcytidine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxy 5-methylcytidine with a 3'-phosphorothioate linkage
  modified_base 13: mod_base = OTHER; note = 2'-deoxyadenosine with a 3'-phosphorothioate linkage
  modified_base 14: mod_base = OTHER; note = Locked thymine with a 3'-phosphorothioate linkage
  modified_base 15: mod_base = OTHER; note = Locked 5-methylcytidine with a 3'-phosphorothioate linkage
  modified_base 16: mod_base = OTHER; note = Locked 5-methylcytidine
gatctagttctcatcc

SEQ ID NO: 81 - DNA, 16 bp, synthetic construct
  source 1..16: mol_type = other DNA
  modified_base 1: mod_base = OTHER; note = Locked adenosine with a 3'-phosphorothioate linkage
  modified_base 2: mod_base = OTHER; note = Locked thymine with a 3'-phosphorothioate linkage
  modified_base 3: mod_base = OTHER; note = Locked 5-methylcytidine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxyadenosine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxythymine with a 3'-phosphorothioate linkage
  modified_base 14: mod_base = OTHER; note = Locked 5-methylcytidine with a 3'-phosphorothioate linkage
  modified_base 15: mod_base = OTHER; note = Locked 5-methylcytidine with a 3'-phosphorothioate linkage
  modified_base 16: mod_base = OTHER; note = Locked adenosine
atctagttctcatcca

SEQ ID NO: 82 - DNA, 16 bp, synthetic construct
  source 1..16: mol_type = other DNA
  modified_base 1: mod_base = OTHER; note = Locked thymine with a 3'-phosphorothioate linkage
  modified_base 2: mod_base = OTHER; note = Locked thymine with a 3'-phosphorothioate linkage
  modified_base 3: mod_base = OTHER; note = Locked 5-methylcytidine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxyguanosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxyguanosine with a 3'-phosphorothioate linkage
  modified_base 14: mod_base = OTHER; note = Locked thymine with a 3'-phosphorothioate linkage
  modified_base 15: mod_base = OTHER; note = Locked 5-methylcytidine with a 3'-phosphorothioate linkage
  modified_base 16: mod_base = OTHER; note = Locked adenosine
ttctgatacattgtca

SEQ ID NO: 83 - DNA, 16 bp, synthetic construct
  source 1..16: mol_type = other DNA
  modified_base 1: mod_base = OTHER; note = Locked thymine with a 3'-phosphorothioate linkage
  modified_base 2: mod_base = OTHER; note = Locked 5-methylcytidine with a 3'-phosphorothioate linkage
  modified_base 3: mod_base = OTHER; note = Locked thymine with a 3'-phosphorothioate linkage
  modified_base 4: mod_base = OTHER; note = 2'-deoxyguanosine with a 3'-phosphorothioate linkage
  modified_base 5: mod_base = OTHER; note = 2'-deoxyadenosine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxy 5-methylcytidine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxythymine with a 3'-phosphorothioate linkage
  modified_base 14: mod_base = OTHER; note = Locked 5-methylcytidine with a 3'-phosphorothioate linkage
  modified_base 15: mod_base = OTHER; note = Locked adenosine with a 3'-phosphorothioate linkage
  modified_base 16: mod_base = OTHER; note = Locked adenosine
tctgatacattgtcaa

SEQ ID NO: 84 - DNA, 16 bp, synthetic construct
  source 1..16: mol_type = other DNA
  modified_base 1: mod_base = OTHER; note = Locked 5-methylcytidine with a 3'-phosphorothioate linkage
  modified_base 2: mod_base = OTHER; note = Locked thymine with a 3'-phosphorothioate linkage
  modified_base 3: mod_base = OTHER; note = Locked guanosine with a 3'-phosphorothioate linkage
  modified_base 4: mod_base = OTHER; note = 2'-deoxyadenosine with a 3'-phosphorothioate linkage
  modified_base 5: mod_base = OTHER; note = 2'-deoxythymine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxy 5-methylcytidine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxythymine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = Locked adenosine with a 3'-phosphorothioate linkage
  modified_base 15: mod_base = OTHER; note = Locked adenosine with a 3'-phosphorothioate linkage
  modified_base 16: mod_base = OTHER; note = Locked adenosine
ctgatacattgtcaaa

SEQ ID NO: 85 - DNA, 16 bp, synthetic construct
  source 1..16: mol_type = other DNA
  modified_base 1: mod_base = OTHER; note = Locked thymine with a 3'-phosphorothioate linkage
  modified_base 2: mod_base = OTHER; note = Locked guanosine with a 3'-phosphorothioate linkage
  modified_base 3: mod_base = OTHER; note = Locked adenosine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxyadenosine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxythymine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxythymine with a 3'-phosphorothioate linkage
  modified_base 12: mod_base = OTHER; note = 2'-deoxy 5-methylcytidine with a 3'-phosphorothioate linkage
  modified_base 13: mod_base = OTHER; note = 2'-deoxyadenosine with a 3'-phosphorothioate linkage
  modified_base 14: mod_base = OTHER; note = Locked adenosine with a 3'-phosphorothioate linkage
  modified_base 15: mod_base = OTHER; note = Locked adenosine with a 3'-phosphorothioate linkage
  modified_base 16: mod_base = OTHER; note = Locked guanosine
tgatacattgtcaaag

SEQ ID NO: 86 - DNA, 16 bp, synthetic construct
  source 1..16: mol_type = other DNA
  modified_base 1: mod_base = OTHER; note = Locked guanosine with a 3'-phosphorothioate linkage
  modified_base 2: mod_base = OTHER; note = Locked adenosine with a 3'-phosphorothioate linkage
  modified_base 3: mod_base = OTHER; note = Locked thymine with a 3'-phosphorothioate linkage
  modified_base 4: mod_base = OTHER; note = 2'-deoxyadenosine with a 3'-phosphorothioate linkage
  modified_base 5: mod_base = OTHER; note = 2'-deoxy 5-methylcytidine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxythymine with a 3'-phosphorothioate linkage
  modified_base 11: mod_base = OTHER; note = 2'-deoxy 5-methylcytidine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyadenosine with a 3'-phosphorothioate linkage
  modified_base 14: mod_base = OTHER; note = Locked adenosine with a 3'-phosphorothioate linkage
  modified_base 15: mod_base = OTHER; note = Locked guanosine with a 3'-phosphorothioate linkage
  modified_base 16: mod_base = OTHER; note = Locked thymine
gatacattgtcaaagt

SEQ ID NO: 87 - DNA, 16 bp, synthetic construct
  source 1..16: mol_type = other DNA
  modified_base 1: mod_base = OTHER; note = Locked adenosine with a 3'-phosphorothioate linkage
  modified_base 2: mod_base = OTHER; note = Locked guanosine with a 3'-phosphorothioate linkage
  modified_base 3: mod_base = OTHER; note = Locked 5-methylcytidine with a 3'-phosphorothioate linkage
  modified_base 4: mod_base = OTHER; note = 2'-deoxyadenosine with a 3'-phosphorothioate linkage
  modified_base 5: mod_base = OTHER; note = 2'-deoxyguanosine with a 3'-phosphorothioate linkage
  modified_base 6: mod_base = OTHER; note = 2'-deoxy 5-methylcytidine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxythymine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxyguanosine with a 3'-phosphorothioate linkage
  modified_base 14: mod_base = OTHER; note = Locked adenosine with a 3'-phosphorothioate linkage
  modified_base 15: mod_base = OTHER; note = Locked guanosine with a 3'-phosphorothioate linkage
  modified_base 16: mod_base = OTHER; note = Locked 5-methylcytidine
agcagcttgaaggagc

SEQ ID NO: 88 - DNA, 16 bp, synthetic construct
  source 1..16: mol_type = other DNA
  modified_base 1: mod_base = OTHER; note = Locked guanosine with a 3'-phosphorothioate linkage
  modified_base 2: mod_base = OTHER; note = Locked 5-methylcytidine with a 3'-phosphorothioate linkage
  modified_base 3: mod_base = OTHER; note = Locked adenosine with a 3'-phosphorothioate linkage
  modified_base 4: mod_base = OTHER; note = 2'-deoxyguanosine with a 3'-phosphorothioate linkage
  modified_base 5: mod_base = OTHER; note = 2'-deoxy 5-methylcytidine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxyadenosine with a 3'-phosphorothioate linkage
  modified_base 14: mod_base = OTHER; note = Locked guanosine with a 3'-phosphorothioate linkage
  modified_base 15: mod_base = OTHER; note = Locked 5-methylcytidine with a 3'-phosphorothioate linkage
  modified_base 16: mod_base = OTHER; note = Locked 5-methylcytidine
gcagcttgaaggagcc

SEQ ID NO: 89 - DNA, 16 bp, synthetic construct
  source 1..16: mol_type = other DNA
  modified_base 1: mod_base = OTHER; note = Locked 5-methylcytidine with a 3'-phosphorothioate linkage
  modified_base 2: mod_base = OTHER; note = Locked adenosine with a 3'-phosphorothioate linkage
  modified_base 3: mod_base = OTHER; note = Locked guanosine with a 3'-phosphorothioate linkage
  modified_base 4: mod_base = OTHER; note = 2'-deoxy 5-methylcytidine with a 3'-phosphorothioate linkage
  modified_base 5: mod_base = OTHER; note = 2'-deoxythymine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxyadenosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = Locked 5-methylcytidine with a 3'-phosphorothioate linkage
  modified_base 15: mod_base = OTHER; note = Locked 5-methylcytidine with a 3'-phosphorothioate linkage
  modified_base 16: mod_base = OTHER; note = Locked guanosine
cagcttgaaggagccg

SEQ ID NO: 90 - DNA, 16 bp, synthetic construct
  source 1..16: mol_type = other DNA
  modified_base 1: mod_base = OTHER; note = Locked adenosine with a 3'-phosphorothioate linkage
  modified_base 2: mod_base = OTHER; note = Locked guanosine with a 3'-phosphorothioate linkage
  modified_base 3: mod_base = OTHER; note = Locked 5-methylcytidine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxythymine with a 3'-phosphorothioate linkage
  modified_base 6: mod_base = OTHER; note = 2'-deoxyguanosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyguanosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyguanosine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = Locked 5-methylcytidine with a 3'-phosphorothioate linkage
  modified_base 15: mod_base = OTHER; note = Locked guanosine with a 3'-phosphorothioate linkage
  modified_base 16: mod_base = OTHER; note = Locked guanosine
agcttgaaggagccgg

SEQ ID NO: 91 - DNA, 16 bp, synthetic construct
  source 1..16: mol_type = other DNA
  modified_base 1: mod_base = OTHER; note = Locked guanosine with a 3'-phosphorothioate linkage
  modified_base 2: mod_base = OTHER; note = Locked 5-methylcytidine with a 3'-phosphorothioate linkage
  modified_base 3: mod_base = OTHER; note = Locked thymine with a 3'-phosphorothioate linkage
  modified_base 4: mod_base = OTHER; note = 2'-deoxythymine with a 3'-phosphorothioate linkage
  modified_base 5: mod_base = OTHER; note = 2'-deoxyguanosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyadenosine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxy 5-methylcytidine with a 3'-phosphorothioate linkage
  modified_base 13: mod_base = OTHER; note = 2'-deoxy 5-methylcytidine with a 3'-phosphorothioate linkage
  modified_base 14: mod_base = OTHER; note = Locked guanosine with a 3'-phosphorothioate linkage
  modified_base 15: mod_base = OTHER; note = Locked guanosine with a 3'-phosphorothioate linkage
  modified_base 16: mod_base = OTHER; note = Locked 5-methylcytidine
gcttgaaggagccggc

SEQ ID NO: 92 - DNA, 20 bp, synthetic construct
  source 1..20: mol_type = other DNA
  modified_base 1: mod_base = OTHER; note = 2'-O-methoxyethyl 5-methylcytidine with a 3'-phosphorothioate linkage
  modified_base 2: mod_base = OTHER; note = 2'-O-methoxyethylthymine
  modified_base 3: mod_base = OTHER; note = 2'-O-methoxyethylthymine with a 3'-phosphorothioate linkage
  modified_base 4: mod_base = OTHER; note = 2'-O-methoxyethylthymine with a 3'-phosphorothioate linkage
  modified_base 5: mod_base = OTHER; note = 2'-O-methoxyethyl 5-methylcytidine with a 3'-phosphorothioate linkage
  modified_base 6: mod_base = OTHER; note = 2'-deoxythymine with a 3'-phosphorothioate linkage
  modified_base 7: mod_base = OTHER; note = 2'-deoxythymine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxythymine with a 3'-phosphorothioate linkage
  modified_base 15: mod_base = OTHER; note = 2'-deoxyadenosine with a 3'-phosphorothioate linkage
  modified_base 16: mod_base = OTHER; note = 2'-O-methoxyethylthymine with a 3'-phosphorothioate linkage
  modified_base 17: mod_base = OTHER; note = 2'-O-methoxyethyladenosine
  modified_base 18: mod_base = OTHER; note = 2'-O-methoxyethylthymine with a 3'-phosphorothioate linkage
  modified_base 19: mod_base = OTHER; note = 2'-O-methoxyethylthymine with a 3'-phosphorothioate linkage
  modified_base 20: mod_base = OTHER; note = 2'-O-methoxyethyl 5-methylcytidine
  misc_feature 1..5: note = RNA
  misc_feature 6..15: note = DNA
  misc_feature 16..20: note = RNA
ctttcttggaaagtatattc

SEQ ID NO: 93 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyl 5-methylcytidine
  modified_base 3: mod_base = OTHER; note = 2'-O-methoxyethylthymine with a 3'-phosphorothioate linkage
  modified_base 4: mod_base = OTHER; note = 2'-O-methoxyethylguanosine with a 3'-phosphorothioate linkage
  modified_base 5: mod_base = OTHER; note = 2'-O-methoxyethyl 5-methylcytidine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyguanosine with a 3'-phosphorothioate linkage
  modified_base 9: mod_base = OTHER; note = 2'-deoxyguanosine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyadenosine with a 3'-phosphorothioate linkage
  modified_base 12: mod_base = OTHER; note = 2'-deoxyadenosine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxyadenosine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethylthymine with a 3'-phosphorothioate linkage
  modified_base 17: mod_base = OTHER; note = 2'-O-methoxyethylthymine
  modified_base 18: mod_base = OTHER; note = 2'-O-methoxyethyl 5-methylcytidine with a 3'-phosphorothioate linkage
  modified_base 19: mod_base = OTHER; note = 2'-O-methoxyethylthymine with a 3'-phosphorothioate linkage
  modified_base 20: mod_base = OTHER; note = 2'-O-methoxyethylthymine
  misc_feature 1..5: note = RNA
  misc_feature 6..15: note = DNA
  misc_feature 16..20: note = RNA
gctgcagggaaagagttctt

SEQ ID NO: 94 - DNA, 20 bp, synthetic construct
  source 1..20: mol_type = other DNA
  modified_base 1: mod_base = OTHER; note = 2'-O-methoxyethylguanosine with a 3'-phosphorothioate linkage
  modified_base 2: mod_base = OTHER; note = 2'-O-methoxyethyladenosine
  modified_base 3: mod_base = OTHER; note = 2'-O-methoxyethylthymine with a 3'-phosphorothioate linkage
  modified_base 4: mod_base = OTHER; note = 2'-O-methoxyethylguanosine with a 3'-phosphorothioate linkage
  modified_base 5: mod_base = OTHER; note = 2'-O-methoxyethylguanosine with a 3'-phosphorothioate linkage
  modified_base 6: mod_base = OTHER; note = 2'-deoxyadenosine with a 3'-phosphorothioate linkage
  modified_base 7: mod_base = OTHER; note = 2'-deoxyguanosine with a 3'-phosphorothioate linkage
  modified_base 8: mod_base = OTHER; note = 2'-deoxyadenosine with a 3'-phosphorothioate linkage
  modified_base 9: mod_base = OTHER; note = 2'-deoxy 5-methylcytidine with a 3'-phosphorothioate linkage
  modified_base 10: mod_base = OTHER; note = 2'-deoxyadenosine with a 3'-phosphorothioate linkage
  modified_base 11: mod_base = OTHER; note = 2'-deoxyguanosine with a 3'-phosphorothioate linkage
  modified_base 12: mod_base = OTHER; note = 2'-deoxythymine with a 3'-phosphorothioate linkage
  modified_base 13: mod_base = OTHER; note = 2'-deoxyguanosine with a 3'-phosphorothioate linkage
  modified_base 14: mod_base = OTHER; note = 2'-deoxythymine with a 3'-phosphorothioate linkage
  modified_base 15: mod_base = OTHER; note = 2'-deoxyguanosine with a 3'-phosphorothioate linkage
  modified_base 16: mod_base = OTHER; note = 2'-O-methoxyethylthymine with a 3'-phosphorothioate linkage
  modified_base 17: mod_base = OTHER; note = 2'-O-methoxyethyladenosine
  modified_base 18: mod_base = OTHER; note = 2'-O-methoxyethylthymine with a 3'-phosphorothioate linkage
  modified_base 19: mod_base = OTHER; note = 2'-O-methoxyethyl 5-methylcytidine with a 3'-phosphorothioate linkage
  modified_base 20: mod_base = OTHER; note = 2'-O-methoxyethylthymine
  misc_feature 1..5: note = RNA
  misc_feature 6..15: note = DNA
  misc_feature 16..20: note = RNA
gatggagacagtgtgtatct

(19) United States
(12) Patent Application Publication
(10) Pub. No.: US 2025/0283084 A1
(43) Pub. Date: Sep. 11, 2025

(54) MAPT ANTISENSE OLIGONUCLEOTIDE

(71) Applicant: ELI LILLY AND COMPANY, Indianapolis, IN (US)

(72) Inventors: Jeremy Simon Louis DESAPHY, Indianapolis, IN (US); Roberto EL KHOURY SEJNAUI, Cambridge, MA (US); Maire Fiona JUNG, Lincoln, MA (US); Michael Payam MOAZAMI, Boston, MA (US); Bum Sok SEO, Quincy, MA (US); Jibo WANG, Carmel, IN (US)

(21) Appl. No.: 19/072,460

(22) Filed: Mar. 6, 2025

Related U.S. Application Data
(60) Provisional application No. 63/562,773, filed on Mar. 8, 2024.

(51) Int. Cl. C12N 15/113; A61P 3/00

(52) U.S. Cl. CPC C12N 15/113; A61P 3/00; C12N 2310/11; C12N 2310/315; C12N 2310/321; C12N 2310/3231; C12N 2310/3341

(57) ABSTRACT

Provided herein are MAPT antisense oligonucleotides and compositions comprising a MAPT antisense oligonucleotide. Also provided herein are methods of using the MAPT antisense oligonucleotides or compositions comprising a MAPT antisense oligonucleotide for reducing MAPT expression and/or treating tauopathy in a subject.

### SEQUENCE LISTING

The present application is being filed along with a Sequence Listing in ST.26 XML format. The Sequence Listing is provided as a file titled “31012_US” created Feb. 6, 2025, and is 726 kilobytes in size. The Sequence Listing information in the ST.26 XML format is incorporated herein by reference in its entirety.

### BACKGROUND

Microtubule associated protein Tau (MAPT) is encoded by the MAPT gene located on chromosome 17. Tau protein interacts with tubulin to stabilize the microtubules and promote tubulin assembly into microtubules. MAPT transcripts are differentially expressed throughout the body, predominantly in the central and peripheral nervous system.

The MAPT gene consists of 16 exons. Alternative mRNA splicing gives rise to multiple MAPT isoforms. At least six Tau isoforms exist in human brain, ranging from 352 to 441 amino acids long. Alternative splicing of exons 2 and/or 3 leads to inclusion of zero, one, or two copies of the N-terminal acidic domain, which are referred to as 0N, 1N, or 2N Tau, respectively. The Tau isoforms that include exon 10, which encodes an additional microtubule-binding domain, are referred to as “4R Tau”, as it has four microtubule-binding domains. The Tau isoforms without exon 10 are referred to as “3R Tau”, as it has three microtubule-binding domains.

Mutations in MAPT and hyperphosphorylation of Tau protein can cause aggregation and deposition of Tau in pathogenic neurofibrillary tangles, causing progressive neurodegenerative disorders such as Alzheimer's disease, frontotemporal dementia (FTD), progressive supranuclear palsy (PSP), and other tauopathies.

Antisense oligonucleotides (ASOs) are powerful and versatile agents being used in a number of applications including mRNA reduction, translation arrest, miRNA inhibition, splicing modulation, and polyadenylation site selection. MAPT antisense oligonucleotides have been described before, e.g., in US 2016/0145617.

There is currently no FDA-approved disease-modifying therapeutic agent specifically for reducing MAPT and treating tauopathies. Accordingly, there remains a need for therapeutic agents that can reduce or adjust MAPT expression for treating tauopathies, e.g., by utilizing antisense oligonucleotides.

### SUMMARY OF INVENTION

Provided herein are MAPT antisense oligonucleotides and compositions comprising a MAPT antisense oligonucleotide. Also provided herein are methods of using the MAPT antisense oligonucleotides or compositions comprising a MAPT antisense oligonucleotide for reducing MAPT expression and/or treating tauopathy in a subject.

In one aspect, provided herein are antisense oligonucleotides targeting MAPT. In some embodiments, provided herein are MAPT antisense oligonucleotides that comprises a nucleic acid sequence selected from any one of SEQ ID NOs: 1-11, 30-32, or 36-63, wherein optionally one or more nucleotides are independently modified nucleotides, and wherein optionally one or more internucleotide linkages are modified internucleotide linkages. In some embodiments, the antisense oligonucleotide is single stranded.

The MAPT antisense oligonucleotides described herein may include modifications. The modifications can be made to one or more nucleotides (e.g., to nucleobase, sugar, or phosphate) or to one or more internucleotide linkages.

In some embodiments, the antisense oligonucleotide provided herein comprises one or more modified nucleotides. In some embodiments, such modified nucleotides comprise a modified nucleobase, e.g., 5-methylcytosine. In some embodiments, each C in the antisense oligonucleotide is a 5-methylcytosine. In some embodiments, such modified nucleotides comprise a modified sugar, e.g., a modified ribose or deoxyribose. In some embodiments, the modified sugar is a 2′-O-methoxyethyl (2′-O-MOE) modified sugar, 2′-O-methyl modified sugar, or 2′-fluoro modified sugar. In some embodiments, the modified sugar is a 2′-O-MOE modified sugar. In some embodiments, the modified sugar is a locked nucleic acid (LNA).

In some embodiments, the antisense oligonucleotides provided herein are 12 to 30 nucleotides in length. In some embodiments, the MAPT antisense oligonucleotides are 15 to 25 nucleotides in length. In some embodiments, the MAPT antisense oligonucleotides are 16 to 20 nucleotides in length. In some embodiments, the MAPT antisense oligonucleotides are 18 to 20 nucleotides in length. In some embodiments, the MAPT antisense oligonucleotides are 20 nucleotides in length. In some embodiments, the MAPT antisense oligonucleotides are 16 nucleotides in length.

In some embodiments, the MAPT antisense oligonucleotide provided herein are gapmers, wherein the first to fifth nucleotides each comprise a 2′-O-MOE modified sugar, wherein the sixth to fifteenth nucleotides each comprise a 2′-deoxynucleoside, and wherein the sixteenth to twentieth nucleotides each comprise a 2′-O-MOE modified sugar.

In some embodiments, the MAPT antisense oligonucleotide provided herein are gapmers, wherein the first to third nucleotides each comprise LNA, wherein the fourth to thirteenth nucleotides each comprise a 2′-deoxynucleoside, and wherein the fourteenth to sixteenth nucleotides each comprise LNA.

In some embodiments, the antisense oligonucleotides provided herein comprise one or more modified internucleotide linkages. In some embodiments, the modified internucleotide linkage is a phosphorothioate linkage. In some embodiments, the antisense oligonucleotides provided herein comprise phosphodiester and phosphorothioate internucleotide linkage. In some embodiments, the internucleotide linkages of the MAPT antisense oligonucleotides are sooosssssssssssooss, or sossssssssssssssoss, from 5′ end to 3′ end, wherein each s is a phosphorothioate linkage and each o is a phosphodiester linkage. In some embodiments, the internucleotide linkages of the MAPT antisense oligonucleotides are all phosphorothioate linkages.

In some embodiments, the antisense oligonucleotides provided herein comprise a sequence selected from any one of SEQ ID NOs: 12-22, 33-35, or 64-94. In some embodiments, the antisense oligonucleotides provided herein consist of a sequence selected from any one of SEQ ID NOs: 12-22, 33-35, or 64-94.

In another aspect, provided herein are pharmaceutical compositions comprising a MAPT antisense oligonucleotide described herein and a pharmaceutically acceptable carrier. Also provided herein are pharmaceutical compositions comprising a means for reducing MAPT expression in a cell and a pharmaceutically acceptable carrier.

In another aspect, provided herein are methods of reducing MAPT expression in a patient in need thereof, and such method comprises administering to the patient an effective amount of a MAPT antisense oligonucleotide or a pharmaceutical composition described herein. The MAPT antisense oligonucleotide can be administered to the patient intrathecally, intravenously, subcutaneously, or via intracisternal magna injection.

In another aspect, provided herein are methods of treating a tauopathy in a patient in need thereof, and such method comprises administering to the patient an effective amount of the MAPT antisense oligonucleotide or a pharmaceutical composition described herein.

Also provided herein are methods of reducing MAPT expression in a cell (e.g., a neuron), and such methods can include contacting the cell with a MAPT antisense oligonucleotide described herein; and incubating the cell for a time sufficient for degradation of MAPT mRNA, thereby reducing MAPT expression in the cell.

In another aspect, provided herein are MAPT antisense oligonucleotides or pharmaceutical compositions comprising a MAPT antisense oligonucleotide for use in reducing MAPT expression. Also provided herein are MAPT antisense oligonucleotides or the pharmaceutical composition comprising a MAPT antisense oligonucleotide for use in a therapy. Also provided herein are MAPT antisense oligonucleotides or pharmaceutical compositions comprising a MAPT antisense oligonucleotide for use in the treatment of a tauopathy. Also provided herein are uses of MAPT antisense oligonucleotides in the manufacture of a medicament for the treatment of a tauopathy.

### DETAILED DESCRIPTION

Provided herein are MAPT antisense oligonucleotides and compositions comprising a MAPT antisense oligonucleotide. Also provided herein are methods of using the MAPT antisense oligonucleotides or compositions comprising a MAPT antisense oligonucleotide for reducing MAPT expression and/or treating tauopathy in a subject.

An antisense oligonucleotide usually comprises a nucleic acid sequence complementary to the sequence of a target nucleic acid, e.g., a target genomic sequence, pre-mRNA, or mRNA sequence. Hybridization occurs when hydrogen bonding (e.g., Watson-Crick, Hoogsteen or reversed Hoogsteen hydrogen bonding) forms between the complementary nucleobases of the antisense oligonucleotide and the target nucleic acid. Non-complementary nucleobases between an antisense oligonucleotide and a target nucleic acid may be tolerated provided that the antisense oligonucleotide remains able to specifically hybridize to a target nucleic acid.

Antisense oligonucleotides can be designed to modulate the expression of a target protein through either RNase H-dependent or RNase H-independent manners (see Watts J K, et al., J Pathol. 2012 January; 226(2): 365-379). When an antisense oligonucleotide comprising a contiguous stretch of DNA hybridizes with a target RNA, the DNA-RNA heteroduplex recruit RNase H, which cleaves the target RNA in the duplex and promotes subsequent degradation of the RNA fragments by cellular nucleases. An antisense oligonucleotide can also decrease target expression independent of RNAse H by sterically blocking pre-mRNA processing or translation of mRNA into protein or modulating splicing of pre-mRNA into mature mRNA.

In one aspect, provided herein are antisense oligonucleotides targeting MAPT. In some embodiments, exemplary nucleic acid sequences of MAPT antisense oligonucleotides are provided in Table 1. In some embodiments, provided herein are MAPT antisense oligonucleotides that comprises a nucleic acid sequence selected from any one of SEQ ID NOs: 1-11, 30-32, or 36-63, wherein optionally one or more nucleotides are independently modified nucleotides, and wherein optionally one or more internucleotide linkages are modified internucleotide linkages. In some embodiments, the antisense oligonucleotide is single stranded. In some embodiments, the antisense oligonucleotides provided herein consist of a sequence selected from any one of SEQ ID NOs: 1-11, 30-32, or 36-63.

TABLE 1
Nucleic Acid Sequences of MAPT Antisense Oligonucleotides (ASO)

| | | | | | Target region |
|---|---|---|---|---|---|
| | | | | | of human |
| | | | | | MAPT sequence |
| | | | | | NG_007398.2 |
| MAPT | | SEQ | | SEQ | (range |
| ASO | | ID | | ID | 5092-138872) |
| No. | Unmodified Sequence | NO | Modified Sequence | NO | (SEQ ID NO: 23) |
| 1 | CTTTCTTGGAAAGTATATTC | 1 | [eCm]seToeToeTo[eCm]sd | 12 | 72350-72369 |
| | | | TsdTsdGsdGsdAsdAsdAsd | | |
| | | | GsdTsdAseToeAoeTseTs | | |
| | | | [eCm] | | |
| 2 | GCTCAGAGCTACAGCAAGTC | 2 | eGs[eCm]oeTo[eCm]oeAs | 13 | 82367-82386 |
| | | | dGsdAsdGs[dCm]sdTsdAs | | |
| | | | [dCm]sdAsdGs[dCm]seAo | | |
| | | | eAoeGseTs[eCm] | | |
| 3 | GCTGCAGGGAAAGAGTTCTT | 3 | eGs[eCm]oeToeGo[eCm]s | 14 | 22592-22611 |
| | | | dAsdGsdGsdGsdAsdAsdA | | |
| | | | sdGsdAsdGseToeTo[eCm] | | |
| | | | seTseT | | |
| 4 | GATGGAGACAGTGTGTATCT | 4 | eGseAoeToeGoeGsdAsdG | 15 | 19124-19143 |
| | | | sdAs[dCm]sdAsdGsdTsdG | | |
| | | | sdTsdGseToeAoeTs[eCm] | | |
| | | | seT | | |
| 5 | GCAGATGGGGCCCAAATCCT | 5 | eGs[eCm]oeAoeGoeAsdTs | 16 | 74462-74481 |
| | | | dGsdGsdGsdGs[dCm]s | | |
| | | | [dCm]s[dCm]sdAsdAseAoeTo | | |
| | | | [eCm]s[eCm]seT | | |
| 6 | GCCACAGTGACAGCAAATTC | 6 | eGs[eCm]o[eCm]oeAo | 17 | 36467-36486 |
| | | | [eCm]sdAsdGsdTsdGsdAs | | |
| | | | [dCm]sdAsdGs[dCm]sdAseAo | | |
| | | | eAoeTseTs[eCm] | | |
| 7 | GCCACAGACTCGAGTATCAG | 7 | eGs[eCm]o[eCm]oeAo | 18 | 45989-46008 |
| | | | [eCm]sdAsdGsdAs[dCm]sdTs | | |
| | | | [dCm]sdGsdAsdGsdTseAo | | |
| | | | eTo[eCm]seAseG | | |
| 8 | CCAAACGTGACTTGGCTCCT | 8 | [eCm]s[eCm]oeAoeAoeAs | 19 | 105626-105645 |
| | | | [dCm]sdGsdTsdGsdAs | | |
| | | | [dCm]sdTsdTsdGsdGs[eCm]o | | |
| | | | eTo[eCm]s[eCm]seT | | |
| 9 | TATGGACACACATGGGTGTA | 9 | eTseAoeToeGoeGsdAs | 20 | 30079-30098 |
| | | | [dCm]sdAs[dCm]sdAs[dCm]s | | |
| | | | dAsdTsdGsdGseGoeToeG | | |
| | | | seTseA | | |
| 10 | ACTAAAGACGTGTCTTTTGG | 10 | eAs[eCm]oeToeAoeAsdAs | 21 | 13464-13483 |
| | | | dGsdAs[dCm]sdGsdTsdGs | | |
| | | | dTs[dCm]sdTseToeToeTse | | |
| | | | GseG | | |
| 11 | GTCATCTCTAGGTGACTTCC | 11 | eGseTo[eCm]oeAoeTs[dC | 22 | 79347-79366 |
| | | | m]sdTs[dCm]sdTsdAsdGs | | |
| | | | dGsdTsdGsdAs[eCm]oeTo | | |
| | | | eTs[eCm]s[eCm] | | |
| 12 | GCCTTTGCGAGCGTGCACAG | 30 | eGs[eCm]o[eCm]oeToeTs | 33 | 898-917 |
| | | | dTsdGs[dCm]sdGsdAsdGs | | |
| | | | [dCm]sdGsdTsdGs[eCm]o | | |
| | | | eAo[eCm]seAseG | | |
| 13 | TTTCTTGTTGGGCAACTGGG | 31 | eTseToeTo[eCm]oeTsdTsd | 34 | 80673-80692 |
| | | | GsdTsdTsdGsdGsdGs[dCm] | | |
| | | | sdAsdAs[eCm]oeToeGs | | |
| | | | eGseG | | |
| 14 | CTGGTGTGGGTGTTATTCCT | 32 | [eCm]seToeGoeGoeTsdGs | 35 | 88009-88028 |
| | | | dTsdGsdGsdGsdTsdGsdTs | | |
| | | | dTsdAseToeTo[eCm]s | | |
| | | | [eCm]seT | | |
| 15 | GTGGATCTAGTTCTCATCCA | 36 | eGseToeGseGseAsdTsdC | 64 | 75985-76004 |
| | | | msdTsdAsdGsdTsdTsdCm | | |
| | | | sdTsdCmseAseTo[eCm]s | | |
| | | | [eCm]seA | | |
| 16 | GGATAGAATCCAGTTAATTA | 37 | eGseGoeAseTseAsdGsdAs | 65 | 42171-42190 |
| | | | dAsdTsdCmsdCmsdAsdGs | | |
| | | | dTsdTseAseAoeTseTseA | | |
| 17 | TGTCACATCTAGAAACCACC | 38 | eTseGoeTs[eCm]seAsdCm | 66 | 79820-79839 |
| | | | sdAsdTsdCmsdTsdAsdGsd | | |
| | | | AsdAsdAs[eCm]s[eCm]oe | | |
| | | | As[eCm]s[eCm] | | |
| 18 | GCCTGGACAGAGGGATGAGC | 39 | eGs[eCm]o[eCm]seTseGsd | 67 | 13357-13376 |
| | | | GsdAsdCmsdAsdGsdAsd | | |
| | | | GsdGsdGsdAseTseGoeAse | | |
| | | | Gs[eCm] | | |
| 19 | AAGCTCCAGGTGATTGAGAC | 40 | eAseAoeGs[eCm]seTsdC | 68 | 124341-124360 |
| | | | msdCmsdAsdGsdGsdTsdG | | |
| | | | sdAsdTsdTseGseAoeGseA | | |
| | | | s[eCm] | | |
| 20 | TTCTGATACATTGTCAAAGT | 41 | eTseTo[eCm]seTseGsdAsd | 69 | 10260-10279 |
| | | | TsdAsdCmsdAsdTsdTsdG | | |
| | | | sdTsdCmseAseAoeAseGse | | |
| | | | T | | |
| 21 | AGCAGCTTGAAGGAGCCGGC | 42 | eAseGo[eCm]seAseGsdC | 70 | 132887-132906 |
| | | | msdTsdTsdGsdAsdAsdGs | | |
| | | | dGsdAsdGs[eCm]s[eCm]o | | |
| | | | eGseGs[eCm] | | |
| 22 | TGGCTGATGATACCATTTGG | 43 | eTseGoeGs[eCm]seTsdGs | 71 | 39953-39972 |
| | | | dAsdTsdGsdAsdTsdAsdC | | |
| | | | msdCmsdAseTseToeTseG | | |
| | | | seG | | |
| 23 | CTTTCTTGGAAAGTAT | 44 | [CmL]s[TL]s[TL]sdTsdC | 72 | 72354-72369 |
| | | | msdTsdTsdGsdGsdAsdAs | | |
| | | | dAsdGs[TL]s[AL]s[TL] | | |
| 24 | TTTCTTGGAAAGTATA | 45 | [TL]s[TL]s[TL]sdCmsdTs | 73 | 72353-72368 |
| | | | dTsdGsdGsdAsdAsdAsdG | | |
| | | | sdTs[AL]s[TL]s[AL] | | |
| 25 | TTCTTGGAAAGTATAT | 46 | [TL]s[TL]s[CmL]sdTsdTs | 74 | 72352-72367 |
| | | | dGsdGsdAsdAsdAsdGsdT | | |
| | | | sdAs[TL]s[AL]s[TL] | | |
| 26 | TCTTGGAAAGTATATT | 47 | [TL]s[CmL]s[TL]sdTsdGs | 75 | 72351-72366 |
| | | | dGsdAsdAsdAsdGsdTsdA | | |
| | | | sdTs[AL]s[TL]s[TL] | | |
| 27 | CTTGGAAAGTATATTC | 48 | [CmL]s[TL]s[TL]sdGsdGs | 76 | 72350-72365 |
| | | | dAsdAsdAsdGsdTsdAsdTs | | |
| | | | dAs[TL]s[TL]s[CmL] | | |
| 28 | GTGGATCTAGTTCTCA | 49 | [GL]s[TL]s[GL]sdGsdAsd | 77 | 75989-76004 |
| | | | TsdCmsdTsdAsdGsdTsdTs | | |
| | | | dCms[TL]s[CmL]s[AL] | | |
| 29 | TGGATCTAGTTCTCAT | 50 | [TL]s[GL]s[GL]sdAsdTsd | 78 | 75988-76003 |
| | | | CmsdTsdAsdGsdTsdTsdC | | |
| | | | msdTs[CmL]s[AL]s[TL] | | |
| 30 | GGATCTAGTTCTCATC | 51 | [GL]s[GL]s[AL]sdTsdCms | 79 | 75987-76002 |
| | | | dTsdAsdGsdTsdTsdCmsd | | |
| | | | TsdCms[AL]s[TL]s[CmL] | | |
| 31 | GATCTAGTTCTCATCC | 52 | [GL]s[AL]s[TL]sdCmsdTs | 80 | 75986-76001 |
| | | | dAsdGsdTsdTsdCmsdTsd | | |
| | | | CmsdAs[TL]s[CmL]s | | |
| | | | [CmL] | | |
| 32 | ATCTAGTTCTCATCCA | 53 | [AL]s[TL]s[CmL]sdTsdAs | 81 | 75985-76000 |
| | | | dGsdTsdTsdCmsdTsdCms | | |
| | | | dAsdTs[CmL]s[CmL]s[AL] | | |
| 33 | TTCTGATACATTGTCA | 54 | [TL]s[TL]s[CmL]sdTsdGs | 82 | 10264-10279 |
| | | | dAsdTsdAsdCmsdAsdTsd | | |
| | | | TsdGs[TL]s[CmL]s[AL] | | |
| 34 | TCTGATACATTGTCAA | 55 | [TL]s[CmL]s[TL]sdGsdAs | 83 | 10263-10278 |
| | | | dTsdAsdCmsdAsdTsdTsd | | |
| | | | GsdTs[CmL]s[AL]s[AL] | | |
| 35 | CTGATACATTGTCAAA | 56 | [CmL]s[TL]s[GL]sdAsdTs | 84 | 10262-10277 |
| | | | dAsdCmsdAsdTsdTsdGsd | | |
| | | | TsdCms[AL]s[AL]s[AL] | | |
| 36 | TGATACATTGTCAAAG | 57 | [TL]s[GL]s[AL]sdTsdAsd | 85 | 10261-10276 |
| | | | CmsdAsdTsdTsdGsdTsdC | | |
| | | | msdAs[AL]s[AL]s[GL] | | |
| 37 | GATACATTGTCAAAGT | 58 | [GL]s[AL]s[TL]sdAsdCms | 86 | 10260-10275 |
| | | | dAsdTsdTsdGsdTsdCmsd | | |
| | | | AsdAs[AL]s[GL]s[TL] | | |
| 38 | AGCAGCTTGAAGGAGC | 59 | [AL]s[GL]s[CmL]sdAsdG | 87 | 132891-132906 |
| | | | sdCmsdTsdTsdGsdAsdAsd | | |
| | | | GsdGs[AL]s[GL]s[CmL] | | |
| 39 | GCAGCTTGAAGGAGCC | 60 | [GL]s[CmL]s[AL]sdGsdC | 88 | 132890-132905 |
| | | | msdTsdTsdGsdAsdAsdGs | | |
| | | | dGsdAs[GL]s[CmL]s[CmL] | | |
| 40 | CAGCTTGAAGGAGCCG | 61 | [CmL]s[AL]s[GL]sdCmsd | 89 | 132889-132904 |
| | | | TsdTsdGsdAsdAsdGsdGsd | | |
| | | | AsdGs[CmL]s[CmL]s[GL] | | |
| 41 | AGCTTGAAGGAGCCGG | 62 | [AL]s[GL]s[CmL]sdTsdTs | 90 | 132888-132903 |
| | | | dGsdAsdAsdGsdGsdAsdG | | |
| | | | sdCms[CmL]s[GL]s[GL] | | |
| 42 | GCTTGAAGGAGCCGGC | 63 | [GL]s[CmL]s[TL]sdTsdGs | 91 | 132887-132902 |
| | | | dAsdAsdGsdGsdAsdGsdC | | |
| | | | msdCms[GL]s[GL]s[CmL] | | |
| 43 | CTTTCTTGGAAAGTATATTC | 1 | [eCm]seToeTseTs[eCm]sd | 92 | 72350-72369 |
| | | | TsdTsdGsdGsdAsdAsdAsd | | |
| | | | GsdTsdAseTseAoeTseTs | | |
| | | | [eCm] | | |
| 44 | GCTGCAGGGAAAGAGTTCTT | 3 | eGs[eCm]oeTseGs[eCm]sd | 93 | 22592-22611 |
| | | | AsdGsdGsdGsdAsdAsdAs | | |
| | | | dGsdAsdGseTseTo[eCm]s | | |
| | | | eTseT | | |
| 45 | GATGGAGACAGTGTGTATCT | 4 | eGseAoeTseGseGsdAsdGs | 94 | 19124-19143 |
| | | | dAs[dCm]sdAsdGsdTsdGs | | |
| | | | dTsdGseTseAoeTs[eCm]se | | |
| | | | T | | |

Abbreviations-“Cm” stands for 5-methylcytidine; “d” indicates 2'-deoxynucleotides; “e” indicates a 2′-O-MOE modified nucleotide; “L indicates a LNA modified nucleotide”; “s” indicates phosphorothioate linkage; “o” indicates phosphodiester linkage.

In some embodiments, the MAPT antisense oligonucleotide described herein can comprise a sequence that has 1, 2, or 3 differences from the sequences listed in Table 1. Such MAPT antisense oligonucleotides may still be capable of binding to the target nucleic acid, with 1, 2, or 3 mismatches, when a sufficient number of nucleobases of the antisense oligonucleotide form hydrogen bond with the corresponding nucleobases of the target nucleic acid.

In some embodiments, the antisense oligonucleotides provided herein are 12 to 30 nucleotides in length. In some embodiments, the MAPT antisense oligonucleotides are 15 to 25 nucleotides in length. In some embodiments, the MAPT antisense oligonucleotides are 16 to 20 nucleotides in length. In some embodiments, the MAPT antisense oligonucleotides are 18 to 20 nucleotides in length. In some embodiments, the MAPT antisense oligonucleotides are 20 nucleotides in length. In some embodiments, the MAPT antisense oligonucleotides are 16 nucleotides in length.

The MAPT antisense oligonucleotides described herein may include modifications. The modifications can be made to one or more nucleotides (e.g., to nucleobase, sugar, or phosphate) or to one or more internucleotide linkages, which are the bonds between two nucleotides. Modifications can improve stability, efficacy, and/or reduce immunogenicity of the antisense oligonucleotides. For example, antisense oligonucleotides can be modified to have increased resistance to nucleases, increased stability, increased half-life, enhanced binding affinity for nucleic acid target, enhanced cellular uptake, and/or increased inhibitory activity when compared to the unmodified oligonucleotides.

In some embodiments, the antisense oligonucleotide provided herein comprises one or more modified nucleotides. In some embodiments, such modified nucleotides comprise a modified nucleobase, e.g., 5-methylcytosine. In some embodiments, each C in the antisense oligonucleotide is a 5-methylcytosine. In some embodiments, such modified nucleotides comprise a modified sugar, e.g., a modified ribose or deoxyribose. In some embodiments, the modified sugar is a 2′-O-methoxyethyl (2′-O-MOE) modified sugar, 2′-O-methyl modified sugar, or 2′-fluoro modified sugar. In some embodiments, the modified sugar is a 2′-O-MOE modified sugar. Oligonucleotides containing 5-methylcytosine or 2′-O-MOE modifications have been shown to exhibit decreased immune stimulation in mice (Henry S. et al., J Pharmacol. Exp Tuer. 2000 February; 292(2):468-79). In some embodiments, the modified sugar is a locked nucleic acid (LNA).

In some embodiments, the antisense oligonucleotide provided herein are gapmers, which comprise a central gap segment consisting of a stretch of contiguous 2′-deoxyribonucleotides, positioned between two 5′ and 3′ wing segments consisting of modified nucleotides. The central gap segment of DNA can serve as the substrate for cellular endonuclease RNAse H, recruit it to the target RNA:DNA heteroduplex and cleave the target RNA in the RNA:DNA duplex. The 5′ and 3′ wing segments with modified nucleotides confer increased resistance to other nuclease degradation. The gapmers can be in “X-Y-Z” format, where “X” represents the 5′ wing region, “Y” represents the central gap region, and “Z” represents the 3′ wing region. In some embodiments, the 5′ and 3′ wing regions comprise 2′-O-MOE modified nucleotides, and the central gap region comprises 2′-deoxynucleotides. In some embodiments, the 5′ and 3′ wing regions comprise LNA modified nucleotides, and the central gap region comprises 2′-deoxynucleotides.

In some embodiments, the MAPT antisense oligonucleotide provided herein are gapmers, wherein the first to fifth nucleotides each comprise a 2′-O-MOE modified sugar, wherein the sixth to fifteenth nucleotides each comprise a 2′-deoxynucleotide, and wherein the sixteenth to twentieth nucleotides each comprise a 2′-O-MOE modified sugar.

In some embodiments, the MAPT antisense oligonucleotide provided herein are gapmers, wherein the first to third nucleotides each comprise LNA, wherein the fourth to thirteenth nucleotides each comprise a 2′-deoxynucleoside, and wherein the fourteenth to sixteenth nucleotides each comprise LNA.

In some embodiments, the antisense oligonucleotides provided herein comprise one or more modified internucleotide linkages. In some embodiments, the modified internucleotide linkage is a phosphorothioate linkage. In some embodiments, the antisense oligonucleotides provided herein comprise phosphodiester and phosphorothioate internucleotide linkage. In some embodiments, the internucleotide linkages of the MAPT antisense oligonucleotides are sooosssssssssssooss, or sossssssssssssssoss, from 5′ end to 3′ end, wherein each s is a phosphorothioate linkage and each o is a phosphodiester linkage. In some embodiments, the internucleotide linkages of the MAPT antisense oligonucleotides are all phosphorothioate linkages.

In some embodiments, the antisense oligonucleotides provided herein comprise a sequence selected from any one of SEQ ID NOs: 12-22, 33-35, or 64-94. In some embodiments, the antisense oligonucleotides provided herein consist of a sequence selected from any one of SEQ ID NOs: 12-22, 33-35, or 64-94.

Antisense oligonucleotide can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm.

The activity of antisense oligonucleotides can be tested in vitro and/or in vivo. For in vitro testing, the antisense oligonucleotides can be introduced into cultured cells by natural uptake, transfection or electroporation. Following a period of treatment, MAPT (tau) expression level in the ASO-treated cells can be determined and compared to MAPT (tau) expression level in the untreated control cells. MAPT expression level can be determined by any appropriate method, for example, by quantitation of MAPT mRNA level, by measuring the quantity of cDNA produced from reverse transcription of MAPT mRNA, or by determining the quantity of tau protein. These methods can be performed on a sample-by-sample basis or modified for high throughput analysis. For example, MAPT mRNA level can also be detected and quantitated by polymerase chain reaction (PCR), using a pair of primers that recognize MAPT transcript. General procedures for PCR are taught in MacPherson et al., PCR: A Practical Approach, (IRL Press at Oxford University Press (1991)). Tau protein level can be quantitated by Western blot analysis (immunoblotting), enzyme-linked immunosorbent assay (ELISA), immunohistochemistry, immunoassays, immunoprecipitation, immunofluorescent assays, immunocytochemistry, fluorescence-activated cell sorting (FACS), radioimmunoassays, immunoradiometric assays, high-performance liquid chromatography (HPLC), mass spectrometry, confocal microscopy, enzymatic assays, or surface plasmon resonance (SPR).

In another aspect, provided herein are pharmaceutical compositions comprising a MAPT antisense oligonucleotide described herein and a pharmaceutically acceptable carrier. Also provided herein are pharmaceutical compositions comprising a means for reducing MAPT expression in a cell and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

In a further aspect, provided herein are methods of reducing MAPT expression in a cell (e.g., a neuron), such methods can include contacting the cell with a MAPT antisense oligonucleotide described herein; and incubating the cell for a time sufficient for degradation of MAPT mRNA, thereby reducing MAPT expression in the cell.

In another aspect, provided herein are methods of reducing MAPT expression in a patient in need thereof, and such method comprises administering to the patient an effective amount of a MAPT antisense oligonucleotide or a pharmaceutical composition described herein. Aggregation of MAPT can be caused by overexpression of the MAPT protein or a mutation that affects the structure of the protein, resulting in an increased tendency of the MAPT protein to self-associate. Therefore, reducing MAPT expression level can be beneficial to the patient with tauopathy.

In another aspect, provided herein are methods of treating a tauopathy in a patient in need thereof, and such method comprises administering to the patient an effective amount of the MAPT antisense oligonucleotide or a pharmaceutical composition described herein. Exemplary tauopathy includes, but are not limited to, Alzheimer's disease (AD), frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's disease, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease (Parkinson-dementia complex of Guam), meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), globular glial tauopathies (GGT). In some embodiments, the tauopathy is Alzheimer's disease (AD), frontotemporal dementia (FTD), or progressive supranuclear palsy (PSP).

The MAPT antisense oligonucleotide can be administered to the patient intrathecally, intravenously, subcutaneously, or via intracisternal magna injection. In some embodiments, the MAPT antisense oligonucleotide is administered to the patient intrathecally.

Dosage regimens of antisense oligonucleotide may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are MAPT antisense oligonucleotides or pharmaceutical compositions comprising a MAPT antisense oligonucleotide for use in reducing MAPT expression. Also provided herein are MAPT antisense oligonucleotides or the pharmaceutical composition comprising a MAPT antisense oligonucleotide for use in a therapy. Also provided herein are MAPT antisense oligonucleotides or pharmaceutical compositions comprising a MAPT antisense oligonucleotide for use in the treatment of a tauopathy. Also provided herein are uses of MAPT antisense oligonucleotides in the manufacture of a medicament for the treatment of a tauopathy.

As used herein, the terms “a,” “an,” “the,” and similar terms used in the context of the present disclosure (especially in the context of the claims) are to be construed to cover both the singular and plural unless otherwise indicated herein or clearly contradicted by the context.

The term “antisense oligonucleotide” as used herein refers to an oligonucleotide comprising a nucleobase sequence that is complementary to a corresponding segment of a target nucleic acid, e.g., a target genomic sequence, pre-mRNA, or mRNA molecule.

As used herein, “complementary” or “complementarity” means a structural relationship between two nucleotides, nucleosides, or nucleobases (e.g., on two opposing nucleic acids or on opposing regions of a single nucleic acid strand e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the canonical Watson-Crick manner, which means adenine pairing with thymine or uracil, and guanine pairing with cytosine, or in any other manner that allows for the formation of stable duplexes. Likewise, two nucleic acids may have regions of multiple nucleotides that are complementary with each other to form regions of complementarity.

An “effective amount” refers to an amount necessary (for periods of time and for the means of administration) to achieve the desired therapeutic result. An effective amount of an antisense oligonucleotide may vary according to factors such as the disease state, age, sex, and weight of the individual, and the ability of the antisense oligonucleotide to elicit a desired response in the individual. An effective amount is also one in which any toxic or detrimental effects of the antisense oligonucleotide are outweighed by the therapeutically beneficial effects.

The term “knockdown” or “expression knockdown” refers to reduced mRNA or protein expression of a gene after treatment of a reagent, e.g., an antisense oligonucleotide.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group, which can serve as a monomeric unit of nucleic acid polymers such as deoxyribonucleic acid (DNA) and ribonucleic acid (RNA).

As used herein, “oligonucleotide” means a polymer of linked nucleotides, each of which can be modified or unmodified. An oligonucleotide is typically less than about 100 nucleotides in length.

The term “patient”, as used herein, refers to a human patient.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Clustal W2.0, Clustal X2.0, or Megalign (DNASTAR) software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

As used herein, the term “tauopathy” refers to a disease associated with abnormal tau protein expression, secretion, phosphorylation, cleavage, and/or aggregation.

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

As used herein, “MAPT” refers to a human MAPT pre-mRNA or mRNA transcript, encoding a microtubule associated protein Tau. The human MAPT gene is located at chromosomal location 17q2 1. 1, and the genomic sequence of human MAPT gene can be found in GenBank at NG_007398.2 (SEQ ID NO: 23). The nucleotide sequences of human MAPT transcript variants and amino acid sequences of human Tau protein isoforms can be found at:
i. MAPT transcript variant 1→Tau protein isoform 1: NM_016835.5 (nucleotide sequence)→NP_058519.3 (amino acid sequence);
ii. MAPT transcript variant 2→Tau protein isoform 2: NM_005910.6 (nucleotide sequence)→NP_005901.2 (amino acid sequence);
iii. MAPT transcript variant 3→Tau protein isoform 3: NM_016834.5 (nucleotide sequence)→NP_058518.1 (amino acid sequence);
iv. MAPT transcript variant 4→Tau protein isoform 4: NM_016841.5 (nucleotide sequence)→NP_058525.1 (amino acid sequence);
v. MAPT transcript variant 5→Tau protein isoform 5: NM_001123067.4 (nucleotide sequence)→NP_001116539.1 (amino acid sequence);
vi. MAPT transcript variant 6→Tau protein isoform 6: NM_001123066.4 (nucleotide sequence)→NP_001116538.2 (amino acid sequence);
vii. MAPT transcript variant 7→Tau protein isoform 7: NM_001203251.2 (nucleotide sequence)→NP_001190180.1 (amino acid sequence);
viii. MAPT transcript variant 8→Tau protein isoform 8: NM_001203252.2 (nucleotide sequence)→NP_001190181.1 (amino acid sequence);
ix. MAPT transcript variant 9→Tau protein isoform 9: NM_001377265.1 (nucleotide sequence)→NP_001364194.1 (amino acid sequence);
x. MAPT transcript variant 10→Tau protein isoform 10: NM_001377266.1 (nucleotide sequence)→NP_001364195.1 (amino acid sequence);
xi. MAPT transcript variant 11→Tau protein isoform 11: NM_001377267.1 (nucleotide sequence)→NP_001364196.1 (amino acid sequence);
xii. MAPT transcript variant 12→Tau protein isoform 4: NM_001377268.1 (nucleotide sequence)→NP_001364197.1 (amino acid sequence).

The nucleotide sequence of the human MAPT transcript variant 6 (encoding 2N4R Tau) can be found at NM_001123066.4 (SEQ ID NO: 24). The corresponding amino acid sequence of human Tau protein isoform 6 can be found at NP_001116538.2 (SEQ ID NO: 25).

The nucleotide sequence of a human MAPT transcript variant 5 (encoding 1N4R Tau) can be found at NM_001123067.4 (SEQ ID NO: 26). The corresponding amino acid sequence of human Tau protein isoform 5 can be found at NP_001116539.1 (SEQ ID NO: 27).

The nucleotide sequence of the human MAPT transcript variant 4 (encoding 0N3R Tau) can be found at NM_016841.5 (SEQ ID NO: 28). The corresponding amino acid sequence of human Tau protein isoform 4 can be found at NP_058525.1 (SEQ ID NO: 29).

### EXAMPLES

### Example 1. Synthesis of MAPT Antisense Oligonucleotides

The oligonucleotide sequences shown in Table 1 were synthesized on solid support via phosphoramidite chemistry using the Shasta Plate Synthesizer (Sierra Biosystems) for 100 nmol scale syntheses and the H8-SE Synthesizer (K&A) for 10 μmol scale syntheses.

Standard reagents were used in the oligonucleotide synthesis (Table 2), where 0.1M DDTT in pyridine was used as the sulfurization reagent. All phosphoramidites were commercially available (ChemGenes) and were prepared at 0.1M in Acetonitrile and contained a molecular sieves trap bag.

Oligonucleotides synthesized at the 0.1 nmol scale on 96-well plates were cleaved and deprotected (C/D) using ammonium hydroxide in the gas phase at 80° C. and at 40 psi. Wells were washed twice with 100 μL of anhydrous acetonitrile and then dried. Oligonucleotides were eluted in 300 μL of water, analyzed for concentration using absorbance at 260 nm recorded by the Lunatic (Unchained Labs), and characterized by LC-QTOF for mass purity of >85%.

Oligonucleotides synthesized at the 10 μmol scale were cleaved and deprotected (C/D) using a 1:1 solution of 25% aqueous ammonium hydroxide and 40% aqueous methylamine at room temperature for 2 hours. The solution containing cleaved oligonucleotide was decanted and concentrated in the Genevac for the ammonia and methylamine removal. The CPG was filtered via 0.45 μm PVDF filter.

The crude oligonucleotides were purified via AKTA™ Pure purification system using anion-exchange (AEX) with a HiScale 16 Capto Q ImpRes column (Cytiva Life Sciences) with MPA: 20 mM NaOH, 20% ACN, pH 12.4 and MPB: 20 mM NaOH, 1.5M NaBr, 20% ACN, pH 12.4.

The purified oligonucleotides were desalted in 15 mL 3K MWCO centrifugal spin tubes by rinsing with RNase free water at 3500×g for ˜30 min. The final desalted oligonucleotides were analyzed for concentration (nano drop at A260), characterized by LC-QTOF for mass purity and UPLC for UV-purity.

TABLE 2
Oligonucleotide Synthesis Reagents
Reagents

Activator Solution (0.45M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
DDTT (0.1M in Pyridine)

### Example 2. In Vitro Characterization of MAPT Antisense Oligonucleotides

Selected MAPT antisense oligonucleotides were tested in vitro for MAPT inhibition.

### Materials and Methods

SH-SY5Y Cell Culture and MAPT Antisense Oligonucleotide Treatment: SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with 10% fetal bovine serum, and cells were incubated at 37° C. in a humidified atmosphere of 5% CO₂. On Day One, antisense oligonucleotide and Lipofectamine RNAiMax transfection reagent (ThermoFisher 13778030) complexes were prepared inside the wells and SH-SY5Y cells were plated in 96 well tissue culture plates and incubated for 24 hrs. On Day Two, analysis of changes in gene expression in the treated SH-SY5Y cells was measured using TaqMan™ Gene Expression Cells-to-CT™ Kits following the manufacturer's protocol (ThermoFisher AM1729). Predesigned gene expression assays (supplied as 60× or 20× mixtures) were selected from TaqMan Real-Time PCR Assays. The efficiencies of these assays for MAPT (ThermoFisher Hs00902193_m1) and TBP (ThermoFisher #4326322E) were characterized with a dilution series of cDNA. RT-QPCR was performed in MicroAmp Optical 384-well reaction plates using QuantStudio 7 Pro system. The delta-delta CT method of normalizing to the transfection only control sample was used to determine relative amounts of MAPT gene expression. GraphPad Prism v10.2 was used to determine absolute or relative IC₅₀ with a four-parameter logistic fit.

Human induced pluripotent stem cells (hiPSCs) derived glutamatergic neurons (iCell GlutanNeurons) (CDI, 01279) was obtained from Fujifilm Cellular Dynamics International (CDI). iCell GlutanNeurons were handled and maintained following CDI's guidelines. Tissue culture treated 96 well plates (Thermo Scientific, 167574) were coated with poly-L-ornithine (PLO) and Matrigel (Corning 354230). 80,000 cells were plated in each well and were grown in Complete BrainPhys Medium consisting of BrainPhys Neuronal Medium (STEMCELL Technologies, 05790), iCell Neural Supplement B (CDI, M1029), iCell Nervous System Supplement (CDI, M1031), N-2 Supplement (Thermo Scientific, 17502-048) and Laminin (Sigma-Aldrich, L2020) in an incubator (37°/5% CO2). Starting the day after plating, half of media was replenished every other day for seven days. On the eighth day, antisense oligonucleotide was serially diluted in Complete BrainPhys Medium, and cells were treated with MAPT antisense oligonucleotide by aspirating half of media from each well (100 μL) and adding 100 μL of 2×MAPT antisense oligonucleotide concentration for a final of 1×MAPT antisense oligonucleotide according to dilutions. Cells were half-fed either every three or four days after treatment by removing half of media and adding back fresh Complete BrainPhys Medium with 2×MAPT antisense oligonucleotide concentration. Cell lysates were harvested after seven days of treatment (14 days post seeding) and RT-qPCR was performed using TaqMan Fast Advanced Cells-to CT Kit (Thermo Scientific, A35377) to determine mRNA knock down level. MAPT probe (ThermoFisher, Hs00902193_m1) was used as the gene of interest while TBP probe was used as the reference gene (ThermoFisher, 4326322E). RT-QPCR was performed in MicroAmp Optical 384-well reaction plates using the QuantStudio 7 Pro system. The delta-delta CT method was employed to normalize to the non-treated control sample and determine relative MAPT gene expression. GraphPad Prism v10.2 was used to calculate or relative IC₅₀ value with a four-parameter logistic fit.

Table 3 summarizes the in vitro activities of selected MAPT antisense oligonucleotides in SH-SY5Y cells. As shown in Table 3, the tested antisense oligonucleotides knock down MAPT expression in vitro in SH-SY5Y cells.

TABLE 3
In vitro activities of selected MAPT antisense
oligonucleotides in SH-SY5Y cells

| | SH-SY5Y 1 d | SH-SY5Y 1 d |
|---|---|---|
| | Absolute IC₅₀ | % KD (knockdown) of MAPT |
| MAPT ASO | (nM), | mRNA at 10 nM, |
| No | Transfection | Transfection |
| 1 | 0.41 | 93.9 ± 0.4 |
| 2 | 2.06 | 90.9 ± 0.6 |
| 3 | 0.73 | 90.3 ± 2.4 |
| 4 | 1.69 | 89.5 ± 2.5 |
| 5 | 0.37 | 89.1 ± 1.8 |
| 6 | 1.23 | 89.1 ± 1.7 |
| 7 | 0.74 | 87.5 ± 1.9 |
| 8 | 21.95 | 86.1 ± 3.9 |
| 9 | 1.94 | 84.8 ± 1.9 |
| 10 | 4.90 | 84.1 ± 2.1 |
| 11 | 0.70 | 84.1 ± 1.9 |
| 12 | 0.25 | 77.41 ± 2.1 |
| 13 | 1.55 | 82.4 ± 3.3 |
| 14 | 10.38 | 83.2 ± 0.5 |

Table 4 summarizes the in vitro activities of selected MAPT antisense oligonucleotides in human iPSC cells. As shown in Table 4, the tested antisense oligonucleotides knock down MAPT expression in human iPSC cells.

TABLE 4
In vitro activities of selected MAPT antisense
oligonucleotides in human iPSC cells

| | iPSC 7 d | iPSC 7 d |
|---|---|---|
| MAPT ASO | Relative IC₅₀ | % KD (knockdown) of MAPT |
| No | (μM) | mRNA at 10 nM |
| 15 | 2.916 | 70.11 |
| 16 | 10.4 | 44.69 |
| 17 | 3.562 | 54.88 |
| 18 | 10.66 | 26.92 |
| 19 | 9.743 | 33.38 |
| 20 | 1.782 | 78.32 |
| 21 | 2.782 | 64.02 |
| 22 | 7.396 | 43.72 |
| 23 | 1.08 | 80.31 |
| 24 | 0.6954 | 81.3 |
| 25 | 2.214 | 64.58 |
| 26 | 0.956 | 79.54 |
| 27 | 0.05902 | 98 |
| 28 | ND* | 94.83 |
| 29 | ND* | 85.99 |
| 30 | ND* | 96.75 |
| 31 | ND* | 61.96 |
| 32 | 0.057 | 97.35 |
| 33 | 0.018 | 99.37 |
| 34 | 0.016 | 98.41 |
| 35 | ND* | 97.04 |
| 36 | ND* | 90.86 |
| 37 | 0.012 | 98.39 |
| 38 | ND* | 68.99 |
| 39 | ND* | 56.67 |
| 40 | ND* | 58.94 |
| 41 | ND* | 34.01 |
| 42 | ND* | 67.05 |
| 43 | 6.04 | 53.89 |
| 44 | 6.64 | 57.38 |
| 45 | 2.19 | 70.57 |

ND* indicates not determined.

### Example 3. In Vivo Characterization of MAPT Antisense Oligonucleotides

Selected MAPT antisense oligonucleotides were tested in vivo for MAPT inhibition in human MAPT transgenic mice.

### Materials and Methods

Mouse Stereotaxic Intracerebroventricular (ICV) Surgery and MAPT Antisense Oligonucleotide Treatment: C57/BL6 transgenic mice expressing human MAPT were placed in an induction chamber (1-5% Isoflurane using a calibrated vaporizer) after administering 5 mg/kg of ketoprofen (subcutaneous). Hair is removed from the surgical site using electric clippers. The surgical area is disinfected with iodine and alcohol three times to create an aseptic surgical field. Intracerebroventricular (ICV) injection was performed, and the animal is returned to its cage. 6 mice per group received ICV injection of 150 μg of the MAPT ASO or PBS (phosphate buffered saline).

After a 14-day incubation, animals were euthanized. The tissues of interest were harvested and weighed. The tissues are then stored in RNAlater™ stabilization solution (Invitrogen™ catalog number AM7024). Once all the tissues were collected, RNA was extracted from the tissues using the protocol for the RNeasy Kit (Qiagen™ catalog number. 74182. Reverse transcription was then performed using the High-Capacity cDNA RT Master mix (Applied Biosystems™ catalog number. 4374967). Analysis of changes in hMAPT expression from the tissue homogenates was measured using Fast-Advanced TaqMan™ Master Mix (Applied Biosystems™ catalog number. 4444557) along with validated pre-designed TaqMan primers for human MAPT (Hs00902193_m1) and mouse GAPDH (Mm99999915_g1) as a housekeeping gene. The qPCR was performed in MicroAmp Optical 384-well reaction plates using QuantStudio 7 Pro system. The delta-delta CT method of normalizing against samples that were treated with PBS control was used to determine relative amounts of MAPT gene expression.

TABLE 5
summarizes the in-vivo activities of selected MAPT antisense
oligonucleotides in different tissues of transgenic
mice expressing the human MAPT gene. Antisense

| MAPT | Frontal | | | Brain | Spinal |
|---|---|---|---|---|---|
| ASO No | Cortex | Striatum | Cerebellum | Stem | Cord |
| PBS | 100.0 ± | 100.0 ± | 100.0 ± | 100.0 ± | 100.0 ± |
| | 7.825 | 8.807 | 5.194 | 4.056 | 3.726 |
| 1 | 40.53 ± | 55.08 ± | 102.3 ± | 57.91 ± | 79.44 ± |
| | 8.643 | 6.910 | 6.262 | 9.432 | 7.965 |
| 2 | 49.14 ± | 65.28 ± | 100.3 ± | 66.55 ± | 91.97 ± |
| | 4.246 | 5.187 | 3.412 | 4.031 | 1.882 |
| 3 | 58.81 ± | 66.55 ± | 91.99 ± | 67.61 ± | 89.58 ± |
| | 4.871 | 6.022 | 4.492 | 5.137 | 5.040 |
| 4 | 56.30 ± | 63.68 ± | 88.19 ± | 65.54 ± | 95.85 ± |
| | 8.123 | 7.089 | 4.695 | 4.362 | 9.168 |
| 5 | 50.69 ± | 46.86 ± | 99.39 ± | 48.22 ± | 98.84 ± |
| | 8.700 | 4.532 | 4.039 | 4.727 | 6.570 |
| 6 | 56.41 ± | 52.97 ± | 101.2 ± | 44.13 ± | 94.31 ± |
| | 5.546 | 3.938 | 4.742 | 3.688 | 2.996 |
| 7 | 95.84 ± | 95.15 ± | 110.8 ± | 137.1 ± | 96.31 ± |
| | 6.839 | 10.50 | 6.958 | 10.63 | 3.672 |
| 8 | 69.89 ± | 74.51 ± | 113.2 ± | 134.6 ± | 101.7 ± |
| | 6.987 | 10.57 | 4.399 | 11.97 | 4.444 |
| 9 | 69.67 ± | 78.68 ± | 124.8 ± | 97.14 ± | 96.24 ± |
| | 6.117 | 6.420 | 7.841 | 13.59 | 6.407 |
| 10 | 76.27 ± | 78.33 ± | 115.9 ± | 91.91 ± | 103.6 ± |
| | 9.847 | 4.310 | 2.247 | 8.329 | 6.060 |
| 11 | 62.31 ± | 61.20 ± | 97.20 ± | 23.91 ± | 71.36 ± |
| | 7.683 | 4.366 | 6.065 | 2.600 | 9.038 |

### Sequence Listing

| SEQ ID NO | Sequence |
|---|---|
| 1 | CTTTCTTGGAAAGTATATTC |
| 2 | GCTCAGAGCTACAGCAAGTC |
| 3 | GCTGCAGGGAAAGAGTTCTT |
| 4 | GATGGAGACAGTGTGTATCT |
| 5 | GCAGATGGGGCCCAAATCCT |
| 6 | GCCACAGTGACAGCAAATTC |
| 7 | GCCACAGACTCGAGTATCAG |
| 8 | CCAAACGTGACTTGGCTCCT |
| 9 | TATGGACACACATGGGTGTA |
| 10 | ACTAAAGACGTGTCTTTTGG |
| 11 | GTCATCTCTAGGTGACTTCC |
| 12 | [eCm]seToeToeTo[eCm]sdTsdTsdGsdGsdAsdAsdAsdGsdTsdAseToeAoeTseTs[eCm] |
| 13 | eGs[eCm]oeTo[eCm]oeAsdGsdAsdGs[dCm]sdTsdAs[dCm]sdAsdGs[dCm]seAoeAoeGseTs[eCm] |
| 14 | eGs[eCm]oeToeGo[eCm]sdAsdGsdGsdGsdAsdAsdAsdGsdAsdGseToeTo[eCm]seTseT |
| 15 | eGseAoeToeGoeGsdAsdGsdAs[dCm]sdAsdGsdTsdGsdTsdGseToeAoeTs[eCm]seT |
| 16 | eGs[eCm]oeAoeGoeAsdTsdGsdGsdGsdGs[dCm]s[dCm]s[dCm]sdAsdAseAoeTo[eCm]s[eCm]seT |
| 17 | eGs[eCm]o[eCm]oeAo[eCm]sdAsdGsdTsdGsdAs[dCm]sdAsdGs[dCm]sdAseAoeAoeTseTs[eCm] |
| 18 | eGs[eCm]o[eCm]oeAo[eCm]sdAsdGsdAs[dCm]sdTs[dCm]sdGsdAsdGsdTseAoeTo[eCm]seAseG |
| 19 | [eCm]s[eCm]oeAoeAoeAs[dCm]sdGsdTsdGsdAs[dCm]sdTsdTsdGsdGs[eCm]oeTo[eCm]s[eCm]seT |
| 20 | eTseAoeToeGoeGsdAs[dCm]sdAs[dCm]sdAs[dCm]sdAsdTsdGsdGseGoeToeGseTseA |
| 21 | eAs[eCm]oeToeAoeAsdAsdGsdAs[dCm]sdGsdTsdGsdTs[dCm]sdTseToeToeTseGseG |
| 22 | eGseTo[eCm]oeAoeTs[dCm]sdTs[dCm]sdTsdAsdGsdGsdTsdGsdAs[eCm]oeToeTs[eCm]s[eCm] |
| 23 | 1 GCAGTCACCG CCACCCACCA GCTCCGGCAC CAACAGCAGC GCCGCTGCCA CCGCCCACCT |
| | 61 TCTGCCGCCG CCACCACAGC CACCTTCTCC TCCTCCGCTG TCCTCTCCCG TCCTCGCCTC |
| | 121 TGTCGACTAT CAGGTAAGCG CCGCGGCTCC GAAATCTGCC TCGCCGTCCG CCTCTGTGCA |
| | 181 CCCCTGCGCC GCCGCCCCTC GCCCTCCCTC TCCGCAGACT GGGGCTTCGT GCGCCGGGCA |
| | 241 TCGGTCGGGG CCACCGCAGG GCCCCTCCCT GCCTCCCCTG CTCGGGGGCT GGGGCCAGGG |
| | 301 CGGCCTGGAA AGGGACCTGA GCAAGGGATG CACGCACGCG TGAGTGCGCG CGTGTGTGTG |
| | 361 TGCTGGAGGG TCTTCACCAC CAGATTCGCG CAGACCCCAG GTGGAGGCTG TGCCGGCAGG |
| | 421 GTGGGGCGCG GCGGCGGTGA CTTGGGGGAG GGGGCTGCCC TTCACTCTCG ACTGCAGCCT |
| | 481 TTTGCCGCAA TGGGCGTGTG TGTGTGTGTG TGTGTGTGTG TGTGTGTGTG TGTGTGTGGA |
| | 541 GGGGTCCGAT AACGACCCCC GAAACCGAAT CTGAAATCCG CTGTCCCTGC CGCTGTTCGC |
| | 601 CATCAGCTCT AAGAAAGACG TGGATCGGGT TCTAGAAAAG ATGACTCCCT GCACGCCCCT |
| | 661 CCCTGCACCT CCCGAGCAGT GATTCCGACA GGGCCTTCAC TGCCCCTGAT TTTAGGCGGG |
| | 721 GGCCGGCCCC CTCCCCTTTT CCTCCTTCAG AAACCCGTAG GGGACATTTG GGGGCTGGGA |
| | 781 GAAATCGAGG AGATGGGGAG GGGTCCACGC GCTGTCACTT TAGTTGCCCT TCCCCCTGCG |
| | 841 CACGCCTGGC ACAGAGACGC GAGCAGCGCC GTGCCTGAGA ACAGTGCGCG GATCCCACTG |
| | 901 TGCACGCTCG CAAAGGCAGG GTTCACCTGG CCTGGCGATG TGGACGGACT CGGCGGCCGC |
| | 961 TGGTCCCCGT TCGCGGGCAC GCACAGCCGC AGCCACGCAC GGATGGGCGC GGGGCTGCAG |
| | 1021 GTGCATCTCG GGGCGGATTT CTTTCTCAGC GCTCGGAGCG CAGGGCGCCC GGCGTGTGCG |
| | 1081 CTCCCTGCCG GAGGCGCGGG GCTGGCGCGC AGGGCTCGCC CCTCACTGCG GCAGTGGGTG |
| | 1141 TGGACCCTGG TGGGCGAGGA AGGGGGAGGA TAGGCTGTGC CTCCTCCCAC TCCCGCCCCC |
| | 1201 AGCCCCCCTT TTTTTCCCCC TCGGAACGCG AGGIGCCATC TTTTTTCGGC GTGTCACGTC |
| | 1261 TTTACGGTGC CATGCCAAAC CGGGTGGCCG GGCTTCATAG GACAGGGCGG GGCCTGGCAT |
| | 1321 TAAAGGGAGG GGGACAATCA GCGCTGAAAT CTTGGCGTTT TGCTGCTGCG GGCGTGAGCA |
| | 1381 CTGGGGGCGT TCGCCCAGCA CCTTCTTCGG GGGCTCTTTG CTTTGTCTGT AGAGGTTACG |
| | 1441 TGATCTGCGC TCCCAGCCCT GGTTTCTGGC TTTTATTCTG AGGGTGTTCA GTCAACCTCC |
| | 1501 CCCCTACGCC CATGCGCCTC TCTTTCCTTT TTCGCTCCTC ATTTCCGAGC CCATTGTTGG |
| | 1561 ATCTCGAGGC TTGCTGGGTT CGATGAACTC GAGTCAACCC CCCGACCCCC GGCACGCATG |
| | 1621 GAACGGGCGT GACCGCGCGC AGCCTCGTCT CGGAGTCTGC CGGCGCCGGG AAGCTTCTGA |
| | 1681 AGGGATGGGA TTCGAGTCTC CGTGCGCGCT GCGGGCGGCG GCAGAGGGAT CTCGCCCCTC |
| | 1741 CCTACACCCC AAGTGTCCTG AGGGCCACGC CACACCAGGT TGCCCAGCGA GGGACGCTGG |
| | 1801 CTACCCATCC GGGGATGGGT GGGGAGCCCT GGCGGGGCCT CTCCGGCTTT ACGCCCTGTT |
| | 1861 GCTTCGCCTG GCCGGAGAAT GTGAGGAAGG GGCATAAGGT TACTGGTGCT TCGGCCACAC |
| | 1921 CCATCTTTCT GAGCCCACTG GACTGGGCGC AGAGGGGGGA TTGCCATGGA AACCACAGGT |
| | 1981 GTCCGGAGAG GGGATCTTGG GGCTGGCCTC ACCCCTTCCC TGCGGAGATT GGGGACCCTG |
| | 2041 GGGTAGGGGG AGCCGCGCCC AGTCGGCCTC CTGGAGGACA CGGGAGGAAG CCCCGAACCC |
| | 2101 CCGCGCCTGA GGCTGTTTCT GATTGGCCCC TGGAGGCCGC AGACACGCAG ATAGGCGGCC |
| | 2161 CTGGGTGTAT TTTTATTAAT ATTATGTCCG TACTGATTAA TATTATTTAT CTTAAATAAA |
| | 2221 TTTCACCCGT GTCCAAGTTC ACCGCGCCCC CAAAACCGAG TCTGGGGCGG CAGGGGGAAC |
| | 2281 TCCTGGCCAA CGAATCCATG CCTCGCCCTC CTGTGATGAA CCTGGTACGC ACGGTTTTCT |
| | 2341 GGTTAATTCT ATCGCTGAAA ACTGGTGCGG GGGGCGCACT TCTGAGACGG AAGAGCATCT |
| | 2401 AGGAGCTGAA TCCTCCACGC GGGTCGCCCA GGTTGATCTG AATTTCTGGG GAATGGCTTG |
| | 2461 GCTGCCCGCC CGGGACCAGG CCGACCCTCC TTGACGGTGG CGTAGAGGGC TGGAGCCTGG |
| | 2521 GTACTGCGAG GCTCCTCGCA TGGCTGGGCC CGCCGCGAGG GGTTGCAGAG CGGCTCAGGG |
| | 2581 ATCGATTCAA GCATCGTCTC TCCTCCCTCG CCCCCAGACA GAGCTGGGCG CGGGGTTCCC |
| | 2641 CTTCCAGATG GAGCGAGGGT CTCGGGGTGG CCCCGGAAAA GGGGAGCCCG CGGCCACGGC |
| | 2701 TACGTATTGC CATCTCGCGA GCAGAGATGT CACCTCCTGC CTTTGGAGGA AAGGGAGCCC |
| | 2761 GGTGGGGATG AGCGCATTTA GCCCAATGCT GGGAACAAAG CGCACTCCGC GCTTCTGCGA |
| | 2821 TTTCGCTCCA TTTTGAAATG TGTTGGCGCT TTGGTGGGGC CGCTGCGGTG GGCAAGGCCG |
| | 2881 GGGGCGCTGT TAATGGAGGA ACCTCAGGGG GACGGTCCTT CGTAGGAAAC TCTATCCTGG |
| | 2941 CTCTGCGCGC GCTTTAAGGA AATGGCTTCC CTCCAGGACC TCGAGGGATG CAGCTTTTGC |
| | 3001 GCGGATGACG GTGGGGTGCT GAACCAGCCG GTGCGCCTCT GGAAATGTCT GGGCACGGAT |
| | 3061 CCTGGGGCCA TCGACGACTC CTCCCCATTC CCAGCAGGCG GGAGCTCTTA CATTCCGAGC |
| | 3121 GAGTGACCCC TCTCACCCTC TGGCGCTCAC ACACCTGTAA CTCCAAACCT CCGTCTCAGA |
| | 3181 ATGGTCCAGG CTGGAAGGGA TGATGGGGGC TCCGACAGCG ACTGCCTAGC TCACCCCTCT |
| | 3241 GCGTGCTCAG GCTCCAGGCT CAGCAGGACC AATTTGAGTT CTATCTGATC CCCCTCGGCC |
| | 3301 CCTTAACTGA CCCATCCTAC AGGAGACAGG GAAATGTCTT TCCTACCGCG GTTGATTCTG |
| | 3361 GGGTGTCATT TTGTGTTTTG TGATGGCTGC TTATATTTAC TGTATAAGCA TTGTATTTAC |
| | 3421 TGTATAAGCA TTGTATTATA ATTACTGTAT AAGCTGCTTA TATTTACTGT ATAAGCATCT |
| | 3481 CCAAATCCTC CCTCTACGTA AACAAATTAA TGGATAAACA GATAAGTGTA TCCCCTGCCC |
| | 3541 CCACCCCTGC TACGCAGGTC CGGAGTGACT CTTGAAGCTC ATACATTCCT TGGCCAAGTT |
| | 3601 TGCTTCTCTA ACAGATGTTT ATATAGCAAT AACCTGGCTT GGCTCTTGGG TTCACCTTTG |
| | 3661 GACGATTTGG GGAAGGGGCT TGTTGGCTTT GCTGGGTTTT GGATGAGTGA CAGTCCATGA |
| | 3721 CTGTTCCTGC TGGAAGGGCG TGACTTTTAA GTGGTTTCTA ATATCAGGCA TTGCTCCTCC |
| | 3781 GACAGGAACA AAAGAAATGG ATACTGCCCA TAAATTGTTA GAAAACTTAG AATCGCTTTG |
| | 3841 ATTGAGGAAA GGTTAGATTT ATTCCGGTTG GAAAAAGTGG CCTTTCTATT AAACGTGCCC |
| | 3901 TTTGACCCTC ATGCCCTTGG AGGTCGGTGC CAGCCTGGAG ATGGGATAAG ATTGTGGTTT |
| | 3961 TCCTTCTGCC TTTTTAACAT CTGTTGTTAC AGTCCATTTG TTGAAAATTT AAAGAAACTG |
| | 4021 TTTTATTCCA CTTTCCCTCA GCATTTATGT GTGTGGTTTC AGTAGCTCTG TGGCTATATG |
| | 4081 TACGAACACG TGTTATTTTT CCAATTGGAC ATGTGATAAT TTTCCAACTG GACCTTGCCT |
| | 4141 TCTATTGATG TATTTATTTA GCATCTTCCT TACTCCCTCC TTGAAAAAGA ATCACTCAAA |
| | 4201 AACAAATAAA AACAGCCGTA GGGGCCTAAT ACAGTGCTAG ACATACAAGA GGTATTCGGT |
| | 4261 CCATACCAAA TGGATTTTAT CCATGAAGGA TAAATGGGGA AATACAGTGG GAAGCAGGTG |
| | 4321 GGAAACTGCG TTTGACTCTG CTCTTTCCTC CACCACCACT TTCCTCATCA CCGTGTTCAG |
| | 4381 AGACCCCCAA AGCCCCCTCA CACTCCCAGA AACACCCCCC TGGCCACTCC TAACTTGCCA |
| | 4441 TGCCCAGGAG TTAGGTGCTT CCACTAGTGA CATGGAGCTG GCGTTTGGGG GGCACCTCAG |
| | 4501 CAGGTGACGG GAAGAGAAGA CCCCAGCCTC ACCAGCTGGG CTGCAGCAGG GAGAGGAGTC |
| | 4561 CTCATGTTCC AGCAGGGACT CTCAGCTGTT TTCCTGTAAA ACCATGGTTC TCAACTGGGG |
| | 4621 GCCACTGAGA TGTCTAGAGA GATGTTTTTG TTTTCACAAC TCGGGGAGGG TGCTACTGAC |
| | 4681 ATCTTGTGGG TAGAGGCCAG GAATGCTGTT AAACATCCTA CAAGGAAGGC ACAGGACAGT |
| | 4741 CTCCTACATC AAAATATGAC CCAGTCCCAA TGTCACCACT GCTGGGGTTG ACACTGGCAC |
| | 4801 TGCTATCTTA ATTACATTCA TTGAGTGTCT TTTAGGAGGC CCTATTCTAA GTGCTTGCTA |
| | 4861 AGATTATCTC ATTTAATCCT CACAACACTT CCGCTATGTA GCAGGTGCTG TTATTATCTC |
| | 4921 CGTGATGGGG AAACTGAAGC ACAGAGAGGG TTAGTAACTT GCTAAAGGTC ACAGAGCCAG |
| | 4981 TGGGTGGTGG AGCTGGTTGC CTGACACTAG TTCCCTCCCC TCTCAGCCAC ATGTGGGTTT |
| | 5041 ACTTGGCCAT TGTGGACTAG TCTGGGAACC CAGATATGAT CTATAACATT GACCCAGTAG |
| | 5101 AATATTGATT CCAAAACCAC TGTCTCACAA ATGAATTTTT ACAAGAGTCT GTAATCGGAG |
| | 5161 CATGACCCAG AATAAGGTTA GGGAGATGTG GAGTTAAAGC TCTCAATTTC TTATCTGGCC |
| | 5221 CCGACACAGA GAGCAAGGCA TTTCACTCTA CATTGGTGCT CTGTTTATAA AACAAAGAGC |
| | 5281 AAATATCTCT TCCTAAGGTC CTTAAACCTC TTCCCCCAAT CCAGGGTTTC TGGACTGCTC |
| | 5341 TGCCATATGA CGGGGCAGCT GGTTTGATTG ACCCAGGGAA GGCTGGAAAT CAAGACTGGG |
| | 5401 GGATCAAGAC GTAGATTCAG TGTGGCCAAG GTCAAGTCTC TGAGGTTTAG GGACATCAGA |
| | 5461 TCCCCAGCTT AGGTTCTGTA CCTCGGCAAG GTGAAAGCGT TGGCGCCCAC TGATGAGGCC |
| | 5521 TGCTCTGAGA TTGTGGGTGT GGGTTGAGTT GGGTGGGCAT AGGCAAGTCC TCTTGTAAGA |
| | 5581 ATCTTTTGGC AAAGATGGGC CTGGGAGGCT TTTCTCACTT CCTGGGGCCC AGGCTTTGCA |
| | 5641 ATAAGTATTC CATTATACTG TGGTACCTTG GGGCTACCTG AGAATCCTCT GTCTCGCCCC |
| | 5701 TGTTGCCTTG CCAAAGAGTT TGCTGTCCAA GAATTCCTTT CCTGTCTCCA GGTGCCATGC |
| | 5761 TCCTGCCACC TCTGCCAGGT TCCCTGCCTG CCCAGATGGC TCCCAACTGA GTGTGAGGAG |
| | 5821 GAATTTGAGA CAGGTTTTGA GCTTTCTGGG TTCTCCAGTT AGGAAACTTT CTGTAAGCAT |
| | 5881 GCAGATAGAA TGGGCTTCAG CAAAATACAA ACTCGAACAA CTTCCATGTA TAGTCCCTTA |
| | 5941 ATTTTCTTTG CTTTTTTCAT ATTTCATCAG GCTCCATGCT GAGCCCAATC AGGGACCCGA |
| | 6001 TAGAAATCCA AACACCATGT CAGCGAGTCC CCAAGAAATG CATTTTGTGC CAAGGCTATT |
| | 6061 CAAGGAAGGT TTGGGAGCAG CTCAAGGGCA GACACTGTTA CCCTCCCCCA GGTCCCCAGT |
| | 6121 GCAGGGCAGT GTTCTGCATG TGGAGGCAGT TTGGCCTAAT GGTTAAGGAG GTAGGCTCTG |
| | 6181 ATCGGGCCTC CTGGGCACAA ATCCCAGCTC CCTGCTCACT GTGAGACCTA AGCCATATTG |
| | 6241 TTTAGCTGCT TGGAGAGTTT TTTGTCATCC ACAACTTGGA GTATGATGGT ACCTGTCTCA |
| | 6301 CGGGTTGCCA TGGGGTTCAC ACAAGCTAAC CCGGTACTCA CTAGGGCCAA GCACATAGTA |
| | 6361 ACTGCTCAGT AAATGGCATC ATCGGCGGTG TCCTGTGGAT GAGTGCTTGT GATTGGCTGA |
| | 6421 ATGACCAGAG GGGTCTAAAG ATCCTGGTGA TGGAATCAGT TGTACAGATA AATTGTTACA |
| | 6481 CTGAGTAGGG ATCAAGATAG GAAAAGTCGG CAACTACCCA GCTCCCCTGC ACCAAACTGG |
| | 6541 GCAGAAGTGG ATCCTCTGAA AATTGCACAC ACCCATGTTT AAATGTACAC ACAGAACTCT |
| | 6601 TGCCACAGGC AAGCGGAGAT TTGTCATCTG CTGTCCCTGC CTCATCTTCT TCCTGAAATC |
| | 6661 CACTCCATGC CAGGAATAAA CTGCATGCTC TCCACCAGCC CAAACTGACC TGCCTTCCCG |
| | 6721 CCAGCCATCC CGGGCAGGGT GACCTGGCTT AGTACATCGG GTTCAGAGAT CTTTCCAGTT |
| | 6781 TACTCGTTGA ATAAAAAGTG AGGGCTGATC GAGAAAGTAA TGGCAGTCAG GGAAGGCGAA |
| | 6841 GGAGGTAAAG AAGAGATTTT ACAAATGAAG TAATTCAACA GAGTGCTGAC ATTGGTAAAC |
| | 6901 TGGCAAACAG ATTTCAGGGT GGTTGGTTGA GAGTAGAGTA GAAAAGGATT AAATAAAGCA |
| | 6961 AACTTGTGGT GTACTGAATC TTAGGAATTC CATGTATCCA ATAAGTATAG TCATTTATGA |
| | 7021 ATTAATAAAT TCGGCCTAAG AAGCCTTCTT ATCGCTTAAA TCAAGACTAA GTAACAATAT |
| | 7081 ATCAGTTTTA AAAAGTCATT ATATCAGAAA ATCATTTAAA TGATACACAT AGATTTCCAA |
| | 7141 GATTTTACTT TAACCGAAAC TATATAAATG TGAATTTGTT CACCCATCTT TTGACACAGG |
| | 7201 GCTCAGGTCT TCTCTTGGTG TCTGGATCAG CCAGTTGAAA TTTCTTGTCT GTTTTGCCTA |
| | 7261 TGCCACATTA ATAATGCACT GTCTGGGTCC TCCGATTTCA GTTTGGATTT TGGGTTTACA |
| | 7321 TTGTGGAGTC ATCTGAATGC AGAATCCTTC AGGGATTTTA CTTTTTTTTT TTTTTTTCAT |
| | 7381 GGTCTTTACC ATCCCATTTG ATAGTAAATA TTACTCACCT TTATGAAGTC TTTCCAAAAC |
| | 7441 ATTCAACTAA ATTTTCTTAA AATCATTGAA TGATTTGAAG AGCTTATTCC TCAGCACTTT |
| | 7501 TACTCCATCA GCTTGCACCT TATTTTTTAA TCTTTTTTTG AGACGGAGTC TCGCTCTATC |
| | 7561 GCCCAGGCTT AAGTGCAATG GCGCGATCTT GGCTCACTGC GACCTCCACC TCCTGGGTTC |
| | 7621 AAGCAATTCC GCCTCAGCCT CCGCCGTAGC CGGGACTACA GGTACACACC ATAATGCTCG |
| | 7681 GCTGATTTTT GTATTTTTGT AGGGATGGGG TATCGCCATG TTGGCCAGGC TGGTCCCGAA |
| | 7741 CTTCTGACCC AAGTGATCCA CCCACCTCGG CCTCCCAAAG TGCTGGGATT ACAGGTGTGA |
| | 7801 GCCACCGCGC CCGGCCAGCT TGCACCTTAT TTAGGATATG TGATTATTAT AGCAAGTCTG |
| | 7861 GTGTACATAC AAGATTTTGA ATGGGCACAG ATGACCTTTA GTAAGTGCTT GGCTGTGATA |
| | 7921 AGAGGCAGTC CTGACTGCAG ATCAGGCTGT GTGGACCCCA GCCTTGCATG TTTACAGACC |
| | 7981 TTCATGTCTT ATTCTTACAG GGTATCAGAA GAACACCTAC TGGGGAAACT TATAAATTAG |
| | 8041 TAAAAGGTGG GCATTCTCCC CGCCCATCTT CTGTCTGTCT GCCAGGACTA GCACAGCACT |
| | 8101 TTGAAGTCAT TCACATAGAA TCCCAACTTA AGAGGGTAAA ATCCTCCTCA ACAGACTGAA |
| | 8161 AATAAGTTTA AATTCCCTTT GCTATATTAA CTCCCCTGAG GAAAGAGTCT TAGATCAATG |
| | 8221 TCCAACACTA AAAACAGTTT TAAATCAGCA AGTGAGAATT AAATCTGAAG CAATTGATAA |
| | 8281 TAATGTTTCA TTCATTCCTC TCCTTTGGCC CCGTCCACCC TACTGCTAAA TCCAGGCATC |
| | 8341 AAAGAGAAGA GGGACATAAT TATCTCTAGT CCCAGCTGCT GGTTTTCCTT CCAGCCTATG |
| | 8401 GCCCAGTTTT CTGTTTTACT GAGAAGGCTG GTGATGTTAT CTTGGGATCT AAGTCTGCAG |
| | 8461 TTTCACCACA AAAAGTCCAG GGATGCACTT TCATGCTTGT GTCCTCCTCC CTGGGATAGC |
| | 8521 AAGGATATTA GAAGACCCCT GGCTCTGTAA TTGCTTGTCA TGTGCTCTAC AGACGCCACA |
| | 8581 GAATGCCAAG AACGAAGTGC TGGGAAGGAC AAATTCATGG AACCGTGGGA CGGTGCTCCT |
| | 8641 CCCCCAGCGT AAAGGACAGC TCCTCCTCCT GAATTGGAGC CAGCGTTCTA AATCATGTGT |
| | 8701 CAACAGAGTT GTCCTGGATC GGATCCAGTT CTGCCATTGA TTTGCAGGTC ATTTCAGTGG |
| | 8761 TACCTGTTTC CAGTTGTTCT TAATTGAACA GTGGCACCAA ACTATTGTCT TGCCTCATCC |
| | 8821 CCCTCCCATG GCCTGTCCCC CAAAAAGAGA CTTCTTGGGT AATTAATCAG GGCAACATCA |
| | 8881 GGCAGTCTGG GCGCGGTGGC TCACGCCTGT AATCCCAGCA CTTTGGGAGG CCGAGGCGGG |
| | 8941 CAGATCATGA GGTTAGGAGA TTGAGACCAT CCTGGCTTTG TGAAACCCCG TCTCTACTAA |
| | 9001 AAATACAAAA AATTAGCCGG GCGTGGTGGC GGGCGCCTGT AGTCCCAGCT ACTCGAGAGG |
| | 9061 CTGAGGCAGG GGAATGGCGT GAACCCGGGA GGTGGAGGTT GCAGTGAGCC GAGATCGCAC |
| | 9121 CACTGCACTC TAGCCTGGGC GACAGAGCTA GACTTCTTCT CAAAAAAAAA AAAAAAAAGG |
| | 9181 AATCTCTTTG GTTTTATATA TATTTTTTTT ATATATATAA TATATATTAA AATATAATAT |
| | 9241 ATATATTTAT ATAATATAAT ATATAAATAT ATTATATATT ATATATTTTA TATATTATAT |
| | 9301 ATTATATATA TTATATATTA TATATTTATA TATTTATATA TTATATATAT TTATATATTA |
| | 9361 TATATTTATA TATATTATAT ATTTATATAT AATATATATT ATATATTATA TATTATATAT |
| | 9421 TATATATTAT ATATTTATAT ATATTATATA TTATATATAT TATATATTAT ATATTTATAT |
| | 9481 ATTATATATT TATATATATT ATATATTATA TATTATATAT TTATATATTA TATATTTATA |
| | 9541 TATTATATAT ATTTATATAT ATTATATATT ATATATTATA TATGTATATA TTATATATGT |
| | 9601 TATATATTAT ATATATTTAT ATATATAATA TATTGTATAT ATTATATATC TAATATATTA |
| | 9661 TATATATTAT ATATATTATA TATTATAATA TATATTATAT ATTATATATT ATATATATTT |
| | 9721 TTATATATAT AATATGTATA ATATATAATA TATATAAAAA CATATATAAT ATATATTATA |
| | 9781 TATTATATAT ATATTATATA TATTATATAT ATTAAATATA TTTTATATAT ATTATATATA |
| | 9841 TATACACATA TATATATATA AATGAGGCCA GGCTCGGTGG CTCACACTTG TAATCCCAGC |
| | 9901 ACTGTGGGAG GATCACTTGA AGCCAGGAGT CTGAGACTAG CCTGGGCAAC AAAACAAGAT |
| | 9961 CCTGTCTCTA CAAAAGGAAA CTGTAAAAAT TAGCTGGGCA TGATGGCATG TGTCTGTAGC |
| | 10021 CCTAGCTACT TGGGAGGCCG AAGCAGGAGG ATCGCTTGAG CCCAGGAGTT CAAGGCTACA |
| | 10081 GTGAGCTATG ATTGTCCCAT AGCACTCCAG CCTGGGTAAC ACAGCAAGGC CCTGTCTCTA |
| | 10141 AACTTTTTTT TTTTAATTCT ATTTATATTT ACATGTATTT AAATGTGAAT ATTCACTACC |
| | 10201 TATTTGTTGC ATGCCTGCAT TTTTTATACT GGGCTTGCCA AAAACCCGAA CAGCTTTCTA |
| | 10261 CTTTGACAAT GTATCAGAAT TTAAATCAGC AATATGTTAA TAAGCCAAGC AAAGGTTATA |
| | 10321 TATGCAAATA AAACTGTTGT CTATAACCTC CTGTTACACT GGGGCACAGC AAAAGTCATG |
| | 10381 GTGTAGTCGC ATGTGAACCT GTCCCTTTCA TAGCTGCTCA TTGCCAGGAA ACATCAGGAA |
| | 10441 TAGCCATTTG GAAGAGTCAT CAGCCCTCCC ACCATCCGTT TTCTGTCTTG TCTTTTCCCT |
| | 10501 ATGAGCAGGG GAAATTCCAC GCTGGCCCCA ATCCCCAGTG CAGCGGCTCA GCCTCTGCCT |
| | 10561 CTGCTGCTGG TCCCCATGAG GCCAGCTTAG AAACGGAGGA TTTTGCAGAA CATCCCTAAA |
| | 10621 TCCGCTTGAA TAATGAAGTG ATCATTCATA AACTCACCTG AACCTTATTA AAACCTATTT |
| | 10681 AATATTTTTC CTGGATAATC CTATAGGGAT AACTTGCCTC CTGGGCTTCT CTCCACCGGG |
| | 10741 TTCAGTTCTT CCTTTAGTGG TGAAGTTCCT CCCTTCTTAG CATCTCAACT GTGCCTGAGA |
| | 10801 AAAGGCCAGT GGCGGCTGCA CTCTGTTCCC TGTGGAGTGT TAATAAAGAC TGAATAAATT |
| | 10861 GAAATAAATC CCTTTCAATG TCATTAAGTG CTATAAATAA TCATGAACCA ATGTTCGATG |
| | 10921 GCTGATGAGA AATGCAAGAA AAAATTTTTA ATCAGTAGGA TTCATAAGTT GACAATCTGG |
| | 10981 GCCAAGTTAA AAAAAATAAA AATAAAAAGA CTTTTAAAAA GATCTTATCG TTTGTTACCA |
| | 11041 GTAAGACTGA ATTCCAGAAG CAAGCTACTC CCTCATTTGT GGGCCCCTGT TATCACTGGC |
| | 11101 TGCTTAGGGT TGCCAAGCCC TGAATTCATT TGTCAACTAA GAGATTTTTG GCCAAGATTA |
| | 11161 AGATTTCCCA TGCCTCCATA TTTCCATCTG AGAAATGGAG ATTATACTGT CTTCCCCCTC |
| | 11221 AGAATGGATG ATAATGTGGT CTCTCTTCTG TTCGCATAGT CATAGAACTG AAATAAAACA |
| | 11281 ACTTAAGAGA ATTCCTTTGA GCTTCTCAGA AGTGCTGCAG GGCTGGGGGA TGCCTCCCAG |
| | 11341 GAGCCGCAGT CAGGTGCTGA TCTGAAGTCT TTGGTGGGCT GACTTTAGCC TGACCTGAAA |
| | 11401 TAGTATAGCT GCTGCCACCT GGCTCCCTTA GCGTCAGTCA GACGGTGCAG CTGGTTCCTA |
| | 11461 GGGGTGAGGG CTGAGCCAGC AGGGTCCGTG CCCAGGAGGG ATGCATGGGT GGCCACAGCC |
| | 11521 CAGCCTGCAC TGATCTTGTC TGTCCCCTTC TTTGGAAGGA AGGAGCCCCA AACCAGGGTG |
| | 11581 CAAGACAGTG GGTGGGGGTG CCTTGAGCAT GACCTCAAGT GATTTCCAGC CCCTGCCAGT |
| | 11641 GCTGACTTCT CTGGGGAAGG GCTGGGACTT CCTTCTGGGC TCAAGTCACG ACCCTTGGAT |
| | 11701 GGAATTTCCT GGGAGCTTTT CTGTTTTTTC TGGAGTTTTC AGTTTTTTCC TAACCAGACA |
| | 11761 GGGACTTGGT ACAGAATCTC ATATTCTAAT TATGCCTAGG AGCAGCCTCT CCCCACCACT |
| | 11821 CACAGTGTTT AGCATGTGAC AGGAATCGAT TAAGGCATGA GTGATTAAAT TAAAGCCAGG |
| | 11881 CATTGACTTG GATGGTGTAA TATTCTGACA TCTGTTTGGT GTCAAAGGCA CGGGGCAGGC |
| | 11941 GCGTTAATTG AACTGCTTGC ACCTGGCATT TGAATTGAGC CAGAGCGGGG CTAAAGTCAG |
| | 12001 TTTGCCTTCA CCCTGTAAAT GGAGGGTTTC TCCGGAGCGT GGATGGTGGG AGGTATTTCA |
| | 12061 GGGTGTATGC ATAACCCCCA CCCTGACAAT GGCCCATCTC TTCTCCAGCG TGGCCAGGTT |
| | 12121 TGAGTGCCAG TCCTGGGTGT CCAGTGGCCC CATAGCCTTG CGTTTTAGTA AAATGCTGCC |
| | 12181 CCCATTACCA CCTGGTCTGT GCACTTCGGT CACTGGAATT TGCCATCTTC CAGTCCCGAA |
| | 12241 TGTGGCAAGC CATGGAGCCT TAAGCTCTTC TCCCTCCACA TCCTGGAACA GACCCGCCAG |
| | 12301 TTTCTTCCAG GCATTGCCTC AGTTTGCCCC TCTGTTTCCA GTCACACTCT CACCAGCGAT |
| | 12361 AAAATGATTT TAGACCTTAT CATCTCACCC TCGGATCCTT ATGGAAACAA TAATGAGTTG |
| | 12421 TTCCCTGTTT CAATTCCAAA ATTCATATCC AATCCGTTTT GCATGCCATT GCCAAATTCC |
| | 12481 TCCCAGAGCA ACCCCGTCAC CTGCCCTGGC CCTCTCCAAG TGTGGTCCTG CCATGGGCAT |
| | 12541 CGCCTGCTAA GCCAAGCTGG CCTCGAGCTG CCTGCCCGGG TCCCCACACC TTGGCTCACC |
| | 12601 TCCCTGCCCA GTCCCGCCTC CTGCCAGCCT GCCCTGTGGC TCCTTCATAG ATGCCGTGCT |
| | 12661 CTTTCTGCCC CTTGCTCACC CATGGCAGCC TTGCCCCTCT CTCCCTGCCC CACCCCCTAT |
| | 12721 TTAAATTGAC CTGACCTTCC TCAGTGTCCA TCTTCCCCGA AGCTTTCCCC AGCCTTGGCA |
| | 12781 CTCAAGGTCC AGAGGCTACG CGTTTCCTCT CACCTGTGGC AGCGCCGTGC TCCCCAGTGC |
| | 12841 CTCACAGTTT CCTTCTTGCC CCCGCTTCCT GTGTAGGACT CATCTGCCCA CAGGTTGCAC |
| | 12901 GTCCTGTGAG GGCAAGGACT GTGTCTTATG TGACTTTCCT TCTCCAGTCA CAGAGCTGGG |
| | 12961 CACATAGATA GCTCAAAACC CTCTTTATTA ACACAGTTGG ATGTTGAGAA ATCAAACAGG |
| | 13021 CCAATGTCAA ATGAGCTCTC CTTATTTAAA TCAAGTCAGT TCTCCACCTC CTAGCACTCA |
| | 13081 GTTCCAGTAC TCTATATACA TGGAAATAAT AAAAAACACA TTTCCTTTGA AACATTCTAT |
| | 13141 AATCGTTCCT TTGCCCTACT TCAGACCAAC TTAACGCACT CCCCATTGGT CCAAATGAGT |
| | 13201 TTTGCTATAC GAAGATGCTG ATAATAATAG CAGCAGTGGA TTATTCTGCT AAAACCATTG |
| | 13261 CCTCGTTAAT CCTCAGTCCC GAGGTGGGGA TTATTATCCT CATTTTGCAG AGAAGCAAAC |
| | 13321 TGAGACTCAG AGATTTCACA GCTGGGGAGG GAGCCAGCTC ATCCCTCTGT CCAGGCCCAA |
| | 13381 GCTCTCTCCC GCTTGCCTTC CTGCCTCTGC AACCTCAGAG CATCCCCCAT CTGGTTCTAC |
| | 13441 TGCCTGTGCT AGTCGTGCAG GAGCCAAAAG ACACGTCTTT AGTGCTAAGG ACTGGAGAAG |
| | 13501 CCATGCCCTC CAGCCTCTGT GAATGGGTCA TATGTAACAT GAGCCTGGAG AAATTATTTG |
| | 13561 AAACCAAAGG CAAGCCTCTA AACCAGGCTG CTGCTTCATG GCGCCGGTGA CGGCAGAACC |
| | 13621 AAATTTAGTG CTGTGGGCAG GTCCACACTT ATCAAATAGA GAAGCTCATT TTTCTTCCGG |
| | 13681 CTCACATCAA GCATGAAAAA TGTTCACACA TACCCCCCAC ACACACATGC TTTCCGGAGG |
| | 13741 GGTCCATGTG GCTAGAGGCT GGAAGATGTG GATGAGAGGA GCCTGGCAGG TAAGCCCAGG |
| | 13801 GAAGATGACA TTCAGCTTCC CAGACAGCAT CTACAGGGAG AAATTTAATT AAAAGTGGGG |
| | 13861 CGGTTTCCCT GAGCAAGGCA GACAAAGTCA GCCCTCTACT GTTAAGAAAA AGGGTCACAG |
| | 13921 TGAGAGGGGA GGTGAGGAGA CTGAGTCTGT ATTTTCTAGT CTGTTGGGCT ACACTACCTG |
| | 13981 ATCCCCCTTC CTCAAAAATC CACTTTACTT TCCCCATGTC TACACCAATG TGGTTCACAC |
| | 14041 TCTGGGACCA GGAAAAGGGG GAGTGATGGG GAACAGAGAA GGGAGGAGCT CACACAGCTG |
| | 14101 AGGCTGGGGT TATGCATATC GAATTACTTA GAATTTGCAA CCTCACAGGG TACTTTCATG |
| | 14161 GCGTTGAAAT ACACTTCCCA CAGCCACCCT CCCTCTAACT AAAAGCAAGA GTCATTTCTC |
| | 14221 AGTTCTGGTC TTGCCTCCCA CGTTCTCCTC CACATTTAAG AAAATCCACC AGCTACAAAG |
| | 14281 TGAAGATACC ATATGTGATA TCCCACCCTA GTTTCTGTTT TATCAGGGTT TGGAGCAGGT |
| | 14341 GGAGCAGGCA GAGGGATCAT TTCAGCCTAT AAATTGTATT AAGGGTGAGT ACTGAGTCAT |
| | 14401 TCTTCAAGAA AAGTTTTAGA AGCATCCAAA ACTGAAGGGT GGAGCCACCT GGAGACAGTA |
| | 14461 TCATCAGTCC TGGCCCCGAG CATGGCCTGC ATAGGCCCCC ATGGATCCCA GCGGGAGCTG |
| | 14521 CAGAGTGCGG GCACCTTGGC ACACAGCCCT GAGTGCAAAA TTAGGAGCTG GGCAGAGGGC |
| | 14581 ATCTCTCTGT CGCCATTGGG CAGCCCAGGG CACACTGGTC ATAGCCTTAG ACCACGAACA |
| | 14641 CCCTGTGCCC GGGGGACAGA TGCAACCAGT GTGCCCTGGG CTGCCCAATG GCAACAGAGA |
| | 14701 GATCGACACC TGGACCCCAT GTCACGGGGA CTCCACTACT AAGGCTCCTA AGACTGCCAC |
| | 14761 CTTCCAGTGG GATAAGCCCT GCCTCCTACT GGGCCCACAA TGTGCAGAGA ACACTTGGGA |
| | 14821 CTACCTGGCT TTCTGGATAC ACAAATATTG ATCCAATCTG GACTAATTAG AAGGTCAGTC |
| | 14881 CCAATAACAA ATCGAAGTCA GCTGGGCGTG ATGGCTCACT CCTATAATCC CAGCACTTTG |
| | 14941 GGAGGCTGAG GTGGGCAGAT CATTTGAAGC CAGAAGTTCA AGACCAGCCT GGGCAACATA |
| | 15001 GCAAAACCCT GTCTCTACTA AAAATACAAA TAATTAGGCT GGGTGTGGTG GCTCATGCCT |
| | 15061 GTAATCCCAA CAGTTTGGGA GGCTGAGGCA GGTGGTCACC TGAGGTCAGG AGTTTGAGAC |
| | 15121 CAGCCTGGCC AACAGGGTGA AACCCCGTGT CTACTAAAAA CATAAAAATT AGCCAAGCAT |
| | 15181 GATGGCATGT GCCTATAATC CTGGCTACTA GGGAGGCTGA GACAGGAGAG AATCGCTTGA |
| | 15241 ATCCAGGAGG TGGTTGCAGT GAGCTGAGAT GGTGCCACTG CACTCCAGCC TGGTTGACAG |
| | 15301 AGCAAGACTC TGTCTCAAAA AAAAAAAAAA AAAAAAAAAA GCCATGCCTG GTGGAGCACT |
| | 15361 ACGTGTAATC TCAGCTATTT GGGAGGCTGA GGCACGAGAA TCACTTGAAC CTGGGAGGCA |
| | 15421 GTGGTTGCAG TGAGCTGAGA TCGCGCCACT GCACTCCAGC CTGGGCGACA GAGTGAGTGA |
| | 15481 GACTCCATTT CAAAAAAATA ATAAATCTGA GTCACTTTAA TATTGTTATT TGGATGTCAA |
| | 15541 CCTCTAGGTG TTTGAGACAG GAGAGTGATA TGGGGGCACT GGAAACACAC AGGCACGGGG |
| | 15601 TGTCCTCACA CTTGGGTAGC CCACACGATG TGATTTCAGG GTGCTGGGAG GTCCCCCCAC |
| | 15661 TCCCCAAATT ACTAACAAGT GGATAGTACT TTACAGTTTA TATGATCTCA TTTGATTCTT |
| | 15721 AACATGAGCC TGTGAGTGAA AAATTCCTTC CCCTCTTCTA CAGATTAGGA CGTTGAGATT |
| | 15781 CAGGGAGGTT CAGAGGGATT CAGGGAAGTC AAGTGGCACC TGGAGTCCCG TGGCTAATTT |
| | 15841 GAGGCCGGTA GGGGATTCGA ACCCAGGATT TGTGCTTCTT ATGCCTGGGC TTCTGCTCCC |
| | 15901 TGGGGCATGG TCTTCCCCCT AGCTTTCCCA TTCACTGCTT TAGCCTAGGG GTCCTACCCT |
| | 15961 TTATTAAACT GCCAGTGCCT CACTGCTTTT CTCCCCCAAA GACAAAAAAA AAGTGTTTTT |
| | 16021 GCTTTTGTTT TGTTTTTCAT GGGCAGAGAC CTGGAATTTC AGCTTGAGAA TTTGTGCCAT |
| | 16081 ATGATAAATA AATCAACAGA TGGCTTTTTC CTTAAAAAAA AAAAAAAAAA AAACTAAGAT |
| | 16141 GTATTTGCAG TGAGGCATAA TTTGTACCAA AAAGTGCTCA CCACACTGTA GTCATGGGGG |
| | 16201 CAGGAGGCAG CCGCGGGTGA AGGGAGAAAT CTTGGAGTCC AGGCAGCCCC CTTCTGGGCT |
| | 16261 GAACTGGGGA GCTGGGGGTG CTGCCAGCCC TGCCAGGTTC TCCTAGGAGG CGGCAGCTCA |
| | 16321 TATGGCTGTG GGAGGAGGCA GAGGGAGCCT CATATGCACC CACATTTCCA GGGATCTAGA |
| | 16381 AGACAGAAGG AGGAAAACCA CCATCATGTT AAAGCAGACA GTTAGGTAAC ACATCCTGTA |
| | 16441 ATACAAGTTA TTTTTTCCAC ATCTAAAGGC TAAAAATAGT TGTTAGAATT TAAAGATAAT |
| | 16501 TGGTAAATGA GTTTCTATCC TTCTAGTTTC ACATCAAATG GAATCATGCT GCCTTCACAT |
| | 16561 CACTAGTGCC CGTTATTTGT GTTTAATTTC CACAATGTTG TCTAATTCCA CTCTTTGGGC |
| | 16621 TTCCCCAGGG ATCCAGCCTC CCTCACTCGC CCATCGCAGG GAGATGCTTT ATTCATCTTT |
| | 16681 GTGTCTTCTG TGCCGGGCAT AGCGCATGGC ACAGAATAAG CACTCAGTAA TTGATTCACG |
| | 16741 AGTGAATAAA TGGATGAGTG GGTGAGTTCA ATATTGACTA CAAAAACCCT AAGGCCACAC |
| | 16801 TGGTGAGTGG CTGCGCCTGT AGTCCCAGCT GCTGGGGAAT CTGAGGCAGG AGGATCTCTT |
| | 16861 GAGCCCAGGA GTTTGAAACT AGCCTGGGCG ATATAGCGAG AACCTGTCTC AAATGACAAA |
| | 16921 AACAGGGCCA GGTGCAGTGG CTCACGCCTG GAATCCCAGC ACTTTAGGAG GCCAAGATGG |
| | 16981 GAGGATCACT TGAGGCCAGG AGTCCGAGAC CAGCCTGGGC AACATAGGGA GACCCTGTCT |
| | 17041 CTACAAAAAA TTTTTTAAAA ATTAGCTGGG CATGGCGGTG TGCGCTTGTA GTCCCAGCTA |
| | 17101 CTCAGGAGGC TGAGGCAGGA GGATCACTTG AGCCCAGGAA ATTGAGGCTG CAGCGAGCCA |
| | 17161 TGATGGCACC ACTGCACTGC AGCCTGGGCG TCAGAACGAG ACCTGCTCTC AAAAAAACAA |
| | 17221 ACAAACAACA AAAAAAAAGG CTTTCTTAAA GAGACTTGAG AACAGAAAGG GGAACAGATA |
| | 17281 CATAACTTAT ATATTTATTT GTTCATCTTT CCACCTTCCT GGAGGGTGGA GGGGAACAGG |
| | 17341 TCTGTATTTG GAGTTTTGAA TGCTAAAAGT GGGAATACAT GTACTGTTTG CCATGATCTG |
| | 17401 TTCAAAAGTT AAGCCAAATG CCTTAGATTC TCCTGAAAAC TGGAATGCCA CTGTAAACTA |
| | 17461 TAAGCCCCAC TTCAAAGATA AAAGATCTTG ATGAACAGGG CTGGGTCTGT GGACTGGGCC |
| | 17521 TCTCCCCACC ACACAAGGAA GGGTGGTGCC AGTTGAAGGA AAATCACTTA AATCCTTGCT |
| | 17581 GTCTCCTAAT AAGGTGTGGT CCCAGGTAGG GCTGTCAGAA TTAGCAAATT AAAACACAGG |
| | 17641 GCATCTGTGA AAATTAGAAT TTCAGATAAC AACAAATAAT TGGCATAGGC TGCATAATGT |
| | 17701 CCCTCAAAGA TATCAGGTCC TAATCTCCAG AACCTGTAAA TGTGATCTTA TTTGGAAAAG |
| | 17761 GGGTCTTTGT AGATGTGGTT AAATTAAGGA TTTTGAGATG GGGGGATTAT CCTGTATTAT |
| | 17821 CTAGGTAGGT CCTAAATGCA GTCACACTCA TCCTTGTAAG AGGAAGGAAG AGAGAGATGG |
| | 17881 AAAACACAGA AGAGAAGACA ATGTGGTGAT GGAGGCAGAG ATTGGAGTGA GGTGGCCACA |
| | 17941 AGCCAAGGAC TGCTGGCAGC TACCAGCAGC CAGAAAAGTC CAGGAACCAA TTCTCTCTTG |
| | 18001 GAGCTCCAGA GGGAGTGTGG CCCTGCTGAC ACCTTAGCTT CAACCTAGTG ATCCTGATTT |
| | 18061 TGGACTTTGG CCTTCAGAAG TGTGAGGGAA TGAATATCTG TTGTTTTAAG CCACCAAGTT |
| | 18121 TATGGTCATT TCCTACAGCA GCCACAGGAA TCAAAAACAG TAAGTATGTC CCATGCAATG |
| | 18181 TTTGTGACAC ACACCAAAAA TATTACTTGT TGTTCACCTG AAATTCAAAT TTAACTGGGT |
| | 18241 CTCCTGTATT TTATTTGGCC AACCTAGTTC CCAGGCCCAA AGAAAGAGGC TTTTGAAATT |
| | 18301 TGCAAGAAAG CTGGTTGGAG CTGTCAGAAA GTGGACTTTG TAAACACAGT ACCACCGAAC |
| | 18361 CAATTTGAAC TGTACTACCT CTAGACAAAA GAGAGGGCAG TCAGACAGTT GTTCGTGATT |
| | 18421 TCTTCTTTCA ACAGTCATTT GAGCACTTAC TACAAAACAG AAGCTATGTG TAAGGGTGGA |
| | 18481 GGCGTTAGCT GTTAATCAGG ACCTCCAGGC TAAGTTTCTG TATTAGTCCG TTTTCACGCT |
| | 18541 GCTGATAAAG ACATACCCGA GACTGGGGAA TTTACAAAAG AAAGAGGTTT AATTGGACTT |
| | 18601 ACAGTTCCAA GTGGCTGGGG AAGCCTCACA ATCATGGCAG AAGGCAAGGA GGAGCAAGCC |
| | 18661 ACATCTTACA TGGATGGCAG CAGACAGACA GGGAGAGAGA GCTTGTGCAG GGGAACTCCT |
| | 18721 CTTTTTAAAA CCATCAGATC TCGTTAGACT TATTCACTAT CAAGAGAACA GCACAGAAAA |
| | 18781 GACCTGCCCC CATGATTCAG TTACTTCCCA CCAGATCCCT CCCACAACAT GTGGGAATTC |
| | 18841 AAGATGAGAT TTGTTACCAT ATCAGTTACC AACCCTTCCA GATAAATCAC GTGAAATATC |
| | 18901 GCCATTAACA GAGTGAGCTC AGGTGGTTCT TCAGTGCATT TCTGATACCT GAACCTTCCC |
| | 18961 TGGGAATTTC ACAGACCATC AGGCTCTCCA CCCTTTGATA GCAGGATAGC AGGGCCCAGG |
| | 19021 TTCTGCAGGA GGAGATGTTA CCACAGGCCT GAAAGGGAGG GAGGGGCAGA TGCTACAGGA |
| | 19081 AGATGCTGGC TCTGGATTCG CTGGAGGAGC TTTCAAGGGA AGTAGATACA CACTGTCTCC |
| | 19141 ATCATTTCAT GTCCATCACA CTCTAAAATG CTTTGGACAA GAAGCAAATG TTAAAGACAA |
| | 19201 ATGTGGCCCA TTTTCCTGTA CAAAGAGGGC TGCTCCCATG CCAGGCTATT GGCACTGGTG |
| | 19261 GGCATGAGGC TTCTCTGCTG CCCTGGCCGG GGGGTTCTCT CACTCACCAT TGGCTCTCTG |
| | 19321 ACACCTGGAG AGACCACCAC CCTTGGGCTT TCATGATGCT CACAGAATCC ACACTGTTGG |
| | 19381 AGCTTTAAGG AGCCTGGATC AACTGGAACA GGCAGGGAGT ACTAGGACAG CCCAGCATTG |
| | 19441 CCCCAAAATA TCCAGGCCTG ATAAAAGAGA AAAACAGGTA GCTCACAGGA AAAGGATAAA |
| | 19501 AAAAGGAGGA GGGATTTAAC ATGAAAAGGT GCTTGATCTC CCTCATAATA AAAAGACTGC |
| | 19561 TGATTCCATC CAGGCAAGTG ACAGAAAAAA AAAAATTAAT TTAAAAAGAC TGCTGATAAA |
| | 19621 ACCACAGCGA GACACTGCTG CTCAGGGATC TGAGGGTGTG GGCAGCCAGG CTGCCACGCA |
| | 19681 TCATGGGTCG GAGAGGAAGA CCACACCCCT GGAGCAGAGG GCGGCTGATC TGTCAGATGC |
| | 19741 CCTTTGACAG CACCTCAGCT TCCAAGAATT AACCCTTTCT ATGTGAGCAG AGGCATCCAT |
| | 19801 GGGGGGACAC ACTGGTGAAT CATCTGTTAT GTAGAAGTCT GGAAAACATC AGGATGGAAC |
| | 19861 TGGTGAAATA AGTGTGGCCT CTGACGGAAT GGAGCGGTCC GTCTGCACTG CTGCGGGTGC |
| | 19921 CCCTCAGATC CTGTGGGTCA GTGAGAAAAG CAGTGAGGAA CAAGGCAGGT ACTGTGTACT |
| | 19981 GTCCTCTGCG TGCAAGGAAG GCCAGCGCAT GCAACAGAGT CCACACAGAC ATAGCCTAAC |
| | 20041 TCTGGAAGGA AGAATGAGAA TGCAGTTTCA GTGGTGGCCT CTGGTGGGGA GAAACTGGGT |
| | 20101 GAAGGGAGAT GTCATTTCCA TTTCTCTACT ATTAATTTTG TATTACCATG CTTAAATGTT |
| | 20161 ACTTTTTACC TTTTTTTTTT TTTTTGAGAC AGGGTCTCTC TCTGTTGCCC AGGCAGGAGT |
| | 20221 GCAGTGGTAC AATCATGGTT CACTGCAGCC TGAACCTCCC AGGCTCAAGC AATCCTCCCA |
| | 20281 CCTCAGCCTC CTGAGTAGCT GGGACTATAG GCACGCATAC CACCGTGCCC AGCTATTTTT |
| | 20341 TTTAATCAAG ATGGAGTTTT TCTATGTTGC CCAGGCTGGT CTCAAGCTCC TGGACTCAAG |
| | 20401 CAATCCTCCT GCCTCAGCCT CCCAAAGGGC TGAGATTAAA ACGTGAGTCA CCCTGCCCAG |
| | 20461 CCAATTGCTT TTTAAAAAAG ATTAAATGCA TGTATACGCT CAGGCATCAG CACACTTGGA |
| | 20521 AAGGATGAAA ATATCCGGAA GAAGGGTTCT TTTAAAAGGC TCCTCAAGTG ATGCTGGCAG |
| | 20581 GCATGACGAA TGTCCCTGGT CACAAAAGCT CTGATCTGGC CTAACCCTGT CATGTTAGAG |
| | 20641 ACTGGAGTGC GTGTGTGTGC GCGCAAAGTG TGGGGGGATG GGGGTGAGTG TGTGTGGTGT |
| | 20701 GTAAGCATGA GTGTGTATGT GTGTGGTGTG GGGGTGTGTG CTGTGTGAGC GTGTGTGAGT |
| | 20761 CTGTGTGTGT AGTGTGTGTG TGAAGTATGT GGTGTGTATG TGTGACGTGA GGTGTGTGTG |
| | 20821 GTGTGTGAGT TGTGTATGGT GTGTGCATGA GCATGTGTGT GGGCATGTGA TGTGTGTGTG |
| | 20881 GTGTGTAAGC ATGTGTGAGT GTGTATGTTT GAGCATGTGT GGTGTGTTGT GATATGTGTG |
| | 20941 TGGTGTGTGA GCATGTGTGT GTGATGTGTC TGTGTGTGGT GTGTGTGAGC ATGTGTGTTG |
| | 21001 TGTGTGTGGT GCATGTGTGT GGCGTGTGAG CGTGTGTGTG CATTGTGTCT GTGAGCATGT |
| | 21061 GTGAGTGTGT GTGTGTTCAG CATATATAAG GCATGTAACT GAACACAGCA CTTTAGAGGG |
| | 21121 CTCTCCTGGA GTCAGAGGGG GTGGGTAGGA GGAGAAGGGA GGTGGGCTAG TGTGCTGAAG |
| | 21181 TATCTACTCC TTGTCATAGT CTGTGACAAC CCAGACTAGC CCATGAGCCA CCCTGTTCCC |
| | 21241 TGCATTTCCA ATGAGACCTC GGTGGACATG TTCCCTGAGG TGAGGCTGAC TGATGTCATT |
| | 21301 TGACGATCTT GATGCCAAAT CCTTTTATAT CAAAAACAAC CAGAACACTC TCTTTTCTCT |
| | 21361 TAGTGCTTTC ACCCAGATGA CCACATTTCA TCCTCCCAGC CACTCTGGGC CAGGTGGCAC |
| | 21421 TGCTGGTTTG AAAGGGAGGT CTCCCCTGGA GTAACTTCCG TGGGCGGATT CACACCCTGC |
| | 21481 CCACAGTCCT GTCCCAGTCA GCCCACCATG GTGGTCTCCG GTTCCTCCAG AATTCCCGCT |
| | 21541 TTTCAGCTCA TCCCCACATT CCCGGAGGGA CTGAGAGCGC AGCCCCAGGG CCCTGCTCTT |
| | 21601 TGGGGGCCGT CTCTACACCC AGAGAAGCAG CAAGGCATTC CTAGGTTTCT CTTTCAGATG |
| | 21661 CAGAACTTCA GTGTTCAGAG ATGTTCCCAC TGGTCCTGAG AGGGCTCAGT TCAGCTTTAA |
| | 21721 TGACTGCGCT GTTGCGTGTG CTCTGCAGAG GGCGGGTGGC CCAGCGTGGC TGACTGCAGT |
| | 21781 TTTCCTGACG TGGAGCCCGA GCCTGCCCCG CTGTTTATTA ATTAAGGATC ACTCTGCTTG |
| | 21841 CAGAACCCTG AACTCCCCAG AACTGTGAGG TGGGAGAACC CCGAGAGGCC ACCTGGCCCC |
| | 21901 ACTTCCCACC TGCTGCCCAA ACCCCCTCTC TGCCTTCCTG ACAGTCACCC CAACTCCCAG |
| | 21961 TGATCCCCAT CAACCATCTG ACAAGGGGAC TGAGAGGGAA GAGAAAGGAG GGGCCCAAAG |
| | 22021 AGGAAGGTAA AACTGTCGGG AACAGCCCCC AAATGTGTGA CAGCCTTCAG TGGAGTTGCC |
| | 22081 CACTTTCCCT TTTCTCCTCC CTGCAGGACC TCCCTTCTCC CCAGTCCTCC CCAACTTCTG |
| | 22141 AGGTTACATT GAGAAAAGTC TGCAGAGAGG TGCCAGCATC ACAAGGTGTT AAGGACCACG |
| | 22201 AGTTTGGCAT TTTAACAGAT GCCAGAGCCA CTTGAGAAAT GTGGTAACTA AGCCCAGAGA |
| | 22261 GGTACAGTTA ACCTCCCCAG AGTCACACAG CAGGTTCATG GCAAAGCTGG ACTAGCACAG |
| | 22321 GTGTCCTTCC CCTGCAGATC CCCTTCTGTG CCCCACATCA CCTCCCTCCA GTGTCTGGGC |
| | 22381 CACCTGGAGA TGGGCCCTCA GACTCACCCG GCCAGAGGTG CCATCTCATG GGAGAGGTCT |
| | 22441 GGCCAGGAAG CATCGATATT TGAGATCCCA AGAAATGAAG ACTTGGCCTG TCAGATGACA |
| | 22501 GACTTCGGTC ATGGGAACAC GTGATCTGTT TTACACATGC GTCCCCTCAG CAGCAGCTTT |
| | 22561 CCAGAACATT CCCACTTTCT TCTGTAGTGA GAAGAACTCT TTCCCTGCAG CCTCCTGCCC |
| | 22621 AACTCCTCCT TCAGTGTCTT TGCTTCAGTG TCTTTGATAA ACCATTCTGC TTTGCAGAGT |
| | 22681 GCGAGCTCTG CCTTGCAGGG TTCGCATCTG CCTGTGCTGA GTAACCAACG CTAAGGTCGA |
| | 22741 GTGGTCGGTC ACCTCTCATA AGAGCTAGGG TTGTCTCATG CTGATGACTA GGACTTGCCC |
| | 22801 TCAAGGAGAA AAATAAATCA AAACAAAAGC AAAAACAGCA AACATGCATC TCTTAAAGAA |
| | 22861 GGCTCTGAGT CCAGGTAAAT TTCCTTCCAC TGAAGCAGCC AGGCTGAATT CGAATTATCT |
| | 22921 TTGCCCCTGC TTAAAAACTA ATGCAAATTT TCCTAGAGAA TATCCACTAA TTCCTGGAGG |
| | 22981 GGGCATGGGC ATTCCTGATG CCCATGAGAG GACCATTTGC TCTTCCCTCA GTATGCTAAA |
| | 23041 TAACAGAAGC GACATTTGTT GCTGGAAAGT ATCAGTGAAG TTAATAAGGT TTTTCTTGCC |
| | 23101 CAGGGTGAGG GAACAGTTCC CAATGACAAA TGCTGTATGG GAAGGGGCTG TAGAACTGCC |
| | 23161 AGCCCCTTTG GTCCATCCGT AAAGTGAACT CTGTGGATCC TGGAGGATTC CAGCGTCTTT |
| | 23221 TTTTTTTTTT CTTTTTTTTT AAGACAGAGC CTTGCTGTCA CCCAGGCTGG AGTGCAGTGG |
| | 23281 CACGATCTCA GTTCACTGCA ACCTCCGCCT CCCGGGTTCA AGCGATTCTC ATGTCTCGGC |
| | 23341 CTCCCGAGCA GCAAGACTAC AGGTGCGCAC CACCATGCCC GACTAATTTT TGTATTATTA |
| | 23401 GTAGAGACGG GGGTTTCACT CTGTTGGCCA GGCTGGTCTC AAACTCCTGA CCTCAGGTGA |
| | 23461 TCCACCCGCC TCAGCCTCCC AAAGTGCTGG GATTACAGGC ATGAGCCACC ATGCCCAGCC |
| | 23521 AGCATCTTTC ATTTTTCTGT CTGCTTTGGC CCTTTCCTCT CTCACTGTCT TCCTTTTCCA |
| | 23581 TTTCCAAAGT CAGTCCATCT CACTATTAGC ACAAAAACTG CTAGAGCGCT TGTCATTGGT |
| | 23641 CATCTCTCCC TGCACCTGGC TGGTCTGTTC TTGGCCACTG AAGCGTTTCC CCCAGCTGTT |
| | 23701 GCTTTAATCA TTTTATTGTT ATTATGCCTT ACTTAAGAAA TGGATATGAG ATGCATTTAC |
| | 23761 CTGTCTCTTC CTGCCACTCT GCAGAGCCAG TAAGATGTGG TGGAAAGGGC CCAGGCTTTG |
| | 23821 GAGGAGGGCT GGCTGGGGTT GGATCTTGGC TGCCCCCTAC TAGCTGTGTG ACCTTGGGTA |
| | 23881 AGTAGCTGGA CCTCTCTGAG CCTGGTTCGG AATCATAGCA CCTCTCTTTC AGGGCTGCTG |
| | 23941 TAAGGAATAG CAGTGGTGTG TATAAAGCAG AGCGCACAGC CAGCAACTGG CCCCTAGCCA |
| | 24001 CACTGCTGAG CACCTACTGT GATAAGCTGC CATTGTGGTG TGTGAAGCAA AGGGGAAACA |
| | 24061 TGCCTGCTGT AGTGAGCTTC CTGTAGGGCA GGTTGTAGAA CCAGAGGTGG GTTCCAAGGT |
| | 24121 TACAAAGGGA CTCTTAGTGT ATTAGTCTGT TCTCACATTA CTATAAAGAC CTACCTGAGA |
| | 24181 CTGGATCATT TATAAAGAAA AGAGGTTTAA TTGGCTCACA TTGGCTGGGT GCGGTGGCTC |
| | 24241 ACGCCTGTAA TCCCAGCATT TTGGGAGGCC AAGGCCGGCG GATCACTTGA GGTCAGGAAT |
| | 24301 TTGAGACCAG CCTGGCCAAC ATGGTGAAAC CCTGTCTCTT CTAAAATAAA ATACAAAAAT |
| | 24361 TAGCTGGCCA TGGTGGTGTG CGCCTGGAAT CCCAGCTACT CAGGAGGCTG AGGTGGAAGA |
| | 24421 ATTGCTTGAG CCCGGGAGGT GGAGGTTGCA GTGAGCCAAG ATCGCCCCAC TGCACTCTAG |
| | 24481 CCTGGGCAGC AGACTGAGAC TCTGTCTCAA TAAAAAAAAA AAAAAAGAAA AGAAAAAGAA |
| | 24541 TTGCAAGAAA TAAATTATTG TTTATGAGCT ATATGGTCTG TGGTACCTTG TTGTGGGACT |
| | 24601 GGGAGTCTTG GCGTCTCCCT GACCCTGCCT GTTGCTGCAG CACCGCTCAG CCCTGCCTGC |
| | 24661 TCCCTACCTG CCTCCCCTCG GCCTCTCCTG CCTCCACCGG GCCCCTGGTG CCTCCTCTAG |
| | 24721 AGACAGTCCT CCTGGGACCG ATTGTGTTCT CACTTACACG AGGCATCCAG GACTACAGAT |
| | 24781 AACCAGAGGA AGGGGCGCCC CCCCCGCCTG CCCTCCTCCC TGGCATCCTC ACGCTGCAGA |
| | 24841 GGTCAGAGCC TCATCCCAGC CCCTTACCTG CCCCTACTCT GTGGAGAACC GTGGTCAGTT |
| | 24901 CGCCAGGCCG GATCCACGAA CGGCCTTGTG GAAGATGGTG AGCTCACACC CAGAGCTGGC |
| | 24961 TCCGATGACC CTGTCTCCTT TACATGTTTC TACCTTCCCC TCCCTACCTT CCCCCACTGC |
| | 25021 TGGGCGCAGA GTGGAGGCAG ATGAGGTTTA AAGCTCAGAA GGGCTTAAAC GGGTTGGGGC |
| | 25081 GCAGTGGCTC ATGCCTGTAA TCCCGGCACT TTGGGAGGCC AAGGCAGAGG ATCACTTGAG |
| | 25141 CCCAGGAGTT CGAGACCAAC CTGAGCAACA TAGTGAGACC GCGTCTCTAC AAAAAATAAA |
| | 25201 ATAAATAAAA TTAGCTTTGC AGGGTGGCAT GCACCTGCAG TCCCTGCTAC TCAGAAGGCT |
| | 25261 GAGGTGGGAG GATCGCTTGT GCCCAGGAGT TTGAGGCTGC AGTGAGCTAT GCTGGCACCA |
| | 25321 CAGCACTCCA GCCTGAGTAA CAGAATGAGA TCCTGTCTCA AAACAAACAA ACAAACAAAC |
| | 25381 AAAAGAAGGC TTAAAGGGGG CTCCAGGTGG GCTTGGCAGC ACAAAGCTAT GAAGTTCTAT |
| | 25441 CTTAGACACA AGTTCTGTTA CTGGGCCTTT GCAGGCTGGC CTGGGTACCT GGCTGCCATA |
| | 25501 GACAGGGAAC CTTCCAGATG AGCTGCAGGC GTGGAGCACA GGAGCCAGGG TGCTCTTCCT |
| | 25561 GGGCTCTGTC CACAGGCAGA ACGTACACAG TCTTTGTACA CGTCCGGCGG CTCTGGTGCC |
| | 25621 TATTTTTGTT TGTGTTTTTC TTTTGTTTGG GGGGATGGAT TTGGTTTCCC CCGAGCCCTC |
| | 25681 TGTCCTCCTG TCACCTGGCT GGTGCTCGGC AATGTTGACC AGCTGCCTGG CTGGAGTTGG |
| | 25741 CAGTGGCTAA GGCTGTGACA GCTAACATGT TCCTGAGTCC TCTCATTTCT TCACCATAAT |
| | 25801 GCCCTGTTGA GTTTGCAGAT ACTGTCTCTG TTTTTATCTC CCGGGGAAAC TGAGGCTCAG |
| | 25861 AGTGGCTAGG CCACCTTCCC ATGGTCCCTC AGCTCATGAG GGCCACACAG GGCATTGCGG |
| | 25921 TGGCCTTCTC CTCAGCCTTG ACCCTCCGGC CCCAGCATTG CTGCCTCAAG GGGTCTCCTC |
| | 25981 TGCTGAGCCG TGCACCTTCT GCCTGGCAGC TCCAACTCTG TGGCTGTGTT CAGTGGCTCA |
| | 26041 GCACTGCCCC TTGACCCTCC CTGGCCTTCT GCGGATGCCA GACTGGAGCA CTCTGACAAG |
| | 26101 GTCTGGGGTG GTTGTATGGG TCCTGTGACC TCTATACACC TCCCAGTGCC TGGGAATCCT |
| | 26161 GCAGATACAC CCTCCTTAGC CGTCCCTAAC CATAGAGGAC ATTTCTGAGG TCCCCGAGAG |
| | 26221 AGTGGGGCAC CCCTGCAGGA TCCAACTGCT GGGCCCAGGA AGGATAGCAG CAGCATGAGG |
| | 26281 GGTTCCATTA GCCACAAACT CACGGCATGG AACCTTCACC CACCTCGCCC CTCATCTGCT |
| | 26341 GTTTAGCACC TGGCACGCCG TGTATACTTA CTGATTATTA CATTTTAATG GCAAATTATA |
| | 26401 GTGGCAAACG TATGCATCTT TGCACAATTG TTGTACAGCA TGATGAACAA GTCATTAATA |
| | 26461 GTAAAGAATA AATGTGAAAG TGAGAAAAAT CTGACTGCCA AAGTTTTTAC TCCTTCCTTC |
| | 26521 CCTCCCCAGA CTTTTAAATG AAAGTTTAGG GATAATCCCT TAGTTGTCCT GCTAGTAGGA |
| | 26581 CTTGCAATTA AAAGAATTGG GCCAAGAACA CTTCTACGCT TCTCCTTTTA GGTTTGGGTG |
| | 26641 TAAATTCGGG GTATTTCTCA CTGATGAAAG CCTGGTGCAG GGCAGACCGT GGGAAGCTTT |
| | 26701 CATTTCCGGA ATGGACCATC AACATCCCTT GGAGAAGAAT TCTCTTCTCC AGACCCAGAC |
| | 26761 CTGGTGTCCT GGCACCCATT GGGCAAGTGG GTCCTAGAAG ACAAACCTGG TCAGAGCCTG |
| | 26821 GAGGCTGCTT AGCATTCCCC ACGCACATTA GCAGCTCGGA GAGCTCAGGA AGCCGCAGCC |
| | 26881 CCTCCTTGCC TCACCAGCCT GGATCAGGAC AGCATCCCCT GGAAGACACA CAGGGCCTGG |
| | 26941 CCTCTGATTA CCCAGCCTGG AGGGAAAGCT CAATCGAGCA TCATGTCACC CGGTGCCCCC |
| | 27001 ATGCAGGGTG GCACTGGTGA GACCCCCAAG CCAATGATAC CACCTCACAG GAGTGCAGGC |
| | 27061 CCATTGTGGC CAGATCATCT TGACTTTTCA AGATAAATCA GAAATCGTAT TTCCATGAGA |
| | 27121 TATCCCTATT TGCAAGTGAT GGTGACTAAA TTAGAAGTTT TTGAATATTG TAACATGTTC |
| | 27181 GTAGGCTGTT TGTCTGGTTT AAACTCTATC TGGAGGAATT CAAGCTAGAC TTCAGGAATA |
| | 27241 ACTTCTTGAG GCAAGGATTT TGAGACCTTA GGGAAAGAAG GACGTCTTGG GGGTATTCTG |
| | 27301 ACTGTTGTCC TCCTGGAAGG GAAGAACAGA GAACTAGAAG ACTGCCCTTA GCGAAGTTCA |
| | 27361 AAGCACCTAA GCCCGGGACC CTCAGCAAGT GTTCTTGAGT CACAGATTCT CCCTGAGGCG |
| | 27421 CCTCTTTCTG GCTCCATAGA ATGGCTGATT CTGTAACTCG GTGAGTTTGC TTTTTTTTTT |
| | 27481 TCCTCCATCA CCCAGGCTGG AGTGCAGTGA AGCTGGAGTG CCGTGGAGCG ATCACTGCAA |
| | 27541 CCTCTGTCTC CCAGGTTCAA GCAATTCTCC TTCCTCAGCC TCCCAAGTAG CTGGGATTAC |
| | 27601 AAGCATGCAG CACCACACCT GGCTAATTTT TGTGTTTTTA ATAGAGACGG CCCGAAGTGC |
| | 27661 TAGGATTACA GGCATGAGCC ACCGCGGCCA GCCATAACTC TGTGACTCTT GTTACAAAGG |
| | 27721 CCTTATATTT TGCTCTTTGA GGGTGGTTTT GGTTTGATGC CTGTTGGTTG CCATCTTTTA |
| | 27781 ACTAGGGATG TTTTATCAAA ATGCCCAGCC AAAGTGTCCA AACAAATTAT ACCTTAAAGT |
| | 27841 TTGAAAATGT CTGGCACTTC TAATTCAATG CCTGTTGTGC CAGGCACTGG GCTGCTGAGG |
| | 27901 AACTGAGTCC CGTCCCTGCA GGCTAGCTAG AGAACACACA CACACACACA CACACACACA |
| | 27961 CACACAGAGT GGTCTTACAA GTCAGTTTTA TATTCTACCT ATATGCAATA AAGGTATTAT |
| | 28021 TATGTTGAGG TGCCTTGATA TAAAAATTTT TCTTAAAGGA GAGGATGCCT AAAACAGGCA |
| | 28081 TTACCTGAAA CCTCCTCTCT CCAGCATTGG TTGTCTTCTG TCATGACTCA GGGTTTTCAC |
| | 28141 TGAGAATGGG ATGGAAATGT GGTCTAAAGA TAGGGCCAAT GTTGGGACTG GATCCCCTCT |
| | 28201 GGGAAGTCAG ACCAGGCTAG GGCAGGTCCT TGAAGCCATC AGGAAAAGCC TCTGGAGCCA |
| | 28261 GAAACAAAAC AAAAAAAAAA TGGTGTTAAC TAAACTCAGT CTCAAATCCT GAATAGGACT |
| | 28321 CAAGTCAAGC AAAATAATTA AAGGAGTTAG CAAAGGGCAA GTCAGAGAGA CCGAGCAACA |
| | 28381 CCAATGTCTT CCGGGAGCCC TGTGGCGAGT GACAGAGCCT GGACTCTGGA GTAGAACTCA |
| | 28441 TCTTGTGTCT TCTTCTGCCA CTCGTTAGCT GGGTGACCTT GAGCCAAGCC CCTTAACCTC |
| | 28501 TTGGACCCTA TGTTCTTATC TCTAAGTAGG GGCTGGTAAT ATCTTCCCCT TTGAGGAATG |
| | 28561 CCCTCTAAGG GGTGTTGTGA AGATTCGGTA AGGTGGCAGG GGTAGGACTC CTGGCCAGAA |
| | 28621 ACAGGCACAT AATAAATGCT AAGTCTCTCC TTCTCTCCAC CTGCTGGATG CTGTAGATAC |
| | 28681 TAAGGATTTC GATGTGAATG AGACAAAACC CCTGCCTTCC AGGAGCCTTT GAGAATCAGA |
| | 28741 GAACTAGACC CATTTCCAGA ACAAGGGGAT GCAGGGTCTG GATAAAGTTT TGGGGATCAA |
| | 28801 TAGAGCAGAG GGCTCCCAGA GGATCCCATA GGGTTGACTC CTAACTCAAG GGCATGAGAC |
| | 28861 AACCCCCAGG AAGGGCACCC TGGAAGGGGT CCGGCTGTCC CTGATTTACT TGTGGGCACT |
| | 28921 GGGGGAATGC CCGGAGCCAT CCAGCCCTCA GGGCTCTGTG TGATTCTGGG TTCCTCCCAT |
| | 28981 AAAAGATAAT CAGATTCTTT CACGTTAATG TCTTTCTCCA CCTCATTGCA CATCATGCAG |
| | 29041 CTATTCATTG ACTCAGCAAG TATCAGCTTT GCATGCGACC TTGGCCTACC CACTTTAGCT |
| | 29101 TTTAGTAATA GCTCCCTTCT TGAATAATAC AACCAGTGGG GAAACAGAAC CTAACTCTTA |
| | 29161 CCTCTGGGAG GCTTATTTGC TTTGAGAACA TATGTCCTGC AGTTTTGTTC ATATGGCAGT |
| | 29221 GAAGTTTCGT GCACACACTC TAGAGCCAGG CAGCCTGGGT TCAAAGCGCA GCTCTGCCAG |
| | 29281 GTCCTAACTG CATGAATTTG GGCAAGTCGC TCAACCTCTC CATGCCTGAG TTTCCTCATC |
| | 29341 TGTAAGATTG GAGCAATGGT AATACCTGCT TTTTAGGGTT GAGAAGAGAA TTAAATGAAT |
| | 29401 TAAGATGGGT AAAGTGCTTA GAGTGGAGCT TTGCAAGTAG TAAGTGCTAT GTAAGTGTTC |
| | 29461 GATTTAAAAT GAAAGACCCT TAAATACATT CTTTGTTCAT TTCACAAGCC CTTCATTTCA |
| | 29521 CAACCTTACA TTTCACAACC AAGCTCTGTC TCCCCTGGAA TCCAGCCATA ACTCTGCTCA |
| | 29581 CAAGTGTGAG ACAGGCCCCA GCAGAGCTGC ACGAAGAGGA GAGAAGGCAG CCCCCCAGAC |
| | 29641 TCCCAACCCC CTGTCCAAGA TGGCAAAACC AGAACACAGC CTCTGTACCA CCCCAGCAGG |
| | 29701 TATTCAGAAT CTGCAATCTC CAAAGCCCAC TTCAATTGTA AATGTAGAGC CACGTGCGCT |
| | 29761 TTAAGTCACC TGTCACTCTG GAGGCTCTTT TGCTCAGTTC CTCACCATTA GCAGGGATGA |
| | 29821 CAGGGAGTGC AGGAGTGCGG TCGACTCCCA GATATTGGAG AGCGCTGGGC TAGCTGCCCA |
| | 29881 TTCTCCCGGC CTCCACTCCT CTTTGCTGTC CAGCCATCAC TTGCTCTTTG AAGGCAAACA |
| | 29941 AAACAGAAAA CAGTGCCAAA AGTATGGGAA GAAAGCCAGC TTCTCCCCTG GGGTGCCTGT |
| | 30001 GATGCCATGC CCACCCTCCC TGACCACGCA GCCCCTGTGG ACCCTCAGGG CCCCAAGCCC |
| | 30061 CCATTTCCAT CACATGCGTA CACCCATGTG TGTCCATAGC CGCCCATCTC AGTCAATAAG |
| | 30121 GCTGCTCCTG CCCACTTGGA ATAGTGGTGA CAACCAGGAG TGGCTTATGG GAACTATCCC |
| | 30181 AATGGCCTGA CAGCATGTCC GCTGCAAACC GCTGAGGTAG GACACTGCCC TCATGTCTAG |
| | 30241 CTGATCAGCA AGAGGCGCAG TTGCTTTCTT AGGTAACATT GCTGCTGTGT CCTGGCCATT |
| | 30301 GCTGGGGGGT GGCACTTAAT CTACACCAGA ATTTTCCCTC CTGTATCTTC CAAGCTGCTT |
| | 30361 GGATCTTGGT GCTGAATTAG GTTGGACTTT GTCTTGTGGG GAAGGGAGGA CTATAGACCC |
| | 30421 TCAACGTAAG CAATGGTCAG ACTATTCTAA GAAAACTCGC CGAATTAAAG CATGAGGTAA |
| | 30481 ATTTAGTTCT GACTTCTGTC CACCCCACTG CCACTGTCCC CTTTTATCCC ATGATCCCTT |
| | 30541 GCTTTTCTTT TCCTCCTCTC TCCCTATCTC TTGTGTTTGA CGCATGATAG GAATTCAGAA |
| | 30601 ATATATGTTT GTGGATTTGT TTATTCACGT AGCAAACCAT TTCTTGAGTG CCTACCATGG |
| | 30661 GCCAGGTAGA ATGGGCGGCC CCGGGCTGCA GTGGTTTCTT CAGCCCCTCT CCAGGGTTTA |
| | 30721 CACTGTGCAA GACGGTTTGT GATGGGTCCT CCCATCGAGG ACCACACTCT TCTTTCTCTG |
| | 30781 TGCCCCTTGG TCCTCAGTCT CTGACCCCAC TTCAAAGGCA GCATTCACTC AGGGAAGCTC |
| | 30841 CCATACAATG CTAGTCAGAG TAAAAGTTTG GACAAATTGC CAGGAAGCAG CTTGTCAGTA |
| | 30901 TGCATAAACA GCCTTTAAAA TATTACTACT CTTTGACCCA GAATTTCACT TCTAGGAATC |
| | 30961 TGTCCTAAGG AAGTAGTCAC ATGCAAAAGA TTTATGTACC AAGATGTTCA TCAAAGTGTT |
| | 31021 GTTTTATAAC AGGAAGTCTC AGAAGCTGGA TAAATATCCA ACCTCTGGAA ATGGTTAGAT |
| | 31081 AGAATAGTAT GTAGCCATTA GAAAATTATG TCTATGGGGT TTAAAATGTC ATGGGAAAAC |
| | 31141 ACTTCTGACA TAAAAGAGCA TGAGAACTGT ATATTTAGCA TAATCTTAAC TATGTTTTAG |
| | 31201 AATGCACAGG AAAAAAATGT ACAAACATAT TCATAGTGAT GTCTCTGGTG GTAGGATTAT |
| | 31261 GATCAGTAAG TACTTCTGTC TCTTCATATT TTCCTGTATT TGATAATACA TGCATATGTT |
| | 31321 GTTTTTAAAA TAAGAAAAAT TTTAAGTTTA AAATTGGAGC TGAAAAGTGT TTTTAGGTCA |
| | 31381 GGCGAGGTGG CTCACACCTG TAATAGCACC ACTTTGGGAG GCTGAGGCAG TCAGATCACT |
| | 31441 TGAGCCCAGG AGTTCGAGAC CAGCCTGGCC AACATGGTGA AACCCCATCT CTACTAAAAA |
| | 31501 TAAAAAAATT AGCCATGTGT GGTGGCACAC ATCTGTAATC CCAGCTACTT GGGAGGCTGA |
| | 31561 GGCATGAGAA TTGCTTGAAC CCAGGAGGTG GAGGTTGCAG TGAGCCAAGA TCGTGCCACT |
| | 31621 GCACTCTAGT CTGGGCAACA GAGTAAGACT CTATGTCAAA GAAAAAAAAA AAAGAAAAGC |
| | 31681 CTTTTTAAAC AGTAGCAGAC ATAACTATAT AATCCTTACT AAGCTGTCGG TCAAATTTTT |
| | 31741 ATTTATATAT TTATTTTATT CATTTATTAT TTTTAGACAG GGTCTCACTC TGTTGCCCAG |
| | 31801 GCTGGAGTAC AGTGGCGTGA TCATGGCTCT CTTCAAACTT GACCTCCCGG GCTCAAGTGA |
| | 31861 TCCTCCCATC TTAGCCTCCC AAGTAGATGG GACCACAGGT GCATACCACC ACACCTGGCT |
| | 31921 AATTTTTTTT ATTTTTTATT TTTAGAGATG GTGTTTACTA TGTTGCCCAG GCTAGTCTCA |
| | 31981 AACTCCTGGG CTCAAGCTAT CCTCCCACCT CGGCCTCCCG AAGTGCTGGG GTTACCAGCA |
| | 32041 TGAGCCACTG TACCCAGCCC TCAAATTTTT AAAAATCTAT AAGAGACATT ATTGGACAAT |
| | 32101 TAGAGAAATT CACATATGGA CTTATAATAG TATCAGAGTG TGTGGTGTGA TGGTTCTGGA |
| | 32161 GGGAATGGAC TTTTTCTTTG GAGACAGGCT TTTCTATGCC CACCCTTTTA TCTTGCTAAC |
| | 32221 TTATCATCAT CCAGGTTCCA GCAGAAACAT TACTTCCCCC AGGAAATTTC TTAAGGGTGC |
| | 32281 AGTATCATGA TGTCTGCAGC AAATTCTCAA ATAGCTCAGG AAAAAAGTAC GTGTGTGGTA |
| | 32341 TGAGTGTGTG TATGTATGTG TGTATATATA TACACATATA TACACATATA TATACATATA |
| | 32401 TGTGTATATA TATACATATA TGTGTATATA TATACACACA CATACACATA TATATACACA |
| | 32461 CACACATACA TACATGTATT TTTATATAAT TATATATGCA GAGAGTGCAA ATGTTGCCAA |
| | 32521 GTTAAAGATT GGTGAGTCTA GGTGAAGGGA ATATGGTATT TATTGTATTA TTTGTGCAAC |
| | 32581 TTTTCTTAAG TTTGAAAATT TTCAAAACAA AAAATTGGAG GAAGAAGGCA TGCCAGTCTA |
| | 32641 CCCCAAGCCC TCCATTGGAA TGCTGAAAAT CTAAACAATG TGATTTGGCA ATTTCATTTC |
| | 32701 TTTTCTGTTG TGGGCCAGTA GTCCTTAGAT GTTGGGGAAG GGGGTAGTCG CTGAGGTGTG |
| | 32761 GTTGACTTAG GATGGAAGAA GCAGAAGTCA AGACTCCCAG GGTCAAAGTG GTTTGCTCTG |
| | 32821 CTGACCCAAG TGTGGGAGGC CCAGAGTCAG CGTTTCAGGT GTGCTAATTC AGCATGGTTC |
| | 32881 TATTCACGGC CAAAGTCCAC CCTGGGCACC TCTCTGGCAG CAATCTTGGG TGACTCTACT |
| | 32941 AAGGCCAGGC CTCCATGACC CTATGTCTGG ATCCCATATC TCCACCTCTC CCACTGTCTC |
| | 33001 AGGAACGGTG CTTAGCTTTT TCTTTTCCCT CTCCTGTCTT CTTTGCCAGC ATGTAGAAAG |
| | 33061 TTTAAATAAT TCCCCTCTTT ACAACAAAAC AAAACATACC CCCTTCAGTC AACCACCCTA |
| | 33121 GCTCTCTTCT CCTTTTCCCA GCCAGATTTT TTTAAAAGCA TCCTAGGCCA GGCGCGGTGA |
| | 33181 CTCACGCCTG TAATTCCAGC ACTTTGGGAG GCCAAGGTGG GTGGATCACA AGGTCAGGAG |
| | 33241 ATCGAGACCA TCCTGGCTAA CATGGTGAAA CCCCATCTCT ACTAAAAATA CAAAAAAGTA |
| | 33301 GCCGGGAGTG GTGGCAGGTG CCTGTAGTCC CAGCTACTCG GGAGGCTGAG GCAGGAGAAT |
| | 33361 GGCGTGAACC TGGTAGGCGG AGGTTGCAGT GAGCCGAGAT GGCGCCACTG CACTCCAGCC |
| | 33421 TGGGTGACAG AGTGAGACTC CGTCTCAGGA AAAAAAAAAA AAAAAAAAAA AAAAGCATCC |
| | 33481 TCAGCACTTT GGCAACTCCA TCTCCTCCCA ACATGTCCCT GTTACTGGAA TCCAGCCAGG |
| | 33541 ACTCAGCCCC GATCTTTCTA CTCTAACCAG TTGTCTCAGT TAACAAGGAC AGGTTTATGC |
| | 33601 TGCAGTGACA AACAAGATCC CAAATTCTTG TGGCTTCACA CATCTGGCAC CACCTCATCT |
| | 33661 TCCAGCCTTA GGAGTCATCT TTTAGTTCCT TGAAAACTCT TTACAGTTTT CTGTTGGGGC |
| | 33721 CTTGTCATAT ACTATTCCCC TGGAATGTTC TTTCCTATCC CCTCCCTTTC ACCTTGCTAA |
| | 33781 CTTGTGCCCA TCCTTCAGGT CTCAGCAGAA ACATCACTTC CTTGGGGAAG TTTTCTCCAA |
| | 33841 CACCCACACT ACACAGGTGT CCCATCTACA CTCCTATGAC TTTGTGGTAC TTGTCTCACT |
| | 33901 TCATTTTCCA CTGCCTTCCC CACAAGGCAC CTGCACAAGG GCAAGGACCG TACCACTGTA |
| | 33961 CCTATGTCAC TCATTGCTGT GGTCACCTGC ACTCTGGCTG CCTACCTTAA CTACACATTA |
| | 34021 GAATCACCTG AGGAGCTTTT AAAGCCACAA TGCAAGACTC CACCCTAGGC CAATTGGATC |
| | 34081 CAAATCCCTG GGGTAGGGCC AGACATCAGT GGAGTTATAT ATACATATAT ATATTTTGTT |
| | 34141 TGTTTGTTTG TTTGTTTTTT GAGACAGAGT TTTGCTCTGT CACCCAGGCT GGAGTGCAGT |
| | 34201 GGCGCGATCT TGGCTCACTG CAAGCTCCGC CTCTCGGGTT CACACCATTC TCCTGCCTCA |
| | 34261 GCCTCCTGAG TGGCTGGAAC TACAAGTGCT CGCCACCACG CCCAGCTAAT TTTTTTGTGT |
| | 34321 TTTTAGTAGA GATGGGGTTT CACCGTGTTA GCCAGGATGG TCTCGATCTC CTGACCTCAT |
| | 34381 GATCTGCCTG CCTCATCAGC CTCCCAGAGT GCTGGGATTA CAGGCATGAG CCACTGCACC |
| | 34441 CGGCCATCAG TGGATATATT TTTAAAGCAC TGCAGAGAAT TCTGTTGCAT CAGCTTGAGA |
| | 34501 ACCACTGATC TGCCTTGTGC TTCACATTTA AAACTTTTTT TTAATGAATA AATAAACCCC |
| | 34561 AAAAAATTAA TCTCCCTAAG CCTCCCTAGA AGATAGGATG GTAAGGATAT TTTCCTAGGT |
| | 34621 AAAAATATGT TAATTTCATA TTTCATGAAA TTTCATGTTT CATTTCAATC AAGCTCTGTC |
| | 34681 ATACACCTTA CATGGGGCAA GCCCAGTGCC TGGGCAGGGT GTAATTATAC TCATTACACA |
| | 34741 GGCAAGGAAA AGTCACATTA GGTGATGGAG CACAAATAGG CAGTTAATGG TTTCAGGGCT |
| | 34801 AGTTAGGATA TGTTTGTCTT TCAATTGCAA GTAATAGAAG CCCAAAGAAA TTGGTTATTT |
| | 34861 ATATAATATA ATTGATTGGT TCCCAAATTT GAAAAATTCA GGAATAGACC CAGCTTAGGT |
| | 34921 ACAGCTGGAT CCAGTCACTC AAACAATGTC ACAAAGAACC CTTTGACAGG AATGTATCCT |
| | 34981 GTGTTGACTC TACTTTGCTC TGAGTAGTCT TTCCCCAGGT GATGATAAAA ATGGTCATCA |
| | 35041 TCGCCAGGCT TGTGTCCTGT TTAGTAGGAA TATACAAGAA GAGCTCAGTA AATGCTGGCC |
| | 35101 CCACCACTAA GCAAAAACAA AACTTTTGTT GTTGTTATTG TTGTTTTAAA TAACAGCTTA |
| | 35161 GACCTTTCTT CTTTCCTTGT TATTCTCTTT CATCTGTAAT CCAGTTTTCT ACTTCTGAAG |
| | 35221 TATAGAATGT TCTGATGATT TATTCTTCAT TACCCACAAC TTGCACATGT TTATTTAAAA |
| | 35281 ATGCCAGGAT TGCCTGGCCG TTGTGTGCTG TTAACCTTTG TTTGCTGTTA GTGGATCCCT |
| | 35341 GAAGTTCAGG CTCCCAGGGG AGCAGATAAT GGGTATCCAG TTCCTGCAAT ATCCACCCTC |
| | 35401 TGGCAAGCCA AGTTCCTTCC TGGGTAAGGT TTTGCCTACC TGCATTCCTA GGGAAGTTTC |
| | 35461 TGGGCCTGAC CACCAAGCCA GCTCTGAGAA GGGGTGCATA AGCCCCACCA TGCTTTGGCT |
| | 35521 CTGTCCCTAT AGAATATTTT ATGTTGTTAC TGAAAACTAA AGGAAGATGG GTGCGGTGGC |
| | 35581 TCATGCCTGT AATCCCAGCA CTTTGGGAGG CCAAGACAGA TTGATCACTC GATGCCAGGA |
| | 35641 GTTCAAGACC AGCCTGGCCA ACATGGTGAA ACCTTGTCTC TACAAAAACA AAACAAAACA |
| | 35701 AAAATTAGCC GGGTATGGTG GCATGCACCT GTGGTACCAG CTACTCAAGA GGCTGAGGCA |
| | 35761 CAAGAATCTC TTGAACCTGG GAGGTAGAGG TTGCAGTGAG CCGAGATCGC ACTACTGCAT |
| | 35821 TCCAGCCTGG GTGACAGAGC AAGATTCTGT CTCCAAAAAA AAAAAAAAAA AGAAAAGGAA |
| | 35881 AGCTAAAGGA GAGAGACTAA AATGATATCA GGTTCCTGGA GAACAAACAG ACATGATTTT |
| | 35941 GCTTCATGGC AGGACAGCCG GAAGAAGTGG GATTATATCC TCACATTACA AATAAGAAAA |
| | 36001 CTGAGACTCA GAATGGTTAA GTCACTTGTC CCAGGCCACA CAGCCAGTAA ATTACAGAAA |
| | 36061 CAGAATTTGA ACCCAAATCT TCCAGCTCCA AAGCTTGTGT TCTTTTCACT ACCTCCTGCT |
| | 36121 TAATTTTTTA ATTTCTAAGA TTAGACCCTT CATCTATCCA TGACACCTGC CTGTCATCCC |
| | 36181 CTGAAAAAAG GTGAACGCCG TTCAGAAATT TTTCTAGCCT GAGCTCACTC CCAGTTCACT |
| | 36241 TATTTTTGCT TTGTCATGGC TGCCCAGTCC CCACTTGTAG ACCAGGAATA GGTCATGGCT |
| | 36301 GCGGGGACTA CACGCTGTCG CTGCTGCAAG GGCCGGCCTC TGTTTCCGGG GCTGAGTGGG |
| | 36361 GGCCAGACCT GCCAGGAGCA CCATCTTCTG TGGGTCCTGC CTGGATGTCA CATCCCGGCC |
| | 36421 CCAAGAAGTC ACTGCAAACC TTCGTATTAT TGAGCTTCAC ATCCTAGAAT TTGCTGTCAC |
| | 36481 TGTGGCTGCT GCATGAAGTT GTCCTGAGAG AAACGGGCAT TGTCATTAAC AGGGAAATTG |
| | 36541 ATGGTCTGGG GGAAAAGTCA TCCTCATTCT CTTGCAGATC TATGGGTGAT TGAGACTGGC |
| | 36601 TGATGTTGAA GGGGTTTCTC AGCCATCGTG TGCCATGTTA TGGAACAGTG GTGTAGCCAG |
| | 36661 CCATTTGACA CCCAGCGCTG ACCTTTGTTT AACAACCTCA CCTATATATG ACAAAATGAT |
| | 36721 TGTCAGAAAT AATCGTGTAA TGAAATGACT GTAATAATGG CCAGAAAAGA AACGCAGATA |
| | 36781 GTAAAATGTT TCTCTTGTTG AACTCTGTAC ATATAATTGC ACCAGGATTT TTTTCAAATA |
| | 36841 AAAAGTAAAT ATTATACTAC AAAAAAGGGA AAAAGCACAA GCATTTATTA AATAGCTTTC |
| | 36901 TATATCTTTC TGAGTTTTGA TCCTTTGATT GCAGACTGAT GTAATATTTT ATGTAAATCA |
| | 36961 TTGCTTGGTT ACTAAGTGAA CTTTAAGAAA AGTGAGACGT CTGCAGAAGT TGCCCATAAT |
| | 37021 TTAGCAGCTA CTGTATTGTA CCATTGATGT ACGGCTTTAT TTTCTTGATT AATTATTTAA |
| | 37081 ACAATATAAT TCACAATTTT AAAATAATAA ATTTCCACTT AAAATGGTAT TTAAACTCAG |
| | 37141 CAAAATATAT CATCTATGAG TAAAATTTGT ATTTACCAAG CAAAAATATT ACAGTTTGTG |
| | 37201 GTTCACATGC TGTCTCACTG TTTTAAATTT TAAATACAAA AACTCCAAGT AGGCTGGGTG |
| | 37261 TGGTGGCTCA CACCTGTAAT CCCAGTACTT TGGGAGGCTG AGGCAGGCAT ATCGCTTGAG |
| | 37321 TTCAGGAGTT CAAGATTTGC CTGGGCAACA TAGTGAGATC CTGTCTCTAC TGAAAACAAT |
| | 37381 TAGCTGGGTG TGGTGGCACA TGCCTGCGGT CCCAGCTACT CAGGAGGCTG AGATAGGAGG |
| | 37441 ATCACTTGAA CCCTGGGGGA CAGAGGTTGC AGTGAGGCAA GATTGCACCA CTGCACTCCA |
| | 37501 GCCTGGGTGA CAGATTGAGA CCCTGTCTCA AAAAAAGAAA AAAAAAAAAG AAACACAAAA |
| | 37561 ACTCCAGGTG GTCGCACAGA ATGACAGGAC TGAAGTAACT TAGCTCCAAT TTCTGTCTTC |
| | 37621 ATAATCACTG TCCTACCATT GTCTGTGCTT AGAATCTACT TGCTTAATGC AGGAACATGT |
| | 37681 GTTCTCACAG AGATGGAAAA TGCAAATGGC GCCAGAAGCA AGCTGGAAAT TCTGAACCAT |
| | 37741 TAAGAATTTA CTCTCTGCCA GGCACGGTGG CTCACGCCTG TAATCCCAGG ACTTTGGGAG |
| | 37801 GCTGAGGCAG GCAGATCATC TGAGGTCAGG AGTTCAAGAC CAGCCTGGCC AACATGGTGA |
| | 37861 AACTTCATCT CTACAAAAAT ACAAAAATTA GCCAGGCATG ATGGTGGGTG CCTGTAATCC |
| | 37921 CAGCTACTCG GGAGGCTGAG GCAGGAGAAT CGCTTGCACC TGAGAGGTGG AGGTTGCAGT |
| | 37981 GAGCCGAGAT CTATCTGCAC CATTGCACTT CAGCCTGGGA GACAGAGTAA GACTCCATCT |
| | 38041 CAAAAAAAAA AAAAAAAAAA AAGAACTTAC TCTCAAAATA AATACGTGTG GCTGACTCCA |
| | 38101 CATATGGTAG GGCCAACTGT ATAACTAGAA GTTCTCCAAA TAACTTCTGT GGAGAAAAAA |
| | 38161 AAGTTTATTA AAGGTTAACT TTTTTAAAGT GCTAACTAGA ACCTTACTAA CACTGAGATC |
| | 38221 GCACCAATTG TTTATAACTT AGACAGGGCC GGGTGCAGTG GCTCATGCCT ATAATCCCAA |
| | 38281 CACTTTGGGA GGCCGAGGCA GGTGGATCAC TTGATGTCAG GAGTTCGAGA CCAGCCTAAC |
| | 38341 CAACATGATG AAACCCCATC TCTACTAAAA ATACAAAAAT TAGCCAGGCA CGGTGGTACA |
| | 38401 CGCCTGTAAT CCCAGCTACT GGGGAGGGTG AGGCAGGAGA ATCTCTTGAA CCCAGGAGGC |
| | 38461 GGAGATTGCA GTGGGCCAAG ATCGCACCAT TGCACTCTAG CCCCAGCAAC AAGAGTGAAA |
| | 38521 CTCTGTTTCA AACAAACAAA CAAAAAAAAA AACCTCTTGG ACCAGGAAAA TATTTTTTAA |
| | 38581 GGGAGGAGTA TTTTATCACT GGCATTGTTT AGGATTGCAG GCACATGATG CTAATGAAAA |
| | 38641 GCAGACTAAC TATTAGTTGG TTTTATTACT GTTTTTGAAC TCTCTCTCTC CCTTTTTTTT |
| | 38701 TTTTTTGAGA CAGAGTCTCT CTCTCTGTCA CCCAGGCTGG AATGCAGTGA CTGCAGTCTC |
| | 38761 AGCTCACTAC ATCCTCTGCC TCCTCAGTTC AAGTGATTCT CGTGCCTCAG CCTCCCGAGT |
| | 38821 AGCTGGGATT ACAGGGCACC ACACCAGGCT AAGTTTTTGT ATTTTTAGTA GAGGCAGGGT |
| | 38881 TTCACCATGT TGCCCAGGCT GGTCTCAAAC TCCTGGCCTC AAGCGATCTG CCCATCTTGA |
| | 38941 CCTCCCAAAG TGTTGGGATT ACAGGCGTGA GCCACCGTGC CTAGCCCTGT TTTTGAACTC |
| | 39001 TCTAGAGACA GTCCAGCCCC TTATTACTTG TCCTGAGGCA GCTGCTCCCT TCACCTGGCC |
| | 39061 CCCCGCATTG TGTTCCGGAC CCTTGTCCTG GTGGTGCTAA AGAATATCTC TGTCGATCCT |
| | 39121 TTGGGGACTG GGGAAACTGA GGCCCAGTGC CACGCGATGC CATTTGTTCA GGGAAGATTA |
| | 39181 GGTCATCTGC TAGGTCCCCA GTCACTTGAC CTTCTTCCCA GACAGGAAGA AGCTGCTCTG |
| | 39241 GGTCTCTCAG TGCTCCACGT GTCTTTGCAC ATTGAAATGT TTTCTGATTT TTTTTTTTTT |
| | 39301 TTTTTGCTGT TACATTTACT TTTAAAAAAT AACAAGCAAT AAAATGTTAC ATTTGAGAAG |
| | 39361 GTTGAAATGA GAATTGATTT GAGTTAAATT CTAGCAGATT TTTCTTAGAA GAATGATATC |
| | 39421 ATCATCTCCA GCTACCTGCA ATTGATCTAC TCTGAATTAA GAAAGAGACT TCCATTTGTT |
| | 39481 GTTTATATTT TGCACTCTTG ATGTGTTTCT TTAAATTATG GTCATGGGCC AGGTGTAGGA |
| | 39541 GCTCACACCT GTAATCCCAG CACCTTGGGA CTCTGAGGAG GGAGGATCAC TGGAGGCCAG |
| | 39601 GAGTTCAAGA CCTCGTCTGT ACAGTAAATT TTAAAAATTA GCCAGGCATG GTAGCATTCA |
| | 39661 CCTGTAGTCT TAGCTACTTG GGAGGCTGAG ATGGGAGGAT TGCTTGAGCC AGAACTTTGA |
| | 39721 GGCTACAGTG AGTTATTTTC ACGCCACTGC CCTCTAGCCT GGCTGACAGA GCAAGACCTG |
| | 39781 CCTCAAAAAA ATAAGTAAAA AATAAATTAA ATTTCAATCA TTAGCAGTCA TTAGGATATT |
| | 39841 TAAATACAGT ATGTTGAATC AAAGTTACGC ATGTGTGTAT TTTTTTTTCC AGAGAGTTGT |
| | 39901 TTATCATGTG GGTTTTAATT TAACTTTAAA AAAATGTTGG CTGGACAGTT GCCCAAATGG |
| | 39961 TATCATCAGC CATTTGGTTG AGAACGTATG TCCTGCGGGC TCCTCTGTCA CTGGAGTTTT |
| | 40021 GCTAGCTGAC AGCCACTGGC TAGTTAGAGA CTGCAGTCAG CACAGATGCA GGCGTGGACT |
| | 40081 TGCGCACGTA ACCATGTCAA TGCAAAGCCA TCACTTCTTA AAAATTCTGA ACCCTGCTGT |
| | 40141 CTGAGATGGT GGTGCAGCGG ATAGAACTCT GCTCTAAGAG GCAGTAGCTA ATTCCATGTC |
| | 40201 TTCTTTGCCC TTGACTAGCT GAGTGACTTT GCACATGGGG CTTGCCTCTC TGTTGCCTTG |
| | 40261 TCTGCAAAGT GGAATCATCT TTTCCTTGCT AGACAGAAGG TGGACCCTGG ACCTATGGCC |
| | 40321 TTTTTGAGTT TCCCCCCCGC TTCTTAGAAG GACCTCTGAT CCTACTGAGT TTAATACCCA |
| | 40381 CGGGTTAATA ATTGGGAAAA GCAAAGGAAG CGCTTCTGTT TAGGTAATTA TATGCATGTT |
| | 40441 TTTGTCTTTT TCTGGCTGGA AAGATATCCA AGCCACTGGG AAGGTCCGTG GCTACCCAGG |
| | 40501 GTAGCCCTCT CTGGGGAGGG CTGCTATATC CAAGAGCCCC TCATGAGAAT TTGAAAATCG |
| | 40561 ACCATGGTAG GGCCTGCTGA CTTTTGACAG CTAATGGTGT GCTGAGAATT GTCCCTCCAA |
| | 40621 AGATGCCTTT CCATTCCCTC GGGAGAGTCT GGGCAGCCCC TACTGGGGGC TGGGATGCTG |
| | 40681 GCTCTTCCCT CAGCCTCCAC CCCAACTGCT CTCTTCCCTC CTCCCCTCCC CAGCCCCCTA |
| | 40741 ATTTCTCTCA CAAGGCTTTG TTCTGCAGCA ACCTTTCCTA ATGCAGTCCT GGCCTCTTCG |
| | 40801 CAGCTTCATT ACATAACCTT CCGTGGACTC CTGGTCCAAG GATCACCCCA GAAAGCCAGT |
| | 40861 CAGAGGTAGG CACGCAGCTG GGGTCCATTT ACTTACCTTC CCCACCCCCT CGGAACTCAG |
| | 40921 AGGTGGTGCA GGAATTTGGA CTCCAAGAAT TAACAGCTCC ACCACCATCA CCAGAGCCAA |
| | 40981 AACTCAGGAT GCATGTGCTT CATCTGCTGC TTATTTCCAG CTGAGAGCCA GTGGTGCCAT |
| | 41041 GGTTCCTTAG GGAGCCGGTC CCCTGATGCC GGCTCCTGGC CCCAAATCTC TCTGATCCGG |
| | 41101 GCTCTTCCAG AATGTCTTGT CTCCACCATC GCCTTTGACC AATGGTGTCC CTTTGCCTGG |
| | 41161 TAATGTCCCC TTTGCCTGAT GATGGCCCTG TCACTCCTCT CTTTAGCACA GAGGAGGCTG |
| | 41221 TTTCATCCCT TCAAGCCTGC CCTCCCTTCA AGTCTTAGCT CAAGTTCACC TTCTCCGCAG |
| | 41281 AGCCTTCTCC AATCTTCTTG ACTACGTCTC CTCTCAGCTC CAGCAACCTC TGTCTCTGGC |
| | 41341 ACTGATTCCT TACTTAGCTA AGAGAATCAC AGACACTTGG GGCTCAGGAC AATCTGCTTT |
| | 41401 CTCTCTTCTT ACCCATGGCC TTGGACTGTG TGTACCTCTT TGTCTCCACT CCCAAACCCA |
| | 41461 ACCCCCAGAG GGCAGAGAGC ATGTTGTCTG TCCCTTTGCT CAGCATGAAG CCATGCGTGT |
| | 41521 GGTAGATCGG CAGAGTTCCA TAACTTGTGT TGACCGAGGG GTCACTTTGC TCTGAAATTA |
| | 41581 CCCCTGTGTC CTTCAGTATT TGCACAGATA GCTTCCTGGC CAGACCGAAT ATATCCAAGG |
| | 41641 GCATGGCCCA CCTCTGCTCC TGTTTCCAGG TCCCTGGTGG GGGTTAGTTC ATGCCTTCCT |
| | 41701 CATAATCTGC CCACTGGCCT GGTCCTCAAG GTCTTCCCAA CTGCTCAGCC AGAGTTGAGA |
| | 41761 AAATGGGTCG CTCCATCCTG TTTGTGTCGT TCTCTCCTTC CTGGCCCACT CTCCTGCCCA |
| | 41821 CAGGTATCCA GGGGCTGCCT GTAGCATTAG AGGACATACA TGCACATGCG TGGGCATGGG |
| | 41881 ACACTCACGT AGCCTCCAAG CACAGCATCA ATAATGCATT CTGTGCTTTA TAGCATGGAA |
| | 41941 AGCTGCTCTA AACTTTATTA CACAGTGGAC ATGTCTGAAG CAGCTCCCAA ATCCACCCCT |
| | 42001 GAGTGTGTTG GAATTGGCAA GCCTATCACT TGGGAGTCTA GTTTTTTTGT TCGTTAATAA |
| | 42061 TAGATGCTTC CTGTGGCCCC AGCTTGGCAA TTTTGATTTA AAGTGATCTT AACTGAAGAG |
| | 42121 ACTAATGGAC GGGTCTGAAT TTGTGCCTTT TAAGCACAAA GTATTGCTCT TAATTAACTG |
| | 42181 GATTCTATCC TTTGAGCAGG CAGAGGCCTT CCCCCAAGGG CGTCATTAAC GATCCACATC |
| | 42241 TGGACATCTT CCAAAGCCTT CTTCTGTTTC AGGCCAACCG CAGGTGTGTT CCTGAACACC |
| | 42301 CAGGAGGCTA TGAGAGCCAC ATATGCCTCC CAAATACACA CAGTGTGCAT GCCCAGGGAC |
| | 42361 ATAGAGCAGT GTGCAAAGTC CCATTCCATC TCTCTCCACC TGGGAGAGGA TGGCTCTTCT |
| | 42421 GTCTGATTCA TGGCTCAAAG TGGTAAAGGA GCTCCCCACT CCCCGTCCCA CGCCTACTCA |
| | 42481 GAGTCTGCAA ATATGTATGC GATATGAGAG CTCGTCAGTT AGCTGTCTTC AGTGTGGCGC |
| | 42541 ACATTTGAGG AGTCTGACTC CCCTCCAGCA CAGGCCAATG TGCACTGCTC TCCTATCTTT |
| | 42601 GTACCCCCAC TGTTGCACTG TGCAGAGGTT GGAGCCATAG AAGTACCAGA GCTGTGAAAG |
| | 42661 GAGAGGCCCC CTCTCACCTC TGCCCTGGTC TCCATCCCCA CTTTCTCTAG GAAGCTAGTA |
| | 42721 GGTGCTGACA GGGGAGAGAA GGGAGGGGAG GGGTCCAGAA ACAGTGGCTC ATGCCTGCAA |
| | 42781 TCCTAGCACT TTGGGAGGCT GAGGCAGGAG GATCATTTGA GGTCAGGAGT TTGAGACCAG |
| | 42841 CCTGGGCAAT GTAGCAAGAC CCTATCTCTA CAAAAAGAAA AAATGTAATT AGCTGGGTGT |
| | 42901 GGTGGTGGGC ACCTGTAGTC CTAGCTACTT GGGAGGATGA GGTGGGAGGA TTGCTTGAGC |
| | 42961 CCAAGAGTTT GAGGTTACAG TAAGCTGTGA TTGCACCACT GCACTCCAGC CTGGGCAACA |
| | 43021 GAGCTGAGAC CCTATCTCAA AAAAAGAAAA AAAAAAAGAA AGGAGAGAGA GAGAAAGAAA |
| | 43081 AGAAAAGAAA AAAAAAAAAG AAGGGAAGGG AAAGCCCAGA AGAGTGTGGG GAGAGGAGGC |
| | 43141 GGCCGTCATT CTGGGGCCCT CAGTGTGCAC AACCAGATAA CACATGCTCT GTGGGCTTTT |
| | 43201 GTACCATTTT GCTTGAGCAT AAAGAAAGGA AGGCTGCCCC TAAATAGAAA GCACTCTGGA |
| | 43261 GGCAAACAAA TCTGACTCCA ATCCTGGCCC TGCCACTTTC CCAGCTGAGG ACTTAGACAA |
| | 43321 GCACCCTAGC CTCTTGGACA TTCTCAGAGC CATCTGCTGC AAGTGGGTGC TGCCATACCC |
| | 43381 ACCTTACTGG GCAGGCTTGG GGGACCAAGG GTGGTAAATG GCTCAGTCTT TCATGATGCG |
| | 43441 GCCACACAGC AGGTGCGCCA TCCAGGTCCA TTTCTTTCCT TCCTTTCCCC CAAATCAAGT |
| | 43501 TGTCATTAAA GTACTAGTCC ACATTAATGA AATCAACTGT ATTAATTTTC TATTTGCTGC |
| | 43561 TATAATAAAT CATCAGAAAT TTAGTGGCTT AAACCAACAC AAATGTATTA CCTTACAGTT |
| | 43621 CTGGAGGCCA GAAGCCCTCC ATAGGTGTCA CTGGGCTGAA ATCAAGGTTT TGGCAAGGTT |
| | 43681 GCGGTCCTTT CTGGAGGGTC CAGGGGAGAA TCCATTTTCT TCCTTTTTCC AGCTTCTAAA |
| | 43741 GGTTTCATGC ATTCCTTGGC TCATGATCTT CTATAGCTAT AGTCAGAAAA ATTTTCCATC |
| | 43801 AATCATCTTC AAAGCCAGCA ATGGCAGGAT GAGTCCTCAC ATCACCTTGC TCTGACACCA |
| | 43861 GTTCTCTGCC TCCCTCTTCC ACATGTCAGG ACCCTCATGA TTACTTTGGG CTCACTCTGA |
| | 43921 TAATCTGGGA TGATCTCTCT ATTTTAGAGT CAGCTGACTG GGAACCTTAA TTCCATCTAC |
| | 43981 AACCCCAATT CCTCTTTGCC ATGTACAGTG ACATATTCAC AGGTTCTGGG GATTAGGACG |
| | 44041 AGCCTGTCTC TGAAAGGCTA CTTTACATGA AAATTCATTT TTTTAATTAA GATTTTTTTT |
| | 44101 TCCTCTTGAG ACAAGGTCTC ACTCTATGGT TCAGGCTGGA GTGCAGTGGT ATGATCACAG |
| | 44161 CTCACTGCAG CCTCGACGTC TCTGGGCTCA GGTGATCCTC CCACCTCAGC TTCCCTAGTA |
| | 44221 GCTGGAACTA CAGGGGTGAG CCCCCATGCC CAGCTAATTT TTTTTTTTTT TTTTTTTTGA |
| | 44281 GACAGAGTCT CACTCAGTCA CCCAGGCTGG TGTGCAGTGG TGCAATCTCA GCTCACAGCA |
| | 44341 ACCTCCGCCT CCTGGGTTCA AGTGATTCTT GTGCCTCAGC CTCCCAAGGA GCTGGGACTA |
| | 44401 CAGGTGTGCA CCACCACGCC CGACTAATTT TTGTATTTTT AGTAAAGATG GGGTTTCACC |
| | 44461 ATGTTGGCCA GGCTGGTCTC AAACTCCTGA TCTCAAGTGA TCCACCAACC TCAGCCTCTC |
| | 44521 AAAGTGCTGG GATTACAGGT GTAAGCCAAC ATGCCCGGCC CCAGCTAATT TTTAAATATT |
| | 44581 TTTTTTGTAG AGATGGGGTT TTACCATTTT GTCTAGGCTG GTCTTGAACT CCTGGGCTCA |
| | 44641 AGCAAACCTC CCACCTTGGT CTCCCAAAGT GCTGGGATTA CAGCATGAGC CACTGCACTC |
| | 44701 GGCCTTAAGA GAAGATTTAA TAATTAATAC TTTACAACAA GATCTGGAAG AGGTGGGATG |
| | 44761 AGTAACTAAA TGAGGATACA AGTAACCCGG GTCATATTTG CTAATACCCT TGGTCACATT |
| | 44821 GAACTTGATA TCTTATCAGA TTTTCCTAAT CAGCTCCTTT AGCAGCAGTG TTGCAGCATC |
| | 44881 TTATCTCATT TTGTTTTTTG TTTTTTTGCC TAGCACATGC CTGTAAATCA CTGGATTGAG |
| | 44941 GTGTTTAGAT GTTTGTTGTC CTTTGGATGC TTCTTATAAA TCCATATTTC ATGGCTCCCT |
| | 45001 GGAAAGTGCT ATGCAAATGA TAAGCTGCAA GGATGGAAAG GAAATTGCAG TGCTCCTGAA |
| | 45061 TTGTAAATGG GCTTTTACGA GGAGGTTTCT AATTACTCGC TCTTTCTCTT GAACTGAGGA |
| | 45121 GTTGAAGTGT AGGTGGCAGA TCCATAACAG ATAATCATGT GTGTGATGTG ACTTCAGCCT |
| | 45181 GAGCGTCGAG GACCAAGTCA CAGAGCAGGA ACAGCCACTC TCCAGTGTCC TTGGGGCTAC |
| | 45241 GTCTGAGGAG AACCTGGGAT TTCATATATG ACCTGCACTG GCTGGGGGGC TCTCTTGACG |
| | 45301 TAACGTGTTC CCTCTGAGCA TGTTACAGAT TCTGACATTC TTATGTTCCT TCTGTGGAGA |
| | 45361 GACATGTACT TAGTGACCTA ACTCACTTTA GCATATTTTT GCTCATCGTT TGTGTAGCTT |
| | 45421 AAAGGAATCA GATAATTACC CCCTCCCCAC TACTTTCGGA AGCACAAATG CAATGCCCTA |
| | 45481 GAATTGTACT GGGGACTCAA AAAGAAAAGA GAGTAGTAAA ATCTATTAAA GGGGACAAAG |
| | 45541 ACAGCCTATA TACTACAAGC TTTCTATTTT TATGGCAGAG AATGCCATTT TCTAAGTAAA |
| | 45601 CAGAGAACTG CATTTGACCT GCAATATCAA ATGCATGGAT TTGATGCTTT GGAAAGCAAC |
| | 45661 TGTTTTCTGC GTTAATCTGG GTGTCTTCCG TGAAATGTCC TCCTGCCTTT GGCTTAAACA |
| | 45721 CTAGCTTTGT CTACAGCCAT TCCATCCTGA ACCTGCCCAA TCTTGTCTGA ATCCTGGTTT |
| | 45781 CACCACTGAC AAGCTGTGTG TCCTTGGGCA AGTTACTTCA CCTGTCTGTG CTTCAGAGTC |
| | 45841 CTCATCTGTG AGTTGGGGAA TCTGGACAGA ATCTACCCCA TAGGGCGTAG TGAGGATGTG |
| | 45901 TTGAATTATC CCAAGTGGCT ACACAGAGTA AGCACTCAAA TGATGTCATC GTTGTCATGA |
| | 45961 TTGCTGTTAC CAGAGCCTAG AGTTCATTCT GATACTCGAG TCTGTGGCCC ATCCAGCCCA |
| | 46021 GGTAAGGAAT AGTTGGAGGA GTTGGGCATG TTCAGCTTGA AGAGGAGACG ACAGGGGATA |
| | 46081 TGGGATAGTT GAATCTGTGA AGGGCCCCCT GGGATGAAGA ACTGGCATGT TCTGTGTGGC |
| | 46141 TCCAGGGCAC TGAGCAGGAC CCATTTGCCA AAGTCTCAGG GACACAGTTT CTAGCTATAG |
| | 46201 ACAGAAAAAT TTTCTGTCAC TCAGAGGATG AAAATAGAAT GAGCCCCCTT AAGAGGTAAT |
| | 46261 GAGCTCCCTG TCATTGGAAG GATTCCAGAA GAGCTAGGTA ACCACTTTAG GTGCTATCAA |
| | 46321 GGGGCTTTTT TCTTTAAAGT CCTTTCCAAA AGCTTCTGAG ATTGCATAAA CAATAGGAAG |
| | 46381 CCATCTTGGT GCTTTAACAC AAACTCTCCC CAGTGATGAG GGTTGAGCCA AAGCCAGATT |
| | 46441 GGCAAGCAGA GAGGAGACTT GTGTACAAGG AGTTCCTCGA GTCAATTGCT TTTTCCTTGT |
| | 46501 TCTAGCCAGC CAGAGGGCTC CTGTTGGAAA ACAGGAGACC GGAGAGGCTG AGGCCTGACC |
| | 46561 AAACCAGCTT CTGCAGGCCA GCTGGGAGGC CACAACTCCT ACCTACGGGA AAACTGAAGG |
| | 46621 GCATCTCTAT TTTTAGATTA GCAAAAGAAA ATAAATTTAA GTTTGAGTCT CCTTTGCAAC |
| | 46681 TTTTAAAAGA CATCTTTATT GAGATGATCA TTCACATTCT ATAAAATTCC CCCACTTTGA |
| | 46741 GTTACAATTC AGTGGTTTTA GTCTTCCTTG ATGATTTTGA TGGTCTTTTC TTAAGGCTCT |
| | 46801 TGGAAGACCC AGAAGCCTCT CAGACACAGG TGGGTGTGGA GGGCGTAGCA CAGAGGCAGA |
| | 46861 CTTCTCATTT CCTGGGTCTC CCCTTTAATG ACTCTCAGAG ACCCCTCCTT CCCCCTGCCC |
| | 46921 CTGGCTTCTA CCCCAGGGGT GTAGAGTTTT GCCATTTTCC AAGCAGAACT TCATTTCCTC |
| | 46981 TTCTGTGTCT ACACTCTTTG TGCTTCTTTC TTGCCAGCTT TTTCTCCTTT GCCCGCCCTT |
| | 47041 CCTTCCTTCC TTCCCTCCCT CCCCCCTTCC CTCCTTCCCT CTTTCCCTCC TTCCCCCCTT |
| | 47101 CCACCCTTCC CCCCTTCCCC CCTTCCCTCC TTCCTTCCTT CCTTCCTGCC TGCCTTCCTT |
| | 47161 CCTTCCTTCC TTCCTTCCTT CCTTCCTTCC TTCCTTCCTT CCTGGTATGT GACTAATTTC |
| | 47221 TGTTTCAGGA CATAAATGTT GTCCAGGCTG TTCTTTGGTC TTTCTGTTGG ATAATGGACA |
| | 47281 TTTGGCATTG AGAGAGGCTG CTTTTTCTGA AATCATGTTC TTGGGGCCCA GAACCTAGGT |
| | 47341 GTGTGCTTCT GACTTTGTTT TCTTCCTGAT CCAAATTCTG ATATGTCCAT TTAAATTGAT |
| | 47401 CTAGACCCAC AGGGCACTGT GGGACAGATC CTCAGTGGAA CATGACTCTG TAACGAGAGC |
| | 47461 ATTTTGTTTT GTCAAAATGA GAACATATTA TTGCCTTTCA TCTGATTGTA AACATAATAC |
| | 47521 ATGTTTATAA AACAGTATAA TGAGACAAAA ATGTAGACAC TAATAAGGGA AAATCTCCCT |
| | 47581 AATTGTATTT CTCTTCACAG AGAAAGCCCC TGTTGGGCAT ATATACTCTA GTTTGTTTAT |
| | 47641 TTGTTTGACT ACACATATAT GTATTCTTTT CTTATGTATA AAAATTCTGA ACATGCACAT |
| | 47701 TTCTGCAACT ACTGTTTTCA CTTGATGATG CATGGACCTC TCTAGAGTGT ACGTTTCTTC |
| | 47761 TTCCTTACAA AGCAGTTGGC TTCGCCCAGG GTGCACCAGG ACACGGTTTT GGCTCTGTCC |
| | 47821 CCAGGGTGTC ACGGGACCAG GGGATGATCT CACAGGGTCT GCCATCTGCC CTGCCTGGCC |
| | 47881 GGAGGCTGCA TCGAGAGGGC CAAGGGGCAC CACGTGTCGT GGGTACTGTC AAACAAGAGC |
| | 47941 CTTCAGAGCC TTCCACAGTC TTTCTTTTGC TTCCCAGCAT TGCTTCCCCG CTGGTGGACT |
| | 48001 CTGAATCTAG AACTAGCTCC AGGCGCCTCT CCAAATTCAG ACGGGAGCTG GGGCACTATT |
| | 48061 ATAATGCAAA TCTAGGCAAA GCCCTCCCAA TACCAGGATC CAGAATGGGG TGGGGCCCTT |
| | 48121 TGCCCTGAAA AGCTGTTTAG TTTGAAAATA CAAACAGGAG ACAGAAAAGT TTGGCTAAAT |
| | 48181 TAATGGATAA AGTTTTAACG ATGGTAACCA TAGTAGGGTT CATCGACAGC CAGCGATGGT |
| | 48241 TCTGAACACT TGACATGTAT TAACTCACCT AATCCCCACA TTTTACAGAC AATGCAAAGG |
| | 48301 AGGCTCTGGG AGGTTGAGTG ACTTGCCCCA AAGTCGCACA GCTCCTAAGT GAAGGATTCG |
| | 48361 GAGTGGACTC CAGGCAGCCT GGTCTGACTC CCTGCACTGC GCTGTGCTTA TCTCTGGCCC |
| | 48421 CAATGCCGCC ATGCAGAAGT GTCTGGGGGC ACTTTGTCTC TGTCAGACAG AATTCGGAGA |
| | 48481 TGTGTATGCT TGCCCTGGTA TGGCACTTCT CTTTTTTTGA GACAGAATCT CACTCTGTCA |
| | 48541 CCCTGGCTGG AGTGCAGTGG CATGATCTCA GCTCACTGCA ACCTCCGCCT CCCAGGTTCA |
| | 48601 AGCAATTCTT GTGCCTCAGC CTCCCAAGTA GCTGGGATTA TAGATGTGCA CCATCGTGCC |
| | 48661 TAGCTAAATT TTTGTACTTT TAGTAAAGAT GTTGTTTTGC TGTGTTGGCC AAGCTGATCT |
| | 48721 CGAACTTTTG GCCTCAAGTG ATCTGCCTAC CTCAGCCTCC CAAAGTGCTG GGATTACAGG |
| | 48781 CATGAGCCAC CATGCCTGGC AGTGTGGCAC TTCTTACGTG TGTTCAGCGG ACACTGTTTA |
| | 48841 TCTTCTGTCC CTCCAAGACG GTGCTGAGCT CAGGTCGTTC ATTACTGGCA GACAACTGCT |
| | 48901 GATTTCCAAC AGAATTGCCA TCCTCTTCTC CCCTGCGACT TTCAGAGTGT GACCTCAGAC |
| | 48961 TCAAAAATTA GAAGTGAAAA CATCTTAAAA ACTATCACCT TTTCTTCCTA ATCCTCCTCT |
| | 49021 CCCCTCCCTG TCTTCCTTGT TGTCCCCATC TAATGAACTA TCATGGCAAA AAGAGCCCAT |
| | 49081 TTCTGGTCAT TTTCTGTGGC CTTTCAAACT CCCACCTACC CCACTGCTCC TGGGTGCATT |
| | 49141 ACCCGAAAGC TGAGACTTCA GTGCAGAAAG TGCCAGGCCC TCTGTCCCCC CAGATCGCCT |
| | 49201 TCCTTGTCTT CCCTGTGCTT GCCTGTCACA TTGTGTGGGT TCCAGCGCTG GAAGGAATGA |
| | 49261 GGAACAGATT CTCTGGTTCT CCTTTTGAAG TTTACCTTCG CTCCACCACT TCTGAGACCT |
| | 49321 TCCCGGAAGT TGCCCCTTGT TTCTCTCCTC TCCAGGGCTG CCCCAGAGCT GCCTCTCACC |
| | 49381 TCTTCCTGCT GTCACCCCAC CACCATCAGG GCAGAAGTTG GGACAAAGCC TCTCCTACTG |
| | 49441 GCTCCTGCTT TTCTCCCTTA GGTCCAGCCT CCTCTTCTCC ATCTTCAGGA GTCTCCTTCT |
| | 49501 CCACTCACAC GTCATGACTT CAGCACCTCG CATCAGTCCA GAATATGACT GCTTGTTCAA |
| | 49561 GTGCCACCTT TCTCATGCAT TTTTTTCTAG TGACAATCAC AGCCACCCTG TGGGGCAGGA |
| | 49621 GTGTCATCAT CCCCATGTTT CAAATGAAGA ATTGCAGTTC AGAGAGGGCA AGTGACTGGC |
| | 49681 CCAGCCTCAA CAGCTAGCCA GTGGACCCCA CCAGGGCTTC TGACTCCAGT CCGGGTTCCC |
| | 49741 TTTCCACCCA AATCCATGGA GGGAGCTGAG CCGAGAACAG GTGTCCTTCA GGAAGACGTG |
| | 49801 AAGCCAAAGC CTCCACCTCC AAACTCAGGG GCCCAGGGAG TCCAGGCACC CATCCACTCA |
| | 49861 CAAGGCTGGA TATGGTGCAT TCCAGGAGAG GGGTTGGGGG CGAGTGGCCT CTCTGTGTAC |
| | 49921 CCGTGGGGAT AGATGCGCAA GTGGCATCGC CACATCGTGA GTCCTGGCTT CATGGGTGAG |
| | 49981 CTCCAGGTCC AACGAGAAGC CAAGCAGGGG GCCCTTCAAG CTCAGCTTTG GGCCCGGGTC |
| | 50041 GGGGTACAGG GTAGAGCGGG CCTCCCCAGC CCCTGCCATG AGGCCAAGGC AGTGCATCGT |
| | 50101 TCGCAGCGTA CATTCAGAAA CCAAAGCCTA GGAGCTGGTT ATCATTCCGG TTTACAGCTG |
| | 50161 ATGGAAGAGC AGGTGCTTCC GAGAACCCAC AGTGCTCTTT GGCCAGTGAC CCAAGGGTGC |
| | 50221 CTCTGAGAGG CCTCGCAGCA CCCGGAGGTG CTGCTGAGGC AACGCCCTGA CTGTAAGAAG |
| | 50281 GACCATTCAT CCTCAGAGAG TGGCCGTGAT GCTGCTGCGA CAGTCCCACC ATCCCTCCCG |
| | 50341 ACTCTCACTC CCAACAGACT TCCCACTGTA AAGCTGAACT CTCCAGCAAA TCACCTCTCG |
| | 50401 CCAGACTCTC TCCTCACTCT CTCTGGGTCC ACTAGAGGTT CCTCAGCCTC TCTTTGCCTT |
| | 50461 GGTTTTCCCA GCTGTAAAAT GGAGCAAAGA GGGCCTATGT ACCCACAAAG GTGTGGTTGG |
| | 50521 AGCGACTCCT CCTACATTAG GGCCTCGAGT GGGGCTTCAT GATTGGTTGG TGGAGGTCTC |
| | 50581 CAAACCCACC CAGTGCCACC GAAGGCTGAG ACTGCAGATG CAATGCCACA GGTGTCCTTC |
| | 50641 CTCAGCCTGG GCAGCTGAAC ATCATGTGTA AAACGGGGAT AATAAGATAA TAACAGCCCC |
| | 50701 TTGCACCTAT GTGGCTGTGA GGATTAAACA AGATAAATGT GTAACAGTGC CTGGCTATAG |
| | 50761 AAATATTTAC TCTTGTTATT AAGGGAAGAA TATGTGTGGC TAAAAAGGGA TCGAAGATGT |
| | 50821 AAAAGCCAAT CCCTCCCCCT CTAGCATATT TAAGGGTAAT GTTGAGTTGG TTTGTGGACC |
| | 50881 ATTTGCTGCC TGTTAGAGCT GGAAGGTAGG GACCCCCTCT CAACAGCGAT GCTACAAATT |
| | 50941 ATACCCATTG GAGGTCAACC AAAAGACAAA GCTTATTGGC TGGACATGGT GGCTCACACC |
| | 51001 TGTAATCCTA GCACTTTGGG AGGCCAAGGC AGGCGGATCA CTTGAGATCA GGAGTTCGAG |
| | 51061 ACCAGCCTGG CCAACATGGT GAAACCCCAT CCCTACTAAA AATACAAAAA TTAGCTGGGC |
| | 51121 GTGGTGGTGC ACACCTGTAA TCCCAGCTAC TCAGGAGGCT GAGGCAGGAG AATCACTAGA |
| | 51181 ACCCAGGAGG TGAAGGTTGC AGTGAGCCGA GATCGCACCA CTGTACTCAA ACCGAGGCAA |
| | 51241 CAGAGGGAGA CGCAATCTCA AAAAAAAAGA AAAAAAGACA AAGCTTGTTA ATACCAGCAT |
| | 51301 ATTGTTAAGG GAATAAAGTA GGCTGCAGAA CAACTGGTGT AATATGGTGC CATGTAGGGA |
| | 51361 AAATTACATG TGTGCATAGG AGAGGGGTCT GCAAGGTTGT GCCCTAAGAT GTTAGAGTGG |
| | 51421 TTCCTTTGCT TTTCTCTTTT ATAATTTTGT ATTTGACTTT TAAATAAGGA CCATAAATCA |
| | 51481 CTTTTATAAA ATACATTCTC TCCAGCCCCT ACTACTCCTT TAAAGAATAA GAGTGGTTTG |
| | 51541 CCCAAGAAAG ACAGTTTTTT TTGCTCTGGT TTTCTTGATT CTGACATCAG AGGAAACTGC |
| | 51601 TTCTCATCCA CTTGGGGCTC TGGGTTCAGG GGATTCATTT CAGGCAGATT AAAGTGGTGA |
| | 51661 CCAGGGGCAT TCGTGGACAC AGGGAGGGAC AGGAGCACCA TCAGTTTGTC TCACACAACC |
| | 51721 ACTGTCATCC TCACTGAAGG CTGTTGCCTG ATCAAAAACA GTATTGGGCC AGGCACGGTG |
| | 51781 GCTCACACCT GTAATACCAC CACTTTGGGA GGCTGAGGTG AGTGGATCAC TTGAGGTCAG |
| | 51841 GAGTTCGAGA TCAACCTGGC CAACATGGTG AAACCTTGTC TCTACTAAAA GTTCAAAAAT |
| | 51901 TAGCCAGGCG TGGTGGGTGC CTGTAGTCCC AGCTACTTGG GAGGCTGAGG CAGGAGAATT |
| | 51961 GCTTGAACCC GAGAGGTAGA GGTTGCAGTG AGCCGAGATG GCACCACCAC ACTCCAGCCT |
| | 52021 GGGCGACCGA GGGGGACTCT GTCTTAAAAA AAAAAAAAAA AAATATATAT ATATATATAT |
| | 52081 ATATATGTCA AAAATGGGGT AGTTTTTAGA TCTATAGTAG TTCTAAAAAC AAAGGCCATC |
| | 52141 CAAGCATGAC AGATTTACAA GCACTATTGG CTATTCCAGT AGTTACAATG GAGGAGAGAA |
| | 52201 GCTTTTAGTT AAAACAAACA AACAACACAA CAAACCCAGA AACCTTAGGT CAAAACCAAA |
| | 52261 ATTGTCCTCT CAGACACAAT CTGGGAATTT TCTCATGACA GTGGGCATTA GCCAACTGAC |
| | 52321 ATCAGCAGCA ACCATCCGTG TGCACACAGT GGCACCACCT CCTCCCAAAA AGCAGCCTTC |
| | 52381 ATCTATGCCC TCATACAATC GTTGATTATT CTCTTTGGAT TGAGGCCCGG AATTATTTAA |
| | 52441 GTTTCTTCTT GCCAGCATGA GTCTTTCCTT TCTGTATGCT CCTTATCTTC TCTCTTTAAT |
| | 52501 TTGGCAGTTC TGCTTGAAAT CTGGGTCTTT CATTAGTAGT AGTTCAATTT GGTTCCAGAA |
| | 52561 CATTCTGTGG TGTGATGCAA TGTGACCAGA GCTCACACTT CAGAGCTCTT CAAGGGCCAG |
| | 52621 TCTTACTGAG CACCTCCCAG TGGCTGCCTG TGTGCTGGGC GCCACTTGTG GTGGGCAGGA |
| | 52681 GAGAGGAGGG GACACAAAAG GAGACACAGC TCCTTCTTAG AAGCTCAAAG TTGGGGACCA |
| | 52741 GCTGCCACAG AAGAGTATGT TTAGCATCTG AGACACCAAG ATCCAGCGTC ACAAGGGTGT |
| | 52801 TTATTAAGCC TCCTCATCTC TTTCTTTTTC TTTTTTTTTT TTTTTTTCCT CAGGCAGTCT |
| | 52861 TACTCTGTCA CCCAGGCTGG AGTGCAGTGG CATGATCTCG GCTCACTGCA TGCAACCACC |
| | 52921 ACCTCCCGGG TTTAAGCAAT TCTCCTGCCT CAGCCTCCCC AGTAGCTGGG ATTACAGGTG |
| | 52981 CCCACCACCA CACCCAGCTA ATTTTTGTGT TTTTAGTAGA GACAGGGTTT CACCATGTTG |
| | 53041 GTCAGGCTGG TCTCGAACTC CTGACCTCAG ATGATTCACC CACCTCGGCC TCCCAGTGTG |
| | 53101 CTGGGATTAC AGGTGTGAGC CACCGCGCCT GGCCTTGCTG TTGATTCATC TATAGTATGT |
| | 53161 TTGACTTGAT GACCTCCAGT TACCTTAGAC AGAGGTTCTC ATCTAAGCTC CAACTTTCCA |
| | 53221 TTTCCTTTGT CCTCGTCTTT CCCCTTAACC CCTCCACATT TCTCTCAAAA TCACCCCACT |
| | 53281 TCTAAAAAAT ACTGTTTATT TTTCTTTTAA ATTTCAAATT ATCTATACTC ATTGAAATAA |
| | 53341 ATCAAAATAG CATGGAATAA GCGAAAAAAA TGGATCCCAC CCTTCCCCAC TCCCATTCCC |
| | 53401 TAGGGCTAAC CATAGTTAAC CATTTAATGA CTAGGTTTTT TTGTTGTTGT TATTTTTTAT |
| | 53461 TTATTTATTT TGAGACAGAG TCTTACTCTG TCACCCAGGC TGGAGTGCAG TGGTGTGATC |
| | 53521 TCGGCTCACT GCAACCTCTG CCTCCCAGGT TCAAGCATTC TCCTGCCTCT GCCTCCTGAG |
| | 53581 TAGCTGGGAT TACAGGTGCC TGCCACCACA CCTGGCTAAT TTTTGTACTT TTGGTAGAGA |
| | 53641 CAGGGTTTCT CAATGTTAGC CAGGCTGGTC TCGAACTCCT GGCCTCAAGT GATCTGCCCA |
| | 53701 CCTTGGCCTT CCAAAATACT GGGATTAAGG TATGAGCCAC CGCACCCAGC CCTCCTGGGC |
| | 53761 TCTTTTCCTT TAGTTGCACT CGCTCCCCGC TCCTGGAGTA GAGGGATTTC CGAGAGACTG |
| | 53821 TGGGCTCCAG CCTTCACCTA GGCCCAGGAC TAGGATGCCT GCCCTAACAT TTATCTTTAT |
| | 53881 ACCTTAAAGC AAAACAGCTG GACCATAAGC ATTCAAGAAC AAACTGTGAA TAAGGAGAAA |
| | 53941 GTTCTCCCAG GAAACAAGAG CTTTAGTTCT GTTGGGCCAG CCCTTATATT CCTTAGCTGT |
| | 54001 TACCAGTCAC TGCTTGATTT AATCTCGGCT ATCACTTGGC CTGACAGGTC TGCTGCTGGT |
| | 54061 GCCAGGATGT CTGGGTTTTG AAGCCTGGCT CCATTACATA CTTCCTGTGT GACCTTGGGC |
| | 54121 AACTTACTCA ACCTGTCTGT TCCTCAGTTT CCCCAGCTGT ATTATGTCAG CATAATAGTT |
| | 54181 TGTTGTGTGA ATTAAATGAG GTAATAACTG GAAATGCTTC AAACATGGTT CCTATCATGA |
| | 54241 GAAATCCTGC TTTCCGCCTA AATGTGCTGG AAAATTCCTG GTGGTGCAGA ACAGGAGACC |
| | 54301 AGAGCAAAGG AAAGACAGGG TGCAGAAGCC AAAAATTACC TTGGAGAACA AAGCGCATGT |
| | 54361 TAAGGTTATT TTTGGATTCT AGGTTTATCT CTGCTTGGTC TTCAGTTACC TGCAAGAGAT |
| | 54421 CCATTTAGGG GATTTTTGTT TGTTTTTAAC GATAGCTTTA TTGAGATATA ATTCATATGC |
| | 54481 CATAAAAGTC ACTCTTTTAA AATGTTTCCG GTATATTCAC AAGGCTGTGC AGCCTTCCCT |
| | 54541 GTCCTTGATT CCAGTCTGAG TTTTTAACTG AAGGGATAAG GAGGACCACG CTTTCCCCAG |
| | 54601 ACCAGAACCG CGGGCCAGGG GGCGATTCTG CTGAGTCACC GCGGGCGCCT GGTGCGCGGC |
| | 54661 GGCGGAGCCC GGGACCTTCC TTGGCTGCCC CCTAGCGAGG GCCGCAGCGC AGCCTGAGAC |
| | 54721 ACCCGCCGGG GCCGCTCCAC GGCCGTCGGA TTTAGACTGG AAGCTCGGTC CAGGTCCCCA |
| | 54781 GCTTGATGCG CCCGCGGTGT AGGAGACCAG CCCGACTCGG GCTTCCCCTG AGCCCCTGGA |
| | 54841 CTCTTGACTC CAGCAGGGCC TGGGTAATGA ACGTCAGCTC CCCTTTCCCA AAGGGGTTGC |
| | 54901 TCTGTTGGGA AGGCACCCGT TTGATACAGT AGCATAGAGA TGGGTTTTAG CATCAAAATA |
| | 54961 TCAGAATTCA AGCCTTGCTC TCTGCTTACT AGCTGTGTGA CCCTAAAAAG GTTTCTGAAC |
| | 55021 GTCTCTGAGC TTCAGTTTCC TCATCATTCC TTCTCACGGG GTGGTTGTGA GCATTACAGA |
| | 55081 GATCCTCTCT GTGAAGCCCC TGTGAGTGGC TCATCCTGAG GGCTGAAATA AACATGTTAT |
| | 55141 TAATAATCCA AAACTGGCAA GGGATGTTGA CTGGTCCCCC TCCCTTGCCC AAGGAGCTTT |
| | 55201 CTAGAACCTG AGTTATCATT ACCAAACTGT ACTGCCTTGA GTAAGAAAGT TAGAAGGAAT |
| | 55261 GGGAAGGATG GTGGCAGGTG GAGGAAGGCG GATTGGTCAT CACCTCCTTG CAGCAAGAAA |
| | 55321 CAGCCCCAGA TCGTGGGAAA CCTACAGACC TGCTAGACAG ACTAGGAGCA AAAGCTGGGG |
| | 55381 CTTTAAGAAT CCCCAGGGAG GTTCTCCTGA GAGAGTAGCC AGTTGGATTT TGTAAGCAGA |
| | 55441 GATTTGTTTG GGGAGGAGGT GACAACGTAG GGAGCAGAGG GGCAAAGCTG TCGGGAATCC |
| | 55501 TGCCTTGAGG GCAGGGATGT GTGTTGGGGG GAGTTGGGTC ACTGGGGCTC GGTGGCCTTG |
| | 55561 GGCAAGTTTC TACCTCTCAG GTCCTTTACC CACCTAGGGT CGCCATCCTG CCCACCTCAC |
| | 55621 AGGTTACAGT GAGCCTGGAT GCACTGTCAT GGGCAGGTGC CCAGGAAAAT GGCAGACATG |
| | 55681 TTCCAAACAG CACGCAGCAT TCCCCAGTGA TGCCCAGGGT CACCTTGGAG GTGGGCGAGA |
| | 55741 TGCCTGGGGT TTCTCGTCCA CCCCACAACA CCTCAGGGGA CAGCCAAAGC TGTCCCTTCA |
| | 55801 GGTAAGCTGC ACAGAAGATG TGAACTCTGC TGCAAAGACT CTATTCTTTG GGAGCAAAAG |
| | 55861 GGACCCAGGG TCTCACCTGC ACATCCCTGT CCCTGAGGGC CTAGGGGTTC TTGGAGGCCC |
| | 55921 CAGCCTTGGC AAAATGAGGA AGAAGGTGAA GGTTGTCTGG GCCCCTGCCA GGCTCCTTCC |
| | 55981 TCGGCCACGC ACTCCCCTTC CTGCACACAC ACCCTTCTCC CTCCACCCCA TCTCCATTGT |
| | 56041 TGTCAGAAAA GTCACAATAA AAAGGTCCAT ATTGTCTAGT TCCCATACTT TTAATTTTTA |
| | 56101 AAATTTTATT TATTTATTTA TTTATGTATT TTTTGAGACA GAGTCTTAAC CCAGGCTGGA |
| | 56161 GTTCAGTGGC ATGATCTAGG CTCACTGCAA CCTCTCCCTC CTGGGTTCAA GTGATTCTCA |
| | 56221 TGCCTCAGCC TCCCGAGTAG CTGAGATTAC AGATATGTGC CACTATGCCC AGCTAATTTT |
| | 56281 TGTATTTTTA GTAGAGACGG GGTTTCACCA TGTTGGCCAG GCTGGTCTCG AACTCCTGGC |
| | 56341 CTCAAGTGAT CTGCCTGCCT GAGCCTCCGG AAGTGCTGGG ATTTCAGGTG TGAGCCACCG |
| | 56401 CACTCGGCTC CACACTTTTC ACTTATTAAA AGACTGTGGT GTCCATCAAT GGATGAATGA |
| | 56461 ATAAACCAAT GTGGACTATC CCTCCCATTA CCCAAGGAAT GAAGCACGGA GCCGTGCCAA |
| | 56521 GATCTGGATT CACAGTGAAA GAAGCCAGTC ACCAAAAGCC ACGTGCTGTG TGACTTCCCT |
| | 56581 TATACGAAAT ATCCAGAAGA GATACATCCA TGGTGACAGA AAGTAGATGA GCAGCTGGGG |
| | 56641 ACTGGCGAAG GGGAGAAGGG GGAGCAGCTG TCTATGAGGT CCAGCCTTTC TTCTGGGTTT |
| | 56701 GGTGAGAATG TTTTGGAACT AGATAGAGGT GATAGTTGTA CAACATTGTG AATGTACTAA |
| | 56761 ATGCCACTGA ATCATTCATT TTAAATCGTT CTTTACGTTG CATGAATTTT AAGTCAATCA |
| | 56821 AAAACAGTTG TTTGAAAAGA GAAAAGCCTA TGGGTAGCGG CAGCAGTGAT TGGATTTATG |
| | 56881 ATTCGATTCC ATGGCTCATC CCTCCCCTGC CTCACCCCCT CGCCCTCCGA CGTCTTCTTC |
| | 56941 TTTTACTCTG AACTGTTATC TTTGTTCTCA TCTCTCTCTC TCTCTCTCAA CCCTGCAGAC |
| | 57001 ACTTTTCCCT TTCTTTGTCT GCCCCCACCC TCCAGATTTC CGTGTCTCCA GTGTCTCCCT |
| | 57061 ACGAGGCATG AATTGAGACT GGGAGGGTGT GATTCTGAAG AAGGCACCAA CAGTGACTCA |
| | 57121 GCTAGCCCCT TCCCCCACCC CGCCCCCCGG GCCTCAATTT AGCTAAAAAA CCACAGGGAC |
| | 57181 GGACTCAGGA GGCAATACCT TTCCAAGGGT CCCTAAAAAA TGTCCCATTT TAGTGTCCAG |
| | 57241 GTTTCACTCA ACTTTAGTGC CTCCCCTAAA ATGTGTTCCT TACCTCCCAC CCCACTGCAT |
| | 57301 CTAAGTCACT GCCTGAGAAA ACAGGATTGA GGAAAGGAGA AAGGAAGAGA GAGAGAGAGG |
| | 57361 AGGAGAGAGA GAGAGAGGGA GGAAGGCTGA TGGATTTAGA AAAGAAGAAA ACAAGTGGTC |
| | 57421 TGAGGAAAAC AGCCTTGGTG TGTTTATTTT CCTGTCTGTG TATCGCTTCT CGGCCTTTTG |
| | 57481 GCTAAGATCA AGTGTATTTT CCTGTCTGTG TGTCTCGCTT AGATTACAGG GATCTGTGGG |
| | 57541 TGATGACACG TCTGGTCCAG GCTGCGTAGT CACCTCAAGG GCATGCTTAT TGATGTGTTT |
| | 57601 TTCAATTCAC TATCTTTGCA TGGGAGTCCC AGGCCAAGAG GCACAGCTGC GCCATTTGTC |
| | 57661 TGTTGGTTTA GATATCCTTT ATCCAGTTCT TCCAGAGAAA TCATCCTGCC CTTCTGGAGG |
| | 57721 AGGTGGGCAG CAGGGGTCAG AGATGGGAGG GAAAGGAAGG AGCCAGGTCC TTGGCTAGGA |
| | 57781 TGCCAGGGTC CCCTGCCTCT CACCTGGCCT GGGCTGGAGG CCTCCTGCTG TCCTGTCACT |
| | 57841 GATCACTACC CCGCCCCAGC CTCCTGAGTT AGAAGACACA GGCTAAAGTA GAGTATTTCT |
| | 57901 TCATTGAAAA ACCCATACAA AATAAAGGTT CATAAAAAAT AAAAATTTAG ACTGGGTGCT |
| | 57961 GTGGCTCACA CCTGTGATCC CAGCACTTTG GGAGGCCAAG GCAGGTGGAT CGCTTGAGCC |
| | 58021 CTGGGGTTCA TGACCAGCCT GGGCAACATA GTGAAACCCC ATCTCTACAA AAAATACAAA |
| | 58081 AAATTAGCCA GGCATGGTGG TGCATACCTG TGGTCCCAGC TTCTCAGCCT ATGGACCCAC |
| | 58141 ATAGAATACA ATGTCAGCAT AAGAAGGGAG CCCTGGGGTC ACCAAATGGT TTGGGCGGCA |
| | 58201 AAGAACCTGA AGGTTGAGAG AAGTGGCTTG GTTACCCAGC TGTTGGATGT GAGACCTGGC |
| | 58261 CACTGCTTCT TCCATACCCT AGACCTGCAC CCTGACATCT CAAGTAAAAA GTTGGGGGAT |
| | 58321 GTTTTATGGT CCAGGATGAA GGAAGGGCAG TGAGGGGCAG CGGAGCATCA CTTTGCATTT |
| | 58381 CTGTCTGCCT CTTACTGGCT GTGTGACCTG GGGCAGGTAA CTTCCCAGAC TCCTGGGAAT |
| | 58441 CATAACACCT ATGATGATGA TGATGATGAT GATGATGATG ACACCTACCT CAAGGATTGC |
| | 58501 CCTGAAGGGT CACAGAGATG CCTGCAAGGC ACCTGCATGG AGCAAGCGCC CCTTCTCTGG |
| | 58561 CAGGTGCTGG GTGAGCACTA CCTGCTGCCA GGCCCTGGGG CTATGGCACT GCGTGACCCT |
| | 58621 GCAAGTCCTA CCTGGCGAAG CTGTCGTTCT TGTGCTCAGT CAGTGTTGGT TGTAAGACTG |
| | 58681 AGAAGAGTCA CTTCATTTTG CTCTCCAGGG ACATCTTTCT GGGTCCTATT TTCTGCCTAT |
| | 58741 GTCAAGTAGC GCCTCAAGGA TGCTCCTGAA AATGGGCTTG TCTTTCTTAA CATGGCAGGT |
| | 58801 AGGTCCCAAA GCATTAGCAT GGGGCAGCTG ACCTAGCCCA GCCAATGCAG TGCAGTGACT |
| | 58861 CTTGCAACCG AGTCTAATCA GAAGGTCCAT GAACCTACGA GCATTTCCTG TCCCAGGATC |
| | 58921 AGGGTGGAGG CTGAGCCTCC CTGCTTAGAG ATTCTTCCCA TGCATTCCAC TTTTTTCCCC |
| | 58981 AAAAGAAAAT ATTGACCCTT GAGAGGCACA CAGTTTATTT ATTTTGCATA GTAAATAGTA |
| | 59041 GCCTGTATTT TAAGGATGAG TTGATTTCTG CATCAGCCCC TGTAGGTCAT CAGCCTTCTA |
| | 59101 TTGGTGCATC TGACTCTCTC TAGCCCTGCA GGGATGGTGG AGGGGGAGGG GAAGGAGGGA |
| | 59161 TCTTTATTGG AAACCAGGAC AGTGAGACTC ATTGCCCTGT CATCTGCTCT GTGGTGCTGA |
| | 59221 ATGAGGCAGC CCAACAGAGA AATACCCTGA GCGAGCATCC CCAGCCTCCA AAACAGTGGC |
| | 59281 GCATTGCCCT GAGTCCTGGG AATGACCTTT GATTCTCCTG CTCCTGACTT GGAACCCATG |
| | 59341 GAAACCTCTA GAAGCAGCTG AGGAAAACCC AACATGAAAA GCAGAACTCC ACACTGAGAA |
| | 59401 TATAGGAGGT GATCGGAACA TACAATGATT CTTGCTAAGA CCGATTCACA GTTTTTCTTT |
| | 59461 TTTTTCGATC GAAGAAATAC TGGAGAAGCC TAAAGAAGGA GTCTAAAAAC TCTGGCACGT |
| | 59521 GGGCCAAAAC TGTCCTTGAG CTAAGAATGA TTTTCACATT TTTAAGTGGT TGAAAAATGA |
| | 59581 AATAAAATAA GATGATGTTT TGTGACACAT GAAAGCTATG GGAAATTCAA ATTCTAATAT |
| | 59641 CTATAAATAG TGTTTTATCA GAACACAGTC ATGCTCATTT ATTTATGCTC GATGGCTGCT |
| | 59701 TTCCCGCTAC AATTACGTTG AGCAGTTACA ACAGAGACCA CGTGGCCCAC AAAGCCTTAC |
| | 59761 AATATTTACT ATCTGGCCCT TTCCAGAAAA AAATGTGCCG ACTCTTGACC TTAACCTCAG |
| | 59821 CAATTTGGGA GGCCGAGGCA GGCGGATCGC TTGAGCTCTG GAGTTCATGA CCAGCCTGGG |
| | 59881 CAACATAGTA AGACTCCATC TCTACAAAAA ATACAAAACA TTAGCCAGGC ATGGTGGTGC |
| | 59941 ACACCTGTGG TCCTAGCCAC TCGGGAGACT GAGGTGGGAG GATCGCCTGA GCCCAGGAAG |
| | 60001 TCGAGGCTGC AGTGAGCTGT GATGGCACCA CTGCACCTCA GCCTGGGCGA CAGAGCAAGA |
| | 60061 CCTTGTCTCC AAATAAATAA ATAATGCAAA GTAAAATAAA TAAAACCATA TAAAAAGGAA |
| | 60121 TCAATTTAAA ATTATAATGA AAGCTGGCCG GGCATGGTGG CTCACGCCTG TAATCCCAGC |
| | 60181 ACTTTGGGAG GCTGAGGTGG GTGGATCACG AGGCCAGGAG ATCGAGACCA TCTTGGCTAA |
| | 60241 CACGGTGAAA CCCCGTCTCT ACTAAAAATA CAAAAAAAAA ATTAGCCGGG CACAGTGGCG |
| | 60301 GGCGCCTGTA GTCCCAGCTA CTCGGGAGGC TGAGGCAGGA GAATGTCTTG AACCCGGGAG |
| | 60361 GTGGAGCTTG CAGTGAGCCG AGATCGTGCC ACTTGCAGTC CAGCCTGGGC GAAAGAGCGA |
| | 60421 GACTCCGTCT CAAAAACAAA AACAAAAACA AAAACAAAAA AAAATTATAA TGAAAGCCAA |
| | 60481 GGGGCATAGT AGAACAAATT TTCTAGAGCT CATTAAGTCA AATGAGTCAC CAGTTAGTAA |
| | 60541 AACGCAGTCA CGGGGAAGAG AGGGCAGGAT TCTTTGAAGC AGCGGCTCTC CTAAAAACAA |
| | 60601 CCCACCCTTG TCCAGCTGCC TTCCCTCCTG AGGGTGTTCC CTTTGACTGT GTGACCCCCA |
| | 60661 TCCCCTATTT CCCAACCGTC CAAGCCCACC TCTAGCATAA TACGAGCTTT TAATCCCTCT |
| | 60721 CCCTGACCCC AACCCGATTT TGAAGCCCAG TCTAGTATTT TCTCAAATAC ACTTCTTGGC |
| | 60781 TCCATTCCTT CCTTTCCATC ACCTCTGCCT TTTCACTGCA TGCTTGGACC ACTGCAGTCA |
| | 60841 GCTCCCTATG AACAGTTGCT CTCTACCCAT CCAATCGGCC CCGCCTGCTG CTGCCAAATT |
| | 60901 CACCGAGGGC ACCTCTGTGG TGCTGCCTGT GGACAAAGTC CAAGCCAGCC ACCTCACCCA |
| | 60961 CCTACAGGTG AGTGGGGAGC AGCCAGCGTG TCCAGTGGTT TACCCCATCG CCACAGACTT |
| | 61021 GGTGATGTGT CGATGTGCAG AGAAGGGGTG TTGGCAGCCA CAACACAAGC AACCCCGCCC |
| | 61081 CATGTGAGAT CTAAGATGGG CGTGCTGGGA GCCACCTCTG AGAATCCAAC AGAAGGCAGA |
| | 61141 GGGGAGAACG GCTCACACGG CACAAACACT CCTTCCTTTT TTTTTTTTCT TTTTCCTTTT |
| | 61201 TGAAAGGAGT CTCACTCTAT TGCCCAGGCA GGAGTGCAGT GGTGCAATCT CAGCTCACTG |
| | 61261 CAACCTCCGC CTCCTAGGTT CAAGCGATTC TCCAGCCTCA GCTTCCCAAG TAGCTGGGAT |
| | 61321 TACAGGTACA CTCCACCATG CCCGGCTAAT TTTTGTGTTT TTAGTAGAGA CGGGGTTTCC |
| | 61381 CTATGTTGGC CAGGCTGGTC TTGAGCTCCT GACCTCAGGT GATCTGCCTG CCTTGGCCTC |
| | 61441 CCAAAGTGCT GGGATTACAG GTGTGAGCCA TGGGGCCTAG CCTCCTTCCA TTTAAATGTA |
| | 61501 TGCCTAATTT GCCCATTGAG AACGGCTGAG ACGCATTTTA AGTGGCCAGG GTCTACTTAG |
| | 61561 AGTTAGTGCT CATGACCAGG CCCAGGTCAA GCCTGGCTGG CCAGATGGTG CCTTTGACCT |
| | 61621 GCTCTGTCTC TGTGCAAAGG AATGAGCTGA AGGATGGGGG TGCAGTGTGT GGGCAGTGGG |
| | 61681 CTGGGGCTGG CAGGACTCAG TGACTAAGGG AAGAGAACTT TCCTCACTAC CAGCCTGTCT |
| | 61741 TTTCAGGGCA CCGCGGGGGG CTTTGGGACT TGGTGATGAA CACAGCACAG AGAGCTGTCC |
| | 61801 AGCATGCGGG TCCCTGGCTT CTCACACTTC CCAGGCTCCT TCAGAGGCTC TCTCCAAAGG |
| | 61861 GAGCTGCTCT CTCTAGAACC CATGAATTTG GAATATAGGC AACCACTGCA TTGGGGACCA |
| | 61921 CTGACCTCAA ACATAGAGAC CAGAGCAAAT GGGGCTCATC ACGTGAAACT CATCTGGAAC |
| | 61981 TCTAGCAGGT TCTTTTATAT ATATATATAT ATATATATAT ATATATATAT ATATATATAT |
| | 62041 ATATATATTT TTTATTATTA TACTTTAAGT TCTAGGGTAC ATGTGCACAA CATGCAGGTT |
| | 62101 TGTTACATAT GTATACATGT GCCATGTTGG TGTGCTGCAC CCATTAATTC ATCATTTACA |
| | 62161 TTAGGTATAT CTCCTAATGC TATCCCTCCC CACTCCCCCC ACCCCACAAC AGGCCCCAGT |
| | 62221 GTGTGATGTT CCCCTTCCTG TGTCCAAGTG TTCTCATTGT TCAATTCCCA CCTACGAGTG |
| | 62281 AGAACATGCT GTGTTTGGTT TTTTTGTCCT TGCGATAGTT TGCTGAGAAT GATGGTTTCC |
| | 62341 AGCTTCATCC ATGTCCCTAC AAAGGACATG AACTCATCAT TTTTTATGGC TGCATAGTAT |
| | 62401 TCCATGGTGT ATATGTGCCA CATTTTCTTA ATCCAGTCTA TCATTGTTGG ACATTTGGGT |
| | 62461 TGGTTCCAAG TCTTTGCTAT TGTGAATAGT GCCGCAATAA ACATACGTGT GCATGTGTCT |
| | 62521 TTATAACAGC ATGATTTATA TTCCTTTGGT TATATACCCA GTAATGAGAT GGCTGGGTCA |
| | 62581 AATGGTATTT CTAGTTCTAG ATCCCTGAGG AATCGCCACA CTGTCTTCCA CAATGGTTGA |
| | 62641 ACTAGTTTAC AGTCCTACCA ACAGTGTAAA AGTGTTCCTA TTTCTCCACA TCCTCTCCAG |
| | 62701 CAGCTGTTGT TTCCTGACTT TTTAATGATC GCCATTCTAA CTGGTGTGAG ATGTTATCTC |
| | 62761 ATGGTGGTTT TGATTTGCAT TTCTCTGATG GCCAGTGATG ATGAGCATTT TTTCACGTGT |
| | 62821 CTGTTGGCGA ACTCTAGCAG CTTCTTTTCA CAAGTTCATG GAGAGAGGTT TCCCACTGAG |
| | 62881 GGAATCACAT CTGTCTGATC AAAAGAGGCT TGGGAAATGG CTCTCCTGTT CATTCCCTGA |
| | 62941 AAACCTCTGA TGGAACCACT GCCACTGTGG CAGCCCCAGC ACTGGCACCC CAGCCATGAT |
| | 63001 TGGTGCCCCA GCCACATCTC TGCTGTGAGC CCCAGAGCCC TGGTTAATTA ATCATCCACG |
| | 63061 TGTTGATGGG GAGAGGCCCA TTCACAAAAG CGACATAAAG CCCAGGGAGA CGTGGCCGTG |
| | 63121 GCAAGAAGGG TGTGGGACTA CATTCCGCCC CCAACTGAGA GATTCAGAAA CCAGAAAAAA |
| | 63181 ATGGAAAAAC ATACTGTGCT CTTGGGTGGG AAAACTAAAT ATCATGAAGG GAGCAATTTT |
| | 63241 TATAGTTTTG GCCTATAATA CAATTCCAGC CGAAATCCCA GTGGAACTTT GAGAATTTGC |
| | 63301 AGGAAAAAAA AAAATGTCTA AAGTACATCT GGAAGACAAA CTTACAAGAA GGTCAAATAA |
| | 63361 TTTTGAAAAA GAAAATGATA TCTAAGCCCA CCTAGAGAAT AAGACTTGAG ATCCAAAGCT |
| | 63421 AAATCAGGAG GCTCTAGCAA AATTGACAGA TAAGCAGGAC AGAGTGCATG GTGCATTCAC |
| | 63481 CTGGGGAAGA GGGCAGATTG GTCTACAAAT AGGCCTGGGT CCACTGACTT TAGCTGTTAT |
| | 63541 ATTTGGGGAG AAACTTTTCA ACCTCACTCC ATCTTAAACC TAAAAATATT CCAGATGAAT |
| | 63601 TAATAAATAT AAAAAATTAG ACCACTAAAA ATGTAGAAGA AAATGGATGA TCTTTCTATA |
| | 63661 CCATAGAGCA ATGGAATAAA TCACAAAGGA AAACAGATTT GACTATATAA AACTTAAACC |
| | 63721 CTGCCCATCA AAAACCATCA GAAACCAAAA TAAAAGGCAA CCAACTGGAG AAGATAGTTG |
| | 63781 CCACAAATAT GATCAAGGGT TAATGTTATT CATAAATTAA GAGCCCACAC AAGTCATTAG |
| | 63841 AATAAGCACT GAGACCTGAA CAGACAAGCA AAAAGAATGA GAGTGGGTCG GCGCGGCGGC |
| | 63901 TCATGCCTGT AATCCCAGCA CTTTGGAAGG CTGAAGCAGG CGGATCACTT GATCCCAGGA |
| | 63961 GTTCCAACAC CAGCCTGAGC AACATGGTGA AACCCTGCCT CTACAAAAGT CATAAATATT |
| | 64021 AGCCGGGTGT GATGGCACAC GCCTGTAGTC CCAGCTACTC AGGAGGCTGA GGTGGGTGGA |
| | 64081 TCACTTGAGC CCGGGAGGTA GAGTCTGCAG TGAGCCAAGA TCACACCGCT GCACTCCAGC |
| | 64141 TGGAGCAACA GAGTGAGACC CTGACTTAAA AGAAAAAAAA AAAAAAAGAG GAGAAAAATG |
| | 64201 CTGATCTCAC TAGTAATTAA AACATCAGGC CAGGCGCAGT GGCTCACACC TTTAATCCCA |
| | 64261 GCACTCTGGG AGGCTGAGGC AGGCAGATCA CTTGAGATCA GGAGTTCTAG ACCAGCTTGG |
| | 64321 CCAACATGGT GAAATCCCGT CTCTACAAAA AATACAAAAA TTCGCCAAGC GTGGTGGCAC |
| | 64381 ATGCCTGTGA TCCCAGCTAC TCGGGAGGCT GAGACAGGAG AATTGCTTGA ACACGGGAGG |
| | 64441 CAGAGGTTGC AGTAAGCTGA GATCGTACCA TTCCAGTCCA GCCTGGGCTA CAGAGCGAGA |
| | 64501 CTCTGTCCCA GAAAAAATTA AAACATCACA TATTTAAACA ACTCTAGGAT ATCATTTAAA |
| | 64561 AAAACATTAA TAGACTGTTT TTTAGAGCAC TTTTAGGTTC ACAGTGAAAC TGAGTGGAAG |
| | 64621 GTACAGAGAC TTCCCGTATG TTCCCTGCCC TCCACGTACA GCCTCCCCCA CTGCCAACGT |
| | 64681 CCTGCACCAG AGTGGTACAC TTGTTACAAC CAATGAATCC TCATTAACAT ATCATTATCA |
| | 64741 CCCAAGTTCA TAGTTTACAT TAGTAAAACA TCATCTTTCA TCTATAAGCA CAAAAATTTT |
| | 64801 TTGGCATTTA TTTAGGTGTA TGATTAACTC AGTGTTGACA AGACTCACAC TTCATACCCA |
| | 64861 CTTGCACTGC ATCTGAGAAG CAATTGGTGT CTACAGCCGC TACACCCTCA ACAAGCCCGA |
| | 64921 TCTTGTTTGA AAAGCAATTG GTGATGCTTC TCAAAATTCT ATGGACAAAG TCAGCCGGGC |
| | 64981 ATGGTGGCTC ATGCCTGTAA TCCCTAAACT TTGGGAGGCC GAGGCAGGCA GATCACCTGA |
| | 65041 GGTCTGGTGA AACCCTGTCT CTACTAAAAA TGCAAAAATT ACCCAGGCAT GGTGGCTGGG |
| | 65101 GCCTGTAATC CCAGCTACTC GGGAGGCTGA GGCAGGAGAA TCGCTTGAAG CAAGGAGGCG |
| | 65161 GAGGTTTCAG TGAGCCAAGA TTGCACCACT GCACTCCAGC CTGGGTGACA AGAGTGAAAC |
| | 65221 TCCATCTAAA AAAAAAAAAT TATGGACAAA GTTTTTCAAA AAGATATTTA ATGCAACTTT |
| | 65281 ATTTGTAATA TTGGAACATC TGAGGCCATT TCAGTGCTAA CTATTAGGGG ATGGTTAGGA |
| | 65341 AAATATGGTA CATATGTGGA AAGGAACATT TGGTAGTTAG TGCCCCTGAT GTTTACAAAG |
| | 65401 GCTTTTAGTG ACCAACAAAT GCTCATGCTA TAATCTTATG TGAAAAAAGC AAGTAGCATA |
| | 65461 ATTGCAACTA TATTTTTAAT GCATAGAATA AAAGGCTAGA AGGAAATATC ACAGATCCTT |
| | 65521 GACATACATT CCCAAACCTT TGTAAATCCG CGGATTCATG AAAACAGACA CATTTGCACA |
| | 65581 AGTGCCTGAT CTTTTCTGTT ATACATTCAT TAGAAGTCAA GCCCTGGTGC CACAAAGTAT |
| | 65641 CTGCCTTTTC AAATGTGATC AGAATGTTCT CTTTTGCTTC AAGGCCATTT TTCACGAAGC |
| | 65701 AGTGGCATTT TTGCCTCTTC ATCAGAGTCA CCGTGTGCCC TGGAGGACTG AGAACAGCAG |
| | 65761 AGCCGTTTTA GGATGGGACA GGGCAGCCAG GAGGATTGGG CTCACTCCCT ACTGAGTGCC |
| | 65821 TCACTCCCGT ACAGCCCCCA TAGAGGAAGA GGGGTTCAAA TTTATTCCTC AGCCAGATGG |
| | 65881 CATGTGCCGC CTGTCCTGGA ATTTCACATC ACTTATGATG GACCAAAATT CCAAAAGCTG |
| | 65941 AATCCATGAT TGTCAAAGTC TGGTATGGCA GGATGTCAAC AGTAATCGTT TCTGGGCAGA |
| | 66001 GGGATGATTT TCTCTTCCCA TCTTGCTTTG TATAAATACA TTTTCTATAA TAAGGTTGTA |
| | 66061 TTACTTTTCT CATCAAGAAA TAGCAAAGTA CTGTTTTACT CAAAATATGA ATAGAGCCAG |
| | 66121 GCATGGTGGC AGCTTATGCC TGTAATCCCA ACACTTTGAG AGGCGGATAT GGGAGGATCA |
| | 66181 CTTTAGCCCA GGAGTTTGAG ACCAGCCTGG GCAACATAGT GAGACCCCCG TCCCCACTCC |
| | 66241 CCCAAAGAAA ACCCACAAAG CATTTATCCT GGATTATTCA CAGGGGCCAA AAAAAAAAAA |
| | 66301 AAAAATTCAG GCCTCCTATA GCCATGAGCT ACGAATATGA AAATATGCAA ATGTGTAAGA |
| | 66361 AAAGCCAGCA CATCCGATTT TTACTTTTAC TTTCACACCT CTGTCCACCA TGTTCCAAGA |
| | 66421 GAAGAAACTT GGTCATTGAA AGGAATAGAT CAAATCCAAA GAACAAAACC ACTGTGCTCA |
| | 66481 TTAAACTTCT TAGTGTTCAC AAAGCTTTAG CTGCAGGTTG AATGGGGCAA CCCGAATTGG |
| | 66541 CTGGCTCACC TGGGCTGCAG GGAGCAGAGA TCGCGACACT GCACTCCAGC CTGGGCAACA |
| | 66601 AAGCGAGACT CTATCTCAAA AAAAAAAAAG TTCATAAATT CAAAGTTATG AATTATTTTT |
| | 66661 AAAATAATAA TAATTTACAA TAAAGATGAG GACAAAGTGT GAGTAAATGG TGGTTTCTAT |
| | 66721 CCAGCTCTGT TGAGCTGAAG TGGCATCTCC CTGCTGGGGC TTTTGGGGAA GAAGGGTGTG |
| | 66781 TGTTGCTCTT CAGATCCCAA GCCTCATGCC CCTACTGGGC CCTGTGGGGT GCTTCTCAGC |
| | 66841 CCACCAGGAG AGCCACCGTT GGAACACACA CGTGGGGGAC CTGGTGGGTG CCGGTGTGGT |
| | 66901 GAATGGGGGC CACAGCCTGA CTCCAGGAAG CCAGCAAACT CGGAGCTGGA GGAGTCAGGA |
| | 66961 CACCCCCGAT GAGTCAAGAG TTGGTTTTGC TGCCAGTTGA CATCTGATTG AACCATCTCT |
| | 67021 TCACTTCTCC GTGCCTCACT TTCCTTACCA GACAGGCTCT GCTGATGCTG TCCCTCTCCT |
| | 67081 GTTCAGTCGT GCCCTCACCG TTAAAGAGAA AGAGCAAACT GCTGGGCAGC AGCATTGATT |
| | 67141 TTTTTAATGA AGTGGAAAGA GAGCTGGGAA TAACAAGTCG GGCCCACCTC ACCTGCCTCA |
| | 67201 CCTGGTGGGT TTATTTGTTT TGTTTTTTTT TTTTTGTTTT GAGACAGAGT TTCACCCTGT |
| | 67261 CACCCAGGCT GGAGTGCAGT GGTGTAATCT CAGCTCACTG CAACCTCCAC CTGCCAGGTT |
| | 67321 CAATTGATTC TCCTGCCTCA GCCTCCCCAG TAGCTGGGAT TACAGGCACC TGCCACATGC |
| | 67381 CTGGCTAATT ATTGTATTTT TAGTAGAGAT GGGGTTTTAC CATGTTGGCC AGGCTGGTCT |
| | 67441 CGATCCCCTG ACCTCAGGTG ATCCACCCAC CTCGGCCTCC CAAAGTGCTG AGATCACAGG |
| | 67501 CGTGAGCCAC CATGCCTGGC CGTCACCTGG TGGTGTTGAA TATGAACTGC TGCGGTGTTG |
| | 67561 GTAAATTAAG CAAGCAGATA GATGTAAATA ACGCTTGGGC AGGAATATGG AGCACGGGAT |
| | 67621 GAGGATGGGC GGCCAACTGT TAGAGAGGGT AGCAGGGAGG CTGAGATCTG CCTGCCATGA |
| | 67681 ACTGGGAGGA GAGGCTCCTC TCTCTCTTCA CCCCCACTCT GCCCCCCAAC ACTCCTCAGA |
| | 67741 ACTTATCCTC TCCTCTTCTT TCCCCAGGTG AACTTTGAAC CAGGATGGCT GAGCCCCGCC |
| | 67801 AGGAGTTCGA AGTGATGGAA GATCACGCTG GGACGTACGG GTTGGGGGAC AGGAAAGATC |
| | 67861 AGGGGGGCTA CACCATGCAC CAAGACCAAG AGGGTGACAC GGACGCTGGC CTGAAAGGTT |
| | 67921 AGTGGACAGC CATGCACAGC AGGCCCAGAT CACTGCAAGC CAAGGGGTGG CGGGAACAGT |
| | 67981 TTGCATCCAG AATTGCAAAG AAATTTTAAA TACATTATTG TCTTAGACTG TCAGTAAAGT |
| | 68041 AAAGCCTCAT TAATTTGAGT GGGCCAAGAT AACTCAAGCA GTGAGATAAT GGCCAGACAC |
| | 68101 GGTGGCTCAC GCCTGTAATC CCAGCACTTT GGAAGGCCCA GGCAGGAGGA TCCCTTGAGG |
| | 68161 CCAGGAATTT GAGACCGGCC TGGGCAACAT AGCAAGACCC CGTCTCTAAA ATAATTTAAA |
| | 68221 AATTAGCCAG GTGTTGTGGT GCATGTCTAT AGTCCTAGCT ACTCAGGATG CTGAGGCAGA |
| | 68281 AGGATCACTT GAGCCCAGGA GTTCAAGGTT GCAGTAAGCT GTGATTATAA AACTGCACTC |
| | 68341 CAGCCTGAGC AACAGAGCAA GACCCTGTCA AAAAAAAAAG AAAAGAAAAA AGAAAGAAAG |
| | 68401 AAATTTACCT TGAGTTACCC ACATGAGTGA ATGTAGGGAC AGAGATTTTA GGGCCTTAAC |
| | 68461 AATCTCTCAA ATACAGGGTA CTTTTTGAGG CATTAGCCAC ACCTGTTAGC TTATAAATCA |
| | 68521 GTGGTATTGA TTAGCATGTA AAATATGTGA CTTTAAACAT TGCTTTTTAT CTCTTACTTA |
| | 68581 GATCAGGCCT GAGTGGCCTC TCTTTAGCAA GAGTTGGTTA GCCCTGGGAT TCTTACTGTA |
| | 68641 GCCACATTAA TAAACAACAT CGACTTCTAA ACATTCTATA ATACCATCTT TTGGCCAAAT |
| | 68701 TGACTTCGCC TCTTCCTCTC TCTTTCCAAA TGAAATGTGT TTCATTTCAC TGTCAGACCA |
| | 68761 CATGGTTGGG GACCCCACAG AGCACACAGC CCTCCCTCTG CCTTCCCATG CTGGCCCTTC |
| | 68821 ACCCACTGCT GGAGTGCCAG GTTGGTCCAA GGGTTGGACC AAGTTGTCTG AGGTTGTCTC |
| | 68881 AAGGTTGGTC GAGGCTGTCT CCGCGCTGGG TTGTGCTACA AGGAGCCCTT CTTTCCATGG |
| | 68941 GTGTGGCTGG CAGTGAGTGC TCACAGCAAC AGCCCACAGT GCAGCCCGAG GGCAGGATGG |
| | 69001 ACTCAGTCCC TGCCTCCATA CCCATTTCTA AGGAGGCAAA ATGGCAAACA CTCTACTTTT |
| | 69061 CTCTTTTAAT GCTAAAAATA AGAAAACACC TTGCAGCCCA GGGTATGGGT AGTGCATGGA |
| | 69121 AGCCGTGGAG TTGTGAGGTG GGAAGTGACC TCTGCTGGAT ATGTCTATTC AGGAAGATTG |
| | 69181 CTGGAGTGGG TGGGGTCTCT GGGAGGTCCC CTGAGTGTGG GAAGCTGGGA CCACCAGCTT |
| | 69241 TCTCGCACAG GGAGTGGCCA TCCCAGCTTG GAGAGGTTCC AGGACTGGTT GGGAGGCACG |
| | 69301 TTTCAGATTT CTATCTGTTG AATCAGCGAA GATATTGGAT TATGAGGAAT TTGGGAATTA |
| | 69361 GGAAAGTGGG TGCAGGTGGG TTGGGGGTAG GTGAAGGAAG ACATGGGCGT ATTGGGGGAG |
| | 69421 CAGGGGCTGC TCAGAGGTGT TCCAGAAGCT CTGGGTGAGG AGGTGAGAGG GACCGGGGAA |
| | 69481 TGCAGCTCGG CCCAGCCTCC CTGCCTGAGG TCAGCCATCA CGTGGTGATG GCAAGATGGA |
| | 69541 AATGTGCTTT CTGACTGCTC CAGCCAGTGC TGCCAGATTC AGCTCCCCAG GGAGGGCACC |
| | 69601 TGAGAGGCTC CAAGCCAGGA GATCTGTTTT CTCCTTTGTT TTGTTTTTTT TTGTTTTGTT |
| | 69661 TTGTTTTATT ATACTTTAAG TTCTAGGGTA CATGTGCACA ACGTGCAGGT TTGTTACATA |
| | 69721 TGTATACATG TGCCATGTTG GTGTGCTGCA CCCATCAACT TGTCATTTAC ATTAGGTATA |
| | 69781 TCTCCTAATG CTATCCCTCC CCCCTCCCCC CACCCCCTGT TTTCTCCTTT GAATCCTTCT |
| | 69841 TAGAGGCCGG GCGCGGTGGC TCACGCCTGT AATCCCAGCA CTTTGGGAGG CTGCGGCAGG |
| | 69901 AGGATTGCTT GAGCCCAGGA GTTCCAGACC AGCCTGGGCA ACATAGTGAG ACCTCGTCTC |
| | 69961 TACAGATAAT AATTTTAAAA ATTATCCGGG CATAGTGGCA TGCACCTATA GTCCCAGCTA |
| | 70021 CTCAAGAGGC AGAGGCAGGA GGATCACTTG AGCCCAGGAG GCGGAGGTTG CCGTGAGCCA |
| | 70081 AGATCCCACC ACTGCACTCC AGCCTGGGCG ACAGAGACCC CCATGTCAAA TAATAATAAT |
| | 70141 AATAAATAAA TCCTTCTCAG TCCCTTCCTC ACTGTGTCCC CCTCCACTGA ATTTTTCCAC |
| | 70201 CTCCTCTCCC ACTTCCCCCA CTCCCGCTTT CCCTCTCCTT CTCTCCCCAC TCCATCTTTT |
| | 70261 TCTTTCTCTG CTGTTTCTCG TCCCTCCCTC CTCTCCATCC CACAACACTG CCTACCCTGT |
| | 70321 CCCTGCCCCA CCCTGGTGCT CAGGATGTGT GAAGTGAGGG GTGGTAGCCC CCAAGACCTC |
| | 70381 AACCCCGAAG GTTAGCCTGT TGAAACCACT TTCTCCCAGC TGCCCCCCTG GCAGTTGGTG |
| | 70441 CTGCTGGGGG AAACTGGGAT TGGGGGCCAG ATTTTGCCTC TTTTCCTGAC AAAGAGAGAT |
| | 70501 GAAGAGTTCT CTCACCAGGT GCCTGGGACT GGGGTGTGGG TGTCCCAGCC TATCCCAGCG |
| | 70561 CATCTGTTCT GCATCATGAT TAATAGTGCT GCTTTCAGCC GGGCGCGGTG GCTCACACCT |
| | 70621 GTAATCCCAG CACTTTGGGA GGCTAAGGTG GGCAGATCAC AAGGTCAGGA GTTCGAGACC |
| | 70681 AGCCTGGCCA ACATGGTGAA ACCTCGTCTC TACTAAAAAT ACAAAAATTA ACCAGGTGTG |
| | 70741 GTGGTGGGTG CCTGTAGTCC CAGCTACTTG GGAGGCTGAG GCAGGAGAAT CACTTGAATC |
| | 70801 TGGGAAGCAG AGGTTGCAGT GAGCCAAGAT CGTGCCACTG CACTCCAGCC TGGGTGACAG |
| | 70861 AGTGAGACTC CGTCCTAAAA AAAAAGGAGT TTTGCTCTGT CGCCCAGGCT GGAGTGTAGT |
| | 70921 GGCGCCATCT CGGCTCACCG CAACCTGCGC CTCCCGGGTG CAAGCGATTC TCCTGCCTCA |
| | 70981 GCCTCCCAAG TAGCTAGGAT TACAGGCGCC TACCACCACG CCTGGCCAGT TCTTGTATTT |
| | 71041 TTAGAAGAGA CGGGGTTTCA CCCTGTTGGC CAGGCTCGTC TGGGACTCCT GACCTCAGGT |
| | 71101 AATCCGCCCA CCTCAGCCTC CCAAAGTGCT GGGATTGCAG GCATGAGCCA CCGTGCCCAG |
| | 71161 TCAACTCCTT CTCAAAAAAA AAAAAATAGT GCTGCTTTCT CTTTCAAGTG TCCTGATTTG |
| | 71221 GGTGATAGTA AATGCCACTC TACTTATAAG GGATCTACCT CAGAATGCTA ATTGGGACAT |
| | 71281 TTTTGTAGCA CTCTACTGTT GGCAGCAGGT GATGCTCACA ACAGCCCGTG AGGGTGGATG |
| | 71341 ACGTCCGCTT CACAGATGAC AAAGGAGCCT CATGCTCAGA CCGTGGGCTG CCAGAGCAGG |
| | 71401 TCCATGGCTG CAGCCCCACA TGGACCATAT TTCCCCCTTG TCACTCTTTC CACCAAGCTC |
| | 71461 CCTTGGAACT TCAGTTATTA AGCTCTCTTG GGTGGAATCC AAGTTAGAAT CACAACATGT |
| | 71521 GCCTCATATG GATTGTGCCA GTGAAAAATG ACATTCTATT TAGAGGCAGG GCAGCCTGGC |
| | 71581 TTAGAGTCAG TTTAAAATAT GTATTATGCT GCAACAAATG TACCATGATC CTGTAAGATG |
| | 71641 TTCACAACAA GGGAACTGGA TGTGGGGTAT ACTGTCTGTA CTAACTTCAC AAGTTTTCTG |
| | 71701 TAAATCTAAA ACTGTTCCAA AATAACAAGT TCGTTTAAAA TTAACTCCAG GAGACCAGGT |
| | 71761 ACGGTAGCTA ATGCCTATAA TCCCAGCACT TCGGAAGGCT GAGGCAGGTG GATTGCTTGA |
| | 71821 GCCCAGGAGT TTGAGACAAG CCTGGGCAAC ATGGTGAAAT CCTGTCTCTA AAAAAAATCA |
| | 71881 CAAAAATTAG CCAGGTGTGG TGGCGCATTC CTGTAGTCCC AGCTACTTGC GGGGCTGAGG |
| | 71941 TGGGAGAATC ATCTGAGCCC AGGAGTTTGA GGCTGCAGTG AGCTGTGATT GTACCACTGC |
| | 72001 ACTCCAACCT GGGCAACAGA GCAAGACCCT GTCTCAAAAA ACAAAAATGA AATAAAGTCC |
| | 72061 AGGAAAGAAG TAGGTTTTAC CACTCTTATT TTCTGAAGAG AAAACTAAAT TTAATGTGTA |
| | 72121 AAGTGAGGAC AAGTTCACCA AGTTAGTGTT TGAGTTGCCT AAAATATGTT TGCTAAAACT |
| | 72181 ATTCAAAGCT TTCACATAAA ACATGATCAG AAGTTCTATG CCAAAACATA TGTGTGTGTA |
| | 72241 TATATATATG CACTATATAT ACTGTATATA AAAATGCAAA ATCTAAATTG CCAACCTTTT |
| | 72301 AGAAATTGCT CTGAAAGGAA AGCATTTCAA GATAATTTGC TTACCCAAAG AATATACTTT |
| | 72361 CCAAGAAAGC AAGTAATACT TAAGGTGTTC ATAATCCTCA TCAAATTAAT TCTTGCTACT |
| | 72421 GAAAGCTTAC AAGGAGCTGT TTTGATGTCG GGTGTGACAG GTTTGACTTG GCAGAAGGTG |
| | 72481 TCACTTTACT AACAACATTT TAAATAAGTG ACAGAAGACA AGAAACTACA CGTTAAATGC |
| | 72541 CAGAACAAAG AGTGTCTAAG TGGATGCTAA GAGTTGAAAT ATGGCTGGAT ACCTGCCCAA |
| | 72601 GAGAGCTGAA AAGTAGATGA AAGTTGGTTA CCTATAAACT AGTGCACCCT AATGAATTAA |
| | 72661 AAGGTGTTGA TGAGTTAACT TGTTATGCCT TCCAGATAAG ACATGCAAAT GGGGCTTCTT |
| | 72721 CCTCCTTCAC TACTTCCAAG GGATTTAACA AGGAGACCAA TGCAAATGAT AAGGACTGTA |
| | 72781 GGGCTCAAGC TGGGGACAGA TTGGGGAAAG GGGGACCATC ATGCCCATAT AGATGTCCCT |
| | 72841 GTGCCCTGGC AGTCAAGGCT GCTGAAAAAT AACAAAACCC AGAAGTCTGC GTGATGCTGC |
| | 72901 CTCTCCATTT GTCCAAAGCC TTCTTGCGGC AGTTTGCAGG CTTTTGCAAA AGCTCCAGGA |
| | 72961 CCAAGGAGCT ATGTTCATGC TGGAAGCTTG TTCAGGATTA GCTGTTCTTT GTGGGATGGG |
| | 73021 TGCAGCCAGG GCCAGGTGTC CAGGGACAGT GTTTTAACAA AGGGCATGAG GTGTCTGATC |
| | 73081 TCACAGTGGA ACTCCACTTG CCTTTTTTTC ATCTTCTCAT TCTGCTTCAT GCACAGAACC |
| | 73141 AGCCCCATCC TGAAACTGAC TCTAAATTAC TCCCGCCCCA GGTGGAGTGC CTTTCTCGGA |
| | 73201 GTTCAACAGA GCCTTCCTGT CGCCCAAGGG ACAACTCCAC TGAATGCCCA AGCCACACCC |
| | 73261 AAAACCTAAC AAGTAAAAAC CAAATTCTGT GCTCCCCCAT CCTGGGCCAT TCCTGGTTTC |
| | 73321 TCTACTGCTG TTGGTGATAC CACCATCAGC TTGTCCATCA TGACCCTGGC CAGTTCCTCC |
| | 73381 CACAACCCTC CACAGCACCC AGGGACCTCA CCTCCATTCC ATCCGACACA GATCTCCTCA |
| | 73441 CCACAAACCT TGGTTTTGCA ACAGCAGCCA TGAGACCTTT ACACCCTCCG CCCTTCATCC |
| | 73501 TGTCCCCCAC TGAGGCCCCA GAGCCATTCC TTAAAGCAGC GCGCCACAAA CTATAACCCA |
| | 73561 CAAGCCAATT CTGGTACCCA GCCTGTTTTG CACAGCCAGT GAACTGACAA TGATCTTTTC |
| | 73621 ATACAGCCAG AAAAACAAAA CAAAACAAAA AACAACAAAA AAAAACCCCA CCATTCTGAG |
| | 73681 CATGTGACTT CCATGTTCAA GATGTCTCAT GTTCAGAAAG GCCCCTGGAA AAGGAGGAAG |
| | 73741 GGGAGCTGGG CACAAAGGGA GACCCTCTCA GCTGAGCTCC TCCCATCCAG ACATTTTCCT |
| | 73801 GGACTTCCTA TCCAATGACT TCCCTTAGCT TCTTATCAGC CACCCCTGTC TGCCCAGGAG |
| | 73861 GCTGGAAGAT GTGGCCTTTT AACTGGGCAC AGCTCTGTCC TCTATCATAT CAGGGCTCTG |
| | 73921 TTCCCAAGGA GGGTAGAGAG AATGGACACC AGGTGGACCC TCAGCAGTCT GTGCCACAGA |
| | 73981 GGGAGTGTTT GCAATTTCCA GACTAAAAGT CCCCATGTGC TTGACGGGGT ATGTGACTAC |
| | 74041 AACGTGATGC TTGACTTTTC CTCATATGAC CAGAGCCACT TTGTCCATCT GGTACAATGT |
| | 74101 CAGCTATCTG CTAGGGGCCC TCCAGGATTC CCAGTCAATT CCATATCTGC ATCACCACCA |
| | 74161 TTGGCACTAA ATAAAATAAA ATACTCAAGT TCCTGCTGGT GAGCATGAGC AGTGCTACAC |
| | 74221 TGGGCCCTTC AACCAAGGTG ACATGATAAT GACTGAAAAT AATCACTGCC ACTTATTGGG |
| | 74281 GACGTCTCAT CTGCCAGGCA TGGTACAAAG TGCTTTAAAT AAGCATTCAA CAATTTCATG |
| | 74341 CTGACAGAAG CCCTGTGAGC CAGTGGAGCT ACTACTATGC CCATTATACA GGGGAGAAAA |
| | 74401 CTGAGGCAGA GAGAGGTTAG GTAATTCGCT CAGCCTCACA CAACCAATAG GTGGTGGAGC |
| | 74461 CAGGATTTGG GCCCCATCTG CCTGACTCTC TAGAGGCTCT ATCTTCCAGT CTTCCAGAGT |
| | 74521 TGAGTCTAAG CCATGAATAG GACAATTAGA CAGCAGAGGA AACCCATTCA GCCACCATGT |
| | 74581 GCATGAAGAG TAAGGAATTT CTGTCATACA GAGGGGAGTG AATTCACTGA GCTGAGAGCT |
| | 74641 GAGGAACCAT TGATCTGATG GCTGAGACAC CACTGGGAAG ACTGGAGAGG CTTTTCTGGG |
| | 74701 CATGCAGTGC CAGGCACAGG AGGAGCTGAG GGAAGATGAC TAAGAGGTAC TGGCAAAGAA |
| | 74761 TTCAGAAATT CTGATGGAAG CTTTACATGT TACCATCACA TCCATCCATC TATCCACCCA |
| | 74821 TCCATCCACC CATATCTTCC TCCCTCCACC CAATCATGCA TACATCCAGT CATCTATACA |
| | 74881 CCACCCACCC ACCCATCCAT CCATCCATCC ATCCCTTCAT CCATCCCATC ATCCATCCAA |
| | 74941 TTATACATAC ATCCAATCAT ATATCTGTAC ATAATCCATT CTTCCCTCGG TTCATCCATC |
| | 75001 CATCCATTCA TCCATCCATC CACCCATCCC TTCCTTCATC CTTCCTATCA TCCATCCAAT |
| | 75061 CATATATCTG TACATAATCC ATTCTTCCCT CGGTTCATCC ATCCATCCAT TCATCCATCC |
| | 75121 ATCCACCCAT CCCTTCCTTC ATCCTTCCTA TCATCCATCC AATCATACAT ATATCCAATC |
| | 75181 ATACATCTGC ACATCACCAG CTCATCCATC TATCCATTTA TCCATCCATC CTTCCTTCCA |
| | 75241 TCCATCATTC ATCCATCATA CATACATCTA ACCATACATC TCTACATCAT TCATTCTTCC |
| | 75301 ATCGATTCAT CCAATTATCC ATCATTCCTT CCTCCATCCA TCCCATTATC CATTTGATCA |
| | 75361 TACATATATC ATCTATACAT CATCCATTCA TCCATCCATC CATCCATCCA CCCATATCTT |
| | 75421 CATCCAATCA ATCATACATA CATCGAATCA TCTACACATC ACCCATCCAT CCATCCATCC |
| | 75481 ATTCATCTAT CCACCCATCC ATCCATCCAT CCATCCATTC ATCTATCCAC CCATCCATCC |
| | 75541 ATCCATCCAT CCATCCATCC ATGTAACCAT CCAGTCATAT ATCCAATTAC ACATCCATCC |
| | 75601 AGTTATACAT TCATACATGC ATCTAATCAT TCAATTATAC ATACACACAT CCATATAATT |
| | 75661 CTACATCCAA TTATACCTCC ATCCAATTAC ACATTCATAC ACCCACCTAA TAAATTATTA |
| | 75721 ATTCATATAT CCATCCATAT AATTATACAT CAATTATACA TCCATCTAAT CATTCAGTAA |
| | 75781 TTCACCCACC ATCCAGTCAT CTATCCAATA ATACATTCAT CCAATCATCC ATCCATCCAT |
| | 75841 CCACCCATTC ATCCATCCAT CCGTCCGTCC ACCCATCATG GTATGAGCCA TGATTTACCA |
| | 75901 CGATGGTCCC CTGTGGACAG CCCAGGTGGG GCAGAACTGA AGGGAAGCCC AGGGCTGCCC |
| | 75961 CCATAAACAT TTGCCTCCTT TACATGGATG AGAACTAGAT CCACATGTAT AAATCCTCAT |
| | 76021 GATTTGAAGG TGCTTTTACC AACATTCACT CATGGGATTC TCCCAGGAGC TCTAGGAGGA |
| | 76081 GGCAGGTAGA GTTGAGGTCA TCTCACGCAT TTTACAGATG AGGAAACGGA GGCCCTGAGA |
| | 76141 GGCAGGTCCA AGGCCACCTG ACCAGAAAGA AGTGGAACTG GGACTTGAAC CCAGCCATCT |
| | 76201 TGCCCCTTGG TCCCATGCTC TCTAGCCTGT AACTCCTGCT TCCTGGTGGG GCATCTCCAG |
| | 76261 GAGGACCCTA TCGGCTGGCC ATGGGCCTGC CCTGGAGTCT TTTGCTCTGT GTGGCCATCC |
| | 76321 TTCCTCCCTC AGGAGAGTGT GTGCTCCCAG AGCACAGGCT GTATCTTCTG AGCATTTTGT |
| | 76381 CCCTTCCCAG TACCTAGCAC TCAGCTCTGT ATACATTGGG CTCTCAAGAA TTCTCAACCT |
| | 76441 TCCAGAGTGT AAGGCCTTGA CCTGCTCAGC CCTGGATACT GCATGATGCA TTGATAAGCC |
| | 76501 CATAAAATAA CCAGGGCAGA TTGACTCCCA GTGGCCAAAG TGCCACAGGG AAGGGACAAT |
| | 76561 TCAGCCCTTC TAGGAGGAGG AGGAGGTAGT TTTCTCATTT CTATTAAGGC AACAAAAGCT |
| | 76621 GCCTTACTAA GGACATTCTT GGTGGAGGGC GTGACTGTCA ACCACTGTGA TCATTTGGGC |
| | 76681 CTCTCTTGCC CAGGCTTCCC ATTCTGAAAG GACAGTTTTA TTGTAGGTAC ACATGGCTGC |
| | 76741 CATTTCAAAT GTAACTCACA GCTTGTCCAT CAGTCCTTGG AGGTCTTTCT ATGAAAGGAG |
| | 76801 CTTGGTGGCG TCCAAACACC ACCCAATGTC CACTTAGAAG TAAGCACCGT GTCTGCCCTG |
| | 76861 AGCTGACTCC TTTTCCAAGG AAGGGGTTGG ATCGCTGAGT GTTTTTCCAG GTGTCTACTT |
| | 76921 GTTGTTAATT AATAGCAATG ACAAAGCAGA AGGTTCATGC GTAGCTCGGC TTTCTGGTAT |
| | 76981 TTGCTGCCCG TTGACCAATG GAAGATAAAC CTTTGCCTCA GGTGGCACCA CTAGCTGGTT |
| | 77041 AAGAGGCACT TTGTCCTTTC ACCCAGGAGC AAACGCACAT CACCTGTGTC CTCATCTGAT |
| | 77101 GGCCCTGGTG TGGGGCACAG TCGTGTTGGC AGGGAGGGAG GTGGGGTTGG TCCCCTTTGT |
| | 77161 GGGTTTGTTG CGAGGCCGTG TTCCAGCTGT TTCCACAGGG AGCGATTTTC AGCTCCACAG |
| | 77221 GACACTGCTC CCCAGTTCCT CCTGAGAACA AAAGGGGGCG CTGGGGAGAG GCCACCGTTC |
| | 77281 TGAGGGCTCA CTGTATGTGT TCCAGAATCT CCCCTGCAGA CCCCCACTGA GGACGGATCT |
| | 77341 GAGGAACCGG GCTCTGAAAC CTCTGATGCT AAGAGCACTC CAACAGCGGA AGGTGGGCCC |
| | 77401 CCCTTCAGAC GCCCCCTCCA TGCCTCCAGC CTGTGCTTAG CCGTGCTTTG AGCCTCCCTC |
| | 77461 CTGGCTGCAT CTGCTGCTCC CCCTGGCTGA GAGATGTGCT CACTCCTTCG GTGCTTTGCA |
| | 77521 GGACAGCGTG GTGGGAGCTG AGCCTTGCGT CGATGCCTTG CTTGCTGGTG CTGAGTGTGG |
| | 77581 GCACCTTCAT CCCGTGTGTG CTCTGGAGGC AGCCACCCTT GGACAGTCCC GCGCACAGCT |
| | 77641 CCACAAAGCC CCGCTCCATA CGATTGTCCT CCCACACCCC CTTCAAAAGC CCCCTCCTCT |
| | 77701 CTCTTTCTTC AGGGGCCAGT AGGTCCCAGA GCAGCCATTT GGCTGAGGGA AGGGGCAGGT |
| | 77761 CAGTGGACAT CTGATCTTGG TTTAGTATCC TTCATTTTGG GGGCTCTGGG TGTGGCCTGG |
| | 77821 GCCTCTGGAC TTTGGCCACG GTGTTTGTTC CAGCCCTTCT CCTAACCTGT CCTTTCCAGA |
| | 77881 CACTCGGCAT CTAGGTTATT AGCACCTCGC ATACTTTCTG ACATGCTCCT CAGTCCTGAT |
| | 77941 TTTGACCATC TTCTCTTGCT TCCCATCTGT GTCAGTCAAG ACTGCATTTG GCTGTAAGAA |
| | 78001 ACAGAAACCC CAACTAACTG TGGCATTTAC ATGAAGAGGT TTACTTTTCT CACATAATCA |
| | 78061 GATGTCTAGA CTTGGCCAGC ACCTCAAGGG TCATTGATGC TCTCCTGTCT TTATTTTCTG |
| | 78121 TCATCTTTAG TGGTTGGATT GTTGCCTCAT GGTTACAAAG TGGCTGCTGC ACTTCCAGGC |
| | 78181 ATCACATCTG CCTTTGAAGC AGGAACAAGT TGCAAAGTAA AGTGGCCAAA AGGGCCCTGA |
| | 78241 AACTAAATGT GTCCCCTTAG GAAAGCAGGA GTTTTCTTGC AAGTGGCAAT CTTCTGCTTA |
| | 78301 TGTCTCATTG GCCAGAGCTG GGTCTTACGG CCACCCCTTG CTGCGAGCAA GGCTGGGACA |
| | 78361 TTGAGCATTT TGCCGTCCAA CCTCTTTAGC AGAATAAACC AAGGGGGAAG AACGTTAATA |
| | 78421 GTGGCTTTTG AGTCACTAGT TGGCAGTATC TGCCCCTCTA TCTTTCCATC CTCCCCATGG |
| | 78481 AGTTTCAAGG TTCCTTTCTC AGTACTTCTT CAGGCTCTGC ACGTTCATTT GGATCTTGTG |
| | 78541 TCTTGGGGTG AAAAACTGGC CCAAGTGTCT CCCCAAGCAT CCACCTTTGG ATTAATTTGG |
| | 78601 AAAATGGCTG TCAAGTGCCC GCCTCTTGCT TGGTATAATG CTACAGCTTT AGAGGACGCA |
| | 78661 GCAGGCATGG GCCTTGCCGC TGAGGTTCTT AGCCTCATGA GAATATCCAG ATCAGATTCT |
| | 78721 CTTGGCTCCT TCTTAGAGCC AGTGATGCAA GACACTTCCT GCTCATCTTG TCGGGACGGT |
| | 78781 TTTACAAGTT GCCTGCCATC CTGAGAAAGT CTACAAAACG ATGCCAGACC TCATGCCAGC |
| | 78841 TTCCCAAGCC TTGACTCTCA GTGCTCCCTC AACAGGATTC TGGAAGAATC TCCCAAACAA |
| | 78901 GTCGCAATGC CCTCTGGACC CTGTGCAGGC ATGAGACTCA AGAGCATTGG CTCCCACCCC |
| | 78961 TGGTGGAGGG AACACTGCTG GGGCTGGGAT CTTGCCTGGT TGCTCCGCCT GCACCCAAGA |
| | 79021 CAACCATAAT TAAAATGTCC TTCATTGAAC TTGGAAAGCC TTCAAAGCTG ACAACTCCTT |
| | 79081 ATGTGTACCC GGAAAGGCCT GGGAGTGTGC CAGGGCATTG CTCGGGAGGG ACGCTGATTT |
| | 79141 GGAAGCATTT ACCTGATGAG AGACTGACAG CAGCTCCTGG TAGCCGAGCT TTCCCTCCTG |
| | 79201 CCTCTGCTGT GAAGGTGGAC CCATCCAACA GTCAAATGCC TGACTCTGGA CAGGAGCGGA |
| | 79261 CCTATTTATT GCCATGCAAG GGACTCTGCA CTTTTGAATT GTGGGTCATG GGCTTGGATT |
| | 79321 TAGGGGTTAG AGCTGGGAGA AGTCTTGGAA GTCACCTAGA GATGACACTG CCATTTTGCA |
| | 79381 GATGAGGAAA CCGTCCAATC AAAATGGACC AAGGACTTGC CCAAAGCCTC ACAGCAAAAC |
| | 79441 CATAGGCCCC CGCACTAACC CCAGAGTCCC TGTGCTGTCT TAAGGATCAT ATAGTTGTAA |
| | 79501 GCAATCATCT GGTTTTCAGT ATTTCTTCTT TTAAAATGCC TGGGGCCATG CCCAGCAGTC |
| | 79561 TGTTTCACTG CAGCGTTTAC ACAGGGCTGC CGGGCTTTCC TGGTGGATGA GCTGGGCGGT |
| | 79621 TCATGAGCCA GAACCACTCA GCAGCATGTC AGTGTGCTTC CTGGGGAGCT GGTAGCAGGG |
| | 79681 GCTCCGGGCC CTACTTCAGG GCTGCTTTCT GGCATATGGC TGATCCCCTC CTCACTCCTC |
| | 79741 CTCCCTGCAT TGCTCCTGCG CAAGAAGCAA AGGTGAGGGG CTGGGTATGG CTCGTCCTGG |
| | 79801 CCCCTCTAAG GTGGATCTCG GTGGTTTCTA GATGTGACAG CACCCTTAGT GGATGAGGGA |
| | 79861 GCTCCCGGCA AGCAGGCTGC CGCGCAGCCC CACACGGAGA TCCCAGAAGG AACCACAGGT |
| | 79921 GAGGGTAAGC CCCAGAGACC CCCAGGCAGT CAAGGCCCTG CTGGGTGCCC CAGCTGACCT |
| | 79981 GTGACAGAAG TGAGGGAGCT TTGCGTGTTT ATCCTCCTGT GGGGCAGGAA CATGGGTGGA |
| | 80041 TTCTGGCTCC TGGGAATCTT GGGTTGTGAG TAGCTCGATG CCTTGGTGCT CAGTTACCTC |
| | 80101 CCTGGCTGCC TGCCAGCCTC TCAGAGCATT TAGGGCCTTC TGGACTTCTA GATGCTCCTC |
| | 80161 ATCTTGCCTC AGTCAGCGCG TCAGTTCCAG AGACTTCTCT GCAGGGTTTT CTGGGGCAGG |
| | 80221 TGGTGGCAGA CCCGTGCCTT CTTGACACCT GAGGTCAGTC CACCCTCCTG CTCAGACTGC |
| | 80281 CCAGCACAGG GTCACCTCCC AAGGGGTGGA CCCCAAGATC ACCTGAGCGC ACAGAGGGTG |
| | 80341 CAGATGACTG GACCACACCT TTTGGTGATC TTAATGAGGT GGTCCCAGAG GAGCTCAGAC |
| | 80401 ATGCAATCTA GCATCCAGTT CTGGGACTCT GTCTCCTTTT CAAACGTATT CATGTAGAAC |
| | 80461 AGGCATGACG AGAATGCCTT GTCAACATGG GTGATGGGGA ATCAATCAGA CAGGGCGCCG |
| | 80521 GGCTCAAGGC TGCAGTCACC CAAGAGTGGC TCAGCCCACC AGGCCCTAGG AAACGCCTGC |
| | 80581 ACAGCCTGGA GCTCCTGGAG TCATTTCCTT CATGTCTTCT TCACTGCACT TACGTAAAGA |
| | 80641 TGCCAGCCAT TGGTTTGGTG ATTTGGAGGG TGCCCAGTTG CCCAACAAGA AATGCAGAAG |
| | 80701 AGGCCTAGCC AGGATTTCAC CAGCAGTGGA GAGTAGAGAA GATGTGGCCA GAAAAGAGTT |
| | 80761 TCCTTTCCCT CCTAAAGATG GTACTCCCTG CAGCTACTGG GGAAGCCTGC AGCATTCTCT |
| | 80821 AGGGCTCTGT GTGTTGAGAG CAGCCCCACC CTGGCCCCTT CTGAGTGCAT TTCTGCTTTG |
| | 80881 TGACTTGATC CGTGAAGTCC CCTGAGATGG GCAGAGGGGA TGTCCTCGAA GCTGGGGCAG |
| | 80941 AGCCTCATCC TTGAACGTGA AGGACGTTTG AAGACTGTGG CATGATCACA GGATGAGATC |
| | 81001 ACAGGGAACT TGAGTTTCTC TCCTCCTCTC CCTTCACAGT TATTTCACTG AGGGAAATCC |
| | 81061 CTCCCCTGCC CAGAATGAAA ACTCTAGCCA ACTCTTGACT TTTCCATCAC TCCAAAGTAG |
| | 81121 TTGAAAGTAC ATTAGTCTCC ACAGTGGCAA AACAGTGTGC AAAAGCTAAA TAATTAGAAC |
| | 81181 AGCCAGTCCC ATGTGACAGT CAAAGCTTCT AACTCCATTC AAAGTTGCAG CCATTCCCCT |
| | 81241 CGAGGGCTGG CAGGGAGGGG AGGGGTAAGA GAAACAGGAA GGTTCTTACT GAGTTGGTCC |
| | 81301 TGGTGTGAGC TGCGTCACAC TCCCTGCAGA GGTTTCAAGG AGACTCTCTC TCTCTCTGTC |
| | 81361 TCCATGGGGA CCTTATTTGA ATTCTTCTAC TCTTACCCCA GCCTGCCATC TCCAGCTATC |
| | 81421 CTCCCCTGAA GAGCCCTTCT GCTGCGCTGG ATTCTGGTGG CCATGTCATC TCCTCGGCCC |
| | 81481 CGTGGGAGTC TGAAGATCTG GCTGCAGCCT CACCTCTGAG GTCCTGCTAG TTGCCACCTC |
| | 81541 TTAAACATGA TCTGAGGCTC CCATGCACTC TGACCTGTGC CCACATGGGG CCCACGGGAA |
| | 81601 ACACGCTGGC AAGCAAACTG TGGGTGTGCA GACGGTTCTC AGGGCTGCAG CACCTGTCCT |
| | 81661 TTGCTCTGCC CCCAAAGCAA GGCCAGCCCA TCTTCCATCC TCTAGTGTTC CTTGGTGGGG |
| | 81721 CCCTGACCAC AGTCCACCAG GTCCCTAACC AGAGGGGACA CACACCAGGT GTCCTCAATG |
| | 81781 TATTGCCTTG AAACAGTTGT GCTGGGACTG TGATGGGGGG TGGCCATGTA GCCACCCCCA |
| | 81841 CCACCCCCAA GCCACTCTCT CCAAGGAAAT CCTCCTAAAG ATCCCTTTAC ATCCTCCATG |
| | 81901 TGGTGGGGAG GTTCTAGAGT TGGGTGCATG TGTCTTCAGC TACTGACAAT GCAGACCTTA |
| | 81961 GTTGGCACCT CGCTCTGGCC TATCCTGTTT GCTGTTCTTG GCGCTCCAGT GAAACTCCCC |
| | 82021 ATGGGCCATC CAGTTGGGGT GCAGTGTGGC CACCCCCTTG CAGGTTCCTG CCTTGCTGGA |
| | 82081 GAGCACAGGG CCCTCCTGGC TCTTGTAAAA CACTCCCCAT GGTACAGAGA GGCCAGCAGT |
| | 82141 GATGTGAGGC CCAACCTCCC TCCATGGTGT TCCCAAGCAG CTCCCTTTCT GGGGTCAAGG |
| | 82201 GGTGGCAAAG ACAGTGCAGC GTCCAATTTC TGACTCAAGC CGGGCCTGGC TATCGCAGCT |
| | 82261 CTGCACTGTG TGTGACAGCA AGGCAACTCA CCCAGTGCCG TGGCAGTGAC CGTGTCCGAG |
| | 82321 GAAGCCTCCT CACACCCTCT GTCTCAAGGA CTCTGGCATT TAGCTGGACT TGCTGTAGCT |
| | 82381 CTGAGCCTTT CTGCCATTGC CATCACCTTG TCAGAAACTC AGGCCGAATC TGCACTCAGA |
| | 82441 GTTGTGCCCA GGCAGTTGAG CCAACACTTG CTCAGCGATA TTGTCACATG ACAAGGCACT |
| | 82501 GTCACCACTG GGCATCGTGG GTAGCGCAGT GTCGGCTGGA TGGACCCGGA GGGTGTCTGT |
| | 82561 GTCATGCTAG TGCTAGTGAT GGGAGCCCCG TGAGCCCATT GCCCGCCCTC CCATGCCCTC |
| | 82621 AGCAGCTGCC TGGGGACAGC CAATGGCCTG GGTGTTTCTG AGGCTACCAC ATGGCTTCCA |
| | 82681 GGAAACTCGA GAACCTTTCT CTCCCTTGCC TACACTCTTC ACACAGGCCT GTGCTGGCCA |
| | 82741 GCGGTGGGGA TCCGGCATTC CTATCTTAGG TGCAGAGAGT GACTGACTCA TTGCAGGCCT |
| | 82801 GGGAGATAAG ACTGATGGCC CAGCCAGCAA GATGTATGGA TTTCTCAGAG GCAGTGGCCT |
| | 82861 CTGTCATTGT CCTCAGGAAA TGCTGGTGAT TCTGGTGGCC TGAGGTCAAT GCATGTCAAC |
| | 82921 GTGGCCAACT TGCCTTATAA ACTTTTTTTC TGGACAATTG CGTGCACTGT CCTGTAACAG |
| | 82981 TGTCCTGTTG TTTATGATGC AGAAATAGGT GTTTTTAAAG CCTATTGATT TTGGTACTAT |
| | 83041 TAATGTGGTC AGGAACTTTC TCAGTCTTTC TTGTTTGGGG TGAGCTGTGG CTTCCTAAAC |
| | 83101 AGGAACCCAA GACACCCCCA AAAGCTGCTC ACCAGCACTG CCAGCCTCCC TCTTACCAAG |
| | 83161 TAGCACCCGT TCAGGACATT CTGCGAAAGG CATTTGCCCA GAAGTTGGGA GGAAGGAAAT |
| | 83221 GTAACATTTT GGGGCACCTA CCATATGCCA GGCACCAGGC TAAACGTGTT CACACAAATT |
| | 83281 CTCTTACTAA CCCTCACCAT CCTTCTACAA GACAAACTAG TATCTTCATC TTGGGGTTCA |
| | 83341 AGATGAGGAA ATGGAGGCTC AGAGAGGTTG AATGAATGCC GGTGCCTGGA TATGAACCCC |
| | 83401 ATCTGCCTGA CTCCGCAACC CAGGCAAAGT CTTTCCTTGA ACTTCCCAGC AGCCACTGCT |
| | 83461 TAGACACAGC CTCCACAACC ATGGCTCAGC AGCAAATTGC TTCTCTGACC TCACTCAGCC |
| | 83521 TGTGTGTCCT TGTTGAGTGA GGCATTCAGG ACCCTGGTCC CAAAGTGGAG AAAGTCTTTC |
| | 83581 CTACTAGGTC ATAGCTACAC CTGCATGTGG GTGCTGTGCC TTTTGTTTAG TGAACTTTTA |
| | 83641 TCACCAGCAT CCTCAGCAAT GACATTTGCA GAGAAGCCAG AGCTGAGGCA CCTTGGTATT |
| | 83701 CTTGGGATGT GACTTTCCTG AATGTTTAAG GGAAAATGCC CGAAGGTACA GAGAGCTTGG |
| | 83761 TTTCTAGTAA ACAATAACTG TCTTGCTTTT ACCCCCCTTC ATTTGCTGAC ACATACACCA |
| | 83821 GCTGAAGAAG CAGGCATTGG AGACACCCCC AGCCTGGAAG ACGAAGCTGC TGGTCACGTG |
| | 83881 ACCCAAGGTC AGTGAACTGG AATTGCCTGC CATGACTTGG GGGTTGGGGG GAGGGACATG |
| | 83941 GGGTGGGCTC TGCCCTGAAA AGATCATTTG GACCTGAGCT CTAATTCACA AGTCCAGGAG |
| | 84001 ATTTTAGGGA GTTGGTTCTT ATCAAAGGTT GGCTACTCAG ATATAGAAAG AGCCCTAGTG |
| | 84061 GTTTTTTTCT AATACCATTT CTGGGTAATT CCTAAGGCAT TTAGTGTTCT GAAAGATGCT |
| | 84121 AGCCTTGTCC AGCCTGGGAG TTGAGAATGA ATGTCTAACA GAAACTCTAG GCCGGGCCTG |
| | 84181 GTGGCTCACG CCTCTAATCC CAGCACTATG GGAGACCCAG GTGGGCAGAT CACCTGAGGT |
| | 84241 CAGGAGTTTG AGACCAGCCT GGCCAACATG TGAAATCCTG TCTCACTACA AATAAAAAAA |
| | 84301 TTAGCCGGGT GTGGTGGTAG GTGCCTATAA TCCCAGCTAC TCAGGAGGCT GAGGCAGGAC |
| | 84361 AATCGCTCGA ACCCAGGAGG TGGACGTTGC AGTGAGCCGA GATCGCATCA TTGCACTCCA |
| | 84421 GCCTGGGCAA CAAAAGCAAA ACTCCGTCTC AAAAAAAAAA AAGAAACTCA AATATGTGTG |
| | 84481 ACAGGCGATT CTCACTGCAG GCTGCCCTGT GGCTGATCCA GGAGCAAGGC CTTAACCATG |
| | 84541 TCATCCCCAA GCGATTGCTT GTAAACTTTC TTCTGTGCAG CCTTCAACCC TTATTATGAT |
| | 84601 TTTCTTCTCA GGAACCAAAC TGCTGTATTC AAGAAAGGCA GCTTTGTGTA ATCATTTATC |
| | 84661 ATAAATATCT TAAGAAAAAT CCTAGAGATT CCTAATTTTA GGAAATGGGA GACCTATGGT |
| | 84721 ACTGATATAA TGTGGGCTGG GCTTGTTTTC TGTCATTTGC TAGATAAATG AACTTGAGAG |
| | 84781 CCTACTGTAA AATGTGGAAG CTTCTAGATT GCAGAAGGGC TGGAAAGACA CTGTTCTTTT |
| | 84841 CTCCCGAGTG ATGGGATCTG TCCAGTATTT AGAGCTGCCT CTGAGGCCAT CTGATTCTAG |
| | 84901 GAGACTCTGC CTCGTTGAGG ATATTTTGAG GCCTAACTAC ACATTCCTGC CCCCAGAGAG |
| | 84961 GTCACAGCCT ATAGCAGGCT GATGTTTCTC ATGTCACATG GCACAGAAAG GCACATTTTC |
| | 85021 GTTCTCAGGC TAACAAAGAG CTTCAAAAAC TATTAGAAGG GACAGTGGCT ATAAGAGAAG |
| | 85081 AACCTCAGTC AATGTGTGAA ATTAACTAGG AACCTGGCTC CTGTTTCTTT TAGGTCATGT |
| | 85141 TTTTCAGCTT AGGTAAAACT AGAGGCTTTG ATAAAGCATG ACCTCTAGAA ATCATTGCTT |
| | 85201 TTCATAAATG GAAGTGGGTT TGAGTTTTTT CTACTGATTG TTAGTGCAGG TGATGTCTAC |
| | 85261 ATGCCCCCAG AACATATTCC ATGCAACAAA AAAAGCCCAG GTCACCGTCT TTGCTGGGAA |
| | 85321 CTTGACTTTT GTGCTCACTG AATTTTAAGC TTTCTGACAG CAGCCTGGAA TCATGGAGGG |
| | 85381 ATAAAGTACC TATTAGTAAG ATGGAAAAAG GTGTTTCAGG TTGGAGCTGC AGTCTGTTGA |
| | 85441 GAGTAAGCTA TGGGAAGGCC TGTATACGAG GGGTGGACTT TTCTTCTGTA AGTGTCCAGA |
| | 85501 GACCAGGCCT CCTGAAGAGG GCATGGGGGC TTAACTTACC TGGACTACTG TGTTTACAAT |
| | 85561 ACTCATTTAT CTTGAACTCC TCCTAACCCC TGAGAATTGC TACATTTAGT ATTTGCTGAG |
| | 85621 TACTTCCTAG CATCCTAGGG AATCAATAGA ACATTCTCCC AACCAGGCTG GGTGCGGTGG |
| | 85681 CTCATGTCTG TAATCCCAGC ACTTTGGGAG GCCAAGGTAG GCAGATCCCT TGAGGCCAGG |
| | 85741 AGTGCAAGAC TAGCCTGGCT GACATGGTGA AACCCCGTCT TTACTAAAAA TACAAAAGTT |
| | 85801 AGCCAGGCAT GGTGGTACAC ACCTGTAATC CCAGCTACAT GGGAGGAGTA GGAGGCAGGA |
| | 85861 GAATTGCTTG AACCTGGGAG GTGGAGGTTG CTGTGAGCCG AGATCATGCC ACTGCACTCC |
| | 85921 AGCCTGGGCG ACAGAGTGAG TGAGACTCTG TTTAAAAAAA AAAAAAAAAA GAACATTCTC |
| | 85981 CTAACCTGGC TTCTTCCTCC AGGGGTGTAA TTAATCATGT CAGTTTCCTC ATTGATACAC |
| | 86041 ACACACACAC ACTACAATCC TGTATCCATT ACTTTTCAAG GTACATTTAC TATTTACGTT |
| | 86101 TGGGGTCCTT GTCTCTTTTT TAATAGTGTT TCTTAAAGTC TTGTATTATA TCAGAGTACA |
| | 86161 GTAACATCCC AGTCAAGAGC ACTCTAGTAA GCTCTAGGAG GAAAGCGACT TCCGGAAGGC |
| | 86221 AGTGGAGACC TGTCCTGTTG GGGCAGCATA GGGGCAGCCC CTGCCTCTGG TCAGTTCTGG |
| | 86281 CGCTCAGGCT CAGGGTTGCC TCTGGGCTGT TCTTCCCAGA GACTGACAAA GGGCTCCCAT |
| | 86341 AAGGCACCTG CAGAGCCTGT GAGAAGCTGA AGTCAATGTT TTCCTGACAC CAGTTGATCT |
| | 86401 GTGCAGGATC CATTGATTTA ACCACCTGCT GTGTGGCATG CACTGTGGTC GATGCCAGGA |
| | 86461 ACAGGAATTG GAGGGGCCCA TGAGCATGGC CAGTATCACA GGCTGGAGGT GCTGCTGCGC |
| | 86521 TCTGACCGGG CCTCTTGGGG ATGAGCCCAT GTCAACCACC TTGCCTCCGA TGGGGTCGGG |
| | 86581 CCCACAGGTT ACCTTTGTGT GTCCATGACC ACACCTTCCT CCCCGACCTC ATCCAAATCT |
| | 86641 CTTTCTTTTC CAAGCCCCTG AATCCTTCAG GGCTGCAGGT TTTGTTTAAA GCAGAGCTGG |
| | 86701 TGAGTTGCAT AGGTTGTTGC ATTGGGACTA GATGGGGTGT TCAAAGAGTT GGGAGTTAAA |
| | 86761 AAACATAAAG GGTATTTATT AGGAGAACCA AGGAGTGTAA TTCTCCTGTT CTTAATATGC |
| | 86821 GGCCAGGTTA ATGAATGTCA CGTGAATGAA CCAGAAAAAA ATGAAGTGTG CCCTTGATCA |
| | 86881 GCTGGGTTGG TGTGCAGCAA GCTGTGTGAC CAGGGGACAG CAGTGGTCCT GAGGGCCGTC |
| | 86941 ACTGTCTGCC GTGCAGAGCC CTTCCTCCCA CGGGGGCCTA CCTCACCTGT GCCAAGGGCT |
| | 87001 TGTCTGTGGT CAGTGACCTG GATAGATCTG AATGGGGCTT CTTTTTCGAG GAGTCTTATG |
| | 87061 GCAGGTCTCT CAGTAAAGAC TCCATTCTTG ATGATCACAC ATTTTGGATT TTCCAAATCT |
| | 87121 GTCAGAGAAT GGGCTTGAGG CGGGGTTTGT GGGCACTAGT TTCACTGGTT TCATTTACCA |
| | 87181 AAAAGGGGAG CAGAAGTCAA GTATGGTGGC TCATCCCTGT AATCCCAGAG GCAAGAGAAT |
| | 87241 TGCTTGAGCC CAGGAGTTCG AGACCAGCCT GAGCAACATA AGGAGACCCC GTCTCCACAA |
| | 87301 AAATGAAAAA TAACATTTTA GTCAGACGTG GTGGCATGCA TCTGTGGTCC CAGCTGCTTG |
| | 87361 GGAGGGTGAG ATGGGAGGGT TGTTTGAGCC CTGGAGTTAA AGTTGCAATG AGCTGTGATT |
| | 87421 GCACCACTGC ACTCTAGCCT GGGTGACAGA ACGAGACCCT GTCTCAAAAA AAAAAAAAAA |
| | 87481 GAAAGAAAGA AAGGAAAAAA AAAACTCATG CCTGTAATCC CAGCACTTTG GGGACCGGGG |
| | 87541 TGGGCAGATC ACGAGGTCAG GAGATCAAGA CTATCCTGGC CAACATGGTG AAACCCCGTT |
| | 87601 TCTACTAAAA ATACAAAAAT TAGCCAGGTG TGGTGGCACG TGCCTGTAAT CCCAGTTACT |
| | 87661 CGGGAGGCTG AGGCAGGAGA ATCGCTTGAA CCAGGGAGTC AGAGGTTGCA GTGAGCTGAG |
| | 87721 ATCGTGCCAC TGTACTCCAG CCTGGGCGAC AGAGTGAGAC TCTGTCTCAA ACCAAAAAAA |
| | 87781 AGGGGTGGGG GGCGGGGGCA GGAGAACAGT GAGAGGTAGG GAGAGGAAAG GGGATTCTCG |
| | 87841 CTACACCCAA ACCAGATACC ATCTAGAGGC TAGAATCTTT GGGAGGCTCA AATTCCCTAG |
| | 87901 AAAGCAGGAG AAGCTTCTGT AGCCCTCCCG CTTTCCCAGT AGATTAAGCC CAGGGCGGCT |
| | 87961 CCAGATGTGT GACATGCTCT GTGCCCAACC AGAGCCCATC ATAGGCAGAG GAATAACACC |
| | 88021 CACACCAGAA GGGCCCTCGG AGGTCACCAC GTCCAAGAAC CCTCTTTACA GATGAGGAAA |
| | 88081 CTGAGGCCCA GAGAGGGGAG AGCCACCTAG CGAGCTGGTG GCGGCTAGAC CAGGAGAGCT |
| | 88141 GTCATTCCAA GCAAGCAAAG GCAACGAGAC GAGCCCAGAG CTGTGCTCCC ATCTCTTTGT |
| | 88201 TAGGGGGCCT GGGATGCCCT CTCAGTGTCA TTTTGTCCAG GATGATGCTC CCTCTCTTAA |
| | 88261 GCGATTAATG CGCCCTTGCT AACCTTTTGC TATCGCTGCC TCTTCAAACC AGAGGAGTTG |
| | 88321 AGAGTTCCGG GCCGGCAGAG GAAGGCGCCT GAAAGGCCCC TGGCCAATGA GATTAGCGCC |
| | 88381 CACGTCCAGC CTGGACCCTG CGGAGAGGCC TCTGGGGTCT CTGGGCCGTG CCTCGGGGAG |
| | 88441 AAAGAGCCAG AAGCTCCCGT CCCGCTGACC GCGAGCCTTC CTCAGCACCG TCCCGTTTGC |
| | 88501 CCAGCGCCTC CTCCAACAGG AGGCCCTCAG GAGCCCTCCC TGGAGTGGGG ACAAAAAGGC |
| | 88561 GGGGACTGGG CCGAGAAGGG TCCGGCCTTT CCGAAGCCCG CCACCACTGC GTATCTCCAC |
| | 88621 ACAGAGCCTG AAAGTGGTAA GGTGGTCCAG GAAGGCTTCC TCCGAGAGCC AGGCCCCCCA |
| | 88681 GGTCTGAGCC ACCAGCTCAT GTCCGGCATG CCTGGGGCTC CCCTCCTGCC TGAGGGCCCC |
| | 88741 AGAGAGGCCA CACGCCAACC TTCGGGGACA GGACCTGAGG ACACAGAGGG CGGCCGCCAC |
| | 88801 GCCCCTGAGC TGCTCAAGCA CCAGCTTCTA GGAGACCTGC ACCAGGAGGG GCCGCCGCTG |
| | 88861 AAGGGGGCAG GGGGCAAAGA GAGGCCGGGG AGCAAGGAGG AGGTGGATGA AGACCGCGAC |
| | 88921 GTCGATGAGT CCTCCCCCCA AGACTCCCCT CCCTCCAAGG CCTCCCCAGC CCAAGATGGG |
| | 88981 CGGCCTCCCC AGACAGCCGC CAGAGAAGCC ACCAGCATCC CAGGCTTCCC AGCGGAGGGT |
| | 89041 GCCATCCCCC TCCCTGTGGA TTTCCTCTCC AAAGTTTCCA CAGAGATCCC AGCCTCAGAG |
| | 89101 CCCGACGGGC CCAGTGTAGG GCGGGCCAAA GGGCAGGATG CCCCCCTGGA GTTCACGTTT |
| | 89161 CACGTGGAAA TCACACCCAA CGTGCAGAAG GAGCAGGCGC ACTCGGAGGA GCATTTGGGA |
| | 89221 AGGGCTGCAT TTCCAGGGGC CCCTGGAGAG GGGCCAGAGG CCCGGGGCCC CTCTTTGGGA |
| | 89281 GAGGACACAA AAGAGGCTGA CCTTCCAGAG CCCTCTGAAA AGCAGCCTGC TGCTGCTCCG |
| | 89341 CGGGGGAAGC CCGTCAGCCG GGTCCCTCAA CTCAAAGGTC TGTGTCTTGA GCTTCTTCGC |
| | 89401 TCCTTCCCTG GGGACCTCCC AGGCCTCCCA GGCTGCGGGC ACTGCCACTG AGCTTCCAGG |
| | 89461 CCTCCCGACT CCTGCTGCTT CTGACGTTCC TAGGACGCCA CTAAATCGAC ACCTGGGTGC |
| | 89521 AGCTGCTCCA CTCCCTCGGC CTCCTCCCGT GCTCAGGCTG TGGCCGCACG CGCCCCTCAC |
| | 89581 GCTTGCCCGC CACTCTGCAT GTCACCAGCA CCCCCGCTCC GTGCTACCCA CCTTGTTTGA |
| | 89641 CTCTCTGGCC ACTTGATTTG TCCACAACGG CCCATCAGCC CACAGGAGGT TTGGTGGGTG |
| | 89701 CCTTCCACCG ACAGGATGAC GGGTGCCCTC ATGGTGTCTA GAACTCTCCA ACCCTCCCAT |
| | 89761 GTAGGCATAA GCAGCCCCAC TTTGCAGATG AGGAAACGGA GGCTCAGAGA AGTACAGTAA |
| | 89821 CTTGCCGAAG GCCAATGAGT AGTAAGTGAC AGAGCCAGGT TTGGGATCCA GGTAGGTTGT |
| | 89881 CTCTGAAAGA CACGCCTGTC CTGCATCCCA CAACGCCTCC CAGGAGGTGC TGGAGTGTGG |
| | 89941 ACGCCTAACA CAGAGATGTG CAGGGCACAC ACAGCAGGTG ACACACACAG CATCCAGAGG |
| | 90001 TGGCCCAGAG CTCATGCTGT GCCTTTGGCC CAGTGCCCTG CCCCCACCCA CTCTGCCTTG |
| | 90061 TGGCAGGAAG ACAAGGAGCA GACACAAGAT CTCCCTGGTC CACATGCCAC CACCTCCCTC |
| | 90121 TGCAGAGGAC AAGGGGATCC TCATGCTGGC ATTGGAGGGG GTTGAGCAGG GCCCACCTTG |
| | 90181 AGCCCTCAGG AGCACGACCA CAGCAGCCCT GCAGGGAGGG ATTGGTGGGA GGAGAGTCCC |
| | 90241 AAGTATCAGG GAGAGGAGAG TTGGTGTCCC ACAGGAGACC TCAGAGCCAC AAGGCGAGCT |
| | 90301 TGTTCATAAA TTTGGGACCC TTAGCATTTC ACAGTTATTT GCAGAGCCCA GAAATGGATG |
| | 90361 TTACTGAAGC TCACAGTTGC AAGCATCTGT TAAATTTTTA TTAGATTTTA CTTTTAGGGA |
| | 90421 AAACTTTGAA ATGCTATAAA GAAGCCTGTG TTTAAAAGTT AAGACAGAGG CTGGGGGCGA |
| | 90481 TGGCTCACGC CTGTAATCTC AGCACTTTGG GAGGCCAAGG CAGGTGGATC ATTTGAGGTT |
| | 90541 AGGAGTTCGA GACCAGCCTG GCCAACATGG TGAGACCCTG TCTCTACTAA AATTACAAAA |
| | 90601 AATTAGCTGG GCGTGGTGGC GGGCACCTGT AGTCCCAGCT ACTGGGGAGG CTGAAGCAGG |
| | 90661 ATAAGTGCTT GAACCCAGGA GGCAGAGGTT ACAGTGAGCC AAGATCACAC CACTGTACCC |
| | 90721 TAAGCCTGGG CGACAGAGTG AGACTCTGTC TCAAAAAATA AAATAAAATA AAGTTAAGAG |
| | 90781 AGAAAAAAAT ATATCCTATA TCCTTTGTTA AATTCCAAAA CAGTAGGGGA CAAATAACTG |
| | 90841 ACTTGACAGG TTACTACAAT ATTTCCTGAA ATGATGTTTT CTTGAATACT GGCCTACTAG |
| | 90901 AGGTTCATAG GTGTGTTTGG ATTAAAAAAG AGTTCCATGG CCCAGTGACT GGGGGAAAAA |
| | 90961 AATAAAAGAC TAAAGTAAGT TAAACAGGCT TTTCTGCTGC AGGACTTGTC AGAGCCTTTA |
| | 91021 ATGTACTAAT GGCCATTGTG ACCCTCTGAG AAGGTCACAG AGTGGGTTTC CCAAACTTAC |
| | 91081 TTGATTCTAC CTGCTAACAT TTCCTGGAGG AAGTTTGGGA AATGCCGATT TAGCAGATTC |
| | 91141 TTTTGTTGTG CCGTGGATGG TGCTGGTTGA TGTGGGCAAA ACAAAGAACA CGTGAGTCAG |
| | 91201 ATCCGCCTGG GGCTCTTACT AAAGTGCAGG TTCCCAGGTG CCACTTTAGG CTTACAGACC |
| | 91261 CAGTTGTGGG GTAAGCCTGG GAGTCTTTTA GCAGGTGATT CTGCCACATA GTATAGTTGG |
| | 91321 AAAACCTCTG GGCATACTCA TTGCTGGTCC CTCTAGAAAT CCAGGTGACA ATAGCCAATG |
| | 91381 AGAAGCTCCA AGAGACCCAG TTGTCCATGG GGTAGAGGGA ATGTGATATT GAAACCAAAG |
| | 91441 AAGAAAATCT ATGATCAGTT TTCAGCAGTG ACTGTCAAGA GAAGGAGAAG GGTGAGTTAG |
| | 91501 CGCTGATGCT GGCTGACAGG TCAGCGGGTT GGTTTCACCA AGGAGTGTGA TGAAGGCTGA |
| | 91561 TGTTGTCTGT GGGAATGTAT GATGGTAACT GGTTTGTAGC TAATTTGGGG AAGCAGTGAG |
| | 91621 AATTCGTGCC CTTTGAAGAC CAGTAAGTGG CAAGAAACCC ACCAGGCCTG GCTCAGGGCT |
| | 91681 GGGCTGGGCT TGGCTCGTCT CAGAGCAGCT GGGGCTGGTG GCCAAAGCCA CCATTAGTGA |
| | 91741 GGGGCAGGCC CTGGGGGTAC AACCAGCAAC TAGGGGACAA AGACAACCCT GCCAGCCTCT |
| | 91801 CCTATTCTGG AGGCGTGTGA CCAGAAATGG AGATGGGTTG GTCAGCATAA GATGGCCAGG |
| | 91861 AAGGTGGAAA TCAGGACTGC TGGCAATCTA GCCACATGGG CAGGGGAGCC GGGTGGTTCC |
| | 91921 AGGCAGTTTC CAAGGCCAAG AGGGTGAGCA GGCACCTCAC AGGGAATCAG GGCCAAGCCT |
| | 91981 GGCTGCAGTG TGGAGACAAT GCACCCACCC CCATCCTTGG ATCTTGCAGG AGGCTGGGTC |
| | 92041 CTCACTGAGC TACCAACATC CATGGCCCTG AGGCTTTTAA AACACCCATC CATGGAGTGG |
| | 92101 GGCTGGTCCC AGTGGGGTGA GGCTGACCCT GGCAGAAACA GGGCAGGAGC CTGTGGGTTA |
| | 92161 GGGAGACTGC ACCTTCCTTA GATAGCCTCC ATGCCATCAT GTCCCCGTGA CAGTTTCTGC |
| | 92221 TGCGTCCCCT CTGCATGGTC CCACCCTCGG CCAGCCTGCT GCCCCCTCTT GCCAGGTTGC |
| | 92281 GCTAATCAGT GACCCCAGTG TGCTGTGTTG ATACTAACAA TGCGAGGCCT AGCAGATTCA |
| | 92341 AGGGAAAAGA GAACCAACTG GGTTTCCACC AGACCCAACT AAACAAACAT GGACCTATCC |
| | 92401 CAGAGAAATC CAGCTTCACC ACAGCTGGCT TTCTGTGAAC AGTGAAAATG GAGTGTGACA |
| | 92461 AGCATTCTTA TTTTATATTT TATCAGCTCG CATGGTCAGT AAAAGCAAAG ACGGGACTGG |
| | 92521 AAGCGATGAC AAAAAAGCCA AGGTAAGCTG ACGATGCCAC GGAGCTCTGC AGCTGGTCAA |
| | 92581 GTTTACAGAG AAGCTGTGCT TTATGTCTGA TTCATTCTCA TATATAATGT GGGGAGTATT |
| | 92641 TGTCACTAAA GTACAGCTGT CATTTAAAGT GCTTTGTATT TTGGGGCAGG CTTTTAAAAA |
| | 92701 GTCCAGCATT TATTAGTTTT GATACTTACC CCAGGGAAGA GCAGTTGGCA GGTTCATGAA |
| | 92761 GTCATGCTCC TAATTCCAGC TTTCTTAGTG TACTTTCAGT GAGACCCTGA CAGTAAATGA |
| | 92821 AGGTGTGTTT GAAAACCAAA CCCAGGACAG TAAATGAAGG TGTGTTTGAA AACCAGCCCT |
| | 92881 AGGACAGTAA ATGAAGCCAT CTTCTCACTG CATAAACTGC ACCCAGATCT TCGCCCATCC |
| | 92941 TTCTCAGTAT TTCACTTCAC CCATTGTTTA CTGTCTCAAT GACTGGGGAA ATGTCTGGGG |
| | 93001 AAATGCTCCC GTAATTGCAC AGTGGCGTTT TTCCTGGAAA ATCCCACCAT GGCTCTAGAT |
| | 93061 AAGACCTATT TTTCTTAAAG GTATCTAAAA TTTCCAGCAT AAATTCTGTC TGAAACACCT |
| | 93121 GAATTTTAAT CAGTACTGGA GCCCGGAGGG CATCTCCAGT TGCCACATAG CTCTGAGCAT |
| | 93181 TCAGTGGTGT GTTGAGGGCT GCTCCCGGAA GTGCCTGCAG AGTCAGGGCT CCCCAGCCTC |
| | 93241 ATCTAGTGAG GCAGTGGAAG GGCCTGTGGG GATTTGGAGA GCTGGCCTGG GTCTCTGAAG |
| | 93301 TGATAGTGAC AGCTGCTTGT CAATCACGGT GCACATTTAG TGCTGGGGGC AGGGGGCAGG |
| | 93361 GAATACCAGC CTCATGCATG CATGCATTCA TTTGTTCCTT CCTTCATTCA TTCATTCAGT |
| | 93421 ACACATGGGT ACAACATCCC TGCCCTGGAG TTGCCCAGAG TCTAGGGAGG GGAAAGATCT |
| | 93481 ATTACCCTGG GCCTCGGCCA GCTGGGGAGT GCTGCTGGTG GAGAGGGGCC GTGTGCAGCG |
| | 93541 AGGGAAGGAG GAGTCGTCAA TACCCCCACC CCAGCTTTGC TTTCTTGTCA TCAGCCCCAG |
| | 93601 GGCCCCAGCC TGTGTCCCTC CTCTCCCATT GCTACTTCAT CTCCTGGGTC CTCCTTACCA |
| | 93661 AGCCTGACCA CACAGAGGGC CTTGGCCGCT TCCATGGGGA ATTGGAAAGC AATAAGATAG |
| | 93721 CATCCCCTAG AAGCCCAGTG AAGTCTGGGA CAGGACCCTT CTCTGAGCTC TGACTTGCTC |
| | 93781 TTGGAAACAC TTCGAGGCTT AGCCTCCCCA CTTTGTTTCC CGAGAGTGTG ACCTGTTCCC |
| | 93841 CTCCAAACAC CCCCTTCTCC TCCAGGGCCA TGCCCACCCG TCAAAATCCC CCACGGGCAG |
| | 93901 GACGAACTGT GGGTGTCAGT CACCATCTAT CCTGCATCCT GGTTCCAGGG CCCCCCCCAG |
| | 93961 CCCCGCCTCC ATAGGGACAG GCGTGCAGAC ACCCGTCCCT GGCTGCTTCC TCTTGTGGAA |
| | 94021 TGGGTTCAAA AGTAAGCAGT GTTGTTTACA CTGACAAACT GAAAAAAAAA GAAAAAGAGA |
| | 94081 TAACATTGGA GGCTTGGCAC AGTGGCTCAT GCCTGTAATC CCAGCACTTT GGGAGGCTAA |
| | 94141 GGTGGGAGGA TGTCCCCAGC CCAAGAGTTC TAGACCAGCC TGGGCAACAT AGCAAGACCC |
| | 94201 CATCTCAAAA AAAAAATTTA ATTGGCCAGG CAGAGGTGGG AGGATCACTT GAACCCAAAG |
| | 94261 GGTGGAGGCT GCAGTGAGCC GTGATGGCAC CACTGCACTC CAGCCAGGGC AACAGAGGGA |
| | 94321 GACCCTGTCT CTAAAACAAA CAAACAAACA AACAAACAAA AGAGTTAACA TTGGCCAGAT |
| | 94381 TAGGATTCAC CAGATAGTGT TAATATTAGT TTGATTTGAG ACTTTAATCA GAAAGCACAT |
| | 94441 GTGTGGTGGG GGTGGGTGTA ACCTAAGTCA GGTAGAATCT TTCCAACTTG GGGGGGGCAC |
| | 94501 ACTCCTGATT GTAGCCATAT GAGTCTGTCA GTGTGGTGGA AGAGGCCATG GGTTAATGGG |
| | 94561 CAGGTAAAAA AGCACCTTGC CTGGAATTGA GTAGAAAGTA AGGCCCTTCA GACCCCGTGA |
| | 94621 CACACTTGGG GACATTTTCT TGAGTAACAT CCTAAGATTC ATGTACCTTG ATGATCTCCA |
| | 94681 TCAACTTACT CATGTGAAGC ACCTTTAAAC CAGTCGTCTC CAAATTCAGG GGCACAGTAA |
| | 94741 CATCCAACAG GCTGGAGAAA GAACGTACTA GAACTTCCAT TCCTTTTTCA TGTCCTCTTC |
| | 94801 TAAAAGCTTT GTCAGGGCCA GGCGCGGTGG CTCACGCCTG TAATCCCAGC ACTTTGGGAG |
| | 94861 GCCGAGACGG GTGGATCACG AGGTCAGGAG ATCGAGACCA TCCTGGCTAA CACAGTGAAA |
| | 94921 CCCCATCTCT ACTAAAAATA CAAAAAAACG AGCCGGGCGT GGTGGTGGGC GCCTGTAGTC |
| | 94981 CCAGCTACTC GGGAGGCTGA GGCAGGAGAA TGGCGTGAAC CCAGGAGGCA GAGCTTGCAG |
| | 95041 TGAGCCGAGA TTGCACCACT GCAGTCCAGC CTGGGCGACA GAGCGAGACT CCGTCTCAAA |
| | 95101 AAAGAAAAAG AAAAAGAAAA AGAACTGTGA TTGGGGAGGA CGGTCACTTT CCTGTTCTTA |
| | 95161 CTGATCAGAA GGGATATTAA GGGTACCTGA TTCAAACAGC CTGGAGATCA CTGCTTTCAA |
| | 95221 CCATTACCTG CCTTATTTAT TTTTAGTTAC TGTCCTTTTT TCAGTTTGTT TCCCTCCTCC |
| | 95281 ATGTGCTGAC TTTTATTTTG ATTTTATTTA TGTTTATGTT TAAGACATCC ACACGTTCCT |
| | 95341 CTGCTAAAAC CTTGAAAAAT AGGCCTTGCC TTAGCCCCAA ACACCCCACT CCTGGTAGCT |
| | 95401 CAGACCCTCT GATCCAACCC TCCAGCCCTG CTGTGTGCCC AGAGCCACCT TCCTCTCCTA |
| | 95461 AATACGTCTC TTCTGTCACT TCCCGAACTG GCAGTTCTGG AGCAAAGGAG ATGAAACTCA |
| | 95521 AGGTAAGGAA ACCACCTTTG AAAAGAACCA GGCTGCTCTG CTGTGGTTTG CAAATGTGGG |
| | 95581 GTTTGTTTAT TTGTTTTTTA GCCTCAAAGA CCTTTCTTCA AATGAGTTCT GGCATAGAAG |
| | 95641 CACCGTGTAA AATAGTTAGA ATTCTGGGCA AAGGGGAAAA GAGAGCTGGG GGCCATCCCT |
| | 95701 CTCAGCACCC CACAGGCTCT CATAGCAGCA GCTCCTAAGA CACCTGGTGG GACCTTGGTT |
| | 95761 TCGAAATCGC TACTCTAAGG CTGGGCACGG TGGCTCACAC CTGTAATCCC AGCTCTTTAG |
| | 95821 GAGGCCGAGG AGGGTGGATC ACCTGAGATC AGGAGTTCGA GACCAGCCTG GCTAACATGG |
| | 95881 CAAAACCCTG TCTCTACTAA AAATACAAAA ATTAGCCGGG CGTGGTGGTA TGCGTGGTGG |
| | 95941 TAATCGCAGC TACTCGGGAG GCTGAGGCAC AAGGATTGCT TGAACCCCAG AGGCAGAGGT |
| | 96001 TGTAGTTAGC TCCAGCTTGG GCGACAGAGC AAGACCCTGT CGCAAAAATT GTTTAAAAAA |
| | 96061 CAAACCCAAA ATTGCTACTC TCATTGGGTT CCTTTGCCCA TTCCTGATTT TGGCAAGAGA |
| | 96121 AATGCTTCCA GATTGCCCTG ATCTGGGTAG GACAGCATCA CGCCATAGCA ACACTGCCCC |
| | 96181 GTGAGCTCAC TGCCCCCTCA ACTAGCTTGT GGTCCTTGGT TAATGTCAGT TTCTTTTTTG |
| | 96241 AGTTTGTGTT ATGTCTAAGG GTCATCTGCT GGGTAACGGA ACCCAGGGAC TGCCCTAGTC |
| | 96301 CCTAGACTGT GCCATGCCCG ACTCTGCCAG CTTTGTCAGT GATGCTGGTG CTCGCCTCCT |
| | 96361 CGGGTGCTCG CCTGGTCTGA GCACACCCAA GGAGTTCTTG AGGCCTTAGG GTTGTTTGCG |
| | 96421 AGAGAATGAA AGAACACGAC CTAGCTCTCT TTAGCATCCT TGGTCAGGTT CAACACTGCC |
| | 96481 CCCAGGGGCC TCTGGTGGAG CCAACCACCA TCAGCCAAAT AAATCCATAA TTAGAGTCAG |
| | 96541 AAAATGGATG TCTGCATATG TGTAGTGCAC TAATGTCCTG CCGATGATTG ACATGGAGTG |
| | 96601 GAGAGTGACC TGATCATTGC TGTGAGCTCT GCTGGCCTTG GCACAACTCA TGCTGATAAC |
| | 96661 TAATGCACAC AGTTCCTCTG GGAGGAAATG TCCTCAGGGA ACTTGGAGTT TGGGTGGGGA |
| | 96721 TGTGGGTTTG TGTGCCCAGC AAGCCCTTGT GGTTGTAGCA GACACTAGTG GCATCTAGGA |
| | 96781 GGCAAAGGGT CACCCCAGTC TTAGCCACGT TTTGAGTCAA GGTGGCGGAG TGGGGCTGGT |
| | 96841 GTTGACTCTT GGTGGCAGTA ACTTTTCCCA ATGGTGAAAA ACCCCTCTAT CATGTTTCAT |
| | 96901 TTACAGGGGG CTGATGGTAA AACGAAGATC GCCACACCGC GGGGAGCAGC CCCTCCAGGC |
| | 96961 CAGAAGGGCC AGGCCAACGC CACCAGGATT CCAGCAAAAA CCCCGCCCGC TCCAAAGACA |
| | 97021 CCACCCAGCT CTGGTAAGAA GAACGTTCTC TTGAATCTTA GAGGAAGCTG AAGCTCTCAG |
| | 97081 AGGTACAGCC TTCATTTTAG GAGGCCTTAG GCCACTGAGA ATGAATAACC CCTGGCAGCT |
| | 97141 GGTCAGCAGC TTGCAGTTTA CTAAGCACTG GAGTCTTCAT TGCCTTCTCA GTCCTTTTGA |
| | 97201 TTTCTGAGGC AAATGTTGAA TCCCTACCTT TTTTTTTTTT TTTCTTTTGA GACAGAGTTT |
| | 97261 CGCTTTTGTT ATCCAGGCCG GAGTGCAGTG GTGTGATCTC AGCTCACTGC ATCCTCCACC |
| | 97321 TCCCAGGTTC AAGCGATTCT CCTACCTCAG CCTCCCTAGT AGCTGGGATT ACAGGCACCT |
| | 97381 GCCACTATGC CCGGCTAATT TTTTGTATTT TTAGTAGAGA CAGGGTTTCA CCATGTTGGC |
| | 97441 CAGGCTGGTC TCGAACGCCT GACCTCAGGT GATCCACCTG CCTCGGCCTC CCAAAGTGCT |
| | 97501 GGGATTACAG GCATGAGCCA CCACTCCCAG CCTGAATCCT CACTTTTTAT CAATGAAGAA |
| | 97561 ATTGAGGCTG ATTCTGCAGC ATGATAAAAA AAAATACAGA AAAAGGAAAA AAAAGAAAGA |
| | 97621 AATCGAGCCT CTGAGAGTTT GCTTGACTGA GTCTAACCAG CTCATTTTAA ACCCGAGGAA |
| | 97681 AATGCAGTCA CATGACTACT AAGTGGCAGC TCTCGGAGCC TCTCTGGCCC CAAGTCCAGG |
| | 97741 GTTCCATAGA GGCAGCCCCA GCATGGCATG TTTTCAGTCC CCAAATGAGA CTCTGGAGAC |
| | 97801 AAATGTCTCT GGAGACAGAG CAGCAGCCTG GATAAGTCAC AATGGGTGAC GTCACTCAGG |
| | 97861 GCTCAACCCC TGGGCAGCTT AACTTGCTAG GGACGTTAGG AGTCTGCTGC AAAACCTGAG |
| | 97921 GGTCTTAGCT GAGCAGTCAC AGGCTGGGCC CGTTGCCCTG GGCTCCTGTG AGTAAAACCC |
| | 97981 AGTCAATTTT GAGTACCCAG TAAGGCATCC ATTGAGTTAT TTTGCAGCCA GGAGTGCTAT |
| | 98041 TAAGAACAGT CGCGGCTGGG CGTGGTGGCT CATGCCTGTA ATCCCAGCAC TTTGGGAGGC |
| | 98101 CAAGGTGGGC GGATCACCTG AGGTCAGGAG TTCGAGACCA GCTTGGCCAA CATGGCAAAA |
| | 98161 CCCCGTCTCT AATAAAAATA CAAAATAATT AGCTGGGCGT GGTGGCGGGC GCCTGTAATC |
| | 98221 CCAGCTTCTC AGGAGGGTGA GGAAGGAGAA TCACTTGAAC CCAGGAGGCA GAGGTTGCAG |
| | 98281 TGAGCTGAGA TCGCACCATT GCACTCCAGC CTGGATGACA AAAGTGAGAT TCCTTCTCAA |
| | 98341 AAAAAAAAAA AAAAAAACAG TCGTCCTCTT TGGGGATTAG GGACAGCCTG CCTGCCTGCC |
| | 98401 CGAGCACTTC TCTCTTCCAT TGCCCCAGTG AAGTATTCCA GGCCCCTGGG TTTAGACTCT |
| | 98461 GCACCATGTA GGGGTGTCTG ACCTGCACTT GCTCCTTGGT GGCACGGGCA GCCTATGGCA |
| | 98521 CTTGCTGCGG GCTGTGACCA AAGCCTGGCC TGGATCTTGG ATCTTGGTGA CTCTGCTTCT |
| | 98581 CCCTGGCCTG AGGGAGCTGC CCAGAGCCTG CCCACCACCT GCTGCGTGTC TTTGCGGTGG |
| | 98641 CATTTCTCGC ACACATGCCG TGCAGTGGCA CCCCCAAGGA TGGCCATTCA CTAAGGCCCA |
| | 98701 TTGTTTTTGT CTTTTCGCTT CGTGTTTTCT GGCCTGGTGT TTTTCTCATA TACATGTGAT |
| | 98761 CCAGGGATAA TTCCCAGAAT TTTGACAGGA TTTTAAGTAG CGTTTGGATC CTGCTGTTTT |
| | 98821 TTTTTCACTT AACATCGGGC CAGTTGACTC ACACTCTGTT TTTTGTTGTT GTTTTTTTGA |
| | 98881 GACGGAGTCT CACTGTGTCA CCCAGGCTGA AGTGCAGTGG CACAATCTTG GCATACTGCA |
| | 98941 ACCTCTGCTT CCCAAATTCA AGCAGTTTTC CTGCCTCAGC CTCCTGAGTA GCTGGGACTA |
| | 99001 CAGGCACAGG CCACCACGCC CTGCTAATTT TTGTATTTTT AGTAAAGACA GGGTTTCACC |
| | 99061 ATTTTGGCCA GCCTAGTCTC GAACTCCTGA CCTCAAGTGA TCCGCCCACC TCGGCCTCCC |
| | 99121 AAAGTGCTGG GATTACAGGG GACTCACACT TTGTAACAAC CTGAAACAAC GTGATGCATT |
| | 99181 TCCCTTTGGG TCTTACCTGC TCTTCGGTGG CTGCCTGCAG GTGGAGAGAC CCTCCCCCTT |
| | 99241 GGGCCCCTCG ACCTTGTTTC AGAATGGGGC CCCTGCTGGG CCAGCTGTGG GTGCCTGCCA |
| | 99301 CGTGAAGGAC TCATTAAGGC CCTGTTTAAG CCTGATGATA ATAAGGCTTT CGTGGATTTT |
| | 99361 TCTCTTTAAG CGACTAAGCA AGTCCAGAGA AGACCACCCC CTGCAGGGCC CAGATCTGAG |
| | 99421 AGAGGTACTC GGGAGCCTAC TTCGCTGGGA GCAGCCTCCC TTTGCGTGTG TGGCCATTCA |
| | 99481 CTGGCTTGTG TTTCTAGAGC CGGGAGGACC CTTTTCTGCA ATGCAGGGTT CACACAGGGT |
| | 99541 TCGCAGCCTG AAGATGGAGC AGTCCGAATT CTCTTCCCTG TGCAGTTTGC GCAGCTGTGT |
| | 99601 TTGTCTGATG GGCTTTCTAA TCCTGTGTGC TCTCCTTGAC TTCAGGGACA ATGGCATTAC |
| | 99661 AGGCATGAGC CACCATGCCT GGCTGTCTCC CTATGTTTCA GATGAAGACA TAGGCTTAAG |
| | 99721 GAGGTCAGGT GACTTGCCCA CGACCACTCT GTAAATAAGA GGCATGAAAA GTATTTGGAG |
| | 99781 CCACCACCAC CAAGCCCACT GGTCACCCTG GGTCTCTGAA GTCAGGGAGG CAGGAGGATG |
| | 99841 GGAGGTCTGA GGAGGCAGAG AGGCTGAGCC TGGAGGCCCT GGAGGCCGAG GCCCCATCTG |
| | 99901 TTGTTTCCTT ATGTGGAAAA TAAGAGGCTT CGTTTGTCCT ATTGCCACAG AGCGTACTAC |
| | 99961 TTCAGGAACA TCCAAGACAT GGAAATCCGC AGGGCACGGT GGCTCACGTC TATAATCCCG |
| | 100021 GCACTTTGGG AGGTTGAGGT GGGAGAATCG CTTGAGGCCA GAAGTTCAAG ACCAGCCTGA |
| | 100081 GCAACATAGT CAGACCCCGT CTCTATAAAA AACATTATTT TTAAAAAAGA CATGGAAGTC |
| | 100141 AAATTCTAAA AACTGGTGCT GGCTGGGTGC GGTGGCTCAT GCCTATAATC CCAGCACTTT |
| | 100201 GGGAGGCCGA GGCGGGTGGA TCACCTGAGG TCAGGAGTTC AAGACCAGCC TGGCCAACAT |
| | 100261 GGTAAAACCT CTACTAAAGA AATCTTTACT GAAAATACAA AAATCCAGTC TCTACTAAAA |
| | 100321 TAAGTCTCTA CTAAAAATAC AAAAATTAGC CAGGCGTGGT GCTGCACACC TGTAATATCA |
| | 100381 GCTACTCGGG AGGCTGAGGC AGGAGACTCG CTTGATCCCA TGCAGCGGAG GTTGCAGTGA |
| | 100441 GCCGAGATCA CGCCATTGCA CTCCAGCCTG GGCATCAGAA TAAGACTCCG TCTCAAAAAA |
| | 100501 AAAACCACAA AAAAACAAAA CAACAACAAA AGAAAACTAG TGCTTATTCG TCACTGGCCA |
| | 100561 AGCTGCCCAT TGGCTACATG GGTGCTTCAA AGAGCTGCCC TTCTCCAGGT CTGGCCAGCA |
| | 100621 GGTATGTGTT ACAGCAAATG CCTGGGGCAG CGGCAGGGGC ATTGCTGCGG GAAGCTTCTG |
| | 100681 GACTTGCAGG AAAGCTAAGT TCTCAGACTG CAGGGGAGCT AAGCACACCT CGGCACAGGG |
| | 100741 TGAGGCCTGC GGTTCTCAGA CTTCAGTCTT TGTGGAGCTT GAGAAAAATG AGGCTTTGCA |
| | 100801 GGTCCCACCC CTAGAGATTC TGCTCTATCC ACTCTTGAAG GGGATCGAGA AATTTGCATT |
| | 100861 TTGCAACTCC CACTTTCCTC CTTGAAAGCT CCGGAGATTC TGACGCAGGG TTCCGTGGGC |
| | 100921 CACACTTTGG AAAATACAGA CCCATGAGAT AGAATACCAG ACTGTTGAAG TGTAACGGGG |
| | 100981 GCCTGGGAAG TGCAGTAACA GAAGCAAGTT TGAGGGTAAA GGACACCCAG AGGAGGGAGG |
| | 101041 GACAGCATCT GCATGGAGAG GAGAAGAGAC CCCCCAGCAG CTTCCAGGGT GTTGGAAGGG |
| | 101101 TGCGCTAGTA ACTGCTATGC ATGGCAGGTG GGGAACTGTA CGTCAGGGCA CAGCAGCATG |
| | 101161 AAGCGGTATG GCTCGTGTGG ACAGCTAGGG ACAGGCAGGC GTGGAGCAGG CATCCTGTTC |
| | 101221 TGAAGGCCAA ATCCCACAGA GGAGCCAGGG TGCTGGCAGG AGCCCTGAAC TAGCCGAACA |
| | 101281 GCTGAACAGC TGAACATTCA CCCTGTGGGG AAAGGGTCAG AAGCGTCCAG GCTTGAGGGC |
| | 101341 ACAGCTGGGT CTCGTCACTG CATCACCCTT ATTTAGGATA AAGGCCCTGA AGAATTGTAT |
| | 101401 TAGAGGTTGG CAAAGCATAT CTACCACCTC CTGGAGCCAC GCTGGCCGCA GGGATTATAA |
| | 101461 TTATTTCCAT TTTCAAATTA AGGCCTCTGA GCTCAGAGAG GGGAAGTTAC TTGTCTGAGG |
| | 101521 CCACACAGCT TGTTGGAGCC CATCTCTTGA CCCAAAGACT GTGGAGCCGA GTTGGCCACC |
| | 101581 TCTCTGGGAG CGGGTATTGG ATGGTGGTTG ATGGTTTTCC ATTGCTTTCC TGGGAAAGGG |
| | 101641 GTGTCTCTGT CCCTAAGCAA AAAGGCAGGG AGGAAGAGAT GCTTCCCCAG GGCAGCCGTC |
| | 101701 TGCTGTAGCT GCGCTTCCAA CCTGGCTTCC ACCTGCCTAA CCCAGTGGTG AGCCTGGGAA |
| | 101761 TGGACCCACG GGACAGGCAG CCCCCAGGGC CTTTTCTGAC CCCACCCACT CGAGTCCTGG |
| | 101821 CTTCACTCCC TTCCTTCCTT CCCAGGTGAA CCTCCAAAAT CAGGGGATCG CAGCGGCTAC |
| | 101881 AGCAGCCCCG GCTCCCCAGG CACTCCCGGC AGCCGCTCCC GCACCCCGTC CCTTCCAACC |
| | 101941 CCACCCACCC GGGAGCCCAA GAAGGTGGCA GTGGTCCGTA CTCCACCCAA GTCGCCGTCT |
| | 102001 TCCGCCAAGA GCCGCCTGCA GACAGCCCCC GTGCCCATGC CAGACCTGAA GAATGTCAAG |
| | 102061 TCCAAGATCG GCTCCACTGA GAACCTGAAG CACCAGCCGG GAGGCGGGAA GGTGAGAGTG |
| | 102121 GCTGGCTGCG CGTGGAGGTG TGGGGGGCTG CGCCTGGAGG GGTAGGGCTG TGCCTGGAAG |
| | 102181 GGTAGGGCTG CGCCTGGAGG TGCGCGGTTG AGCGTGGAGT CGTGGGACTG TGCATGGAGG |
| | 102241 TGTGGGGCTC CCCGCACCTG AGCACCCCCG CATAACACCC CAGTCCCCTC TGGACCCTCT |
| | 102301 TCAAGGAAGT TCAGTTCTTT ATTGGGCTCT CCACTACACT GTGAGTGCCC TCCTCAGGCG |
| | 102361 AGAGAACGTT CTGGCTCTTC TCTTGCCCCT TCAGCCCCTG TTAATCGGAC AGAGATGGCA |
| | 102421 GGGCTGTGTC TCCACGGCCG GAGGCTCTCA TAGTCAGGGC ACCCACAGCG GTTCCCCACC |
| | 102481 TGCCTTCTGG GCAGAATACA CTGCCACCCA TAGGTCAGCA TCTCCACTCG TGGGCCATCT |
| | 102541 GCTTAGGTTG GGTTCCTCTG GATTCTGGGG AGATTGGGGG TTCTGTTTTG ATCAGCTGAT |
| | 102601 TCTTCTGGGA GCAAGTGGGT GCTCGCGAGC TCTCCAGCTT CCTAAAGGTG GAGAAGCACA |
| | 102661 GACTTCGGGG GCCTGGCCTG GATCCCTTTC CCCATTCCTG TCCCTGTGCC CCTCGTCTGG |
| | 102721 GTGCGTTAGG GCTGACATAC AAAGCACCAC AGTGAAAGAA CAGCAGTATG CCTCCTCACT |
| | 102781 AGCCAGGTGT GGGCGGGTGG GTTTCTTCCA AGGCCTCTCT GTGGCCGTGG GTAGCCACCT |
| | 102841 CTGTCCTGCA CCGCTGCAGT CTTCCCTCTG TGTGTGCTCC TGGTAGCTCT GCGCATGCTC |
| | 102901 ATCTTCTTAT AAGAACACCA TGGCAGCTGG GCGTAGTGGC TCACGCCTAT AATCCCAGCA |
| | 102961 CTTTGGGAGG CTGAGGCAGG CAGATCACGA GGTCAGGAGT TCGAGACCAA CCTGACCAAC |
| | 103021 AGGGTGAAAC CTCGTCTCTA CTAAAAATAC AAAAATACCT GGGCGTGGTG GTGGTGCGCG |
| | 103081 CCTATAATCC CAGCTACTCA GGAGGCTGAG GCAGGAGAAT CGCTTGAACC CAGGAGGCAG |
| | 103141 AGGTTGCAGT GAGCCGAGAT AGTGCCACTG CACTCCAGTT TGAGCAACAG AGCGAGACTC |
| | 103201 TGTCTCAAAA CAAAATAAAA CAAACCAAAA AAACCCACCA TGGCTTAGGG CCCAGCCTGA |
| | 103261 TGACCTCATT TTTCACTTAG TCACCTCTCT AAAGGCCCTG TCTCCAAATA GAGTCACATT |
| | 103321 CTAAGGTACG GGGGTGTTGG GGAGGGGGGT TAGGGCTTCA ACATGTGAAT TTGCGGGGAC |
| | 103381 CACAATTCAG CCCAGGACCC CGCTCCCGCC ACCCAGCACT GGGGAGCTGG GGAAGGGTGA |
| | 103441 AGAGGAGGCT GGGGGTGAGA AGGACCACAG CTCACTCTGA GGCTGCAGAT GTGCTGGGCC |
| | 103501 TTCTGGGCAC TGGGCCTCGG GGAGCTAGGG GGCTTTCTGG AACCCTGGGC CTGCGTGTCA |
| | 103561 GCTTGCCTCC CCCACGCAGG CGCTCTCCAC ACCATTGAAG TTCTTATCAC TTGGGTCTGA |
| | 103621 GCCTGGGGCA TTTGGACGGA GGGTGGCCAC CAGTGCACAT GGGCACCTTG CCTCAAACCC |
| | 103681 TGCCACCTCC CCCCACCCAG GATCCCCCCT GCCCCCGAAC AAGCTTGTGA GTGCAGTGTC |
| | 103741 ACATCCCATC GGGATGGAAA TGGACGGTCG GGTTAAAAGG GACGCATGTG TAGACCCTGC |
| | 103801 CTCTGTGCAT CAGGCCTCTT TTGAGAGTCC CTGCGTGCCA GGCGGTGCAC AGAGGTGGAG |
| | 103861 AAGACTCGGC TGTGCCCCAG AGCACCTCCT CTCATCGAGG AAAGGACAGA CAGTGGCTCC |
| | 103921 CCTGTGGCTG TGGGGACAAG GGCAGAGCTC CCTGGAACAC AGGAGGGAGG GAAGGAAGAG |
| | 103981 AACATCTCAG AATCTCCCTC CTGATGGCAA ACGATCCGGG TTAAATTAAG GTCCGGCCTT |
| | 104041 TTCCTGCTCA GGCATGTGGA GCTTGTAGTG GAAGAGGCTC TCTGGACCCT CATCCACCAC |
| | 104101 AGTGGCCTGG TTAGAGACCT TGGGGAAATA ACTCACAGGT GACCCAGGGC CTCTGTCCTG |
| | 104161 TACCGCAGCT GAGGGAAACT GTCCTGCGCT TCCACTGGGG ACAATGCGCT CCCTCGTCTC |
| | 104221 CAGACTTTCC AGTCCTCATT CGGTTCTCGA AAGTCGCCTC CAGAAGCCCC ATCTTGGGAC |
| | 104281 CACCGTGACT TTCATTCTCC AGGGTGCCTG GCCTTGGTGC TGCCCAAGAC CCCAGAGGGG |
| | 104341 CCCTCACTGG CCTTTCCTGC CTTTTCTCCC ATTGCCCACC CATGCACCCC CATCCTGCTC |
| | 104401 CAGCACCCAG ACTGCCATCC AGGATCTCCT CAAGTCACAT AACAAGCAGC ACCCACAAGG |
| | 104461 TGCTCCCTTC CCCCTAGCCT GAATCTGCTG CTCCCCGTCT GGGGTTCCCC GCCCATGCAC |
| | 104521 CTCTGGGGGC CCCTGGGTTC TGCCATACCC TGCCCTGTGT CCCATGGTGG GGAATGTCCT |
| | 104581 TCTCTCCTTA TCTCTTCCCT TCCCTTAAAT CCAAGTTCAG TTGCCATCTC CTCCAGGAAG |
| | 104641 TCTTCCTGGA TTCCCCTCTC TCTTCTTAAA GCCCCTGTAA ACTCTGACCA CACTGAGCAT |
| | 104701 GTGTCTGCTG CTCCCTAGTC TGGGCCATGA GTGAGGGTGG AGGCCAAGTC TCATGCATTT |
| | 104761 TTGCAGCCCC CACAAGACTG TGCAGGTGGC CGGCCCTCAT TGAATGCGGG GTTAATTTAA |
| | 104821 CTCAGCCTCT GTGTGAGTGG ATGATTCAGG TTGCCAGAGA CAGAACCCTC AGCTTAGCAT |
| | 104881 GGGAAGTAGC TTCCCTGTTG ACCCTGAGTT CATCTGAGGT TGGCTTGGAA GGTGTGGGCA |
| | 104941 CCATTTGGCC CAGTTCTTAC AGCTCTGAAG AGAGCAGCAG GAATGGGGCT GAGCAGGGAA |
| | 105001 GACAACTTTC CATTGAAGGC CCCTTTCAGG GCCAGAACTG TCCCTCCCAC CCTGCAGCTG |
| | 105061 CCCTGCCTCT GCCCATGAGG GGTGAGAGTC AGGCGACCTC ATGCCAAGTG TAGAAAGGGG |
| | 105121 CAGATGGGAG CCCCAGGTTA TGACGTCACC ATGCTGGGTG GAGGCAGCAC GTCCAAATCT |
| | 105181 ACTAAAGGGT TAAAGGAGAA AGGGTGACTT GACTTTTCTT GAGATATTTT GGGGGACGAA |
| | 105241 GTGTGGAAAA GTGGCAGAGG ACACAGTCAC AGCCTCCCTT AAATGCCAGG AAAGCCTAGA |
| | 105301 AAAATTGTCT GAAACTAAAC CTCAGCCATA ACAAAGACCA ACACATGAAT CTCCAGGAAA |
| | 105361 AAAGAAAAAG AAAAATGTCA TACAGGGTCC ATGCACAAGA GCCTTTAAAA TGACCCGCTG |
| | 105421 AAGGGTGTCA GGCCTCCTCC TCCTGGACTG GCCTGAAGGC TCCACGAGCT TTTGCTGAGA |
| | 105481 CCTTTGGGTC CCTGTGGCCT CATGTAGTAC CCAGTATGCA GTAAGTGCTC AATAAATGTT |
| | 105541 TGGCTACAAA AGAGGCAAAG CTGGCGGAGT CTGAAGAATC CCTCAACCGT GCCGGAACAG |
| | 105601 ATGCTAACAC CAAAGGGAAA AGAGCAGGAG CCAAGTCACG TTTGGGAACC TGCAGAGGCT |
| | 105661 GAAAACTGCC GCAGATTGCT GCAAATCATT GGGGGAAAAA CGGAAAACGT CTGTTTTCCC |
| | 105721 CTTTGTGCTT TTCTCTGTTT TCTTCTTTGT GCTTTTCTCT GTTTTCAGGA TTTGCTACAG |
| | 105781 TGAACATAGA TTGCTTTGGG GCCCCAAATG GAATTATTTT GAAAGGAAAA TGCAGATAAT |
| | 105841 CAGGTGGCCG CACTGGAGCA CCAGCTGGGT AGGGGTAGAG ATTGCAGGCA AGGAGGAGGA |
| | 105901 GCTGGGTGGG GTGCCAGGCA GGAAGAGCCC GTAGGCCCCG CCGATCTTGT GGGAGTCGTG |
| | 105961 GGTGGCAGTG TTCCCTCCAG ACTGTAAAAG GGAGCACCTG GCGGGAAGAG GGAATTCTTT |
| | 106021 TAAACATCAT TCCAGTGCCC GAGCCTCCTG GACCTGTTGT CATCTTGAGG TGGGCCTCCC |
| | 106081 CTGGGTGACT CTAGTGTGCA GCCTGGCTGA GACTCAGTGG CCCTGGGTTC TTACTGCTGA |
| | 106141 CACCTACCCT CAACCTCAAC CACTGCGGCC TCCTGTGCAC CCTGATCCAG TGGCTCATTT |
| | 106201 TCCACTTTCA GTCCCAGCTC TATCCCTATT TGCAGTTTCC AAGTGCCTGG TCCTCAGTCA |
| | 106261 GCTCAGACCC AGCCAGGCCA GCCCCTGGTT CCCACATCCC CTTTGCCAAG CTCATCCCCG |
| | 106321 CCCTGTTTGG CCTGCGGGAG TGGGAGTGTG TCCAGACACA GAGACAAAGG ACCAGCTTTT |
| | 106381 AAAACATTTT GTTGGGGCCA GGTGTGGTGG CTCACACCTA ATCCCAACAC CTGGGGAGGC |
| | 106441 CAAGGCAGAA GGATCACTTG AGTCCAGGAG TTCAAGACCA GCCTGGGCAA CATAGGGAGA |
| | 106501 CCCTGTCTCT ACAATTTTTT TTTTAATTAG CTGGGCCTGT TGGCACTCTC CTGTAGTTCC |
| | 106561 AGCTACTCTA GAGGCTGAGG TGGGAGGACT GCTTGAGCCT GGGAGGTCAG GGCTGCAATG |
| | 106621 AGCCATGTTC ACACCACTGA ACGCCAGCCT GGGCGAGACC CTGTATCAAA AAAGTAAAGT |
| | 106681 AAAATGAATC CTGTACGTTA TATTAAGGTG CCCCAAATTG TACTTAGAAG GATTTCATAG |
| | 106741 TTTTAAATAC TTTTGTTATT TAAAAAATTA AATGACTGCA GCATATAAAT TAGGTTCTTA |
| | 106801 ATGGAGGGGA AAAAGAGTAC AAGAAAAGAA ATAAGAATCT AGAAACAAAG ATAAGAGCAG |
| | 106861 AAATAAACCA GAAAACACAA CCTTGCACTC CTAACTTAAA AAAAAAAATG AAGAAAACAC |
| | 106921 AACCAGTAAA ACAACATATA ACAGCATTAA GAGCTGGCTC CTGGCTGGGC GCGGTGGCGC |
| | 106981 ATGCCTGTAA TCCCAACACT TTGGGAGGCC GATGCTGGAG GATCACTTGA GACCAGGAGT |
| | 107041 TCAAGGTTGC AGTGAGCTAT GATCATACCA CTACACCCTA GCCTGGGCAA CACAGTGAGA |
| | 107101 CTGAGACTCT ATTAAAAAAA AAATGCTGGT TCCTTCCTTA TTTCATTCCT TTATTCATTC |
| | 107161 ATTCAGACAA CATTTATGGG GCACTTCTGA GCACCAGGCT CTGTGCTAAG AGCTTTTGCC |
| | 107221 CCCAGGGTCC AGGCCAGGGG ACAGGGGCAG GTGAGCAGAG AAACAGGGCC AGTCACAGCA |
| | 107281 GCAGGAGGAA TGTAGGATGG AGAGCTTGGC CAGGCAAGGA CATGCAGGGG GAGCAGCCTG |
| | 107341 CACAAGTCAG CAAGCCAGAG AAGACAGGCA GACCCTTGTT TGGGACCTGT TCAGTGGCCT |
| | 107401 TTGAAAGGAC AGCCCCCACC CGGAGTGCTG GGTGCAGGAG CTGAAGGAGG ATAGTGGAAC |
| | 107461 ACTGCAACGT GGAGCTCTTC AGAGCAAAAG CAAAATAAAC AACTGGAGGC AGCTGGGGCA |
| | 107521 GCAGAGGGTG TGTGTTCAGC ACTAAGGGGT GTGAAGCTTG AGCGCTAGGA GAGTTCACAC |
| | 107581 TGGCAGAAGA GAGGTTGGGG CAGCTGCAAG CCTCTGGACA TCGCCCGACA GGACAGAGGG |
| | 107641 TGGTGGACGG TGGCCCTGAA GAGAGGCTCA GTTCAGCTGG CAGTGGCCGT GGGAGTGCTG |
| | 107701 AAGCAGGCAG GCTGTCGGCA TCTGCTGGGG ACGGTTAAGC AGGGGTGAGG GCCCAGCCTC |
| | 107761 AGCAGCCCTT CTTGGGGGGT CGCTGGGAAA CATAGAGGAG AACTGAAGAA GCAGGGAGTC |
| | 107821 CCAGGGTCCA TGCAGGGCGA GAGAGAAGTT GCTCATGTGG GGCCCAGGCT GCAGGATCAG |
| | 107881 GAGAACTGGG GACCCTGTGA CTGCCAGCGG GGAGAAGGGG GTGTGCAGGA TCATGCCCAG |
| | 107941 GGAAGGGCCC AGGGGCCCAA GCATGGGGGG GCCTGGTTGG CTCTGAGAAG ATGGAGCTAA |
| | 108001 AGTCACTTTC TCGGAGGATG TCCAGGCCAA TAGTTGGGAT GTGAAGACGT GAAGCAGCAC |
| | 108061 AGAGCCTGGA AGCCCAGGAT GGACAGAAAC CTACCTGAGC AGTGGGGCTT TGAAAGCCTT |
| | 108121 GGGGCGGGGG GTGCAATATT CAAGATGGCC ACAAGATGGC AATAGAATGC TGTAACTTTC |
| | 108181 TTGGTTCTGG GCCGCAGCCT GGGTGGCTGC TTCCTTCCCT GTGTGTATTG ATTTGTTTCT |
| | 108241 CTTTTTTGAG ACAGAGTCTT GCTGGGTTGC CCAGGCTGGA GTGCAGTGGT GCGATCATAG |
| | 108301 CTCACTGCAG CCTTGAAGTC CTGAGCTCAA GAGATCCTTC CACCTCAGCC TCCTGAGTAG |
| | 108361 TTGGGACCAC AGGCTTGCAC CACAGTGCCC AACTAATTTC TTATATTTTT TGTAGAGATG |
| | 108421 GGGTTTCACT GTGTCGCCCA GGATGGTCTT GAACTCCTGG GCTCAAGTGA TCCTCCTGCC |
| | 108481 TCAGCCTCGC AAATTGCTGG GATTACAGGT GTGAGCCACC ATGCCCGACC TTCTCTTTTT |
| | 108541 AAGGGCGTGT GTGTGTGTGT GTGTGTGTGG GCGCACTCTC GTCTTCACCT TCCCCCAGCC |
| | 108601 TTGCTCTGTC TCTACCCAGT CACCTCTGCC CATCTCTCCG ATCTGTTTCT CTCTCCTTTT |
| | 108661 ACCCCTCTTT CCTCCCTCCT CATACACCAC TGACCATTAT AGAGAACTGA GTATTCTAAA |
| | 108721 AATACATTTT ATTTATTTAT TTTGAGACAG AGTCTCACTC TGTCACCCAG GCTGGAGTGC |
| | 108781 AGTGGTGCAA TCTCGGCTCA CTGCAACCTC CGCCTCCCAG GTTGAAGCAA CTCTCCTGCC |
| | 108841 TCAGCCTCCC TAGTAGCTGG GATTACAAGC ACACACCACC ATGCCTAGCA AATTTTTATA |
| | 108901 TTTTTAGTAG AGGAGGGGTG TCACCATGTT TGCCAAGCTG GTCTCAAACT CCTGGCCTCA |
| | 108961 GGTGATCTGC CTACCTTGGT CTCCCAAAGT GCTGGGATTA CAGGTGTGAG CCACCACGCC |
| | 109021 TGCCCTTAAA AATACATTAT ATTTAATAGC AAAGCCCCAG TTGTCACTTT AAAAAGCATC |
| | 109081 TATGTAGAAC ATTTATGTGG AATAAATACA GTGAATTTGT ACGTGGAATC GTTTGCCTCT |
| | 109141 CCTCAATCAG GGCCAGGGAT GCAGGTGAGC TTGGGCTGAG ATGTCAGACC CCACAGTAAG |
| | 109201 TGGGGGGCAG AGCCAGGCTG GGACCCTCCT CTAGGACAGC TCTGTAACTC TGAGACCCTC |
| | 109261 CAGGCATCTT TTCCTGTACC TCAGTGCTTC TGAAAAATCT GTGTGAATCA AATCATTTTA |
| | 109321 AAGGAGCTTG GGTTCATCAC TGTTTAAAGG ACAGTGTAAA TAATTCTGAA GGTGACTCTA |
| | 109381 CCCTGTTATT TGATCTCTTC TTTGGCCAGC TGACTTAACA GGACATAGAC AGGTTTTCCT |
| | 109441 GTGTCAGTTC CTAAGCTGAT CACCTTGGAC TTGAAGAGGA GGCTTGTGTG GGCATCCAGT |
| | 109501 GCCCACCCCG GGTTAAACTC CCAGCAGAGT ATTGCACTGG GCTTGCTGAG CCTGGTGAGG |
| | 109561 CAAAGCACAG CACAGCGAGC ACCAGGCAGT GCTGGAGACA GGCCAAGTCT GGGCCAGCCT |
| | 109621 GGGAGCCAAC TGTGAGGCAC GGACGGGGCT GTGGGGCTGT GGGGCTGCAG GCTTGGGGCC |
| | 109681 AGGGAGGGAG GGCTGGGCTC TTTGGAACAG CCTTGAGAGA ACTGAACCCA AACAAAACCA |
| | 109741 GATCAAGGTC TAGTGAGAGC TTAGGGCTGC TTTGGGTGCT CCAGGAAATT GATTAAACCA |
| | 109801 AGTGGACACA CACCCCCAGC CCCACCTCAC CACAGCCTCT CCTTCAGGGT CAAACTCTGA |
| | 109861 CCACAGACAT TTCTCCCCTG ACTAGGAGTT CCCTGGATCA AAATTGGGAG CTTGCAACAC |
| | 109921 ATCGTTCTCT CCCTTGATGG TTTTTGTCAG TGTCTATCCA GAGCTGAAGT GTAATATATA |
| | 109981 TGTTACTGTA GCTGAGAAAT TAAATTTCAG GATTCTGATT TCATAATGAC AACCATTCCT |
| | 110041 CTTTTCTCTC CCTTCTGTAA ATCTAAGATT CTATAAACGG TGTTGACTTA ATGTGACAAT |
| | 110101 TGGCAGTAGT TCAGGTCTGC TTTGTAAATA CCCTTGTGTC TATTGTAAAA TCTCACAAAG |
| | 110161 GCTTGTTGCC TTTTTTGTGG GGTTAGAACA AGAAAAAGCC ACATGGAAAA AAAATTTCTT |
| | 110221 TTTTGTTTTT TTGTTTGCTT GTTTTTTTGA GACAGAGTTT CACTCTGTCG CCCAGGCTGG |
| | 110281 AGTGCAGTGG TGCGATCTCC GCCCACTGCA AGCTCCACCT CCCGGGTTCA TGCTATTCTC |
| | 110341 CTGTCTCAGC CTCCCAAGTA GCTGGGACTG CAGGTGCCCG CCACCACACC TGGCTAATTT |
| | 110401 TTTTGTATTT TTAGTAGAGA CGGGGTTTCA CCGTGTTAGC CAGGATGGTC TCAATCTCCT |
| | 110461 GACCTCGTCA TCTGCCTGCC TCGGCCTCCC AAAGTGCTGA GATTACAGGC GTGAGCCACC |
| | 110521 GTGCCCGGCC AGAAAAAAAC ATTTCTAAGT ATGTGGCAGA TACTGAATTA TTGCTTAATG |
| | 110581 TCCTTTGATT CATTTGTTTA ATTTCTTTAA TGGATTAGTA CAGAAAACAA AGTTCTCTTC |
| | 110641 CTTGAAAAAC TGGTAAGTTT TCTTTGTCAG ATAAGGAGAG TTAAATAACC CATGACATTT |
| | 110701 CCCTTTTTGC CTCGGCTTCC AGGAAGCTCA AAGTTAAATG TAATGATCAC TCTTGTAATT |
| | 110761 ATCAGTGTTG ATGCCCTTCC CTTCTTCTAA TGTTACTCTT TACATTTTCC TGCTTTATTA |
| | 110821 TTGTGTGTGT TTTCTAATTC TAAGCTGTTC CCACTCCTTT CTGAAAGCAG GCAAATCTTC |
| | 110881 TAAGCCTTAT CCACTGAAAA GTTATGAATA AAAAATGATC GTCAAGCCTA CAGGTGCTGA |
| | 110941 GGCTACTCCA GAGGCTGAGG CCAGAGGACC ACTTGAGCCC AGGAATTTGA GACCTGGGCT |
| | 111001 GGGCAGCATA GCAAGACTCT ATCTCCATTA AAACTATTTT TTTTTATTTA AAAAATAATC |
| | 111061 CGCAAAGAAG GAGTTTATGT GGGATTCCTT AAAATCGGAG GGTGGCATGA ATTGATTCAA |
| | 111121 AGACTTGTGC AGAGGGCGAC AGTGACTCCT TGAGAAGCAG TGTGAGAAAG CCTGTCCCAC |
| | 111181 CTCCTTCCGC AGCTCCAGCC TGGGCTGAGG CACTGTCACA GTGTCTCCTT GCTGGCAGGA |
| | 111241 GAGAATTTCA ACATTCACCA AAAAGTAGTA TTGTTTTTAT TAGGTTTATG AGGCTGTAGC |
| | 111301 CTTGAGGACA GCCCAGGACA ACTTTGTTGT CACATAGATA GCCTGTGGCT ACAAACTCTG |
| | 111361 AGATCTAGAT TCTTCTGTGG CTGCTTCTGA CCTGAGAAAG TTGCGGAACC TCAGCGAGCC |
| | 111421 TCACATGGCC TCCTTGTCCT TAACGTGGGG ACGGTGGGCA AGAAAGGTGA TGTGGCACTA |
| | 111481 GAGATTTATC CATCTCTAAA GGAGGAGTGG ATTGTACATT GAAACACCAG AGAAGGAATT |
| | 111541 ACAAAGGAAG AATTTGAGTA TCTAAAAATG TAGGTCAGGC GCTCCTGTGT TGATTGCAGG |
| | 111601 GCTATTCACA ATAGCCAAGA TTTGGAAGCA ACCCAAGTGT CCATCAACAG ACAAATGGAT |
| | 111661 AAAGAAAATG TGGTGCATAT ACACAATGGA ATACTATTCA GCCATGAAAA AGAATGAGAA |
| | 111721 TCTGTCATTT GAAACAACAT GGATGGAACT GGAGGACATT ATGTTAAGTG AAATAAGCCA |
| | 111781 GACAGAAGGA CAGACTTCAC ATGTTCTCAC ACATTTGTGG GAGCTAAAAA TTAAACTCAT |
| | 111841 GGAGATAGAG AGTAGAAGGA TGGTTACCAG AGGCTGAGGA GGGTGGAGGG GAGCAGGGAG |
| | 111901 AAAGTAGGGA TGGTTAATGG GTACAAAAAC GTAGTTAGCA TGCATAGATC TAGTATTGGA |
| | 111961 TAGCACAGCA GGGTGACGAC AGCCAACAGT AATTTATAGT ACATTTAAAA ACAACTAAAA |
| | 112021 GAGTGTAATT GGACTGGCTA ACATGGTGAA ACCCCGTCTC TACTAAAAAT ACAAAAATTA |
| | 112081 GCTGGGCATG GTGGCTCACG CCTGTAATCC CAGCACTTTG GGAGGCCGAG GCGGGCCGAT |
| | 112141 CACGAGGTCA GGAGATCGAG ACCATCCTAG CTAACATGGT GAAACCCCGT CTCTACTACA |
| | 112201 AATACAAAAA AAAGAAAAAA TTAGCCGGGC ATGGTGGTGG GCGCCTGTAG TCCCAGCTAC |
| | 112261 TCGGGAGGCT GAGGCAGGAG AATGGCGTGA ACCCGGGAGG CGGAGCTTGC AGTGAGCCGA |
| | 112321 GATCGCGCCA CTGCACTCCA GCCTGGGCGA CAAGGCAAGA TTCTATCTCA AAAAAATAAA |
| | 112381 AATAAAATAA AATAAAATAA TAAAATAAAA TAAAATAAAA TAAAATAAAT AAAATAAAAT |
| | 112441 AAAATGTATA ATTGGAATGT TTATAACACA AGAAATGATA AATGCTTGAG GTGATAGATA |
| | 112501 CCCCATTCAC CGTGATGTGA TTATTGCACA ATGTATGTCT GTATCTAAAT ATCTCATGTA |
| | 112561 CCCCACAAGT ATATACACCT ACTATGTACC CATATAAATT TAAAATTAAA AAATTATAAA |
| | 112621 ACAAAAATAA ATAAGTAAAT TAAAATGTAG GCTGGACACC GTGGTTCACG CCTGTAATCC |
| | 112681 CAGTGCTTTG TGAGGCTGAG GTGAGAGAAT CACTTGAGCC CAGGAGTTTG AGACCGGCCT |
| | 112741 GGGTGACATA GCGAGACCCC ATCATCACAA AGAATTTTTA AAAATTAGCT GGGCGTGGTA |
| | 112801 GCACATACCG GTAGTTCCAG CTACTTGGGA GACCGAGGCA GGAGGATTGC TTGAGCCCAG |
| | 112861 GAGTTTAAGG CTGCAGTGAG CTACGATGGC GCCACTGCAT TCCAGCCTGG GTGACAGAGT |
| | 112921 GAGAGCTTGT CTCTATTTTA AAAATAATAA AAAGAATAAA TAAAAATAAA TTAAAATGTA |
| | 112981 AATATGTGCA TGTTAGAAAA AATACACCCA TCAGCAAAAA GGGGGTAAAG GAGCGATTTC |
| | 113041 AGTCATAATT GGAGAGATGC AGAATAAGCC AGCAATGCAG TTTCTTTTAT TTTGGTCAAA |
| | 113101 AAAAATAAGC AAAACAATGT TGTAAACACC CAGTGCTGGC AGCAATGTGG TGAGGCTGGC |
| | 113161 TCTCTCACCA GGGCTCACAG GGAAAACTCA TGCAACCCTT TTAGAAAGCC ATGTGGAGAG |
| | 113221 TTGTACCGAG AGGTTTTAGA ATATTTATAA CTTTGACCCA GAAATTCTAT TCTAGGACTC |
| | 113281 TGTGTTATGA AAATAACCCA TCATATGGAA AAAGCTCCTT TCAGAAAGAG GTTCATGGGA |
| | 113341 GGCTGTTTGT ATTTTTTTTT TCTTTGCATC AAATCCAGCT CCTGCAGGAC TGTTTGTATT |
| | 113401 ATTGAAGTAC AAAGTGGAAT CAATACAAAT GTTGGATAGC AGGGGAACAA TATTCACAAA |
| | 113461 ATGGAATGGG ACATAGTATT AAACATAGTG CTTCTGATGA CCGTAGACCA TAGACAATGC |
| | 113521 TTAGGATATG ATATCACTTC TTTTGTTGTT TTTTGTATTT TGAGACGAAG TCTCATTCTG |
| | 113581 TCACCCAGGC TGGAGTTCAG TGGCGCCATC TCAGCTCACT GCAACCTCCA TCTCCCGGGT |
| | 113641 TCAAGCTATT CTCCTTCCTC AACCTCCCGA GTAGCTGGGT TGCGCACCAC CATGCCTGGC |
| | 113701 TAACTTTTGT ATTTTTAGTA CAGACGGGGT TTCACCACGT TGGCCAGGCT GCTCTTGAAC |
| | 113761 TCCTGACGTC AGGTGATCCA CCAGCCTTGA CCTCCCAAAG TGCTAGGATT ACAGGAGCCA |
| | 113821 CTGTACCCAG CCTAGGATAT GATATCACTT CTTAGAGCAA GATACAAAAT TGCATGTGCA |
| | 113881 CAATAATTCT ACCAAGTATA GGTATACAGG GGTAGTTATA TATAAATGAG ACTTCAAGGA |
| | 113941 AATACAACAA AATGCAATCG TGATTGTGTT AGGGTGGTAA GAAAACGGTT TTTGCTTTGA |
| | 114001 TGAGCTCTGT TTTTTAAAAT CGTTATATTT TCTAATAAAA ATACATAGTC TTTTGAAGGA |
| | 114061 ACATAAAAGA TTATGAAGAA ATGAGTTAGA TATTGATTCC TATTGAAGAT TCAGACAAGT |
| | 114121 AAAATTAAGG GGAAAAAAAA CGGGATGAAC CAGAAGTCAG GCTGGAGTTC CAACCCCAGA |
| | 114181 TCCGACAGCC CAGGCTGATG GGGCCTCCAG GGCAGTGGTT TCCACCCAGC ATTCTCAAAA |
| | 114241 GAGCCACTGA GGTCTCAGTG CCATTTTCAA GATTTCGGAA GCGGCCTGGG CACGGCTGGT |
| | 114301 CCTTCACTGG GATCACCACT TGGCAATTAT TTACACCTGA GACGAATAAA AACCAGAGTG |
| | 114361 CTGAGATTAC AGGCATGGTG GCTTACGCTT GTAATCGGCT TTGGGAAGCC GAGGTGGGCT |
| | 114421 GATTGCTTGA GCCCAGGAGT TTCAAACTAT CCTGGACAAC ATAGCATGAC CTCGTCTCTA |
| | 114481 CAAAAAATAC AAAAAATTTG CCAGGTGTGG TGGCATGTGC CTGTGGTCCC AGCTACTTGG |
| | 114541 GAGGCTGAAG TAGGAGAATC CCCTGAGCCC TGGGAAGTCG AGGCTGCACT GAGCCGTGAT |
| | 114601 GGTGTCACTG CACTCCAGCC TGGGTGACAA AGTGAGACCC TATCTCACAA AGAAAAAAAA |
| | 114661 CAAAACAAAA AACCCAAAGC ACACTGTTTC CACTGTTTCC AGAGTTCCTG AGAGGAAAGG |
| | 114721 TCACCGGGTG AGGAAGACGT TCTCACTGAT CTGGCAGAGA AAATGTCCAG TTTTTCCAAC |
| | 114781 TCCCTAAACC ATGGTTTTCT ATTTCATAGT TCTTAGGCAA ATTGGTAAAA ATCATTTCTC |
| | 114841 ATCAAAACGC TGATATTTTC ACACCTCCCT GGTGTCTGCA GAAAGAACCT TCCAGAAATG |
| | 114901 CAGTCGTGGG AGACCCATCC AGGCCACCCC TGCTTATGGA AGAGCTGAGA AAAAGCCCCA |
| | 114961 CGGGAGCATT TGCTCAGCTT CCGTTACGCA CCTAGTGGCA TTGTGGGTGG GAGAGGGCTG |
| | 115021 GTGGGTGGAT GGAAGGAGAA GGCACAGCCC CCCCTTGCAG GGACAGAGCC CTCGTACAGA |
| | 115081 AGGGACACCC CACATTTGTC TTCCCCACAA AGCGGCCTGT GTCCTGCCTA CGGGGTCAGG |
| | 115141 GCTTCTCAAA CCTGGCTGTG TGTCAGAATC ACCAGGGGAA CTTTTCAAAA CTAGAGAGAC |
| | 115201 TGAAGCCAGA CTCCTAGATT CTAATTCTAG GTCAGGGCTA GGGGCTGAGA TTGTAAAAAT |
| | 115261 CCACAGGTGA TTCTGATGCC CGGCAGGCTT GAGAACAGCC GCAGGGAGTT CTCTGGGAAT |
| | 115321 GTGCCGGTGG GTCTAGCCAG GTGTGAGTGG AGATGCCGGG GAACTTCCTA TTACTCACTC |
| | 115381 GTCAGTGTGG CCGAACACAT TTTTCACTTG ACCTCAGGCT GGTGAACGCT CCCCTCTGGG |
| | 115441 GTTCAGGCCT CACGATGCCA TCCTTTTGTG AAGTGAGGAC CTGCAATCCC AGCTTCGTAA |
| | 115501 AGCCCGCTGG AAATCACTCA CACTTCTGGG ATGCCTTCAG AGCAGCCCTC TATCCCTTCA |
| | 115561 GCTCCCCTGG GATGTGACTC AACCTCCCGT CACTCCCCAG ACTGCCTCTG CCAAGTCCGA |
| | 115621 AAGTGGAGGC ATCCTTGCGA GCAAGTAGGC GGGTCCAGGG TGGCGCATGT CACTCATCGA |
| | 115681 AAGTGGAGGC GTCCTTGCGA GCAAGCAGGC GGGTCCAGGG TGGCGTGTCA CTCATCCTTT |
| | 115741 TTTCTGGCTA CCAAAGGTGC AGATAATTAA TAAGAAGCTG GATCTTAGCA ACGTCCAGTC |
| | 115801 CAAGTGTGGC TCAAAGGATA ATATCAAACA CGTCCCGGGA GGCGGCAGTG TGAGTACCTT |
| | 115861 CACACGTCCC ATGCGCCGTG CTGTGGCTTG AATTATTAGG AAGTGGTGTG AGTGCGTACA |
| | 115921 CTTGCGAGAC ACTGCATAGA ATAAATCCTT CTTGGGCTCT CAGGATCTGG CTGCGACCTC |
| | 115981 TGGGTGAATG TAGCCCGGCT CCCCACATTC CCCCACACGG TCCACTGTTC CCAGAAGCCC |
| | 116041 CTTCCTCATA TTCTAGGAGG GGGTGTCCCA GCATTTCTGG GTCCCCCAGC CTGCGCAGGC |
| | 116101 TGTGTGGACA GAATAGGGCA GATGACGGAC CCTCTCTCCG GACCCTGCCT GGGAAGCTGA |
| | 116161 GAATACCCAT CAAAGTCTCC TTCCACTCAT GCCCAGCCCT GTCCCCAGGA GCCCCATAGC |
| | 116221 CCATTGGAAG TTGGGCTGAA GGTGGTGGCA CCTGAGACTG GGCTGCCGCC TCCTCCCCCG |
| | 116281 ACACCTGGGC AGGTTGACGT TGAGTGGCTC CACTGTGGAC AGGTGACCCG TTTGTTCTGA |
| | 116341 TGAGCGGACA CCAAGGTCTT ACTGTCCTGC TCAGCTGCTG CTCCTACACG TTCAAGGCAG |
| | 116401 GAGCCGATTC CTAAGCCTCC AGCTTATGCT TAGCCTGCGC CACCCTCTGG CAGAGACTCC |
| | 116461 AGATGCAAAG AGCCAAACCA AAGTGCGACA GGTCCCTCTG CCCAGCGTTG AGGTGTGGCA |
| | 116521 GAGAAATGCT GCTTTTGGCC CTTTTAGATT TGGCTGCCTC TTGCCAGGAG TGGTGGCTCG |
| | 116581 TGCCTGTAAT TCCAGCACTT TGGGAGACTA AGGCGGGAGG TTCGCTTGAG CCCAGGAGTT |
| | 116641 CAAGACCAGC CTGGGCAACA ATGAGACCCC TGTGTCTACA AAAAGAATTA AAATTAGCCA |
| | 116701 GGTGTGGTGG CACGCACCTG TAGTCCCAGC TACTTGGGAG GCTGAGGTGG GAGGATTGCC |
| | 116761 TGAGTCCGGG AGGCGGAAGT TGCAAGGAGC CATGATCGCG CCACTGCACT TCAACCTAGG |
| | 116821 CAACAGAGTG AGACTTTGTC TCAAAAAACA ATCATATAAT AATTTTAAAA TAAATAGATT |
| | 116881 TGGCTTCCTC TAAATGTCCC CGGGGACTCC GTGCATCTTC TGTGGAGTGT CTCCGTGAGA |
| | 116941 TTCGGGACTC AGATCCTCAA GTGCAACTGA CCCACCCGAT AAGCTGAGGC TTCATCATCC |
| | 117001 CCTGGCCGGT CTATGTCGAC TGGGCACCCG AGGCTCCTCT CCCACCAGCT CTCTTGGTCA |
| | 117061 GCTGAAAGCA AACTGTTAAC ACCCTGGGGA GCTGGACGTA TGAGACCCTT GGGGTGGGAG |
| | 117121 GCGTTGATTT TTGAGAGCAA TCACCTGGCC CTGGCTGGCA GTACCGGGAC ACTGCTGTGG |
| | 117181 CTCCGGGGTG GGCTGTCTCC AGAAAATGCC TGGCCTGAGG CAGCCACCCG CATCCAGCCC |
| | 117241 AGAGGGTTTA TTCTTGCAAT GTGCTGCTGC TTCCTGCCCT GAGCACCTGG ATCCCGGCTT |
| | 117301 CTGCCCTGAG GCCCCTTGAG TCCCACAGGT AGCAAGCGCT TGCCCTGCGG CTGCTGCATG |
| | 117361 GGGCTAACTA ACGCTTCCTC ACCAGTGTCT GCTAAGTGTC TCCTCTGTCT CCCACGCCCT |
| | 117421 GCTCTCCTGT CCCCCCAGTT TGTCTGCTGT GAGGGGACAG AAGAGGTGTG TGCCGCCCCC |
| | 117481 ACCCCTGCCC GGGCCCTTGT TCCTGGGATT GCTGTTTTCA GCTGTTTGAG CTTTGATCCT |
| | 117541 GGTTCTCTGG CTTCCTCAAA GTGAGCTCGG CCAGAGGAGG AAGGCCATGT GCTTTCTGGT |
| | 117601 TGAAGTCAAG TCTGGTGCCC TGGTGGAGGC TGTGCTGCTG AGGCGGAGCT GGGGAGAGAG |
| | 117661 TGCACACGGG CTGCGTGGCC AACCCCTCTG GGTAGCTGAT GCCCAAAGAC GCTGCAGTGC |
| | 117721 CCAGGACATC TGGGACCTCC CTGGGGCCCG CCCGTGTGTC CCGCGCTGTG TTCATCTGCG |
| | 117781 GGCTAGCCTG TGACCCGCGC TGTGCTCGTC TGCGGGCTAG CCTGTGTCCC GCGCTCTGCT |
| | 117841 TGTCTGCGGT CTAGCCTGTG ACCTGGCAGA GAGCCACCAG ATGTCCCGGG CTGAGCACTG |
| | 117901 CCCTCTGAGC ACCTTCACAG GAAGCCCTTC TCCTGGTGAG AAGAGATGCC AGCCCCTGGC |
| | 117961 ATCTGGGGGC ACTGGATCCC TGGCCTGAGC CCTAGCCTCT CCCCAGCCTG GGGGCCCCTT |
| | 118021 CCCAGCAGGC TGGCCCTGCT CCTTCTCTAC CTGGGACCCT TCTGCCTCCT GGCTGGACCC |
| | 118081 TGGAAGCTCT GCAGGGCCTG CTGTCCCCCT CCCTGCCCTC CAGGTATCCT GACCACCGGC |
| | 118141 CCTGGCTCCC ACTGCCATCC ACTCCTCTCC TTTCTGGCCG TTCCCTGGTC CCTGTCCCAG |
| | 118201 CCCCCCTCCC CCTCTCACGA GTTACCTCAC CCAGGCCAGA GGGAAGAGGG AAGGAGGCCC |
| | 118261 TGGTCATACC AGCACGTCCT CCCACCTCCC TCGGCCCTGG TCCACCCCCT CAGTGCTGGC |
| | 118321 CTCAGAGCAC AGCTCTCTCC AAGCCAGGCC GCGCGCCATC CATCCTCCCT GTCCCCCAAC |
| | 118381 GTCCTTGCCA CAGATCATGT CCGCCCTGAC ACACATGGGT CTCAGCCATC TCTGCCCCAG |
| | 118441 TTAACTCCCC ATCCATAAAG AGCACATGCC AGCTGACACC AAAATAATTC GGGATGGTTC |
| | 118501 CAGTTTAGAC CTAAGTGGAA GGAGAAACCA CCACCTGCCC TGCACCTTGT TTTTTGGTGA |
| | 118561 CCTTGATAAA CCATCTTCAG CCATGAAGCC AGCTGTCTCC CAGGAAGCTC CAGGGCGGTG |
| | 118621 CTTCCTCGGG AGCTGACTGA TAGGTGGGAG GTGGCTGCCC CCTTGCACCC TCAGGTGACC |
| | 118681 CCACACAAGG CCACTGCTGG AGGCCCTGGG GACTCCAGGA ATGTCAATCA GTGACCTGCC |
| | 118741 CCCCAGGCCC CACACAGCCA TGGCTGCATA GAGGCCTGCC TCCAAGGGAC CTGTCTGTCT |
| | 118801 GCCACTGTGG AGTCCCTACA GCGTGCCCCC CACAGGGGAG CTGGTTCTTT GACTGAGATC |
| | 118861 AGCTGGCAGC TCAGGGTCAT CATTCCCAGA GGGAGCGGTG CCCTGGAGGC CACAGGCCTC |
| | 118921 CTCATGTGTG TCTGCGTCCG CTCGAGCTTA CTGAGACACT AAATCTGTTG GTTTCTGCTG |
| | 118981 TGCCACCTAC CCACCCTGTT GGTGTTGCTT TGTTCCTATT GCTAAAGACA GGAATGTCCA |
| | 119041 GGACACTGAG TGTGCAGGTG CCTGCTGGTT CTCACGTCCG AGCTGCTGAA CTCCGCTGGG |
| | 119101 TCCTGCTTAC TGATGGTCTT TGCTCTAGTG CTTTCCAGGG TCCGTGGAAG CTTTTCCTGG |
| | 119161 AATAAAGCCC ACGCATCGAC CCTCACAGCG CCTCCCCTCT TTGAGGCCCA GCAGATACCC |
| | 119221 CACTCCTGCC TTTCCAGCAA GATTTTTCAG ATGCTGTGCA TACTCATCAT ATTGATCACT |
| | 119281 TTTTTCTTCA TGCCTGATTG TGATCTGTCA ATTTCATGTC AGGAAAGGGA GTGACATTTT |
| | 119341 TACACTTAAG CGTTTGCTGA GCAAATGTCT GGGTCTTGCA CAATGACAAT GGGTCCCTGT |
| | 119401 TTTTCCCAGA GGCTCTTTTG TTCTGCAGGG ATTGAAGACA CTCCAGTCCC ACAGTCCCCA |
| | 119461 GCTCCCCTGG GGCAGGGTTG GCAGAATTTC GACAACACAT TTTTCCACCC TGACTAGGAT |
| | 119521 GTGCTCCTCA TGGCAGCTGG GAACCACTGT CCAATAAGGG CCTGGGCTTA CACAGCTGCT |
| | 119581 TCTCATTGAG TTACACCCTT AATAAAATAA TCCCATTTTA TCCTTTTTGT CTCTCTGTCT |
| | 119641 TCCTCTCTCT CTGCCTTTCC TCTTCTCTCT CCTCCTCTCT CATCTCCAGG TGCAAATAGT |
| | 119701 CTACAAACCA GTTGACCTGA GCAAGGTGAC CTCCAAGTGT GGCTCATTAG GCAACATCCA |
| | 119761 TCATAAACCA GGTAGCCCTG TGGAAGGTGA GGGTTGGGAC GGGAGGGTGC AGGGGGTGGA |
| | 119821 GGAGTCCTGG TGAGGCTGGA ACTGCTCCAG ACTTCAGAAG GGGCTGGAAA GGATATTTTA |
| | 119881 GGTAGACCTA CATCAAGGAA AGTGTTGAGT GTGAAACTTG CGGGAGCCCA GGAGGCGTGG |
| | 119941 TGGCTCCAGC TCGCTCCTGC CCAGGCCATG CTGCCCAAGA CAAGGTGAGG CGGGAGTGAA |
| | 120001 GTGAAATAAG GCAGGCACAG AAAGAAAGCA CATATTCTCG GCCGGGCGCT GTGGCTCACG |
| | 120061 CCTGTAATTC CAGCACTTTG GGAGGCCAAG GTGGGTGGAT CATGAGGTCA GGAGATTGAG |
| | 120121 ACCATCCTGG CTAACACAGT GAAACCCCGT CTCTACTAAA AATACAAAAA ATTAGCCGGG |
| | 120181 CGTGGTGGTG GGCGCCTGTA GTCCCAGCTA CTCCGGAGGC TGAGGCAGGA AAATGGCGTG |
| | 120241 AACCCGGAAG GCGGAGCTTG CAGTGAGCGG AGTGAGCAGA GATCGCGCCA CTGCACTCCA |
| | 120301 GCCTGGGCGA CAGAGCGAGA CTCCGTCTCA AAAAAAAAAA GCACATGTTC TCGCTTCTTT |
| | 120361 GTGGGATCCA GGAGATAGAG AATAGAAGGA TGGTTACCAG AGGCTGGGAA GGGTAGTGAG |
| | 120421 GGGATGGTGG GGGGATGGTC AATGGGTACA AAAAAAATAG AATAAGACCT AGTATTTGAT |
| | 120481 AGTGCAACAG GGTGACTATA GTCAATAATA ATTTAATTGT ACATTTAAAA ATAACTAAAA |
| | 120541 GATAGCCGGG TGCAGTGGCT TACGTCTGTA ATCCCAGTAC TTTGGGAGGC TGAGGTGGGC |
| | 120601 GTTTGAGACC AGCCTGGCCA ACATGGTGAA ACCCCATCTC TACTAAAAAT ACAAAAATTA |
| | 120661 GCCAGGCATG GTGGCGGGCG CCTGTAATCC CAGCTACTCG GGAGGCTGAG GCAGGAGAAT |
| | 120721 CACTTGAACC TGGGAGGCAG AGGTTGCAGT GAGCCGAGAT CTTGCCACTG CACTCCAGCC |
| | 120781 TGGGTGACAG TGAAACTCCG TCTCAAAAAT AAAAATAAAA ATACAGCTGG GCACGGTGGC |
| | 120841 TCACGCCTGT AATCCCAGCA CTTTGGGAGG CCGAGGCGAG CGGATCACAA GGTCAGGAGA |
| | 120901 TATAGACCAT CCTGGCTAAC ACGGTGAAAC CCGGTCTCTA CTAAAAATAC AAAAAATTAG |
| | 120961 CCAGGCGTGG TGGCAGGTGC CTATAGTCCC AGCTACTCAC AAGGCTGAGG CAGGAGAATG |
| | 121021 GCATGAACCT GGGAGGCGGA GCTTGCAGTG AGCCGAGATT GTGCCACTGC ACTCCAGCCT |
| | 121081 GGGCGAGAGA GTGAGACTCC GTCTCAAAAC AAAAACAAAA ACAAAAACAA AAACAAACAC |
| | 121141 ACAACAAAAA CCTAAAAGAA TATAAATGGA TTGTTTGTAA CACAAAGGAC AAATGTTTGA |
| | 121201 GGGGATGGAT ACCCCATTTT CCATGATGTG ATTATTATAC ATTGTGTGTC TGTATCAAAA |
| | 121261 CATCTCATGA GCCCCATAAA TATATACACC TAACTATGTA CCCACAAAAA TTAAAAAAAT |
| | 121321 ATATTTTTTA AGGTGAAGAG GGAGGCGAGA TGCTGGCCTT AACCCCTAAC CCGTTGTTCT |
| | 121381 CCCTGCAAGC TGTCCACAGG GCCTCTCAGA CTCGAGGTTC AGCTATATGG ATGCATGAGC |
| | 121441 TTGGTCCCCA GCCAACATGG GAGACACTTC ACCATCGGCA GCAGCTACAG CACAGGAACC |
| | 121501 CTGGGTCACT GCCATGTCCC CTCTGTGACT TTGTTTAAAC AGAAAATGAT GCTCTGGGCC |
| | 121561 GGCTGTGGTG GCCCACACCT ATAATCCCAG CACCTTGGGA GGCGGGGGTG GGCAGATTGC |
| | 121621 CTGAGGTCAG GAGTTGGAGA TCAGCCTGGC CGACATGGCG AAACCCCATG TCTACTAAAA |
| | 121681 ATACAAAAAC TAGCCAGGCA TGGTGGCACA TGCCTGTAAT CCCAGCTACT TGGGAGGCTG |
| | 121741 AAGCAGGAGA ATCACTTGAA CCCAGGAGGC AGAGGCTGAG TGAGCCAAGA TCGTGCCAAT |
| | 121801 GCACTCCAGC TTGGGTGAGG GAGTGAGACT CCGTCTCAAA AAAAAAAAAA AAGAAAGAAA |
| | 121861 AAGAAAAGAA AGTGATCCTA CTGGAACCAT GCTTACTCCC CTCCCCACCT CACACTGTGT |
| | 121921 AGAAATTAGT GCTGTCGGCC AGGCGCGGTG GCTCATGCCT GTAATCGCAG CACTTTGGGA |
| | 121981 GGCCAAGGCA GGCGGATCAC GAGGTCAGGA GATCAAGACC ATCCTGGCTA ACACAGTGAA |
| | 122041 ACCCTGTCTC TACTAAAAAT ACAAAAAATT AGCCGGGCAT GGTGGCAGGC ACCTGTAGTC |
| | 122101 CCAACTACTT GGGAGGCTGA GGCAGGAGAA TGGCATGAAC CTGGGAGGCG GAGCTTGCAG |
| | 122161 TGAGCCAAGA TCGCGCCACT GCATACCAGC CTAGGTGACA GAGTGAGACT CAGCAAAAAA |
| | 122221 AGAAAGAAAG AAAGAAAGAA ATCAGTGCTG TCTATACTTC TTTCTGCAGT GATGGAAATA |
| | 122281 TTCTGTATCT GTGCTGTCCA GTATAGTAGC CACTAGCTAC ATGTGGCACT TGAAACATGG |
| | 122341 CTGGTACAGT TGAGGAAGAG TGGCTGCCAT ATCGGACGAC ACAGCTATAG ATTCTGTCAC |
| | 122401 CCCACCCCGA GAGTCCAGAG CGGGGACTTC TGCCTTAGGC CCTATTCAGG GCTGATTTTT |
| | 122461 ACTTGAACCC TTACTGTGGG AAGAGAAGGC CATGAGAAGT TCAGTCTAGA ATGTGACTCC |
| | 122521 TTATTTTCTG GCTCCCTTGG ACACTTTGTG GGATTTAGTC TCCCTGTGGA AAGTATTCCA |
| | 122581 CAAGTGGTGC CACCACCCCA GCTGTGAGAG CAGCTGGGAG CTGCTTTTGT CATCTTTCCC |
| | 122641 TGGAAAGTCC TGTGGGCTGT CTCTTCCTCA TGCCTTGTCC CATGCTTGGG CATGGTGTCA |
| | 122701 AGCGTCAGGA GGGAGAAAGG GTCCTTATTT ATTTATTTAG AGAGGGACCC TTCTTCTGTT |
| | 122761 CCCAGGCTGG AGTGCAGTGG TGCGATCTCG GCTCACTGCA ACCTCCGCCT CCTGGGTTCA |
| | 122821 AGTGATTCTC CTGCCTCAGC CTCCTGAGTA GCTGAGATTA CAGGCACATG CCAACATGCC |
| | 122881 TGGCTAATTT TTTTTTTTTT TTTTTTTTTT TTTTTTTGAG ATGGAGTTGT ACTCTCATTG |
| | 122941 CCCAGGCTGG AATGTAATGG CACAATCTCG GCTCACTGCA ACCTCCACCT CCTGGATTCA |
| | 123001 AGCAATTCTC CTGTCTCAGC TTCCCAAGTA GCTGGGATTA CAGGTGCCCG CCACCATGCT |
| | 123061 CAACTAATTT TTGTATTTTT TTTTTAGTAG AGACGAGGTT TCACCATGTT GGTCAGACTG |
| | 123121 GTCTCAAACT CCTGACCTCA GGTGATCCAC CTGCCTCGGC CTCCCAAAGT GCTAGGATTA |
| | 123181 CAGGCATGAG CCACCACGCC CGGCCTGAAA GGGTTCTTAT TTAGTGTGCA TTTTGACATT |
| | 123241 CAATTTAATT CCAAGGTCTT GTGGGGTCAT GGTTTACAGG ATGTTGATAT AGAAAAGACT |
| | 123301 TCACTTAATG GGCCGGGCGC AGTGGCTCAT GCCTGTAATC CCAGCACTTT GGGAGGCCGA |
| | 123361 GGCAGGCAGA TCAGGAGGTC AGGAGATTGA GACCATCCTG GCTAACACAG TGAAACCCCA |
| | 123421 TCTCTACTGA AAATACAAAA AATTAGCTGG GCGTGGTGGC AGGCACCTGT AGTCCCAGCC |
| | 123481 ACTCGGTTGG CTGAGGCAGG AGAATGGCAT GAACCCGGGA GGCGGAGCTT GCAGTGAGCA |
| | 123541 GAGACCATGC CACTGCACTC CAGCCTGGGC GACAGAGCAA GACTCTGTCT CAAGAAAAAA |
| | 123601 AAAAAAAAAA CAGACTTTAC TTACTGGAAG CCAACCAATG TATATTTAGA GTAATTTTTC |
| | 123661 CTGGGCTGAG CTGTCATTTA CTTTTGCAGT ATCTCAAGAA GAAGAGTTTA CAGTGTAAAT |
| | 123721 ATTTGATGCA CACTTTGATT ATATAGATGA AGCAAACTAT TTTCAAGAGC TTTGCAAGGA |
| | 123781 CTTACTTGTA TCCAAACACC ATTCTAAAGG AGTCTTACCT ACTTCTAAAG GCTGGTCTCT |
| | 123841 ACTTGGAACC ACTTGCTTGG CCCTGGTTCA AGTCCTGCTG CAAACCTGGA AGTCCTGTCA |
| | 123901 TTGTCTTCTT CCCTCCAGAG CAGTGGCACC CAATCTAATT TTTGCTGTGC CCCAGCAGCC |
| | 123961 CCTGGCACTT TGCCCTGTAG ACTGCAGACC TCATGTAATG TATGTTAAGT CCACAGAACC |
| | 124021 ACAGAAGATG ATGGCAAGAT GCTCTTGTGT GTGTTGTGTT CTAGGAGGTG GCCAGGTGGA |
| | 124081 AGTAAAATCT GAGAAGCTTG ACTTCAAGGA CAGAGTCCAG TCGAAGATTG GGTCCCTGGA |
| | 124141 CAATATCACC CACGTCCCTG GCGGAGGAAA TAAAAAGGTA AAGGGGGTAG GGTGGGTTGG |
| | 124201 ATGCTGCCCT TGGGTATATG GGCATTAATC AAGTTGAGTG GACAAAGGCT GGTCCAGTTC |
| | 124261 CCAGAGGAGG AAAACAGAGG CTTCTGTGTT GACTGGCTGG ATGTGGGCCC TCAGCAGCAT |
| | 124321 CCAGTGGGTC TCCACTGCCT GTCTCAATCA CCTGGAGCTT TAGCACGTTT CACACCTGGG |
| | 124381 CCCCAACCTG GAGAGGCTGA CCAATGGGTC TCAGGGGCAG CTCGGTTGCT GGAGTTTTTG |
| | 124441 TTTTTATTTA TTTTTATGTA TTTAAGGCAG GGTCTCTGTA TTAGTCCATT CTCACACTGC |
| | 124501 TAATAAAGAC ATACCCAAGA CTGGGTAATT TATAAAGGAA AGAGGTTTAA TGGACTCACA |
| | 124561 GTTCCACATG GCTGGGGAGG CCTCAAAATC ATGGCGGAAG GCAAAGGAGA AGCAAAGGCA |
| | 124621 TTTCTTACAT GGCGACAGGC AAGAGAGCGT GTGCAGGGGA ACTCCCATTT ATAAAACCAT |
| | 124681 CAGACCTCAT GAGATTTATT CACTATCATG AGAACAGCAT GGGAAAGACC CGCCCCCATG |
| | 124741 ATTCAGTTAC CTCCCACTGG GTCCCTCCCA TGACACATGG AATTATGGGA GCTACAATTC |
| | 124801 AAGATGAGAT TTGGGTGGGG ACACAGCCAA ACCATATCAG TCTCCCTCTG TCATCCAGGC |
| | 124861 TGGAGTGCAC TGGCATGATC TCGGCTCACT GCAGCCTCTA CCTCCCTGGG TCAGGTGATC |
| | 124921 TTCCCACCTC AGCCTCCCAG GTAGCTGGAA CTACAGGTAC CTGCCACTAT GCCTGGCTAA |
| | 124981 ATATTTTGTA TTTCCTGTGG AGACGAGGTT TTGCCACGTT GCCCAGGCTG GTCTTGAACT |
| | 125041 CCTGAGGTCA AGCAATATGC CCACCTCGGC CTCCCAAGGT GCTGGGATTA CAGGTGTGAG |
| | 125101 CCACAGTGCT CGGCCTAAGT CACTGCAGTT TTTAAAGCTC CCAGGTGATT CTTCAGTGCA |
| | 125161 GTCAAAAGTG AGAACTGGCT GGGTGCGGTG GCTCATGCCT GTAATCCCAG CACCTTGGGA |
| | 125221 GGCGAAGGTG GGCAGATGGC TTGAGGTCAG GAGTTCAAGA CCAGCCTGGC CAACATGGTA |
| | 125281 AAACCCCATC TCTACTAAAA ATACAAAAGT TAGCTGGGTG TGGTGGTGCG TGCCTGTAAT |
| | 125341 CCCAGCTACT TGGGAGGCTG AGGCATGAGA ATTGCTTGAA CCCAGGGGAC AGAGGTTGTA |
| | 125401 GTGAGCCGAG ATCGTGCCAC TGCACTCCAG CCTGGGCAAC AGAGTGAGAT TCCATCTCAC |
| | 125461 AAAAAAAAAA AAAAAAGCGA GAACCACTGT CCTAGGCCCT GATGTTTGCA GGCAACTAAA |
| | 125521 AAAGGAAGTG GACATCCCCA GTCAGCTGTG GCGCACCAAG AACAAGTCAT GGGAACATAA |
| | 125581 CCTAATTTTC TAAATGGGTT ACTAGGCACT TAGAGCAAAA CAATGATGCC GAAATCCTGA |
| | 125641 TTTCAGCAAA GCCTCTGCCT GCCTGTCTTG GAAGTATCCA CATGAGGCTG CTGGGGCCTT |
| | 125701 GGTGTCCCCA GCAGTTTCTA GTCTCTAGGT CTTGCTGTGG GTGTCTGTGC AGTGAGGGTG |
| | 125761 TGTGTGGCGC TGGGTGAGCT CTGTCTAGGC CTGGCACAGG ATGCGGTCTG GTAGCTGCTG |
| | 125821 CTTCTCTTCT GCAGAAGCGC AGCCAAGCAC CCTCTGGGGT TTCAGGCCCA CACCCAGCCT |
| | 125881 GAAGTTCTGG GAGTGGCTCA CTTTCCAACC TTCAGGGTCT CCCAGCAGCT GACTGGGGAG |
| | 125941 TGGTGGAGGG AAAAGGGATT GTATTAGTCC GTTTTCACGC CGCTGATGAA GACATACCCG |
| | 126001 ATACTGGGCA GTCTAAAAGA TAGAGGTCTG ATGGACTCAC AGTTCCACGT GACTGGGGAG |
| | 126061 GCCTGACAAT CATGGTGGAA GGTGAAAGGC TTGTCTCACA CGGTGGCAGA CAAGAGAAAA |
| | 126121 GAGCTTGTGC AGGGGAACTC CCCTTTATAA AACCATCAGA TCTCGGGAGA CTTATTCACT |
| | 126181 ATCATGAGAA CAGCACGGGA AAGACCCTCC TCTATGATTC AATTACCTCC CACCAGGTCC |
| | 126241 CTCCCACAAC ATGTAGGAAT TGTGGGAACT ACAATTCAAG ATGACATTTG GGTGGGGACA |
| | 126301 CAGCCAAACC ATATCAGGGC GTCCCAGAAA GGGTATAGGG TCTGAGACCC AAGTCAGCAT |
| | 126361 GAGAAAGTAT GCTTCTCATG GTGGCCCAGT TGGGTGGAAG TGGCAGCCGG GCCGTCTTTC |
| | 126421 CACCAGGCCA CTCAAGTAGC AGCTGAGAGA CCCCTGCCCT GGCCAGTCCC CGCCCTCCCC |
| | 126481 TCTTGCCACT GCCTCTGGTT CTGAACAGAT GGGCACCCTC ATCTTGTATT TGTGATTAAT |
| | 126541 GTCTAACAAT GTAGTTTTGT GAGAAGGGTT TGCTGATACA GCCTTGCTGC AGATGCTGCG |
| | 126601 AACTGTGGCC TGGGGCAGAC CTTACCTCCA GACACGCCCT GAGGCAGGGG AGGGCACTGG |
| | 126661 CCCGTAGCTG GCCGAGAGCT CTCGGGTTGC GCGACAGGGA TACTTTTCAG CGGCTGGGTC |
| | 126721 GCTATCCAAA GTGAGAAAAC GAGGAGGGAC CAGGAGGCTG TCCGCCTCAA GAGATGTGGG |
| | 126781 GGCCAGGTCC AGTTATCTGG GGAAGCAGTA AGCTTCTCTG CTGTTTCTAA CCCCAGGCCT |
| | 126841 CCCCTGGTCT AAGGCAGGGC CTCCCAGCCT CGGGGCACTT TAAAGATATC TGGGCCTGGC |
| | 126901 CCCATCCCCA CAGTCTGACT GAGTGGGTCT GGATAGGGCC TGAGCATTGG TGATTTCCTG |
| | 126961 GGTGAAAGGA GGCCCCTCAC AGTCTCTGGA AGCTTCTCTG TGTTAGGAAA AGCTCTGGGC |
| | 127021 TTGACTCTGC TTTGAAAGTC AAGATCCGCA AATCCTCTCA GCCTCAGTTT CTCCTTCAGC |
| | 127081 AAGATGAAAT GGAAATGCTG TACCTACGTC CCGGGGTGGT TGTGAGACCC AAAAAAGACA |
| | 127141 ATGTTCTGGA AGGTTCCTGG TGCGTTGCAG TCCTCTAAGA ACCTGAGTTA GAGCCACGCT |
| | 127201 GAGTCTCAGC TTCTTGGCTC CTTCTGTTTC AAACTCGTCC ATGTGATAGC TCAGGAAGGG |
| | 127261 TAGGCAGGGC CCTGCCCCCT ACTCAGAAAA CACCATCCTG GTCCTGGGGA TCCCCGCAGC |
| | 127321 ATTAGTCCCC TGTTTTCCCA GTGTATTGAG AAAAATTGCT AACAAGCAGT GGGGCACACC |
| | 127381 ACCAGCCTCC TGGGTTCCTT TCAGTTTGGG GATTTTTGGA CATTCCCAGG AATGTCTTAA |
| | 127441 AAAACACTTC AAAAAACATT AACATAAATA TTTTTATCAA AGCCTGTATT AAATGGTCTT |
| | 127501 TCAAGAAAAT ACAGTAACAG GTCAGGCATG GTGGCTCATG CCTGTAACCC CAGCACTTTG |
| | 127561 GGAGGCCAAG GCAGGCAGAT CACCTGAAAT CAGGAGTTCA AGACCAACCT GGCCAACACA |
| | 127621 GCCAAATCCC ATCTCTACAA AAAATACAAA AATTAGCTGG GTGTGGTGGC ACACACCTGT |
| | 127681 AGTCCCAGCT ACTTGGGAGG CCGAGGCAGG AGAATTGCTT GATCCCGGAG GCGGAGGTTG |
| | 127741 CAGTGAGCTG AGATCGTGCC ACTGCACTCC AGCGTGGGTG ACAAGGTGAA TCTTTGTCTC |
| | 127801 AAAAAAAAAA AAAAAAAAAA GATAAAATAC AGTATACAGT AATAGAGAAC AATCCTTTTT |
| | 127861 TCAAAGTAGT GACCCCAAAT GAACAAAATA TGCATCTAGC TTAAATGCGA ACCTGGTTTT |
| | 127921 CTCTACGCCC ATTCAAGCCC CTGCAATAGG GGCCCTTCAC CCCGCATCCA TGGACTCCTA |
| | 127981 AAATTATATG GAAAATGGCT GTGTGTGAGT GTGGATGGAC ATGTGCACAC ATATTTTTGG |
| | 128041 CTTTACCAGA TGCTCAAAGA GCCTAGGACC CAAAAAGGGC TGAGAATGAC CGTGTCGGCC |
| | 128101 ACTTCAGGGT CATCAGGAAT TGCTGTGCAC TGCTCACTTC TCCAGTGAAC ACTTTCTGCT |
| | 128161 TCTGTGTTTC CTGGTATCCT TTGGGACTCC TGGCTAGGTC ATGTGTTTCT CTACTTTCAA |
| | 128221 AAGGGCTTCA GCCAGGCACG ATGGCATGAG CCTGTAGTCC CAGTTGCTCT GGAGGTTAAG |
| | 128281 GTGGGAAGAT TGCTTGAGCC CAGGAATTTG AGGCCAGCCT GGGCAAGTAG ATAGGTAGAT |
| | 128341 GATTGATAGA TAGATAGATA GATAAATAGA TGGATAGATA AGTCGCTAGA CAGTCATCCA |
| | 128401 TCCACCCATC CACACATAAA AAGGCCTTTG TCATGTCATG TTTTGTGGCC CACCTGCCAG |
| | 128461 TGTTGCCCAC AGTTGCTGCC CCTCCAAACT CATCAGTCAC TGGCAAACAG GAGGAATGTG |
| | 128521 TGGCTCATGT CTGGGCATCA GTGGCTGTGG GAGACATCCT TGATCTTCTC CAGCTTCTCC |
| | 128581 TTCCACATTT TCCTTTGCAA TCTGGCAATA TCTATTAAAA TAAAATGTGC ATGCCTTTTG |
| | 128641 ACCTAAGAGC TTCACTTCTA GGACCCACTT ACACGTGTGT GACATGATGT TCATACGGGT |
| | 128701 TTATTTATCT GAGGTTGTTC ATACACACCA TTGCCTGTAA TCACTAAAGG CGGGAGCAGC |
| | 128761 CTACACATCC ATCCACAGAG GAGTAGATGC CTTTTGGTAC ATCCGTGGCG ACGGAATACT |
| | 128821 AAGCAGCCTG TGTATCTATA CACTCACACG TGTTTGTTTA TGTGTGGAAT ATCTCTGGAG |
| | 128881 GGTACACAAG AAACTTAAAA TGATCACTGT CTCTGGGGAG GGTACCTGGG TGCCTGGGAG |
| | 128941 GCAGGTCAGG GAAGGAGTGG GCACAGGTAT TACCAATTGG AAGACAATAA AAACAACAGC |
| | 129001 TCCTGGCCAG GCGCAGTGGC TCACGCCTGT AATGGCAGCA CTCTGAGAGG CTGAGGCGGG |
| | 129061 CAGATTGCTT GCGTCCAGGA GTTCAAGACC AGCCTGGGCA ACATAGCAAA ACCCCGTTTC |
| | 129121 TATTAAAAAT ACAAAAAATT AGCCAGGTGT GGTGGCATGC ACCTGTAATC CCAGCTACTC |
| | 129181 GGGAGGCTGA GGTGGGAGAA TCACCTGAGC CTGGGAGGTC AAGGCTGCAG TGAGGTGAGA |
| | 129241 TTGTGCCACC GCACTCTAGC CTGGGCGATA GAGCAAGACC CTGTCTCAAA AACAAACAAA |
| | 129301 AAACAGTCCC TGGCACTCTG GGCCAGGCCT GGCAGGGCAG TTGGCAGGGC TGGTCTTTCT |
| | 129361 CTGGCACTTC ATCTCACCCT CCCTCCCTTC CTCTTCTTGC AGATTGAAAC CCACAAGCTG |
| | 129421 ACCTTCCGCG AGAACGCCAA AGCCAAGACA GACCACGGGG CGGAGATCGT GTACAAGTCG |
| | 129481 CCAGTGGTGT CTGGGGACAC GTCTCCACGG CATCTCAGCA ATGTCTCCTC CACCGGCAGC |
| | 129541 ATCGACATGG TAGACTCGCC CCAGCTCGCC ACGCTAGCTG ACGAGGTGTC TGCCTCCCTG |
| | 129601 GCCAAGCAGG GTTTGTGATC AGGCCCCTGG GGCGGTCAAT AATTGTGGAG AGGAGAGAAT |
| | 129661 GAGAGAGTGT GGAAAAAAAA AGAATAATGA CCCGGCCCCC GCCCTCTGCC CCCAGCTGCT |
| | 129721 CCTCGCAGTT CGGTTAATTG GTTAATCACT TAACCTGCTT TTGTCACTCG GCTTTGGCTC |
| | 129781 GGGACTTCAA AATCAGTGAT GGGAGTAAGA GCAAATTTCA TCTTTCCAAA TTGATGGGTG |
| | 129841 GGCTAGTAAT AAAATATTTA AAAAAAAACA TTCAAAAACA TGGCCACATC CAACATTTCC |
| | 129901 TCAGGCAATT CCTTTTGATT CTTTTTTCTT CCCCCTCCAT GTAGAAGAGG GAGAAGGAGA |
| | 129961 GGCTCTGAAA GCTGCTTCTG GGGGATTTCA AGGGACTGGG GGTGCCAACC ACCTCTGGCC |
| | 130021 CTGTTGTGGG GGTGTCACAG AGGCAGTGGC AGCAACAAAG GATTTGAAAC TTGGTGTGTT |
| | 130081 CGTGGAGCCA CAGGCAGACG ATGTCAACCT TGTGTGAGTG TGACGGGGGT TGGGGTGGGG |
| | 130141 CGGGAGGCCA CGGGGGAGGC CGAGGCAGGG GCTGGGCAGA GGGGAGAGGA AGCACAAGAA |
| | 130201 GTGGGAGTGG GAGAGGAAGC CACGTGCTGG AGAGTAGACA TCCCCCTCCT TGCCGCTGGG |
| | 130261 AGAGCCAAGG CCTATGCCAC CTGCAGCGTC TGAGCGGCCG CCTGTCCTTG GTGGCCGGGG |
| | 130321 GTGGGGGCCT GCTGTGGGTC AGTGTGCCAC CCTCTGCAGG GCAGCCTGTG GGAGAAGGGA |
| | 130381 CAGCGGGTAA AAAGAGAAGG CAAGCTGGCA GGAGGGTGGC ACTTCGTGGA TGACCTCCTT |
| | 130441 AGAAAAGACT GACCTTGATG TCTTGAGAGC GCTGGCCTCT TCCTCCCTCC CTGCAGGGTA |
| | 130501 GGGGGCCTGA GTTGAGGGGC TTCCCTCTGC TCCACAGAAA CCCTGTTTTA TTGAGTTCTG |
| | 130561 AAGGTTGGAA CTGCTGCCAT GATTTTGGCC ACTTTGCAGA CCTGGGACTT TAGGGCTAAC |
| | 130621 CAGTTCTCTT TGTAAGGACT TGTGCCTCTT GGGAGACGTC CACCCGTTTC CAAGCCTGGG |
| | 130681 CCACTGGCAT CTCTGGAGTG TGTGGGGGTC TGGGAGGCAG GTCCCGAGCC CCCTGTCCTT |
| | 130741 CCCACGGCCA CTGCAGTCAC CCCGTCTGCG CCGCTGTGCT GTTGTCTGCC GTGAGAGCCC |
| | 130801 AATCACTGCC TATACCCCTC ATCACACGTC ACAATGTCCC GAATTCCCAG CCTCACCACC |
| | 130861 CCTTCTCAGT AATGACCCTG GTTGGTTGCA GGAGGTACCT ACTCCATACT GAGGGTGAAA |
| | 130921 TTAAGGGAAG GCAAAGTCCA GGCACAAGAG TGGGACCCCA GCCTCTCACT CTCAGTTCCA |
| | 130981 CTCATCCAAC TGGGACCCTC ACCACGAATC TCATGATCTG ATTCGGTTCC CTGTCTCCTC |
| | 131041 CTCCCGTCAC AGATGTGAGC CAGGGCACTG CTCAGCTGTG ACCCTAGGTG TTTCTGCCTT |
| | 131101 GTTGACATGG AGAGAGCCCT TTCCCCTGAG AAGGCCTGGC CCCTTCCTGT GCTGAGCCCA |
| | 131161 CAGCAGCAGG CTGGGTGTCT TGGTTGTCAG TGGTGGCACC AGGATGGAAG GGCAAGGCAC |
| | 131221 CCAGGGCAGG CCCACAGTCC CGCTGTCCCC CACTTGCACC CTAGCTTGTA GCTGCCAACC |
| | 131281 TCCCAGACAG CCCAGCCCGC TGCTCAGCTC CACATGCATA GTATCAGCCC TCCACACCCG |
| | 131341 ACAAAGGGGA ACACACCCCC TTGGAAATGG TTCTTTTCCC CCAGTCCCAG CTGGAAGCCA |
| | 131401 TGCTGTCTGT TCTGCTGGAG CAGCTGAACA TATACATAGA TGTTGCCCTG CCCTCCCCAT |
| | 131461 CTGCACCCTG TTGAGTTGTA GTTGGATTTG TCTGTTTATG CTTGGATTCA CCAGAGTGAC |
| | 131521 TATGATAGTG AAAAGAAAAA AAAAAAAAAA AAAGGACGCA TGTATCTTGA AATGCTTGTA |
| | 131581 AAGAGGTTTC TAACCCACCC TCACGAGGTG TCTCTCACCC CCACACTGGG ACTCGTGTGG |
| | 131641 CCTGTGTGGT GCCACCCTGC TGGGGCCTCC CAAGTTTTGA AAGGCTTTCC TCAGCACCTG |
| | 131701 GGACCCAACA GAGACCAGCT TCTAGCAGCT AAGGAGGCCG TTCAGCTGTG ACGAAGGCCT |
| | 131761 GAAGCACAGG ATTAGGACTG AAGCGATGAT GTCCCCTTCC CTACTTCCCC TTGGGGCTCC |
| | 131821 CTGTGTCAGG GCACAGACTA GGTCTTGTGG CTGGTCTGGC TTGCGGCGCG AGGATGGTTC |
| | 131881 TCTCTGGTCA TAGCCCGAAG TCTCATGGCA GTCCCAAAGG AGGCTTACAA CTCCTGCATC |
| | 131941 ACAAGAAAAA GGAAGCCACT GCCAGCTGGG GGGATCTGCA GCTCCCAGAA GCTCCGTGAG |
| | 132001 CCTCAGCCAC CCCTCAGACT GGGTTCCTCT CCAAGCTCGC CCTCTGGAGG GGCAGCGCAG |
| | 132061 CCTCCCACCA AGGGCCCTGC GACCACAGCA GGGATTGGGA TGAATTGCCT GTCCTGGATC |
| | 132121 TGCTCTAGAG GCCCAAGCTG CCTGCCTGAG GAAGGATGAC TTGACAAGTC AGGAGACACT |
| | 132181 GTTCCCAAAG CCTTGACCAG AGCACCTCAG CCCGCTGACC TTGCACAAAC TCCATCTGCT |
| | 132241 GCCATGAGAA AAGGGAAGCC GCCTTTGCAA AACATTGCTG CCTAAAGAAA CTCAGCAGCC |
| | 132301 TCAGGCCCAA TTCTGCCACT TCTGGTTTGG GTACAGTTAA AGGCAACCCT GAGGGACTTG |
| | 132361 GCAGTAGAAA TCCAGGGCCT CCCCTGGGGC TGGCAGCTTC GTGTGCAGCT AGAGCTTTAC |
| | 132421 CTGAAAGGAA GTCTCTGGGC CCAGAACTCT CCACCAAGAG CCTCCCTGCC GTTCGCTGAG |
| | 132481 TCCCAGCAAT TCTCCTAAGT TGAAGGGATC TGAGAAGGAG AAGGAAATGT GGGGTAGATT |
| | 132541 TGGTGGTGGT TAGAGATATG CCCCCCTCAT TACTGCCAAC AGTTTCGGCT GCATTTCTTC |
| | 132601 ACGCACCTCG GTTCCTCTTC CTGAAGTTCT TGTGCCCTGC TCTTCAGCAC CATGGGCCTT |
| | 132661 CTTATACGGA AGGCTCTGGG ATCTCCCCCT TGTGGGGCAG GCTCTTGGGG CCAGCCTAAG |
| | 132721 ATCATGGTTT AGGGTGATCA GTGCTGGCAG ATAAATTGAA AAGGCACGCT GGCTTGTGAT |
| | 132781 CTTAAATGAG GACAATCCCC CCAGGGCTGG GCACTCCTCC CCTCCCCTCA CTTCTCCCAC |
| | 132841 CTGCAGAGCC AGTGTCCTTG GGTGGGCTAG ATAGGATATA CTGTATGCCG GCTCCTTCAA |
| | 132901 GCTGCTGACT CACTTTATCA ATAGTTCCAT TTAAATTGAC TTCAGTGGTG AGACTGTATC |
| | 132961 CTGTTTGCTA TTGCTTGTTG TGCTATGGGG GGAGGGGGGA GGAATGTGTA AGATAGTTAA |
| | 133021 CATGGGCAAA GGGAGATCTT GGGGTGCAGC ACTTAAACTG CCTCGTAACC CTTTTCATGA |
| | 133081 TTTCAACCAC ATTTGCTAGA GGGAGGGAGC AGCCACGGAG TTAGAGGCCC TTGGGGTTTC |
| | 133141 TCTTTTCCAC TGACAGGCTT TCCCAGGCAG CTGGCTAGTT CATTCCCTCC CCAGCCAGGT |
| | 133201 GCAGGCGTAG GAATATGGAC ATCTGGTTGC TTTGGCCTGC TGCCCTCTTT CAGGGGTCCT |
| | 133261 AAGCCCACAA TCATGCCTCC CTAAGACCTT GGCATCCTTC CCTCTAAGCC GTTGGCACCT |
| | 133321 CTGTGCCACC TCTCACACTG GCTCCAGACA CACAGCCTGT GCTTTTGGAG CTGAGATCAC |
| | 133381 TCGCTTCACC CTCCTCATCT TTGTTCTCCA AGTAAAGCCA CGAGGTCGGG GCGAGGGCAG |
| | 133441 AGGTGATCAC CTGCGTGTCC CATCTACAGA CCTGCAGCTT CATAAAACTT CTGATTTCTC |
| | 133501 TTCAGCTTTG AAAAGGGTTA CCCTGGGCAC TGGCCTAGAG CCTCACCTCC TAATAGACTT |
| | 133561 AGCCCCATGA GTTTGCCATG TTGAGCAGGA CTATTTCTGG CACTTGCAAG TCCCATGATT |
| | 133621 TCTTCGGTAA TTCTGAGGGT GGGGGGAGGG ACATGAAATC ATCTTAGCTT AGCTTTCTGT |
| | 133681 CTGTGAATGT CTATATAGTG TATTGTGTGT TTTAACAAAT GATTTACACT GACTGTTGCT |
| | 133741 GTAAAAGTGA ATTTGGAAAT AAAGTTATTA CTCTGATTAA A |
| 24 | 1 GCAGTCACCG CCACCCACCA GCTCCGGCAC CAACAGCAGC GCCGCTGCCA CCGCCCACCT |
| | 61 TCTGCCGCCG CCACCACAGC CACCTTCTCC TCCTCCGCTG TCCTCTCCCG TCCTCGCCTC |
| | 121 TGTCGACTAT CAGGTGAACT TTGAACCAGG ATGGCTGAGC CCCGCCAGGA GTTCGAAGTG |
| | 181 ATGGAAGATC ACGCTGGGAC GTACGGGTTG GGGGACAGGA AAGATCAGGG GGGCTACACC |
| | 241 ATGCACCAAG ACCAAGAGGG TGACACGGAC GCTGGCCTGA AAGAATCTCC CCTGCAGACC |
| | 301 CCCACTGAGG ACGGATCTGA GGAACCGGGC TCTGAAACCT CTGATGCTAA GAGCACTCCA |
| | 361 ACAGCGGAAG ATGTGACAGC ACCCTTAGTG GATGAGGGAG CTCCCGGCAA GCAGGCTGCC |
| | 421 GCGCAGCCCC ACACGGAGAT CCCAGAAGGA ACCACAGCTG AAGAAGCAGG CATTGGAGAC |
| | 481 ACCCCCAGCC TGGAAGACGA AGCTGCTGGT CACGTGACCC AAGAGCCTGA AAGTGGTAAG |
| | 541 GTGGTCCAGG AAGGCTTCCT CCGAGAGCCA GGCCCCCCAG GTCTGAGCCA CCAGCTCATG |
| | 601 TCCGGCATGC CTGGGGCTCC CCTCCTGCCT GAGGGCCCCA GAGAGGCCAC ACGCCAACCT |
| | 661 TCGGGGACAG GACCTGAGGA CACAGAGGGC GGCCGCCACG CCCCTGAGCT GCTCAAGCAC |
| | 721 CAGCTTCTAG GAGACCTGCA CCAGGAGGGG CCGCCGCTGA AGGGGGCAGG GGGCAAAGAG |
| | 781 AGGCCGGGGA GCAAGGAGGA GGTGGATGAA GACCGCGACG TCGATGAGTC CTCCCCCCAA |
| | 841 GACTCCCCTC CCTCCAAGGC CTCCCCAGCC CAAGATGGGC GGCCTCCCCA GACAGCCGCC |
| | 901 AGAGAAGCCA CCAGCATCCC AGGCTTCCCA GCGGAGGGTG CCATCCCCCT CCCTGTGGAT |
| | 961 TTCCTCTCCA AAGTTTCCAC AGAGATCCCA GCCTCAGAGC CCGACGGGCC CAGTGTAGGG |
| | 1021 CGGGCCAAAG GGCAGGATGC CCCCCTGGAG TTCACGTTTC ACGTGGAAAT CACACCCAAC |
| | 1081 GTGCAGAAGG AGCAGGCGCA CTCGGAGGAG CATTTGGGAA GGGCTGCATT TCCAGGGGCC |
| | 1141 CCTGGAGAGG GGCCAGAGGC CCGGGGCCCC TCTTTGGGAG AGGACACAAA AGAGGCTGAC |
| | 1201 CTTCCAGAGC CCTCTGAAAA GCAGCCTGCT GCTGCTCCGC GGGGGAAGCC CGTCAGCCGG |
| | 1261 GTCCCTCAAC TCAAAGCTCG CATGGTCAGT AAAAGCAAAG ACGGGACTGG AAGCGATGAC |
| | 1321 AAAAAAGCCA AGACATCCAC ACGTTCCTCT GCTAAAACCT TGAAAAATAG GCCTTGCCTT |
| | 1381 AGCCCCAAAC ACCCCACTCC TGGTAGCTCA GACCCTCTGA TCCAACCCTC CAGCCCTGCT |
| | 1441 GTGTGCCCAG AGCCACCTTC CTCTCCTAAA TACGTCTCTT CTGTCACTTC CCGAACTGGC |
| | 1501 AGTTCTGGAG CAAAGGAGAT GAAACTCAAG GGGGCTGATG GTAAAACGAA GATCGCCACA |
| | 1561 CCGCGGGGAG CAGCCCCTCC AGGCCAGAAG GGCCAGGCCA ACGCCACCAG GATTCCAGCA |
| | 1621 AAAACCCCGC CCGCTCCAAA GACACCACCC AGCTCTGCGA CTAAGCAAGT CCAGAGAAGA |
| | 1681 CCACCCCCTG CAGGGCCCAG ATCTGAGAGA GGTGAACCTC CAAAATCAGG GGATCGCAGC |
| | 1741 GGCTACAGCA GCCCCGGCTC CCCAGGCACT CCCGGCAGCC GCTCCCGCAC CCCGTCCCTT |
| | 1801 CCAACCCCAC CCACCCGGGA GCCCAAGAAG GTGGCAGTGG TCCGTACTCC ACCCAAGTCG |
| | 1861 CCGTCTTCCG CCAAGAGCCG CCTGCAGACA GCCCCCGTGC CCATGCCAGA CCTGAAGAAT |
| | 1921 GTCAAGTCCA AGATCGGCTC CACTGAGAAC CTGAAGCACC AGCCGGGAGG CGGGAAGGTG |
| | 1981 CAGATAATTA ATAAGAAGCT GGATCTTAGC AACGTCCAGT CCAAGTGTGG CTCAAAGGAT |
| | 2041 AATATCAAAC ACGTCCCGGG AGGCGGCAGT GTGCAAATAG TCTACAAACC AGTTGACCTG |
| | 2101 AGCAAGGTGA CCTCCAAGTG TGGCTCATTA GGCAACATCC ATCATAAACC AGGAGGTGGC |
| | 2161 CAGGTGGAAG TAAAATCTGA GAAGCTTGAC TTCAAGGACA GAGTCCAGTC GAAGATTGGG |
| | 2221 TCCCTGGACA ATATCACCCA CGTCCCTGGC GGAGGAAATA AAAAGATTGA AACCCACAAG |
| | 2281 CTGACCTTCC GCGAGAACGC CAAAGCCAAG ACAGACCACG GGGCGGAGAT CGTGTACAAG |
| | 2341 TCGCCAGTGG TGTCTGGGGA CACGTCTCCA CGGCATCTCA GCAATGTCTC CTCCACCGGC |
| | 2401 AGCATCGACA TGGTAGACTC GCCCCAGCTC GCCACGCTAG CTGACGAGGT GTCTGCCTCC |
| | 2461 CTGGCCAAGC AGGGTTTGTG ATCAGGCCCC TGGGGCGGTC AATAATTGTG GAGAGGAGAG |
| | 2521 AATGAGAGAG TGTGGAAAAA AAAAGAATAA TGACCCGGCC CCCGCCCTCT GCCCCCAGCT |
| | 2581 GCTCCTCGCA GTTCGGTTAA TTGGTTAATC ACTTAACCTG CTTTTGTCAC TCGGCTTTGG |
| | 2641 CTCGGGACTT CAAAATCAGT GATGGGAGTA AGAGCAAATT TCATCTTTCC AAATTGATGG |
| | 2701 GTGGGCTAGT AATAAAATAT TTAAAAAAAA ACATTCAAAA ACATGGCCAC ATCCAACATT |
| | 2761 TCCTCAGGCA ATTCCTTTTG ATTCTTTTTT CTTCCCCCTC CATGTAGAAG AGGGAGAAGG |
| | 2821 AGAGGCTCTG AAAGCTGCTT CTGGGGGATT TCAAGGGACT GGGGGTGCCA ACCACCTCTG |
| | 2881 GCCCTGTTGT GGGGGTGTCA CAGAGGCAGT GGCAGCAACA AAGGATTTGA AACTTGGTGT |
| | 2941 GTTCGTGGAG CCACAGGCAG ACGATGTCAA CCTTGTGTGA GTGTGACGGG GGTTGGGGTG |
| | 3001 GGGCGGGAGG CCACGGGGGA GGCCGAGGCA GGGGCTGGGC AGAGGGGAGA GGAAGCACAA |
| | 3061 GAAGTGGGAG TGGGAGAGGA AGCCACGTGC TGGAGAGTAG ACATCCCCCT CCTTGCCGCT |
| | 3121 GGGAGAGCCA AGGCCTATGC CACCTGCAGC GTCTGAGCGG CCGCCTGTCC TTGGTGGCCG |
| | 3181 GGGGTGGGGG CCTGCTGTGG GTCAGTGTGC CACCCTCTGC AGGGCAGCCT GTGGGAGAAG |
| | 3241 GGACAGCGGG TAAAAAGAGA AGGCAAGCTG GCAGGAGGGT GGCACTTCGT GGATGACCTC |
| | 3301 CTTAGAAAAG ACTGACCTTG ATGTCTTGAG AGCGCTGGCC TCTTCCTCCC TCCCTGCAGG |
| | 3361 GTAGGGGGCC TGAGTTGAGG GGCTTCCCTC TGCTCCACAG AAACCCTGTT TTATTGAGTT |
| | 3421 CTGAAGGTTG GAACTGCTGC CATGATTTTG GCCACTTTGC AGACCTGGGA CTTTAGGGCT |
| | 3481 AACCAGTTCT CTTTGTAAGG ACTTGTGCCT CTTGGGAGAC GTCCACCCGT TTCCAAGCCT |
| | 3541 GGGCCACTGG CATCTCTGGA GTGTGTGGGG GTCTGGGAGG CAGGTCCCGA GCCCCCTGTC |
| | 3601 CTTCCCACGG CCACTGCAGT CACCCCGTCT GCGCCGCTGT GCTGTTGTCT GCCGTGAGAG |
| | 3661 CCCAATCACT GCCTATACCC CTCATCACAC GTCACAATGT CCCGAATTCC CAGCCTCACC |
| | 3721 ACCCCTTCTC AGTAATGACC CTGGTTGGTT GCAGGAGGTA CCTACTCCAT ACTGAGGGTG |
| | 3781 AAATTAAGGG AAGGCAAAGT CCAGGCACAA GAGTGGGACC CCAGCCTCTC ACTCTCAGTT |
| | 3841 CCACTCATCC AACTGGGACC CTCACCACGA ATCTCATGAT CTGATTCGGT TCCCTGTCTC |
| | 3901 CTCCTCCCGT CACAGATGTG AGCCAGGGCA CTGCTCAGCT GTGACCCTAG GTGTTTCTGC |
| | 3961 CTTGTTGACA TGGAGAGAGC CCTTTCCCCT GAGAAGGCCT GGCCCCTTCC TGTGCTGAGC |
| | 4021 CCACAGCAGC AGGCTGGGTG TCTTGGTTGT CAGTGGTGGC ACCAGGATGG AAGGGCAAGG |
| | 4081 CACCCAGGGC AGGCCCACAG TCCCGCTGTC CCCCACTTGC ACCCTAGCTT GTAGCTGCCA |
| | 4141 ACCTCCCAGA CAGCCCAGCC CGCTGCTCAG CTCCACATGC ATAGTATCAG CCCTCCACAC |
| | 4201 CCGACAAAGG GGAACACACC CCCTTGGAAA TGGTTCTTTT CCCCCAGTCC CAGCTGGAAG |
| | 4261 CCATGCTGTC TGTTCTGCTG GAGCAGCTGA ACATATACAT AGATGTTGCC CTGCCCTCCC |
| | 4321 CATCTGCACC CTGTTGAGTT GTAGTTGGAT TTGTCTGTTT ATGCTTGGAT TCACCAGAGT |
| | 4381 GACTATGATA GTGAAAAGAA AAAAAAAAAA AAAAAAGGAC GCATGTATCT TGAAATGCTT |
| | 4441 GTAAAGAGGT TTCTAACCCA CCCTCACGAG GTGTCTCTCA CCCCCACACT GGGACTCGTG |
| | 4501 TGGCCTGTGT GGTGCCACCC TGCTGGGGCC TCCCAAGTTT TGAAAGGCTT TCCTCAGCAC |
| | 4561 CTGGGACCCA ACAGAGACCA GCTTCTAGCA GCTAAGGAGG CCGTTCAGCT GTGACGAAGG |
| | 4621 CCTGAAGCAC AGGATTAGGA CTGAAGCGAT GATGTCCCCT TCCCTACTTC CCCTTGGGGC |
| | 4681 TCCCTGTGTC AGGGCACAGA CTAGGTCTTG TGGCTGGTCT GGCTTGCGGC GCGAGGATGG |
| | 4741 TTCTCTCTGG TCATAGCCCG AAGTCTCATG GCAGTCCCAA AGGAGGCTTA CAACTCCTGC |
| | 4801 ATCACAAGAA AAAGGAAGCC ACTGCCAGCT GGGGGGATCT GCAGCTCCCA GAAGCTCCGT |
| | 4861 GAGCCTCAGC CACCCCTCAG ACTGGGTTCC TCTCCAAGCT CGCCCTCTGG AGGGGCAGCG |
| | 4921 CAGCCTCCCA CCAAGGGCCC TGCGACCACA GCAGGGATTG GGATGAATTG CCTGTCCTGG |
| | 4981 ATCTGCTCTA GAGGCCCAAG CTGCCTGCCT GAGGAAGGAT GACTTGACAA GTCAGGAGAC |
| | 5041 ACTGTTCCCA AAGCCTTGAC CAGAGCACCT CAGCCCGCTG ACCTTGCACA AACTCCATCT |
| | 5101 GCTGCCATGA GAAAAGGGAA GCCGCCTTTG CAAAACATTG CTGCCTAAAG AAACTCAGCA |
| | 5161 GCCTCAGGCC CAATTCTGCC ACTTCTGGTT TGGGTACAGT TAAAGGCAAC CCTGAGGGAC |
| | 5221 TTGGCAGTAG AAATCCAGGG CCTCCCCTGG GGCTGGCAGC TTCGTGTGCA GCTAGAGCTT |
| | 5281 TACCTGAAAG GAAGTCTCTG GGCCCAGAAC TCTCCACCAA GAGCCTCCCT GCCGTTCGCT |
| | 5341 GAGTCCCAGC AATTCTCCTA AGTTGAAGGG ATCTGAGAAG GAGAAGGAAA TGTGGGGTAG |
| | 5401 ATTTGGTGGT GGTTAGAGAT ATGCCCCCCT CATTACTGCC AACAGTTTCG GCTGCATTTC |
| | 5461 TTCACGCACC TCGGTTCCTC TTCCTGAAGT TCTTGTGCCC TGCTCTTCAG CACCATGGGC |
| | 5521 CTTCTTATAC GGAAGGCTCT GGGATCTCCC CCTTGTGGGG CAGGCTCTTG GGGCCAGCCT |
| | 5581 AAGATCATGG TTTAGGGTGA TCAGTGCTGG CAGATAAATT GAAAAGGCAC GCTGGCTTGT |
| | 5641 GATCTTAAAT GAGGACAATC CCCCCAGGGC TGGGCACTCC TCCCCTCCCC TCACTTCTCC |
| | 5701 CACCTGCAGA GCCAGTGTCC TTGGGTGGGC TAGATAGGAT ATACTGTATG CCGGCTCCTT |
| | 5761 CAAGCTGCTG ACTCACTTTA TCAATAGTTC CATTTAAATT GACTTCAGTG GTGAGACTGT |
| | 5821 ATCCTGTTTG CTATTGCTTG TTGTGCTATG GGGGGAGGGG GGAGGAATGT GTAAGATAGT |
| | 5881 TAACATGGGC AAAGGGAGAT CTTGGGGTGC AGCACTTAAA CTGCCTCGTA ACCCTTTTCA |
| | 5941 TGATTTCAAC CACATTTGCT AGAGGGAGGG AGCAGCCACG GAGTTAGAGG CCCTTGGGGT |
| | 6001 TTCTCTTTTC CACTGACAGG CTTTCCCAGG CAGCTGGCTA GTTCATTCCC TCCCCAGCCA |
| | 6061 GGTGCAGGCG TAGGAATATG GACATCTGGT TGCTTTGGCC TGCTGCCCTC TTTCAGGGGT |
| | 6121 CCTAAGCCCA CAATCATGCC TCCCTAAGAC CTTGGCATCC TTCCCTCTAA GCCGTTGGCA |
| | 6181 CCTCTGTGCC ACCTCTCACA CTGGCTCCAG ACACACAGCC TGTGCTTTTG GAGCTGAGAT |
| | 6241 CACTCGCTTC ACCCTCCTCA TCTTTGTTCT CCAAGTAAAG CCACGAGGTC GGGGCGAGGG |
| | 6301 CAGAGGTGAT CACCTGCGTG TCCCATCTAC AGACCTGCAG CTTCATAAAA CTTCTGATTT |
| | 6361 CTCTTCAGCT TTGAAAAGGG TTACCCTGGG CACTGGCCTA GAGCCTCACC TCCTAATAGA |
| | 6421 CTTAGCCCCA TGAGTTTGCC ATGTTGAGCA GGACTATTTC TGGCACTTGC AAGTCCCATG |
| | 6481 ATTTCTTCGG TAATTCTGAG GGTGGGGGGA GGGACATGAA ATCATCTTAG CTTAGCTTTC |
| | 6541 TGTCTGTGAA TGTCTATATA GTGTATTGTG TGTTTTAACA AATGATTTAC ACTGACTGTT |
| | 6601 GCTGTAAAAG TGAATTTGGA AATAAAGTTA TTACTCTGAT TAAA |
| 25 | 1 MAEPRQEFEV MEDHAGTYGL GDRKDQGGYT MHQDQEGDTD AGLKESPLQT PTEDGSEEPG |
| | 61 SETSDAKSTP TAEDVTAPLV DEGAPGKQAA AQPHTEIPEG TTAEEAGIGD TPSLEDEAAG |
| | 121 HVTQEPESGK VVQEGFLREP GPPGLSHQLM SGMPGAPLLP EGPREATRQP SGTGPEDTEG |
| | 181 GRHAPELLKH QLLGDLHQEG PPLKGAGGKE RPGSKEEVDE DRDVDESSPQ DSPPSKASPA |
| | 241 QDGRPPQTAA REATSIPGFP AEGAIPLPVD FLSKVSTEIP ASEPDGPSVG RAKGQDAPLE |
| | 301 FTFHVEITPN VQKEQAHSEE HLGRAAFPGA PGEGPEARGP SLGEDTKEAD LPEPSEKQPA |
| | 361 AAPRGKPVSR VPQLKARMVS KSKDGTGSDD KKAKTSTRSS AKTLKNRPCL SPKHPTPGSS |
| | 421 DPLIQPSSPA VCPEPPSSPK YVSSVTSRTG SSGAKEMKLK GADGKTKIAT PRGAAPPGQK |
| | 481 GQANATRIPA KTPPAPKTPP SSATKQVQRR PPPAGPRSER GEPPKSGDRS GYSSPGSPGT |
| | 541 PGSRSRTPSL PTPPTREPKK VAVVRTPPKS PSSAKSRLQT APVPMPDLKN VKSKIGSTEN |
| | 601 LKHQPGGGKV QIINKKLDLS NVQSKCGSKD NIKHVPGGGS VQIVYKPVDL SKVTSKCGSL |
| | 661 GNIHHKPGGG QVEVKSEKLD FKDRVQSKIG SLDNITHVPG GGNKKIETHK LTFRENAKAK |
| | 721 TDHGAEIVYK SPVVSGDTSP RHLSNVSSTG SIDMVDSPQL ATLADEVSAS LAKQGL |
| 26 | 1 GCAGTCACCG CCACCCACCA GCTCCGGCAC CAACAGCAGC GCCGCTGCCA CCGCCCACCT |
| | 61 TCTGCCGCCG CCACCACAGC CACCTTCTCC TCCTCCGCTG TCCTCTCCCG TCCTCGCCTC |
| | 121 TGTCGACTAT CAGGTGAACT TTGAACCAGG ATGGCTGAGC CCCGCCAGGA GTTCGAAGTG |
| | 181 ATGGAAGATC ACGCTGGGAC GTACGGGTTG GGGGACAGGA AAGATCAGGG GGGCTACACC |
| | 241 ATGCACCAAG ACCAAGAGGG TGACACGGAC GCTGGCCTGA AAGAATCTCC CCTGCAGACC |
| | 301 CCCACTGAGG ACGGATCTGA GGAACCGGGC TCTGAAACCT CTGATGCTAA GAGCACTCCA |
| | 361 ACAGCGGAAG CTGAAGAAGC AGGCATTGGA GACACCCCCA GCCTGGAAGA CGAAGCTGCT |
| | 421 GGTCACGTGA CCCAAGCTCG CATGGTCAGT AAAAGCAAAG ACGGGACTGG AAGCGATGAC |
| | 481 AAAAAAGCCA AGGGGGCTGA TGGTAAAACG AAGATCGCCA CACCGCGGGG AGCAGCCCCT |
| | 541 CCAGGCCAGA AGGGCCAGGC CAACGCCACC AGGATTCCAG CAAAAACCCC GCCCGCTCCA |
| | 601 AAGACACCAC CCAGCTCTGG TGAACCTCCA AAATCAGGGG ATCGCAGCGG CTACAGCAGC |
| | 661 CCCGGCTCCC CAGGCACTCC CGGCAGCCGC TCCCGCACCC CGTCCCTTCC AACCCCACCC |
| | 721 ACCCGGGAGC CCAAGAAGGT GGCAGTGGTC CGTACTCCAC CCAAGTCGCC GTCTTCCGCC |
| | 781 AAGAGCCGCC TGCAGACAGC CCCCGTGCCC ATGCCAGACC TGAAGAATGT CAAGTCCAAG |
| | 841 ATCGGCTCCA CTGAGAACCT GAAGCACCAG CCGGGAGGCG GGAAGGTGCA GATAATTAAT |
| | 901 AAGAAGCTGG ATCTTAGCAA CGTCCAGTCC AAGTGTGGCT CAAAGGATAA TATCAAACAC |
| | 961 GTCCCGGGAG GCGGCAGTGT GCAAATAGTC TACAAACCAG TTGACCTGAG CAAGGTGACC |
| | 1021 TCCAAGTGTG GCTCATTAGG CAACATCCAT CATAAACCAG GAGGTGGCCA GGTGGAAGTA |
| | 1081 AAATCTGAGA AGCTTGACTT CAAGGACAGA GTCCAGTCGA AGATTGGGTC CCTGGACAAT |
| | 1141 ATCACCCACG TCCCTGGCGG AGGAAATAAA AAGATTGAAA CCCACAAGCT GACCTTCCGC |
| | 1201 GAGAACGCCA AAGCCAAGAC AGACCACGGG GCGGAGATCG TGTACAAGTC GCCAGTGGTG |
| | 1261 TCTGGGGACA CGTCTCCACG GCATCTCAGC AATGTCTCCT CCACCGGCAG CATCGACATG |
| | 1321 GTAGACTCGC CCCAGCTCGC CACGCTAGCT GACGAGGTGT CTGCCTCCCT GGCCAAGCAG |
| | 1381 GGTTTGTGAT CAGGCCCCTG GGGCGGTCAA TAATTGTGGA GAGGAGAGAA TGAGAGAGTG |
| | 1441 TGGAAAAAAA AAGAATAATG ACCCGGCCCC CGCCCTCTGC CCCCAGCTGC TCCTCGCAGT |
| | 1501 TCGGTTAATT GGTTAATCAC TTAACCTGCT TTTGTCACTC GGCTTTGGCT CGGGACTTCA |
| | 1561 AAATCAGTGA TGGGAGTAAG AGCAAATTTC ATCTTTCCAA ATTGATGGGT GGGCTAGTAA |
| | 1621 TAAAATATTT AAAAAAAAAC ATTCAAAAAC ATGGCCACAT CCAACATTTC CTCAGGCAAT |
| | 1681 TCCTTTTGAT TCTTTTTTCT TCCCCCTCCA TGTAGAAGAG GGAGAAGGAG AGGCTCTGAA |
| | 1741 AGCTGCTTCT GGGGGATTTC AAGGGACTGG GGGTGCCAAC CACCTCTGGC CCTGTTGTGG |
| | 1801 GGGTGTCACA GAGGCAGTGG CAGCAACAAA GGATTTGAAA CTTGGTGTGT TCGTGGAGCC |
| | 1861 ACAGGCAGAC GATGTCAACC TTGTGTGAGT GTGACGGGGG TTGGGGTGGG GCGGGAGGCC |
| | 1921 ACGGGGGAGG CCGAGGCAGG GGCTGGGCAG AGGGGAGAGG AAGCACAAGA AGTGGGAGTG |
| | 1981 GGAGAGGAAG CCACGTGCTG GAGAGTAGAC ATCCCCCTCC TTGCCGCTGG GAGAGCCAAG |
| | 2041 GCCTATGCCA CCTGCAGCGT CTGAGCGGCC GCCTGTCCTT GGTGGCCGGG GGTGGGGGCC |
| | 2101 TGCTGTGGGT CAGTGTGCCA CCCTCTGCAG GGCAGCCTGT GGGAGAAGGG ACAGCGGGTA |
| | 2161 AAAAGAGAAG GCAAGCTGGC AGGAGGGTGG CACTTCGTGG ATGACCTCCT TAGAAAAGAC |
| | 2221 TGACCTTGAT GTCTTGAGAG CGCTGGCCTC TTCCTCCCTC CCTGCAGGGT AGGGGGCCTG |
| | 2281 AGTTGAGGGG CTTCCCTCTG CTCCACAGAA ACCCTGTTTT ATTGAGTTCT GAAGGTTGGA |
| | 2341 ACTGCTGCCA TGATTTTGGC CACTTTGCAG ACCTGGGACT TTAGGGCTAA CCAGTTCTCT |
| | 2401 TTGTAAGGAC TTGTGCCTCT TGGGAGACGT CCACCCGTTT CCAAGCCTGG GCCACTGGCA |
| | 2461 TCTCTGGAGT GTGTGGGGGT CTGGGAGGCA GGTCCCGAGC CCCCTGTCCT TCCCACGGCC |
| | 2521 ACTGCAGTCA CCCCGTCTGC GCCGCTGTGC TGTTGTCTGC CGTGAGAGCC CAATCACTGC |
| | 2581 CTATACCCCT CATCACACGT CACAATGTCC CGAATTCCCA GCCTCACCAC CCCTTCTCAG |
| | 2641 TAATGACCCT GGTTGGTTGC AGGAGGTACC TACTCCATAC TGAGGGTGAA ATTAAGGGAA |
| | 2701 GGCAAAGTCC AGGCACAAGA GTGGGACCCC AGCCTCTCAC TCTCAGTTCC ACTCATCCAA |
| | 2761 CTGGGACCCT CACCACGAAT CTCATGATCT GATTCGGTTC CCTGTCTCCT CCTCCCGTCA |
| | 2821 CAGATGTGAG CCAGGGCACT GCTCAGCTGT GACCCTAGGT GTTTCTGCCT TGTTGACATG |
| | 2881 GAGAGAGCCC TTTCCCCTGA GAAGGCCTGG CCCCTTCCTG TGCTGAGCCC ACAGCAGCAG |
| | 2941 GCTGGGTGTC TTGGTTGTCA GTGGTGGCAC CAGGATGGAA GGGCAAGGCA CCCAGGGCAG |
| | 3001 GCCCACAGTC CCGCTGTCCC CCACTTGCAC CCTAGCTTGT AGCTGCCAAC CTCCCAGACA |
| | 3061 GCCCAGCCCG CTGCTCAGCT CCACATGCAT AGTATCAGCC CTCCACACCC GACAAAGGGG |
| | 3121 AACACACCCC CTTGGAAATG GTTCTTTTCC CCCAGTCCCA GCTGGAAGCC ATGCTGTCTG |
| | 3181 TTCTGCTGGA GCAGCTGAAC ATATACATAG ATGTTGCCCT GCCCTCCCCA TCTGCACCCT |
| | 3241 GTTGAGTTGT AGTTGGATTT GTCTGTTTAT GCTTGGATTC ACCAGAGTGA CTATGATAGT |
| | 3301 GAAAAGAAAA AAAAAAAAAA AAAAGGACGC ATGTATCTTG AAATGCTTGT AAAGAGGTTT |
| | 3361 CTAACCCACC CTCACGAGGT GTCTCTCACC CCCACACTGG GACTCGTGTG GCCTGTGTGG |
| | 3421 TGCCACCCTG CTGGGGCCTC CCAAGTTTTG AAAGGCTTTC CTCAGCACCT GGGACCCAAC |
| | 3481 AGAGACCAGC TTCTAGCAGC TAAGGAGGCC GTTCAGCTGT GACGAAGGCC TGAAGCACAG |
| | 3541 GATTAGGACT GAAGCGATGA TGTCCCCTTC CCTACTTCCC CTTGGGGCTC CCTGTGTCAG |
| | 3601 GGCACAGACT AGGTCTTGTG GCTGGTCTGG CTTGCGGCGC GAGGATGGTT CTCTCTGGTC |
| | 3661 ATAGCCCGAA GTCTCATGGC AGTCCCAAAG GAGGCTTACA ACTCCTGCAT CACAAGAAAA |
| | 3721 AGGAAGCCAC TGCCAGCTGG GGGGATCTGC AGCTCCCAGA AGCTCCGTGA GCCTCAGCCA |
| | 3781 CCCCTCAGAC TGGGTTCCTC TCCAAGCTCG CCCTCTGGAG GGGCAGCGCA GCCTCCCACC |
| | 3841 AAGGGCCCTG CGACCACAGC AGGGATTGGG ATGAATTGCC TGTCCTGGAT CTGCTCTAGA |
| | 3901 GGCCCAAGCT GCCTGCCTGA GGAAGGATGA CTTGACAAGT CAGGAGACAC TGTTCCCAAA |
| | 3961 GCCTTGACCA GAGCACCTCA GCCCGCTGAC CTTGCACAAA CTCCATCTGC TGCCATGAGA |
| | 4021 AAAGGGAAGC CGCCTTTGCA AAACATTGCT GCCTAAAGAA ACTCAGCAGC CTCAGGCCCA |
| | 4081 ATTCTGCCAC TTCTGGTTTG GGTACAGTTA AAGGCAACCC TGAGGGACTT GGCAGTAGAA |
| | 4141 ATCCAGGGCC TCCCCTGGGG CTGGCAGCTT CGTGTGCAGC TAGAGCTTTA CCTGAAAGGA |
| | 4201 AGTCTCTGGG CCCAGAACTC TCCACCAAGA GCCTCCCTGC CGTTCGCTGA GTCCCAGCAA |
| | 4261 TTCTCCTAAG TTGAAGGGAT CTGAGAAGGA GAAGGAAATG TGGGGTAGAT TTGGTGGTGG |
| | 4321 TTAGAGATAT GCCCCCCTCA TTACTGCCAA CAGTTTCGGC TGCATTTCTT CACGCACCTC |
| | 4381 GGTTCCTCTT CCTGAAGTTC TTGTGCCCTG CTCTTCAGCA CCATGGGCCT TCTTATACGG |
| | 4441 AAGGCTCTGG GATCTCCCCC TTGTGGGGCA GGCTCTTGGG GCCAGCCTAA GATCATGGTT |
| | 4501 TAGGGTGATC AGTGCTGGCA GATAAATTGA AAAGGCACGC TGGCTTGTGA TCTTAAATGA |
| | 4561 GGACAATCCC CCCAGGGCTG GGCACTCCTC CCCTCCCCTC ACTTCTCCCA CCTGCAGAGC |
| | 4621 CAGTGTCCTT GGGTGGGCTA GATAGGATAT ACTGTATGCC GGCTCCTTCA AGCTGCTGAC |
| | 4681 TCACTTTATC AATAGTTCCA TTTAAATTGA CTTCAGTGGT GAGACTGTAT CCTGTTTGCT |
| | 4741 ATTGCTTGTT GTGCTATGGG GGGAGGGGGG AGGAATGTGT AAGATAGTTA ACATGGGCAA |
| | 4801 AGGGAGATCT TGGGGTGCAG CACTTAAACT GCCTCGTAAC CCTTTTCATG ATTTCAACCA |
| | 4861 CATTTGCTAG AGGGAGGGAG CAGCCACGGA GTTAGAGGCC CTTGGGGTTT CTCTTTTCCA |
| | 4921 CTGACAGGCT TTCCCAGGCA GCTGGCTAGT TCATTCCCTC CCCAGCCAGG TGCAGGCGTA |
| | 4981 GGAATATGGA CATCTGGTTG CTTTGGCCTG CTGCCCTCTT TCAGGGGTCC TAAGCCCACA |
| | 5041 ATCATGCCTC CCTAAGACCT TGGCATCCTT CCCTCTAAGC CGTTGGCACC TCTGTGCCAC |
| | 5101 CTCTCACACT GGCTCCAGAC ACACAGCCTG TGCTTTTGGA GCTGAGATCA CTCGCTTCAC |
| | 5161 CCTCCTCATC TTTGTTCTCC AAGTAAAGCC ACGAGGTCGG GGCGAGGGCA GAGGTGATCA |
| | 5221 CCTGCGTGTC CCATCTACAG ACCTGCAGCT TCATAAAACT TCTGATTTCT CTTCAGCTTT |
| | 5281 GAAAAGGGTT ACCCTGGGCA CTGGCCTAGA GCCTCACCTC CTAATAGACT TAGCCCCATG |
| | 5341 AGTTTGCCAT GTTGAGCAGG ACTATTTCTG GCACTTGCAA GTCCCATGAT TTCTTCGGTA |
| | 5401 ATTCTGAGGG TGGGGGGAGG GACATGAAAT CATCTTAGCT TAGCTTTCTG TCTGTGAATG |
| | 5461 TCTATATAGT GTATTGTGTG TTTTAACAAA TGATTTACAC TGACTGTTGC TGTAAAAGTG |
| | 5521 AATTTGGAAA TAAAGTTATT ACTCTGATTA AA |
| 27 | 1 MAEPRQEFEV MEDHAGTYGL GDRKDQGGYT MHQDQEGDTD AGLKESPLQT PTEDGSEEPG |
| | 61 SETSDAKSTP TAEAEEAGIG DTPSLEDEAA GHVTQARMVS KSKDGTGSDD KKAKGADGKT |
| | 121 KIATPRGAAP PGQKGQANAT RIPAKTPPAP KTPPSSGEPP KSGDRSGYSS PGSPGTPGSR |
| | 181 SRTPSLPTPP TREPKKVAVV RTPPKSPSSA KSRLQTAPVP MPDLKNVKSK IGSTENLKHQ |
| | 241 PGGGKVQIIN KKLDLSNVQS KCGSKDNIKH VPGGGSVQIV YKPVDLSKVT SKCGSLGNIH |
| | 301 HKPGGGQVEV KSEKLDFKDR VQSKIGSLDN ITHVPGGGNK KIETHKLTER ENAKAKTDHG |
| | 361 AEIVYKSPVV SGDTSPRHLS NVSSTGSIDM VDSPQLATLA DEVSASLAKQ GL |
| 28 | 1 GCAGTCACCG CCACCCACCA GCTCCGGCAC CAACAGCAGC GCCGCTGCCA CCGCCCACCT |
| | 61 TCTGCCGCCG CCACCACAGC CACCTTCTCC TCCTCCGCTG TCCTCTCCCG TCCTCGCCTC |
| | 121 TGTCGACTAT CAGGTGAACT TTGAACCAGG ATGGCTGAGC CCCGCCAGGA GTTCGAAGTG |
| | 181 ATGGAAGATC ACGCTGGGAC GTACGGGTTG GGGGACAGGA AAGATCAGGG GGGCTACACC |
| | 241 ATGCACCAAG ACCAAGAGGG TGACACGGAC GCTGGCCTGA AAGCTGAAGA AGCAGGCATT |
| | 301 GGAGACACCC CCAGCCTGGA AGACGAAGCT GCTGGTCACG TGACCCAAGC TCGCATGGTC |
| | 361 AGTAAAAGCA AAGACGGGAC TGGAAGCGAT GACAAAAAAG CCAAGGGGGC TGATGGTAAA |
| | 421 ACGAAGATCG CCACACCGCG GGGAGCAGCC CCTCCAGGCC AGAAGGGCCA GGCCAACGCC |
| | 481 ACCAGGATTC CAGCAAAAAC CCCGCCCGCT CCAAAGACAC CACCCAGCTC TGGTGAACCT |
| | 541 CCAAAATCAG GGGATCGCAG CGGCTACAGC AGCCCCGGCT CCCCAGGCAC TCCCGGCAGC |
| | 601 CGCTCCCGCA CCCCGTCCCT TCCAACCCCA CCCACCCGGG AGCCCAAGAA GGTGGCAGTG |
| | 661 GTCCGTACTC CACCCAAGTC GCCGTCTTCC GCCAAGAGCC GCCTGCAGAC AGCCCCCGTG |
| | 721 CCCATGCCAG ACCTGAAGAA TGTCAAGTCC AAGATCGGCT CCACTGAGAA CCTGAAGCAC |
| | 781 CAGCCGGGAG GCGGGAAGGT GCAAATAGTC TACAAACCAG TTGACCTGAG CAAGGTGACC |
| | 841 TCCAAGTGTG GCTCATTAGG CAACATCCAT CATAAACCAG GAGGTGGCCA GGTGGAAGTA |
| | 901 AAATCTGAGA AGCTTGACTT CAAGGACAGA GTCCAGTCGA AGATTGGGTC CCTGGACAAT |
| | 961 ATCACCCACG TCCCTGGCGG AGGAAATAAA AAGATTGAAA CCCACAAGCT GACCTTCCGC |
| | 1021 GAGAACGCCA AAGCCAAGAC AGACCACGGG GCGGAGATCG TGTACAAGTC GCCAGTGGTG |
| | 1081 TCTGGGGACA CGTCTCCACG GCATCTCAGC AATGTCTCCT CCACCGGCAG CATCGACATG |
| | 1141 GTAGACTCGC CCCAGCTCGC CACGCTAGCT GACGAGGTGT CTGCCTCCCT GGCCAAGCAG |
| | 1201 GGTTTGTGAT CAGGCCCCTG GGGCGGTCAA TAATTGTGGA GAGGAGAGAA TGAGAGAGTG |
| | 1261 TGGAAAAAAA AAGAATAATG ACCCGGCCCC CGCCCTCTGC CCCCAGCTGC TCCTCGCAGT |
| | 1321 TCGGTTAATT GGTTAATCAC TTAACCTGCT TTTGTCACTC GGCTTTGGCT CGGGACTTCA |
| | 1381 AAATCAGTGA TGGGAGTAAG AGCAAATTTC ATCTTTCCAA ATTGATGGGT GGGCTAGTAA |
| | 1441 TAAAATATTT AAAAAAAAAC ATTCAAAAAC ATGGCCACAT CCAACATTTC CTCAGGCAAT |
| | 1501 TCCTTTTGAT TCTTTTTTCT TCCCCCTCCA TGTAGAAGAG GGAGAAGGAG AGGCTCTGAA |
| | 1561 AGCTGCTTCT GGGGGATTTC AAGGGACTGG GGGTGCCAAC CACCTCTGGC CCTGTTGTGG |
| | 1621 GGGTGTCACA GAGGCAGTGG CAGCAACAAA GGATTTGAAA CTTGGTGTGT TCGTGGAGCC |
| | 1681 ACAGGCAGAC GATGTCAACC TTGTGTGAGT GTGACGGGGG TTGGGGTGGG GCGGGAGGCC |
| | 1741 ACGGGGGAGG CCGAGGCAGG GGCTGGGCAG AGGGGAGAGG AAGCACAAGA AGTGGGAGTG |
| | 1801 GGAGAGGAAG CCACGTGCTG GAGAGTAGAC ATCCCCCTCC TTGCCGCTGG GAGAGCCAAG |
| | 1861 GCCTATGCCA CCTGCAGCGT CTGAGCGGCC GCCTGTCCTT GGTGGCCGGG GGTGGGGGCC |
| | 1921 TGCTGTGGGT CAGTGTGCCA CCCTCTGCAG GGCAGCCTGT GGGAGAAGGG ACAGCGGGTA |
| | 1981 AAAAGAGAAG GCAAGCTGGC AGGAGGGTGG CACTTCGTGG ATGACCTCCT TAGAAAAGAC |
| | 2041 TGACCTTGAT GTCTTGAGAG CGCTGGCCTC TTCCTCCCTC CCTGCAGGGT AGGGGGCCTG |
| | 2101 AGTTGAGGGG CTTCCCTCTG CTCCACAGAA ACCCTGTTTT ATTGAGTTCT GAAGGTTGGA |
| | 2161 ACTGCTGCCA TGATTTTGGC CACTTTGCAG ACCTGGGACT TTAGGGCTAA CCAGTTCTCT |
| | 2221 TTGTAAGGAC TTGTGCCTCT TGGGAGACGT CCACCCGTTT CCAAGCCTGG GCCACTGGCA |
| | 2281 TCTCTGGAGT GTGTGGGGGT CTGGGAGGCA GGTCCCGAGC CCCCTGTCCT TCCCACGGCC |
| | 2341 ACTGCAGTCA CCCCGTCTGC GCCGCTGTGC TGTTGTCTGC CGTGAGAGCC CAATCACTGC |
| | 2401 CTATACCCCT CATCACACGT CACAATGTCC CGAATTCCCA GCCTCACCAC CCCTTCTCAG |
| | 2461 TAATGACCCT GGTTGGTTGC AGGAGGTACC TACTCCATAC TGAGGGTGAA ATTAAGGGAA |
| | 2521 GGCAAAGTCC AGGCACAAGA GTGGGACCCC AGCCTCTCAC TCTCAGTTCC ACTCATCCAA |
| | 2581 CTGGGACCCT CACCACGAAT CTCATGATCT GATTCGGTTC CCTGTCTCCT CCTCCCGTCA |
| | 2641 CAGATGTGAG CCAGGGCACT GCTCAGCTGT GACCCTAGGT GTTTCTGCCT TGTTGACATG |
| | 2701 GAGAGAGCCC TTTCCCCTGA GAAGGCCTGG CCCCTTCCTG TGCTGAGCCC ACAGCAGCAG |
| | 2761 GCTGGGTGTC TTGGTTGTCA GTGGTGGCAC CAGGATGGAA GGGCAAGGCA CCCAGGGCAG |
| | 2821 GCCCACAGTC CCGCTGTCCC CCACTTGCAC CCTAGCTTGT AGCTGCCAAC CTCCCAGACA |
| | 2881 GCCCAGCCCG CTGCTCAGCT CCACATGCAT AGTATCAGCC CTCCACACCC GACAAAGGGG |
| | 2941 AACACACCCC CTTGGAAATG GTTCTTTTCC CCCAGTCCCA GCTGGAAGCC ATGCTGTCTG |
| | 3001 TTCTGCTGGA GCAGCTGAAC ATATACATAG ATGTTGCCCT GCCCTCCCCA TCTGCACCCT |
| | 3061 GTTGAGTTGT AGTTGGATTT GTCTGTTTAT GCTTGGATTC ACCAGAGTGA CTATGATAGT |
| | 3121 GAAAAGAAAA AAAAAAAAAA AAAAGGACGC ATGTATCTTG AAATGCTTGT AAAGAGGTTT |
| | 3181 CTAACCCACC CTCACGAGGT GTCTCTCACC CCCACACTGG GACTCGTGTG GCCTGTGTGG |
| | 3241 TGCCACCCTG CTGGGGCCTC CCAAGTTTTG AAAGGCTTTC CTCAGCACCT GGGACCCAAC |
| | 3301 AGAGACCAGC TTCTAGCAGC TAAGGAGGCC GTTCAGCTGT GACGAAGGCC TGAAGCACAG |
| | 3361 GATTAGGACT GAAGCGATGA TGTCCCCTTC CCTACTTCCC CTTGGGGCTC CCTGTGTCAG |
| | 3421 GGCACAGACT AGGTCTTGTG GCTGGTCTGG CTTGCGGCGC GAGGATGGTT CTCTCTGGTC |
| | 3481 ATAGCCCGAA GTCTCATGGC AGTCCCAAAG GAGGCTTACA ACTCCTGCAT CACAAGAAAA |
| | 3541 AGGAAGCCAC TGCCAGCTGG GGGGATCTGC AGCTCCCAGA AGCTCCGTGA GCCTCAGCCA |
| | 3601 CCCCTCAGAC TGGGTTCCTC TCCAAGCTCG CCCTCTGGAG GGGCAGCGCA GCCTCCCACC |
| | 3661 AAGGGCCCTG CGACCACAGC AGGGATTGGG ATGAATTGCC TGTCCTGGAT CTGCTCTAGA |
| | 3721 GGCCCAAGCT GCCTGCCTGA GGAAGGATGA CTTGACAAGT CAGGAGACAC TGTTCCCAAA |
| | 3781 GCCTTGACCA GAGCACCTCA GCCCGCTGAC CTTGCACAAA CTCCATCTGC TGCCATGAGA |
| | 3841 AAAGGGAAGC CGCCTTTGCA AAACATTGCT GCCTAAAGAA ACTCAGCAGC CTCAGGCCCA |
| | 3901 ATTCTGCCAC TTCTGGTTTG GGTACAGTTA AAGGCAACCC TGAGGGACTT GGCAGTAGAA |
| | 3961 ATCCAGGGCC TCCCCTGGGG CTGGCAGCTT CGTGTGCAGC TAGAGCTTTA CCTGAAAGGA |
| | 4021 AGTCTCTGGG CCCAGAACTC TCCACCAAGA GCCTCCCTGC CGTTCGCTGA GTCCCAGCAA |
| | 4081 TTCTCCTAAG TTGAAGGGAT CTGAGAAGGA GAAGGAAATG TGGGGTAGAT TTGGTGGTGG |
| | 4141 TTAGAGATAT GCCCCCCTCA TTACTGCCAA CAGTTTCGGC TGCATTTCTT CACGCACCTC |
| | 4201 GGTTCCTCTT CCTGAAGTTC TTGTGCCCTG CTCTTCAGCA CCATGGGCCT TCTTATACGG |
| | 4261 AAGGCTCTGG GATCTCCCCC TTGTGGGGCA GGCTCTTGGG GCCAGCCTAA GATCATGGTT |
| | 4321 TAGGGTGATC AGTGCTGGCA GATAAATTGA AAAGGCACGC TGGCTTGTGA TCTTAAATGA |
| | 4381 GGACAATCCC CCCAGGGCTG GGCACTCCTC CCCTCCCCTC ACTTCTCCCA CCTGCAGAGC |
| | 4441 CAGTGTCCTT GGGTGGGCTA GATAGGATAT ACTGTATGCC GGCTCCTTCA AGCTGCTGAC |
| | 4501 TCACTTTATC AATAGTTCCA TTTAAATTGA CTTCAGTGGT GAGACTGTAT CCTGTTTGCT |
| | 4561 ATTGCTTGTT GTGCTATGGG GGGAGGGGGG AGGAATGTGT AAGATAGTTA ACATGGGCAA |
| | 4621 AGGGAGATCT TGGGGTGCAG CACTTAAACT GCCTCGTAAC CCTTTTCATG ATTTCAACCA |
| | 4681 CATTTGCTAG AGGGAGGGAG CAGCCACGGA GTTAGAGGCC CTTGGGGTTT CTCTTTTCCA |
| | 4741 CTGACAGGCT TTCCCAGGCA GCTGGCTAGT TCATTCCCTC CCCAGCCAGG TGCAGGCGTA |
| | 4801 GGAATATGGA CATCTGGTTG CTTTGGCCTG CTGCCCTCTT TCAGGGGTCC TAAGCCCACA |
| | 4861 ATCATGCCTC CCTAAGACCT TGGCATCCTT CCCTCTAAGC CGTTGGCACC TCTGTGCCAC |
| | 4921 CTCTCACACT GGCTCCAGAC ACACAGCCTG TGCTTTTGGA GCTGAGATCA CTCGCTTCAC |
| | 4981 CCTCCTCATC TTTGTTCTCC AAGTAAAGCC ACGAGGTCGG GGCGAGGGCA GAGGTGATCA |
| | 5041 CCTGCGTGTC CCATCTACAG ACCTGCAGCT TCATAAAACT TCTGATTTCT CTTCAGCTTT |
| | 5101 GAAAAGGGTT ACCCTGGGCA CTGGCCTAGA GCCTCACCTC CTAATAGACT TAGCCCCATG |
| | 5161 AGTTTGCCAT GTTGAGCAGG ACTATTTCTG GCACTTGCAA GTCCCATGAT TTCTTCGGTA |
| | 5221 ATTCTGAGGG TGGGGGGAGG GACATGAAAT CATCTTAGCT TAGCTTTCTG TCTGTGAATG |
| | 5281 TCTATATAGT GTATTGTGTG TTTTAACAAA TGATTTACAC TGACTGTTGC TGTAAAAGTG |
| | 5341 AATTTGGAAA TAAAGTTATT ACTCTGATTA AA |
| 29 | 1 MAEPRQEFEV MEDHAGTYGL GDRKDQGGYT MHQDQEGDTD AGLKAEEAGI GDTPSLEDEA |
| | 61 AGHVTQARMV SKSKDGTGSD DKKAKGADGK TKIATPRGAA PPGQKGQANA TRIPAKTPPA |
| | 121 PKTPPSSGEP PKSGDRSGYS SPGSPGTPGS RSRTPSLPTP PTREPKKVAV VRTPPKSPSS |
| | 181 AKSRLQTAPV PMPDLKNVKS KIGSTENLKH QPGGGKVQIV YKPVDLSKVT SKCGSLGNIH |
| | 241 HKPGGGQVEV KSEKLDFKDR VQSKIGSLDN ITHVPGGGNK KIETHKLTFR ENAKAKTDHG |
| | 301 AEIVYKSPVV SGDTSPRHLS NVSSTGSIDM VDSPQLATLA DEVSASLAKQ GL |
| 30 | GCCTTTGCGAGCGTGCACAG |
| 31 | TTTCTTGTTGGGCAACTGGG |
| 32 | CTGGTGTGGGTGTTATTCCT |
| 33 | eGs[eCm]o[eCm]oeToeTsdTsdGs[dCm]sdGsdAsdGs[dCm]sdGsdTsdGs[eCm]oeAo[eCm]seAseG |
| 34 | eTseToeTo[eCm]oeTsdTsdGsdTsdTsdGsdGsdGs[dCm]sdAsdAs[eCm]oeToeGseGseG |
| 35 | [eCm]seToeGoeGoeTsdGsdTsdGsdGsdGsdTsdGsdTsdTsdAseToeTo[eCm]s[eCm]seT |
| 36 | GTGGATCTAGTTCTCATCCA |
| 37 | GGATAGAATCCAGTTAATTA |
| 38 | TGTCACATCTAGAAACCACC |
| 39 | GCCTGGACAGAGGGATGAGC |
| 40 | AAGCTCCAGGTGATTGAGAC |
| 41 | TTCTGATACATTGTCAAAGT |
| 42 | AGCAGCTTGAAGGAGCCGGC |
| 43 | TGGCTGATGATACCATTTGG |
| 44 | CTTTCTTGGAAAGTAT |
| 45 | TTTCTTGGAAAGTATA |
| 46 | TTCTTGGAAAGTATAT |
| 47 | TCTTGGAAAGTATATT |
| 48 | CTTGGAAAGTATATTC |
| 49 | GTGGATCTAGTTCTCA |
| 50 | TGGATCTAGTTCTCAT |
| 51 | GGATCTAGTTCTCATC |
| 52 | GATCTAGTTCTCATCC |
| 53 | ATCTAGTTCTCATCCA |
| 54 | TTCTGATACATTGTCA |
| 55 | TCTGATACATTGTCAA |
| 56 | CTGATACATTGTCAAA |
| 57 | TGATACATTGTCAAAG |
| 58 | GATACATTGTCAAAGT |
| 59 | AGCAGCTTGAAGGAGC |
| 60 | GCAGCTTGAAGGAGCC |
| 61 | CAGCTTGAAGGAGCCG |
| 62 | AGCTTGAAGGAGCCGG |
| 63 | GCTTGAAGGAGCCGGC |
| 64 | eGseToeGseGseAsdTsdCmsdTsdAsdGsdTsdTsdCmsdTsdCmseAseTo[eCm]s[eCm]seA |
| 65 | eGseGoeAseTseAsdGsdAsdAsdTsdCmsdCmsdAsdGsdTsdTseAseAoeTseTseA |
| 66 | eTseGoeTs[eCm]seAsdCmsdAsdTsdCmsdTsdAsdGsdAsdAsdAs[eCm]s[eCm]oeAs[eCm]s[eCm] |
| 67 | eGs[eCm]o[eCm]seTseGsdGsdAsdCmsdAsdGsdAsdGsdGsdGsdAseTseGoeAseGs[eCm] |
| 68 | eAseAoeGs[eCm]seTsdCmsdCmsdAsdGsdGsdTsdGsdAsdTsdTseGseAoeGseAs[eCm] |
| 69 | eTseTo[eCm]seTseGsdAsdTsdAsdCmsdAsdTsdTsdGsdTsdCmseAseAoeAseGseT |
| 70 | eAseGo[eCm]seAseGsdCmsdTsdTsdGsdAsdAsdGsdGsdAsdGs[eCm]s[eCm]oeGseGs[eCm] |
| 71 | eTseGoeGs[eCm]seTsdGsdAsdTsdGsdAsdTsdAsdCmsdCmsdAseTseToeTseGseG |
| 72 | [CmL]s[TL]s[TL]sdTsdCmsdTsdTsdGsdGsdAsdAsdAsdGs[TL]s[AL]s[TL] |
| 73 | [TL]s[TL]s[TL]sdCmsdTsdTsdGsdGsdAsdAsdAsdGsdTs[AL]s[TL]s[AL] |
| 74 | [TL]s[TL]s[CmL]sdTsdTsdGsdGsdAsdAsdAsdGsdTsdAs[TL]s[AL]s[TL] |
| 75 | [TL]s[CmL]s[TL]sdTsdGsdGsdAsdAsdAsdGsdTsdAsdTs[AL]s[TL]s[TL] |
| 76 | [CmL]s[TL]s[TL]sdGsdGsdAsdAsdAsdGsdTsdAsdTsdAs[TL]s[TL]s[CmL] |
| 77 | [GL]s[TL]s[GL]sdGsdAsdTsdCmsdTsdAsdGsdTsdTsdCms[TL]s[CmL]s[AL] |
| 78 | [TL]s[GL]s[GL]sdAsdTsdCmsdTsdAsdGsdTsdTsdCmsdTs[CmL]s[AL]s[TL] |
| 79 | [GL]s[GL]s[AL]sdTsdCmsdTsdAsdGsdTsdTsdCmsdTsdCms[AL]s[TL]s[CmL] |
| 80 | [GL]s[AL]s[TL]sdCmsdTsdAsdGsdTsdTsdCmsdTsdCmsdAs[TL]s[CmL]s[CmL] |
| 81 | [AL]s[TL]s[CmL]sdTsdAsdGsdTsdTsdCmsdTsdCmsdAsdTs[CmL]s[CmL]s[AL] |
| 82 | [TL]s[TL]s[CmL]sdTsdGsdAsdTsdAsdCmsdAsdTsdTsdGs[TL]s[CmL]s[AL] |
| 83 | [TL]s[CmL]s[TL]sdGsdAsdTsdAsdCmsdAsdTsdTsdGsdTs[CmL]s[AL]s[AL] |
| 84 | [CmL]s[TL]s[GL]sdAsdTsdAsdCmsdAsdTsdTsdGsdTsdCms[AL]s[AL]s[AL] |
| 85 | [TL]s[GL]s[AL]sdTsdAsdCmsdAsdTsdTsdGsdTsdCmsdAs[AL]s[AL]s[GL] |
| 86 | [GL]s[AL]s[TL]sdAsdCmsdAsdTsdTsdGsdTsdCmsdAsdAs[AL]s[GL]s[TL] |
| 87 | [AL]s[GL]s[CmL]sdAsdGsdCmsdTsdTsdGsdAsdAsdGsdGs[AL]s[GL]s[CmL] |
| 88 | [GL]s[CmL]s[AL]sdGsdCmsdTsdTsdGsdAsdAsdGsdGsdAs[GL]s[CmL]s[CmL] |
| 89 | [CmL]s[AL]s[GL]sdCmsdTsdTsdGsdAsdAsdGsdGsdAsdGs[CmL]s[CmL]s[GL] |
| 90 | [AL]s[GL]s[CmL]sdTsdTsdGsdAsdAsdGsdGsdAsdGsdCms[CmL]s[GL]s[GL] |
| 91 | [GL]s[CmL]s[TL]sdTsdGsdAsdAsdGsdGsdAsdGsdCmsdCms[GL]s[GL]s[CmL] |
| 92 | [eCm]seToeTseTs[eCm]sdTsdTsdGsdGsdAsdAsdAsdGsdTsdAseTseAoeTseTs[eCm] |
| 93 | eGs[eCm]oeTseGs[eCm]sdAsdGsdGsdGsdAsdAsdAsdGsdAsdGseTseTo[eCm]seTseT |
| 94 | eGseAoeTseGseGsdAsdGsdAs[dCm]sdAsdGsdTsdGsdTsdGseTseAoeTs[eCm]seT |

1. A MAPT antisense oligonucleotide comprises a nucleic acid sequence selected from any one of SEQ ID NOs: 1-11, 30-32, or 36-63, wherein optionally one or more nucleotides are independently modified nucleotides, and wherein optionally one or more internucleotide linkages are modified internucleotide linkages.

2. The MAPT antisense oligonucleotide of claim 1, wherein the antisense oligonucleotide is single stranded.

3. The MAPT antisense oligonucleotide of claim 1, wherein the antisense oligonucleotide comprises one or more modified nucleotides.

4. The MAPT antisense oligonucleotide of claim 1, wherein the one or more modified nucleotides comprise a modified nucleobase.

5. The MAPT antisense oligonucleotide of claim 4, wherein the modified nucleobase is a 5-methylcytosine.

6. The MAPT antisense oligonucleotide of claim 5, wherein each C in the antisense oligonucleotide is a 5-methylcytosine.

7. The MAPT antisense oligonucleotide of claim 1, wherein the one or more modified nucleotides comprise a modified sugar.

8. The MAPT antisense oligonucleotide of claim 7, wherein the modified sugar is a 2′-O-methoxyethyl (2′-O-MOE) modified sugar, 2′-O-methyl modified sugar, 2′-fluoro modified sugar, or a locked nucleic acid (LNA).

9. The MAPT antisense oligonucleotide of claim 8, wherein the modified sugar is a 2′-O-MOE modified sugar.

10. The MAPT antisense oligonucleotide of claim 8, wherein the modified sugar is LNA.

11. The MAPT antisense oligonucleotide of claim 1, wherein the antisense oligonucleotide is 16-20 nucleotides in length.

12. The MAPT antisense oligonucleotide of claim 1, wherein the first to fifth nucleotides each comprise a 2′-O-MOE modified sugar, wherein the sixth to fifteenth nucleotides each comprise a 2′-deoxynucleotide, and wherein the sixteenth to twentieth nucleotides each comprise a 2′-O-MOE modified sugar.

13. The MAPT antisense oligonucleotide of claim 1, wherein the first to third nucleotides each comprise LNA, wherein the fourth to thirteenth nucleotides each comprise a 2′-deoxynucleoside, and wherein the fourteenth to sixteenth nucleotides each comprise LNA.

14. The MAPT antisense oligonucleotide of claim 1, wherein the antisense oligonucleotide comprises one or more modified internucleotide linkages.

15. The MAPT antisense oligonucleotide of claim 14, wherein the one or more modified internucleotide linkages are phosphorothioate linkages.

16. The MAPT antisense oligonucleotide of claim 14, wherein the internucleotide linkages of the antisense oligonucleotide are sooosssssssssssooss, or sossssssssssssssoss, from 5′ end to 3′ end, wherein each s is a phosphorothioate linkage and each o is a phosphodiester linkage.

17. A MAPT antisense oligonucleotide comprises a sequence selected from any one of SEQ ID NOs: 12-22, 33-35, or 64-94.

18. The MAPT antisense oligonucleotide of claim 17, wherein the antisense oligonucleotide consists of a sequence selected from any one of SEQ ID NOs: 12-22, 33-35, or 64-94.

19. A pharmaceutical composition comprising the MAPT antisense oligonucleotide of claim 1 and a pharmaceutically acceptable carrier.

20. A pharmaceutical composition comprising the MAPT antisense oligonucleotide of claim 17 and a pharmaceutically acceptable carrier.

21. A method of reducing MAPT expression in a patient in need thereof, the method comprising administering to the patient an effective amount of the MAPT antisense oligonucleotide of claim 1.

22. A method of reducing MAPT expression in a patient in need thereof, the method comprising administering to the patient an effective amount of the MAPT antisense oligonucleotide of claim 17.

23. A method of treating a tauopathy in a patient in need thereof, the method comprising administering to the patient an effective amount of the MAPT antisense oligonucleotide of claim 1.

24. The method of claim 23, wherein the tauopathy is selected from Alzheimer's disease (AD), frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's disease, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

25. The method of claim 23, wherein the MAPT antisense oligonucleotide is administered to the patient intrathecally, intravenously, subcutaneously, or via intracisternal magna injection.

26. A method of treating a tauopathy in a patient in need thereof, the method comprising administering to the patient an effective amount of the MAPT antisense oligonucleotide of claim 17.

27. The method of claim 26, wherein the tauopathy is selected from Alzheimer's disease (AD), frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's disease, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

28. The method of claim 26, wherein the MAPT antisense oligonucleotide is administered to the patient intrathecally, intravenously, subcutaneously, or via intracisternal magna injection.

29. A method of reducing MAPT expression in a cell, the method comprising:
contacting the cell with the MAPT antisense oligonucleotide of claim 1; and
incubating the cell for a time sufficient for degradation of MAPT mRNA, thereby reducing MAPT expression in the cell.

30. A method of reducing MAPT expression in a cell, the method comprising:
contacting the cell with the MAPT antisense oligonucleotide of claim 17; and
incubating the cell for a time sufficient for degradation of MAPT mRNA, thereby reducing MAPT expression in the cell.
